(19) United States
(12) Patent Application Publication
(10) Pub. No.: US 2026/0233010 A1
(43) Pub. Date: Aug. 13, 2026

(54) AUTOMATIC DETECTION-THERAPY SYSTEMS FOR NEUROPSYCHIATRIC DISORDERS

(71) Applicant: David C. Shaw, Torrance, CA (US)

(72) Inventor: David C. Shaw, Torrance, CA (US)

(21) Appl. No.: 19/637,692

(22) Filed: Apr. 2, 2026

Related U.S. Application Data
(63) Continuation-in-part of application No. 19/191,555, filed on Apr. 28, 2025, now Pat. No. 12,642,486.
(60) Provisional application No. 63/732,819, filed on Sep. 25, 2024.
(60) Provisional application No. 63/732,962, filed on Oct. 12, 2024.
(60) Provisional application No. 63/833,151, filed on Oct. 26, 2024.
(60) Provisional application No. 64/028,515, filed on Jan. 29, 2026.

(51) Int. Cl. A61N 1/36; A61B 5/00; A61B 5/291; G16H 20/30; G16H 40/63; G16H 80/00

(52) U.S. Cl. CPC A61N 1/36139; A61B 5/291; A61B 5/4836; A61B 5/6817; A61N 1/36053; G16H 20/30; G16H 40/63; G16H 80/00

(57) ABSTRACT

An extended auricular electroencephalogram (EEG) monitoring system has a processing unit configured to analyze EEG data to detect neuropsychiatric disorders and impending neuropsychiatric disorders. An automatic detection-therapy system includes the auricular EEG monitoring system and one or more neuromodulation units. When presence of EEG signals suggestive of a neuropsychiatric disorder or an impending neuropsychiatric disorder is detected by the processing unit, the neuromodulation unit is configured to immediately start neuromodulating electric stimulation automatically to one or various combinations of the following: auricular branch of vagus nerve, supraorbital nerve, infraorbital nerve, auriculotemporal nerve, occipital nerve and greater auricular nerve to alleviate or abort the condition. A combined neuromodulation system having two or three neuromodulating components with synergistic effect is useful for health maintenance and prophylaxis of neuropsychiatric disorders. These earbud-shaped systems, with automated setup designs, are wearable, user-installable, user-removable and freely ambulatory.

### CROSS REFERENCE TO RELATED APPLICATIONS

This application is a continuation-in-part application of U.S. Non-Provisional application Ser. No. 19/191,555, filed on Apr. 28, 2025, entitled “Auricular Electroencephalogram (EEG) and Automatic Remedy Systems for Neuropsychiatric Disorders”, which claims priority to and the benefit of the filing date of: U.S. Provisional Application No. 63/732,819 filed on Sep. 25, 2024, entitled “Automatic auricular anti-seizure device”; U.S. Provisional Application No. 63/732,962, filed on Oct. 12, 2024, entitled “Automatic auricular detection-remedy system”; and U.S. Provisional Application No. 63/833,151, filed on Oct. 26, 2024, entitled “Automatic auricular detection-remedy system for neuropsychiatric disorders”, which are all hereby incorporated by reference in their entirety.

### FIELD OF THE INVENTION

This patent specification relates to the field of neuropsychiatric disorders, electroencephalogram (EEG), automatic EEG analysis and the field of wearable electroencephalogram systems. This invention also relates to the field of neuromodulation, including transcutaneous auricular vagus nerve stimulation (taVNS), trigeminal nerve stimulation, occipital nerve stimulation and greater auricular nerve stimulation which may be used in treatment of neuropsychiatric disorders.

### BACKGROUND

The human ear is very close to the brain and recent studies have shown that electrodes placed in the external ear canal can do electroencephalogram (EEG). Traditional EEG (full-scalp EEG) is done with multiple (mostly 21) electrodes attached to the scalp and connected with the EEG machine through multiple wires. It is very time-consuming to set up and to remove the multiple EEG electrodes. It is quite non-ambulatory (extremely cumbersome and difficult for wearer to move around). In traditional full-scalp EEG, the electrodes are attached or glued to the scalp by certified technologists. Traditional EEG is usually for short-term use only (half an hour to a couple days) because the electrodes often become detached after a short time. There exists a great need to have a miniature nice-looking device which can record EEG on a long-term (weeks or months or even years) basis. Previously EEG has been used primarily in the medical field or biomedical research field. Nowadays, in the artificial intelligence and robotics era, EEG or modified EEG is also heavily used in the brain-computer interface (BCI). Advancement in electronics technology, miniaturization trend, and dry electrode technology, together with sophisticated EEG interpretation algorithms, have enormously widened the potential usefulness of EEG in various medical fields.

In recent years, wireless in-ear EEG had been reported. For example, as published on Aug. 2, 2024, in Nature Communications, Ryan Kaveh et al reported using wireless miniature dry ear electrodes for in-ear EEG, with wireless electronics and offline classification algorithms, to monitor drowsiness of pilots and drivers. They described the design of earpieces for EEG, the neural recording hardware, the electrode materials and multi-sensor array. The recorded EEG data are digitized and transmitted to a processing unit for offline processing. They also describe manufacturing methods for in-ear EEG sensors. Two contralaterally worn earpieces can provide up to 11 channels with a common reference. Either right or left cymba concha electrode can be used as a reference. Prior to this report, earpieces with wet (hydrogel coated) electrodes were often used for EEG. These in-ear EEGs have been shown to successfully monitor drowsiness, seizure and sleep etc.

Various EEG analysis algorithms for decoding and processing of EEG data have been developed and described in many studies. With these EEG analysis algorithms and long-term EEG monitoring, they were able to accurately detect seizure (seizure also called epilepsy), migraine, cluster headache, neurodegenerative diseases, major depressive disorder, bipolar disorder, schizophrenia, obsessive-compulsive disorder, attention deficit hyperactivity disorder, autism spectrum disorder, post-traumatic stress disorder, anxiety disorder and panic disorder. They also found changes of EEG patterns suggestive of impending seizure, migraine, cluster headache, major depressive disorder, bipolar disorder, schizophrenia, obsessive-compulsive disorder, attention deficit hyperactivity disorder, autism spectrum disorder, post-traumatic stress disorder, anxiety disorder and panic disorder. Artificial intelligence, including machine learning (ML) and deep learning (DL) algorithms were applied to EEG data for processing.

In-ear EEG can overcome a lot of the limitations of traditional full-scalp EEG (limitations of full-scalp EEG includes: time-consuming to set up and to remove, bulky and non-ambulatory, short-time use, not suitable for long-term monitoring, requiring certified technologist to set up or remove, etc.). In-ear EEG (ear-EEG) has been developed in recent years with the help of advancement in electronic technology including the miniaturization trend and dry electrode technology. Like the traditional full-scalp EEG, the in-ear EEG can assess the electrical activities of the brain to monitor various neurological and psychological conditions. In-ear EEG has a very important advantage that it is much more suitable than traditional full-scalp EEG for long-term ambulatory monitoring. The newly developed sophisticated EEG interpretation algorithms have further widened the potential usefulness of the in-ear EEG.

As compared with traditional full-scalp EEG, in-ear EEG has been shown to provide very high performance for detecting seizures, particularly generalized seizures, temporal lobe seizures and focal-onset seizures, although it cannot fully match the sensitivity of full-scalp EEG in detecting frontal lobe seizures. In-ear EEG can detect ictal and interictal abnormalities for temporal lobe seizures with sensitivity matching that of full-scalp EEG. In-ear EEG is less effective in identifying frontal dominant interictal discharges. Overall, the sensitivity of in-ear EEG is around 86.4% to 94.5%, in detecting seizure. A National Institute of Health (NIH) study confirms that in-ear EEG offer a reliable alternative method to traditional scalp EEG. In-ear EEG is easier and much faster to setup and offer more comfort for patients than full-scalp EEG. The biggest advantage of in-ear EEG is that it is ambulatory and allow easy long-term monitoring. Long-term (or extended) EEG monitoring is highly effective for detecting and capturing seizures that routine full-scalp EEG might miss. By recording for 24 hours or more, it can identify abnormal EEG patterns and enable better seizure classification, localization and prediction of imminent seizure events minutes in advance. A study found that by analyzing prolonged EEG signals some models can predict up to 98% of seizures minutes in advance by identifying pre-ictal states (impending seizure). Long-term monitoring for 24-72 hours or longer will be very valuable for patients with suspected seizures or patients at high risk of status epilepticus. Long-term monitoring will also be very helpful to detect ictal and interictal epileptiform discharges during sleep.

Various EEG analysis algorithms for decoding and processing of EEG data have been developed and described in many studies. With these EEG analysis algorithms and long-term EEG monitoring, they were able to accurately detect many neuropsychiatric disorders. They also found changes of EEG patterns suggestive of impending neuropsychiatric disorders. Artificial intelligence, including machine learning (ML) and deep learning (DL) algorithms were applied to EEG data for processing. Several different methods with very high accuracy for seizure detection were described by Maham Saeidi, et al, in a published article in Brain Science on Nov. 18, 2021.

In 2021, Y. Ech-Choudany et al described a method for seizure detection from EEG using dissimilarity-based time-frequency distribution for seizure detection. They reported 98% accuracy. In 2020, M. Savadkoohi et al described another method with machine learning approach for seizure detection from EEG. They reported 100% accuracy. In 2024, Ayda Gokturk and Jacklyn Luu described utilizing machine learning on EEG data for seizure detection. They compared 5 machine learning models—Logistic Regression, K Nearest Neighbors (kNN), Random Forest, Neural Network, and Support Vector Machine (SVM). They found that the SVM model outperforms the other 4 models, achieving an accuracy of 96.77%, precision of 94.27%, and recall of 88.87%.

Seizure (epilepsy) affect about 3-4 million people in the USA. The mainstream treatment for epilepsy is with anti-epileptic medications. However, there are substantial percentage (some estimate: up to 30%) of epilepsy patients who do not respond adequately to medications (They are called refractory or drug-resistant epilepsy patients). Some of these refractory epilepsy patients require surgery; others might consider adjunct seizure therapies, such as neuromodulation with vagus nerve stimulation, trigeminal nerve stimulation (including supraorbital nerve stimulation, infraorbital nerve stimulation, and auriculotemporal nerve stimulation), occipital nerve stimulation and greater auricular nerve stimulation. Other options might include sounds-music therapy and electromagnetic modulation.

Migraine affects millions of people in the USA. There are many medicines which can help migraine. The triptans are one of the mainstream medicines being used for migraine. Triptans can be taken orally, intranasally or by subcutaneous injection. There are also several prophylactic medicines available. Even with all of these, migraine headaches still produce major impact on the patients' lives and huge economic loss. Migraine can occur suddenly. A reliable method for pre-migraine management is in great need. Previously, migraine attacks were considered unpredictable making preemptive interventions not feasible. However, it was found that there are neurophysiological changes 24-48 hours before migraine attacks. These neurophysiological changes can be detected using long-term EEG monitoring. In 2020, Isabel Martin et al reported using EEG to predict future migraine attacks. They found that 24 hours before migraine onset, there was a statistically significant modulation of the EEG, with decrease of relative power in the delta waves and increase of beta wave frequency bands, at rest. There was also a notable reduction of the amplitude and coherence measures of an attention event-related brain potential (P300). There were other studies regarding migraine and cluster headache with EEG changes. In 2016 Cao Z. et al reported that resting state EEG power and coherence vary between migraine phases. In 2023, Ning Zhang et al reported a review of using modern EEG data processing and analysis for EEG-based migraine analysis.

In 2024, Thomas Van den Hoek reported another review article regarding EEG and migraine. Traditional EEG did not help much in the management or prediction of migraine. One of the most important reasons is due to the fact that traditional EEG was mostly done only short-term on cross-sectional basis. Recently, it was found that a more long-term longitudinal EEG can make subtle changes in EEG much more detectable. Advancement in data processing and analysis also help to find EEG changes during migraine. Furthermore, it was also found that EEG changes can be detected even before onset of acute migraine attack. Longitudinal studies have been able to identify some pre-ictal changes in spontaneous EEG features compared to the inter-ictal phase, and also between migraine patients and controls. These differences include EEG slowing, alpha and theta band asymmetry, enhanced EEG spectral power, and coherence. The observation that EEG power and coherence were reduced during the acute migraine as compared to the before migraine period could be used to predict migraine before an attack. Raghuraman L. and Joshi S. reported in 2024 another review article regarding EEG and migraine. They reported EEG changes in migraine, including cortical hyperexcitability and habituation deficit to sensory stimuli and alpha oscillations. Spectral analysis of EEG waves often showed more reliable and consistent results than features read off the EEG directly. EEG microstate was found to be the most promising method showing characteristic identifiable features for diagnosis of migraine. It would be very valuable if patients can be notified of impending migraine and can start taking prophylactic measures preemptively.

Tension headache generally does not show specific diagnostic changes detectable on regular EEG or extended EEG. Using extended EEG to detect migraine will greatly help patients to differentiate between migraine and tension headache.

Even though the traditional full-scalp EEG can detect migraine and predict impending migraine, very few migraine patients benefited from it because the traditional EEG is not suitable for long-term ambulatory use. With the help of various EEG analysis algorithms, together with machine learning, deep learning and artificial intelligence, novel long-term EEG monitoring with in-ear EEG as disclosed in this invention can be used to detect migraine and impending migraine. After detecting migraine or impending migraine, it would be very valuable to have a novel wearable convenient device that can provide therapeutic actions automatically and instantly in response to the EEG detection of migraine or impending migraine.

Many neuropsychiatric disorders or conditions can be detected with EEG, including migraine, cluster headache, seizure, major depressive disorder, bipolar disease, autism, schizophrenia, tinnitus, dyslexia, stroke, etc. In 2023 Hao Zhang et al published a review article regarding principles of EEG analysis methods in neuroscience and clinical neurology. As reported by Maham Saeidi et al and Hao Zhang et al, these researches in EEG data analysis have leaded to detection of stroke, autism, dyslexia and tasks including emotion recognition, mental workload, motor imagery, neurodegenerative diseases, sleep stages scoring and seizure detection, etc. In 2024, Thomas Van den Hoek reported another review article regarding EEG and migraine.

Cluster headache is another type of headache which affects millions of people in USA. It is also called trigeminal autonomic cephalgia. Cluster headache is usually shorter (15 to 180 minutes) than migraine, but is much more intense and can occur more often (up to 8 times a day). The pain with cluster headache is so severe that patients are often incapacitated during the attack. It can keep recurring for weeks or even months. Cluster headache is extremely painful and is characterized by severe, unilateral head pain, often located behind one eye. Cluster headache often occurs in clusters daily for weeks or months and then followed by remission (pain-free) periods (possibly for few months). Each cluster headache attack can occur suddenly lasting 15 minutes to 3 hours. It could occur in the night and waking the patients up at night. Cluster headache is typically one-sided and on the same side patients often have a drooping eyelid (ptosis), tearing (watery eye), nasal congestion or runny nose, facial sweating, and a flushed face.

Cluster headache is quite different from migraine. Migraine sufferers usually prefer to lie down in dark rooms, whereas cluster headache suffers are often agitated, restless and pacing around. The exact cause of cluster headache is unknown. Cluster headaches are believed to be linked to hypothalamic dysfunction and the trigeminal nerve. They are not generally hereditary. Because of the extreme severity and the very rapid onset, standard pain-relieving medicines are rarely effective for cluster headache. High-flow oxygen through a facial mask is an effective treatment. Sumatriptan injection is another effective treatment. Greater occipital nerve block has also been found to be helpful. However, oxygen tank and injectable sumatriptan are often not immediately available for the suffers. Preventive medication, such as verapamil, may be useful to prevent or lessen future attacks. It is possible to have both cluster headaches and migraines, a condition where both types of severe headaches co-exist, with studies suggesting around 10 to 17 percent of people with cluster headaches also experience migraines. While they have distinct characteristics—cluster headaches are typically short, very agonizing, and cause restlessness, whereas migraines are often longer, throbbing, and cause a need for stillness—a patient can suffer from both independently.

Previously, it was believed that there were not much significant EEG changes before or during cluster headache. Recent studies have reported EEG changes associated with cluster headaches. These changes often occur before or during headache episodes, indicating potential alterations in brain activity related to the condition. EEGs taken between attacks may show abnormalities, including focal slowing or spikes, which could indicate underlying pathophysiological processes. Some studies have noted increased theta wave activity in the frontal regions of the brain during cluster headache attacks, suggesting heightened cortical excitability.

More recent studies with extended EEG have found EEG changes before and during cluster headache, even though there were less reports in this regard as compared with migraine. Some studies suggest a slight increase of non-specific abnormalities or slow waves during pre-cluster headache periods. Some reports have observed focal EEG abnormalities like transient theta-delta slowing on the ipsilateral side during cluster headache. Unlike the clear cortical spreading depression (CSD) and related EEG changes in migraine, typical cluster headache attacks do not exhibit these specific cortical changes, though some overlap with migraine symptoms (like aura) has been reported. In 2022, Padmarathy N. et al reported using EEG to identify cluster headache and migraine.

Cluster headache is a primary neurovascular headache, typically accompanied by significant neurovascular changes, including dilation of blood vessels in the brain and around the eye. These neurovascular changes are driven by the activation of the trigeminal-autonomic reflex, which connects the trigeminal nerve to facial parasympathetic nerves. It causes vasodilatation leading to autonomic symptoms on the affected side, including red, watery eye (conjunctival injection and lacrimation), swollen eyelid, droopy eyelid (ptosis) and nasal congestion. The increased blood flow may also result in temperature changes.

It has been found that many psychiatric disorders or conditions can be detected with EEG, including major depressive disorder, bipolar disorder, schizophrenia, obsessive-traumatic stress disorder, anxiety disorder and panic disorder. In 2023 Hao Zhang et al published a review article titled “The applied principles of EEG analysis methods in neuroscience and clinical neurology” regarding principles of EEG analysis methods. As reported by Maham Saeidi et al and Hao Zhang et al, these researches in EEG data analysis have leaded to detection of stroke, autism, dyslexia and tasks including emotion recognition, mental workload, motor imagery, neurodegenerative diseases, sleep stages scoring and seizure detection, etc.

The advancement in EEG technology and EEG analysis with machine learning have helped EEG in detection of major depressive disorder. As reported by C. Wu et al in 2021, four common EEG features were studied, including: band power (BP), coherence, Higuchi's fractal dimension, and Katz's fractal dimension. They found that coherence-based connectivity is a reliable feature to detect major depressive disorder with high accuracy. Sara Yasin et al reported in 2021, using EEG for detection of major depressive disorder and bipolar disorder. They reviewed recent researches using artificial neural networks (shallow and deep learning-based) approaches and was able to successfully detect major depressive disorder and bipolar disorder using EEG.

With extended EEG, including in-ear EEG monitoring, major depressive disorder can be detected by its brainwave changes, including altered spectral power with decreased alpha with alpha power asymmetry, increased theta/delta activities and frontal alpha asymmetry (lower-than-normal alpha activities in left frontal lobe as compared with right frontal lobe). When combined with machine learning and deep learning, high classification accuracy (up to 91.3% in some studies) as compared with healthy control can be achieved.

Extended EEG monitoring has been found to help detect bipolar disorder and distinguish it from other psychiatric conditions including schizophrenia, major depressive disorder and others. With the help of machine learning, specific EEG abnormalities, including increased delta/decrease alpha activities, spectral and connectivity abnormalities can help in detection of bipolar disorder. EEG monitoring can also identify cognitive processing disruptions which may indicate impending bipolar disorder even during remission. In-ear EEG was found to be comparable to full-scalp EEG for detection of bipolar disorder and impending bipolar disorder.

Extended EEG monitoring has been found to have strong potential for detecting schizophrenia and detecting individuals in a high-risk mental state of impending schizophrenia. EEG biomarkers detectable by in-ear EEG include mismatch negativity, and P3a which are associated with auditory processing problems and cognitive dysfunction seen in schizophrenia. Deep learning models, such as Convolutional Neural Networks have achieve high accuracy (over 90%) in distinguish schizophrenia from healthy people. Studies further found that extended in-ear EEG can detect changes in brain network functions for prediction of impending (at-risk) mental state before transiting into schizophrenic psychosis.

Studies have also found that extended EEG monitoring with in-ear EEG can help to detect obsessive-compulsive disorder (OCD) by identifying specific EEG patterns, including abnormal delta/alpha band activity, connectivity problems, increased, abnormal or nonlinear EEG patterns. Extended EEG can also offer high temporal resolution to capture quick changes in EEG as related to obsession or compulsions. Researches also found that in-ear EEG can help to predict impending OCD.

Another psychiatric disorder that can be detected by extended EEG is autism spectrum disorder (ASD). Studies have shown that extended EEG (for example in-ear EEG) can identify early neural biomarkers such as delta/gamma power changes, neural connectivity disruptions (local over-connectivity and long-distance under-connectivity) in infants for early detection and detection of children in their first year of life who are at risk for future development of ASD.

Post traumatic stress disorder (PTSD) is another example that extended EEG (for example in-ear EEG) can detect by identifying specific EEG markers, such as alpha power asymmetries and theta band connectivity, and decreased low frequency waves in right temporo-parietal regions of the brain. Increased functional connectivity in the theta band is found to be related to greater severity of PTSD symptoms. The extended EEG monitoring can identify, in real-time brain waves that are related to hyperarousal and attention deficits, aiding in detection of PTSD and identification of impending PTSD.

Similarly, anxiety disorder and panic disorder may also be detected by extended EEG (e.g. in-ear EEG) by identifying specific neural markers, including increased high beta waves and decreased theta waves. The EEG monitoring can detect in real-time the brain's transition from calm to high-arousal states and provides potential for early detection. During panic attacks, EEG shows increased frontal beta activities and decreased theta activities. Frontal paroxysmal changes (rapid bursts of activities) often occur just before panic attack, offering early detection of impending panic attacks. Utilization of modern EEG interpretation algorithms, including machine learning and deep learning, can help detection of all of the above psychiatric disorders and impending psychiatric disorders.

The mainstream treatment for epilepsy (seizure) is with anti-epileptic medications. However, there are substantial percentage (some estimate: up to 30%) of epilepsy patients who do not respond adequately to medications (refractory epilepsy). Some of these refractory epilepsy patients require surgery; others might consider adjunct seizure therapies. The adjunct seizure therapy options include vagus nerve stimulation, trigeminal nerve stimulation, occipital nerve stimulation, greater auricular nerve stimulation, sounds or music therapy, electromagnetic modulation and others. The anti-epileptic medications can be given on a fixed schedule. Additional medications or extra dose of medications can also be given on an as needed basis when patients have epilepsy or break-through epilepsy. Self-administration of as needed medications to be given orally immediately at onset of acute seizures is often impractical because the patients might not be able to recognize having seizure, or seizure occurring during sleep, or unable to swallow medicines, or unable to reach medicines. As needed medications can be given by injections in ambulances, emergency rooms, hospitals or clinics. However, there would be substantial delay before the seizure is recognized and before ambulance arrives. During acute seizures, treatment should be started as soon as possible because prolonged seizures could be very detrimental to human health and could be even life-threatening. There exists a great need to have seizures detected immediately and automatic treatment or therapy started instantly at onset of seizures. It is also very important if prophylactic therapy can be started during period of impending epilepsy.

The human ear is the only location where the vagus nerve reaches the body surface (the skin), through auricular branch of vagus nerve. The cymba concha has almost exclusive vagal nerve innervation. Cavum concha (the lower, larger bowl-shaped part) is located around the opening of the external ear canal. Cavum concha also receives significant innervation from the auricular branch of vagus nerve. While cavum concha is largely innervated by the vagus, the anterior cavum concha receives mixed innervation from the auriculotemporal nerve (a branch of the mandibular division of the trigeminal nerve). The facial, glossopharyngeal, and cervical nerves (e.g. greater auricular nerve) also innervate part of the cavum concha. The inner and posterior tragus is innervated by vagus nerve, while the anterior outer part of tragus is innervated by trigeminal nerve (auriculotemporal nerve) and some from greater auricular nerve (which is from cervical nerve). The posterior and inferior walls of the external ear canal receive innervation from the vagus nerve, while the anterior and superior walls of the external ear canal is innervated by the auriculotemporal nerve. The greater auricular nerve (C2-C3) innervates the lower concha (inferior concha) and parts of the cavum concha via its posterior (mastoid) branch. These unique proximity or mixed innervation patterns make the ear an ideal location to give stimulation to the auricular branch of vagus nerve, the auriculotemporal nerve and the greater auricular nerve.

The vagus nerve innervated auricular skin includes inner tragus, cymba-concha, cavum-concha, posterior inferior walls of the external ear canal and small adjacent regions of the external ear. Thus, transcutaneous auricular vagus nerve stimulation (taVNS) can be placed on the vagus innervated auricular skin to stimulate the vagus nerve. The vagus nerve has huge influence on various human body functions, including brain, heart, breathing, emotions, blood pressure, GI system and metabolism etc. The cavum concha is heavily innervated by the auricular branch of vagus nerve although the auriculotemporal nerve (a branch of mandibular division of trigeminal nerve) also provides overlapping innervation for anterior superior part of cavum concha. The inner (medial) posterior tragus is primarily innervated by the vagal nerve although the anterior outer part of tragus is innervated by the auriculotemporal nerve.

The human ear is a convenient location to place a stimulating electrode for auriculotemporal nerve. The auriculotemporal nerve mainly supplies the anterior-superior part of the pinna (including anterior-superior helix), anterior outer tragus, part of the inner tragus (inner/medial surface of tragus, facing the external ear canal), anterior-superior region of cavum concha and anterior-superior walls of external ear canal. The auriculotemporal nerve innervated auricular skin includes anterior outer tragus, part of the inner tragus (inner/medial surface of tragus), anterior and superior part of pinna (including anterior-superior helix), anterior and superior walls of the external ear canal and the anterior-superior region of cavum concha.

The US Food and Drug Administration (FDA) approved vagus nerve stimulation only for left vagus nerve when stimulated at neck region, due to concern of possible bradycardia when right vagus nerve is stimulated. The practice of using transcutaneous auricular vagus nerve stimulation to treat seizures was reviewed by Yu Wang et al in 2020. They reviewed other studies and found that bilateral auricular vagus nerve stimulation is safe with no increase of side effects as compared with left-sided stimulation. They also stated that bilateral stimulation is more effective. After reviewing many studies, they found that the stimulation parameters for seizures have a wide range of variation. The most common stimulation frequency is 20 Hz or 25 Hz (range 0.5-120 Hz). The common stimulation pulse width is 1 ms or 0.25 ms (range 0.02-1 ms). They did find that pulse width of 500 microsecond is the most biological active. They reported that the stimulation intensity is often adjusted by the patients according to their tolerance.

Vagus nerve stimulation (VNS) therapy has been approved by US Food and Drug Administration (FDA) as an adjunct treatment for drug-resistant epilepsy. The placement of a traditional VNS device requires a surgery. The VNS device is usually placed under the skin in the chest with a wire going to the neck to wrap around or attach to the left vagus nerve in the neck. The VNS can give electrical stimuli to the vagus nerve. Various settings of stimulating patterns, strength, duration, frequency and intervals have been studied. For example, a setting with stimuli given in cycles of 30 seconds on and 5 minutes off is commonly used. The stimulation could be given 3-4 times per day and each time 1-4 hours. The stimulation strength varies from low (around 0.25 mA) to high (around 1.75 mA). In some studies, patients were allowed to adjust the stimulation strength to maximum tolerable strength. The VNS has been shown to help prevent impending seizures before they start and help to stop or shorten them if they do. It also helps to decrease symptoms in the post-ictal phase. The VNS decreases seizures by sending regular mild pulse of electrical stimuli to the vagus nerve. If a person is aware of a seizure happening, the person can manually swipe a magnet on the VNS to send extra burst of electric stimuli and this often helps to stop the seizure or decrease the severity of seizure. Similarly, various settings of stimulating patterns, strength, duration, frequency and intervals of the extra burst of electric stimuli have been studied to find the most effective stimulating setting. However, there is a problem that the person might not be aware of occurrence of a breakthrough seizure or an impending seizure, or might be unable to respond to it or too late to respond to it, or seizure occurring during sleep.

Similar to VNS, many studies had shown effectiveness of using transcutaneous auricular vagus nerve stimulation (taVNS) for seizures. In 2000, Ventureya proposed using taVNS for seizures therapy. Since then, more studies have shown that ta VNS can help to decrease seizure frequency, duration and severity, similar to that with VNS. In 2021 Breanne Fisher et al published another review article showing effectiveness of using vagus nerve stimulation to treat drug-resistant epilepsy. The practice of using transcutaneous auricular vagus nerve stimulation for seizures was also reviewed by Marios Lampros et al in 2021. They reported mean seizure frequency reduction varied from 30-65% with only mild adverse effects. Transcutaneous auricular vagus nerve stimulation (taVNS) has shown effectiveness as an adjunctive therapy for impending seizure for patients with refractory (drug-resistant) seizures. A model of implanted VNS allows patients to manually swipe a magnet to activate the VNS stimulation when the patient feel seizure coming or when a sudden increase of heart rate is detected (imminent seizure could be accompanied by sudden heart rate increase). This manual-emergency use has been shown to stop, shorten, or lessen the severity of the seizure. Although this model is for implanted VNS, it is believed that taVNS will be similarly effective in helping impending seizure. By regular, automated stimulation, both implanted VNS and ta VNS have been found to be effective in seizure prevention by reducing overall seizure frequency and intensity. Beyond reducing seizures, they can improve quality of life and reduce anxiety and depression for patients with refractory seizures.

Trigeminal nerve stimulation (TNS) has been found to help reduce seizure frequency in patients with drug-resistant seizure. Trigeminal nerve stimulation may be delivered via supraorbital nerve or auriculotemporal nerve or infraorbital nerve. Up to 90% reduction in seizure was found in some patients, and about 40% of patients experiencing significant improvement. Occipital nerve stimulation (ONS) was also found to be helpful in reducing seizures. The trigeminal nerve stimulation and taVNS were both found to be effective in intercepting impending seizure, especially during aura. Preliminary results from studies suggest that combining trigeminal verve stimulation and occipital nerve stimulation may provide synergistic effects, showing superior therapeutic results compared to single-nerve stimulation alone.

In 2018, C. Wu et al reported that transcutaneous auricular vagus nerve stimulation (taVNS) was effective for treatment of major depressive disorder (MDD). In 2020, Yonathan Yap reported that taVNS can provide therapeutic effect similar to that of VNS. The taVNS treatment is effective for bipolar disorder, schizophrenia, major depressive disorder and other neuropsychiatric disorders. In this review article, they also discussed the stimulation parameters, stimulation sites, and available devices.

In 2023 Ashraf Gerges reported an article reviewing the application of taVNS to various types of neuropsychiatric diseases. They also reported the various electric stimulation parameters. The stimulation parameters include pulse frequency, pulse width, pulse-pause ratio (on/off timing), electrode type, device used, electric current type, electrode location etc. These data were reported in their figures and tables. For example, in 68% of the studies, the taVNS stimulation intensity was set at a level above the individual's sensory threshold and below the pain threshold. The Intensity values ranged from 0.5 to 50 mA. The electrode size ranged from 2-200 mm. Pulse frequency of either 20 Hz or 25 Hz was used in 74% of the studies. The most common pulse width ranged from 0.05 to 1.0 ms, with either 0.20, or 0.25 ms, being the most commonly used. In their review, 62% of the studies stimulated only the left auricular branch of vagus nerve, while bilateral vagus nerve stimulation was done in 27% of the studies.

TaVNS has been found to be effective in treating various neuropsychiatric disorders, including major depressive disorder (MDD). There is strong evidence suggesting that taVNS can significantly reduce depressive symptoms by modulating the default mode network, cognitive control network and salience network with efficacy (HAMD scores) comparable to that of antidepressant medications and fewer side effects. Studies have shown that taVNS is effective for moderate MDD, relieving symptoms like anxiety, insomnia, and hopelessness. For impending MDD, taVNS can improve mood, potentially helping before a severe episode. Studies also shown that trigeminal nerve stimulation (TNS) (including supraorbital nerve stimulation and auriculotemporal nerve stimulation) can help MDD by modulating brain areas like the amygdala and insular cortex. Combining taVNS with other methods such as TNS or occipital nerve stimulation (ONS), is found to be safe and well-tolerated, and can lead to a greater reduction in depression symptoms than each therapy alone due to the synergistic effect. It was also found that combining TNS and ONS also seems to help depression symptom of MDD.

The FDA has approved implanted cervical vagus nerve stimulation (VNS) for treatment-resistant bipolar depression. The cervical VNS reduces depressive symptoms, suicidal ideation, and has a rapid, durable antidepressant effect, with sustained improvement without triggering mania and impending bipolar disorder. There are evidences suggestive that taVNS is potentially effective for reducing symptoms of bipolar disorder and impending bipolar disorder by modulating brain networks like the default mode network (DMN), and it may help with anxiety, sleep disturbance, and hopelessness associated with bipolar depression. TNS, taVNS, and ONS all have effect for bipolar disorder, particularly the depressive phase of bipolar disorder, by modulating mood-regulating brain circuits. Studies also indicate that combining TNS and ONS has synergistic effect and provides superior, rapid results compared to stimulating either nerve alone. Though there were limited studies so far, it is believed that ta VNS and TNS are effective in treating impending bipolar disorder.

Regarding schizophrenia, a 2025 study found that taVNS is very effective and can significantly improve negative symptoms of apathy and social withdrawal in treatment-resistant schizophrenia, with effects linked to reductions in inflammation markers and its effect on the prefrontal cortex. Trigeminal nerve stimulation (TNS) also shows potential for treating negative symptoms of schizophrenia. Occipital nerve stimulation (ONS) combined with other treatments can help with cognitive performance in schizophrenia. Study data are encouraging, although less specific, about their effects on impending schizophrenia or prodromal phase of schizophrenia. Emerging research indicates that combining transcutaneous vagus nerve stimulation (taVNS) with trigeminal nerve stimulation (TNS) or occipital nerve stimulation (ONS), can lead to improved results compared to single-nerve stimulation.

For neurodevelopmental disorders, including attention deficit hyperactivity disorder (ADHD) and obsessive-compulsive disorder (OCD), there are evidence that taVNS can help clinical outcomes of these disorders. Research shown that taVNS can enhance the efficacy of Exposure and Response Prevention (ERP) therapy for OCD, specifically targeting fear extinction and anxiety regulation. TaVNS can help modulating brain function and reducing inflammation by improving neuroplasticity and stress regulation and significantly improve symptoms of OCD. TNS has been found to have efficacy in reducing OCD symptoms. In case studies, it has shown potential in reducing obsessive-compulsive symptoms. TNS can improve cognitive measures and manage comorbid anxiety. TNS is known as a potential intervention for stress management and cognitive enhancement, which can help in managing OCD-related anxiety. ONS, especially when combined with TNS, also shows potential to help OCD. Trigeminal nerve stimulation (TNS), occipital nerve stimulation (ONS) and taVNS are all believed to possibly help impending OCD, although more research is needed.

As for autism spectrum disorder (ASD), emerging research findings suggest that taVNS can help ASD symptoms, by helping to improve cognitive function, behavior mood, reduce anxiety and increase social interaction and sensory processing and help to reduce future development of ASD symptoms for at-risk children, potentially acting through immune-modulating pathways. Clinical trials suggest taVNS can improve social-emotional understanding, attention, and reduce anxiety and sleepiness in individuals with ASD. Preliminary studies showed TNS have possible effect for ASD. There were studies showing promising result of TNS for ASD patients having ADHD symptoms such as inattention and hyperactivity. Evidence for ONS specifically for ASD is limited, though the neuromodulation from ONS might generally improve brain network connectivity. While taVNS and TNS might have synergistic effect since both are known to modulate brainstem regions including the locus coeruleus (located at the pons and is the primary source of norepinephrine) to increase brain arousal and function, there are limited direct research specifically evaluating their combined effect in human ASD.

For post-traumatic stress disorder (PTSD), studies have shown promising results that taVNS can activate specific neurons in the anterior cingulate cortex and helps to reduce or alleviate PTSD symptoms such as hyperarousal and somatic anxiety by reducing inflammation (reduce IL-6) and increase parasympathetic activity. TNS helps reset neural networks associated with mood and anxiety, showing promise for treating PTSD, particularly for reducing anger and improving sleep. Simultaneous use of both taVNS and trigeminal nerve stimulation (TNS) have shown potential for synergistic effects for both of them and a 2025 study suggested long-term benefits in PTSD. Occipital nerve stimulation (ONS) alone or combined with TNS might have potential in modulating pain and stress-related pathways for patients with PTSD although studies are limited. These interventions show potential as preventative, or early intervention, for impending PTSD to manage acute stress and prevent the development of full-blown PTSD by controlling the initial inflammatory and autonomic response to trauma.

For anxiety disorder and panic disorder, taVNS has been found to be quite promising in alleviating symptoms of anxiety disorder and panic disorder by regulating the default mode network, cognitive control network, and salience network and modulating brain areas involved in stress and fear. Likewise, TNS also helps to decrease symptoms of anxiety disorder and panic disorder. Combination of taVNS and TNS has synergistic effect in helping symptoms of anxiety disorder and panic disorder. ONS is mostly known for its effect for headache and there is limited study about ONS effect on anxiety disorder and panic disorder. However, it has been known that combination or simultaneous use of ONS with TNS often has synergistic effect in therapeutic result. These therapy methods (taVNS, TNS and ONS) work by modulating the central nervous system to reduce panic symptoms, with studies showing effect for impending anxiety and panic with >50% reduction in attack frequency for certain neurostimulation techniques.

The Food and Drug Administration has approved trigeminal nerve stimulation (TNS) for pediatric ADHD. TNS (including supraorbital nerve stimulation and auriculotemporal nerve stimulation) can target the brain region associated with attention. TNS is an FDA-cleared treatment for ADHD in children ages 7-12. Earlier studies had shown improvement of symptoms by stimulating brain regions related to attention for attention deficit hyperactivity disorder. However more recent study shown some doubt about that. It is hypothesized that taVNS might improve attention and behavioral regulation, but research is still developing. Studies about ONS for ADHD is limited.

For depression, Ashraf Gerges reported significantly favorable result with bilateral or unilateral vagus nerve stimulation with intensity at non-painful level (above sensory threshold). The other parameters include: intensity ranging from 0.5-6 mA, pulse frequency ranging from 20-25 Hz and pulse width ranging from 0.2-1 ms. Treatment timing ranged from 60-240 min/day, delivered 5-7 days/week for 4-12 weeks. They also disclosed the stimulation parameters for other neuropsychiatric conditions.

Trigeminal nerve stimulation (TNS) has been studied and found to be an effective neuromodulation method that helps reduce seizure frequency, particularly in drug-resistant epilepsy. TNS can directly inhibit neurons and act on brain regions involved in seizure generation. Studies have shown that TNS can reduce seizures, with one report of 47.9% reduction in seizure frequency in patients with refractory seizures. TNS acts via trigeminal-parasympathetic reflex that can inhibit neuron firing. Another report showed 56% to 66% reduction in seizures over 3 to 6 months. The trigeminal nerve has several branches. The most easily targeted and commonly used trigeminal nerve branches for neurostimulation include the supraorbital nerve (V1 or ophthalmic division) and infraorbital nerve (V2 or maxillary division) because they are superficial, making them easily accessible for electrode placement. The other good choice is the auriculotemporal nerve (V3 or mandibular division). The auriculotemporal nerve has a superficial temporal branch which provides sensory innervation to the skin of temple and an auricular branch which provides innervation to anterior-superior pinna (including tragus and anterior-superior helix), anterior part of cavum concha, the anterior and superior walls of external auditory canal and outer surface of the tympanic membrane. The auriculotemporal nerve (ATN) has unique characteristics that its innervated auricular skin is in close proximity and overlapping with the vagus-innervated auricular skin. (The vagus-innervated auricular skin includes inner posterior portion of tragus, cymba-concha, cavum-concha, posterior inferior walls of the external ear canal and small adjacent regions of the external ear.) (The auriculotemporal nerve innervated auricular skin includes anterior outer tragus, anterior and superior part of pinna including anterior-superior helix, anterior and superior walls of the external ear canal and the anterior-superior part of cavum concha.)

Both trigeminal nerve stimulation (TNS) and transcutaneous auricular vagus nerve stimulation (taVNS) can help regulate the autonomic nervous system, improve blood flow to the brain and increase stability of large-scale functional brain networks. While direct, large-scale clinical trials combining TNS and taVNS for seizure control are quite limited, conceptually, these two methods could be synergistic since they target different, yet overlapping, afferent brain pathways (taVNS targets the solitary tract located in the brainstem, while TNS targets the trigeminal system which has extensive brainstem connections). They can also be combined with pharmacological treatments.

Supraorbital nerve stimulation is a type of trigeminal nerves stimulation. The supraorbital nerve stimulation has been shown to help refractory seizures. Examples of transcutaneous supraorbital nerve stimulators include a Cefaly device which is FDA-approved for migraine. The Cafaly device is not yet FDA-cleared for seizure, although studies have shown that it is quite promising. The Cefaly device is a wearable, non-invasive therapy for seizure by stimulating the supraorbital nerve. The supraorbital nerve is a branch of the trigeminal nerve. The Cefaly device is mounted on the forehead and uses pre-determined electrical impulses on the forehead for neuromodulation. Studies have shown that supraorbital nerve stimulation can decrease the frequency of seizures by 36-57% of refractory seizures.

Auriculotemporal nerve (ATN) stimulation is another type of trigeminal nerve stimulation. An example of a ATN stimulation unit is a device called the Roo™ Therapy System by Spark Biomedical which is FDA-designated. The auriculotemporal nerve (ATN) stimulation unit is sometimes combined with auricular vagus nerve stimulation unit (taVNS) due to anatomical proximity or overlapping of the auriculotemporal nerve and auricular branch of vagal nerve in the ear around tragus, cavum concha and external ear canal. The auriculotemporal nerve stimulation unit and taVNS unit may share a same housing with a same stimulating electrode. However, they often use their own optimized stimulating electrodes with their own distinct stimulating parameters. The primary targets for taVNS stimulating electrode are cymba concha (100% vagal), cavum concha, inner-posterior portion of tragus (mixed vagal/trigeminal) and posterior-inferior walls of external ear canal for autonomous nerve modulation. The primary targets for auriculotemporal nerve include anterior outer part of tragus, anterior-superior helix, anterior portion of cavum concha and anterior-superior walls of external ear canal. If the anterior-superior helix is selected as the target for the auriculotemporal nerve, the stimulating electrode may be attached via a clip electrode. The auriculotemporal nerve stimulation unit uses a setup similar to a transcutaneous electrical nerve stimulator (TENS). Both ATN stimulation and taVNS stimulation can be applied concurrently to the ear with about 30 minutes stimulation session. There are various stimulating parameters. For example, a common stimulating parameter for taVNS is 20-25 Hz frequency, 200-500 microsecond pulse width with intensity adjusted to a comfortable sensory level, usually below 5 mA. The stimulating parameters for ATN (auriculotemporal nerve stimulator) are often different, with higher frequency (around 100 Hz) to modulate different analgesic receptors. The stimulating strength for both of them can be titrated to the user's individual maximal tolerable level without pain. Combining these techniques can have synergistic effect to produce a more potent inhibitory effect on seizure.

Furthermore, the auriculotemporal nerve (ATN) stimulation unit may be combined with auricular vagus nerve stimulation unit (taVNS) and greater auricular nerve (GAN) stimulation unit due to anatomical proximity or overlapping of the ATN, the auricular branch of vagal nerve and the GAN in the ear around tragus, cavum concha and external ear canal. (The GAN innervates the ear, including cavum concha, posterior auricle, the lobule, etc.)

The occipital nerve stimulation (ONS) is an adjunct therapy for neuropsychiatric disorders. The stimulating electrode for occipital nerve stimulator is usually implanted surgically near the occipital nerves at the base of the skull. By impacting the convergence of cervical and trigeminal nerves, stimulation of occipital nerve can help to modulate the brain. The stimulating parameters for occipital nerve are often similar to or overlapping with those for the trigeminal nerve, focusing on delivering comfortable, non-painful stimulation that induces mild tingling (paresthesia). Occipital nerve stimulation (ONS) was found to be helpful for headaches and refractory seizures, although occipital nerve is not a branch of the trigeminal nerve. The occipital nerves arise from the upper cervical spine, including the C2 and C3 nerve roots. Each occipital nerve is divided into three nerves that provide sensation to the back of the head: the greater occipital nerve (mostly C2), the lesser occipital nerve (mostly C2/C3), and the third occipital nerve (C3). The ONS is believe to help seizure by its neuromodulating processes at upper brainstem or diencephalon. It can help to reduce frequency and severity of seizure although ONS is not as common as other neuromodulation. Although ONS is primarily used for refractory pain control, there is some evidence indicating that it may help reduce seizures in refractory seizure patients as an adjunct therapy. Specific seizure reduction percentage by ONS is sparse, but related occipital-focused neurostimulation (RNS) has shown 60%-70% seizure reduction in some studies.

The greater auricular nerve (GAN) stimulation has also shown promise for various neuropsychiatric conditions. GAN stimulation may help alleviate symptoms of depression by modulating neural circuits involved in mood regulation. GAN stimulation has shown potential to reduce anxiety symptoms and post-traumatic stress disorder (PTSD). GAN stimulation has also shown some therapeutic benefits in reducing seizure frequency. GAN stimulation can benefit chronic pain, including headaches. Studies have shown GAN stimulation has potential to enhance neural plasticity and improve cognitive functions. GAN stimulation is also found to enhance parasympathetic activity, which can lead to improved heart rate variability and reduced stress responses. This modulation of the autonomic nervous system may contribute to its therapeutic effects across various neuropsychiatric conditions

For neuropsychiatric disorders (including seizure, migraine, cluster headache and psychiatric disorders), the commonly used stimulation parameters for taVNS, supraorbital nerve stimulation unit, infraorbital nerve stimulation unit, auriculotemporal nerve stimulation unit, occipital nerve stimulation unit and greater auricular nerve stimulation unit may be somewhat different. Examples for these stimulation parameters are:
Common Stimulation Parameters (General Approach)
   (1). Intensity: Individually fitted to create a strong “tingling” but non-painful sensation, often described as below the pain threshold.
   (2). Pulse width: 250-500
   (3.) Frequency: 1 Hz (often used for modulation) or 20-30 Hz (commonly used for seizure/neurological).
   (4.) Waveform: Often dense-sparse waves.
Examples for disease-specific parameters for taVNS (also sometimes used for auriculotemporal nerve stimulation unit and greater auricular nerve stimulation unit):
   (a). For migraine and cluster headache:
      (1). Frequency: 1 Hz (found to have better results for daily prevention) or 25 Hz.
      (2). Duration: 4 hours/day (often split into 1-4 hours sessions).
      (3). Intensity: Adjusted by the patient to a comfortable, tingling sensation.
   (b). For seizure:
      (1). Frequency: 20 Hz (most commonly used).
      (2). Intensity: adjusted to the highest tolerable limit (4-12 mA range).
      (3). Duty Cycle: 30 seconds on/5 minutes off, or 30 s on/30 s off to avoid habituation.
   (c). For psychiatric disorders:
      (1). Frequence: around 20 Hz.
      (2). Duration: 20 min to 1 hour daily.
      (3). Intensity: Maximum, yet non-painful (4-6 mA range reported).

There are other reported examples of stimulation parameters for trigeminal nerve stimulation (TNS) (including supraorbital nerve stimulation, infraorbital nerve stimulation and auriculotemporal nerve stimulation): a high-frequency, low-intensity pulse is usually used, for example a 100-120 Hz frequency and a 250 microseconds pulse width. The typical stimulation parameters for trigeminal nerve stimulation are:
(1). Intensity/Strength: 2-4 mA (with a range of 1-10 mA, maximum of 16 mA) for migraine, adjusted for comfort to a mild tingling sensation without pain.
(2). Frequency: 60-120 Hz
(3). Pulse width (Duration) 200 to 250 microseconds.
(4). Stimulation cycle: commonly 30 seconds on/30 seconds off or continuous for 20-60 minutes depending on condition.
(5.) Session duration: 7-9 hours (overnight) for seizure
(6.) Waveform: usually biphasic pulses.

### BRIEF SUMMARY OF THE INVENTION

According to one aspect consistent with the principles of the invention, an auricular electroencephalogram (EEG) monitoring system is provided. In some embodiments, an auricular electroencephalogram (EEG) monitoring system may include an auricular EEG recording module configured to be linked to a wearer's first ear or the peri-auricular area around the first ear. The EEG recording module may include a plurality of (at least two, but preferably more than two) EEG sensor electrodes and an optional reference electrode. These EEG sensor electrodes and the optional reference electrode are configured to contact separate areas of the wearer's first ear or the peri-auricular area around the wearer's first ear. The areas of the ear or the peri-auricular area that the EEG sensor electrodes and the optional reference electrode are configured to contact may be selected from at least one of the following: an external portion of the wearer's first ear, an external ear canal of the wearer's first ear, and peri-auricular area around the wearer's first ear. The peri-auricular area refers to a portion of the head around the auricle (pinna) and this portion of the head is typically hairless. The peri-auricular area includes a portion of the head in front of the auricle (pre-auricular area) and a portion of the head above and behind the auricle (post-auricular area). The pre-auricular area is small, about one inch wide and about two inches long and curved along the anterior edge of the auricle. The post-auricular area is also small, approximately one inch wide and about three inches long and curved along the superior and posterior edges of the auricle (pinna). The post-auricular area is where a behind-the-ear hearing aid is usually located. The mastoid (mastoid process) is located at the post-auricular area. These small pre-auricular area and post-auricular area together will be called “peri-auricular area” hereinafter. (Anterior, posterior, superior, in front of and behind etc. all refer to the directions relative to the wearer's head when the wearer is in an upright position.) The auricular EEG recording module may be configured to record EEG data of the wearer.

The auricular electroencephalogram (EEG) monitoring system may include a network interface which may be configured to generate a notification to a client device or a client device of the wearer's healthcare provider. A processing unit may be in electronic communication with the auricular EEG recording module and the network interface. The processing unit may be configured to analyze the EEG data recorded by the auricular EEG recording module to detect presence or cessation of EEG signals suggestive of neuropsychiatric disorders. The processing unit may be also configured to analyze the EEG data recorded by the auricular EEG recording module to detect presence or cessation of EEG signals suggestive of impending neuropsychiatric disorders. When the presence of EEG signals suggestive of at least one neuropsychiatric disorder is detected, the processing unit may be configured to immediately send signals to the network interface to generate a notification to the client device. When the presence of EEG signals suggestive of at least one impending neuropsychiatric disorder is detected, the processing unit may be configured to immediately send signals to the network interface to generate a notification to the client device. When the cessation of EEG signals suggestive of at least one neuropsychiatric disorder is detected, the processing unit may be configured to immediately send signals to the network interface to generate a notification to the client device. Likewise, when the cessation of EEG signals suggestive of at least one impending neuropsychiatric disorder is detected, the processing unit may be further configured to immediately send signals to the network interface to generate a notification to the client device.

According to another aspect consistent with the principles of the invention, automatic detection-therapy systems for neuropsychiatric disorders are disclosed. In some embodiments, an automatic detection-therapy system may include an auricular electroencephalogram (EEG) monitoring system, having an auricular EEG recording module configured to be linked to a wearer's first ear or a peri-auricular area around the wearer's first ear. The EEG recording module may include a plurality of (at least two, but preferably more than two) EEG sensor electrodes and an optional reference electrode. These EEG sensor electrodes and the optional reference electrode are configured to contact separate areas of the wearer's first ear or peri-auricular area around the wearer's first ear. The areas that the EEG sensor electrodes are configured to contact may be selected from at least one of the following: an external portion of the wearer's first ear, an external ear canal of the wearer's first ear, and a peri-auricular area around the wearer's first ear. Preferably, all of the EEG sensor electrodes may be configured to contact the skin of the external ear canal of the wearer's first ear, while the optional reference electrode may be configured to contact the skin of the tragus-concha bowl of the wearer's first ear or the mastoid behind the wearer's first ear. The auricular EEG recording module may be configured to record EEG data of the wearer. The automatic detection-therapy system may further include a neuromodulation unit. The neuromodulation unit comprises at least one of the following components: a transcutaneous auricular vagus nerve stimulation unit (taVNS unit), a supraorbital nerve stimulation unit, an auriculotemporal nerve (ATN) stimulation unit, an occipital nerve stimulation unit, a greater auricular nerve (GAN) stimulation unit and an infraorbital nerve stimulation unit, or various combinations of these components. A processing unit may be in electronic communication with the auricular EEG monitoring system and each component of the neuromodulation unit.

In some embodiments, an automatic detection-therapy system for neuropsychiatric disorders may include a neuromodulation unit that include at least one component. A component that the neuromodulation unit has may be configured as a first transcutaneous auricular vagus nerve stimulation unit (first taVNS unit). The first taVNS unit has a first taVNS stimulating electrode configured to contact vagus innervated auricular skin of the wearer's first ear. The vagus innervated auricular skin includes external ear canal, tragus, cymba-concha, cavum-concha and small adjacent areas. (More precisely, the vagus-innervated auricular skin includes inner posterior portion of tragus, cymba-concha, cavum-concha, posterior inferior walls of the external ear canal and small adjacent regions of the external ear.) The vagus innervated auricular skin that the first taVNS stimulating electrode is configured to contact may be selected from at least one of the following: tragus, cymba-concha, cavum-concha and the external ear canal of the wearer's first ear. A processing unit may be in electronic communication with the auricular EEG recording module and in electronic communication with the first taVNS unit. The processing unit may be configured to analyze the EEG data recorded by the EEG monitoring system with the help of advanced EEG analysis algorithms together with machine learning, deep learning and artificial intelligence to detect the presence or cessation of EEG signals suggestive of at least one neuropsychiatric disorder. When the presence of EEG signals suggestive of at least one neuropsychiatric disorder is detected by the processing unit, the processing unit may be configured to immediately send signals to the first taVNS unit to prompt it to automatically start sending pre-determined electric stimulation to the vagus innervated auricular skin of the wearer's first ear to which the first taVNS stimulating electrode is in contact with. When cessation of EEG signals suggestive of at least one neuropsychiatric disorder is detected by the processing unit, the processing unit may be further configured to immediately send signals to the first taVNS unit to automatically stop sending pre-determined electric stimulation to the vagus innervated auricular skin of the wearer's first ear to which the first ta VNS stimulating electrode is in contact with. The processing unit may be further configured to analyze the EEG data recorded by the EEG monitoring system to detect presence or cessation of EEG signals suggestive of at least one impending neuropsychiatric disorder. When the presence of EEG signals suggestive of at least one impending neuropsychiatric disorder is detected by the processing unit, the processing unit may be configured to immediately send signals to the first ta VNS unit to prompt it to automatically start sending predetermined electric stimuli to the vagus innervated auricular skin of the wearer's first ear to which the first taVNS stimulating electrode is in contact with. When cessation of EEG signals suggestive of at least one impending neuropsychiatric disorder is detected by the processing unit, the processing unit may be further configured to immediately send signals to the first taVNS unit to prompt it to automatically stop sending pre-determined electric stimulation to the vagus innervated auricular skin of the wearer's first ear. In some embodiments, the neuromodulation unit may further comprise at least one of the following components: a supraorbital nerve stimulation unit, an auriculotemporal nerve stimulation unit, an occipital nerve stimulation unit, an infraorbital nerve stimulation unit and a greater auricular nerve stimulation unit, and their setups are similar to the aforementioned descriptions for taVNS. They will be described in more details hereinafter, including various combinations of them.

In another aspect consistent with the principles of this invention, an automatic detection-therapy system for neuropsychiatric disorders may include an auricular electroencephalogram (EEG) monitoring system, a network interface and at least one component of the neuromodulation unit. The auricular electroencephalogram (EEG) monitoring system may include an auricular EEG recording module configured to be linked to a wearer's (or user's) first ear or a peri-auricular area around the first ear. The EEG recording module may include a plurality of (at least two, but preferably more than two) EEG sensor electrodes and an optional reference electrode. These EEG sensor electrodes and the reference electrode may be configured to contact separate areas of the wearer's first ear or the peri-auricular area around the wearer's first ear. Preferably, all of the EEG sensor electrodes may be configured to contact the skin of the external ear canal of the wearer's first ear, while the optional reference electrode may be configured to contact the skin of the tragus-concha bowl of the wearer's first ear or the mastoid behind the wearer's first ear. A processing unit may be in electronic communication with the auricular EEG monitoring system and the network interface. The processing unit may be configured to analyze the EEG data recorded by the auricular EEG monitoring system with the help of advanced EEG analysis algorithms together with machine learning, deep learning and artificial intelligence to detect presence or cessation of EEG signals suggestive of neuropsychiatric disorders or impending neuropsychiatric disorders. When the presence of EEG signals suggestive of at least one neuropsychiatric disorder or at least one impending neuropsychiatric disorder is detected, the processing unit may be configured to immediately send signals to the network interface to generate a notification to the client device of the wearer or a client device of a healthcare provider of the wearer. When cessation of EEG signals suggestive of at least one neuropsychiatric disorder or at least one impending neuropsychiatric disorder is detected, the processing unit may be further configured to immediately send signals to the network interface to generate a notification to the client device of the wearer or a client device of a healthcare provider of the wearer.

In some embodiments, an automatic detection-therapy system for neuropsychiatric disorders may include a neuromodulation unit that may have a transcutaneous auricular vagus nerve stimulation unit (taVNS unit). The taVNS unit includes a vagus nerve stimulating electrode configured to contact vagus innervated auricular skin of the wearer's first ear. The vagus innervated auricular skin includes external ear canal, tragus, cymba-concha, cavum-concha and small adjacent areas. The vagus innervated auricular skin that the ta VNS stimulating electrode is configured to contact is selected from at least one of the following: inner-posterior tragus, cymba-concha, cavum-concha and the posterior-inferior walls of external ear canal of the wearer's first ear. A processing unit is in electronic communication with the auricular EEG monitoring system and the taVNS unit. The processing unit is configured to analyze the EEG data recorded by the EEG monitoring system to detect the presence or cessation of EEG signals suggestive of at least one neuropsychiatric disorder. When the presence of EEG signals suggestive of at least one neuropsychiatric disorder is detected by the processing unit, the processing unit is configured to immediately send signals to the taVNS unit to prompt it to automatically start sending pre-determined electric stimulation to the vagus innervated auricular skin of the wearer's first ear to which the first taVNS stimulating electrode is in contact with. When cessation of EEG signals suggestive of at least one neuropsychiatric disorder is detected by the processing unit, the processing unit is further configured to immediately send signals to the taVNS unit to automatically stop sending pre-determined electric stimulation to the vagus innervated auricular skin of the wearer's first ear. The processing unit is further configured to analyze the EEG data recorded by the auricular EEG monitoring system to detect presence or cessation of EEG signals suggestive of at least one impending neuropsychiatric disorder. When the presence of EEG signals suggestive of at least one impending neuropsychiatric disorder is detected by the processing unit, the processing unit is configured to immediately send signals to the taVNS unit to automatically start sending predetermined electric stimuli to the vagus innervated auricular skin of the wearer's first ear. When cessation of EEG signals suggestive of the at least one impending neuropsychiatric disorder is detected by the processing unit, the processing unit is further configured to immediately send signals to the taVNS unit to automatically stop sending pre-determined electric stimuli to the vagus innervated auricular skin of the wearer's first ear.

For the automatic detection-therapy system for neuropsychiatric disorders, examples of the general taVNS neuromodulating stimulation parameters for neuropsychiatric disorders and impending neuropsychiatric disorders are shown in Tables 1 and 2.

TABLE 1
Example of taVNS unit 30 electric stimuli output
parameters for neuropsychiatric disorders.

| Output | Parameter |
|---|---|
| Power supply | Direct current 3-9 volts |
| Pulse width | 0.05-1.0 milliseconds (ms) |
| Frequency | 0.5-200 Hz |
| Modes | Continuous wave or sparse-dense wave |
| Intensity | 0.1-15 milliamperes (mA) |

TABLE 2
Example of taVNS unit 30 electric stimuli output
parameters for impending neuropsychiatric disorders.

| Output | Parameter | |
|---|---|---|
| Power supply | Direct current 3-9 | volts |
| Pulse width | 0.05-0.9 | milliseconds (ms) |
| Frequency | 0.5-150 | Hz |
| Modes | Continuous wave or | |
| | sparse-dense wave | |
| Intensity | 0.1-10 | milliamperes (mA) |

For the automatic detection-therapy system for seizure and impending seizure, examples for the taVNS neuromodulating stimulation parameters are shown in Tables 3 and 4:

TABLE 3
Example of taVNS unit 30 electric stimuli
output parameters for seizure (epilepsy).

| Output | Parameter | |
|---|---|---|
| Power supply | Direct current | |
| | 3-9 | volts |
| Pulse width | 0.25-0.5 | ms |
| | (range 0.13-1 | ms) |
| Frequency | 10-25 | Hz |
| Modes | Continuous wave or | |
| | sparse-dense wave | |
| Intensity | 0.25-1.75 | mA |
| On/off time | 30 | seconds (s) on/ |
| | 3 | minutes (min) off |

| | |
|---|---|
| | (range 7 s-120 s on/ |
| | 18 s-30 min off) |
| Laterality | Bilateral or alternating |
| | between left and right |

TABLE 4
Example of taVNS unit 30 electric stimuli output
parameters for impending seizure (epilepsy)

| Output | Parameter | |
|---|---|---|
| Power supply | Direct current | |
| | 3-9 | volts |
| Pulse width | 0.25-0.5 | ms |
| | (range 0.13-1 | ms) |
| Frequency | 10-25 | Hz |
| Modes | Continuous wave or | |
| | sparse-dense wave | |
| Intensity | 0.25-1.25 | mA |
| On/off time | 30 | seconds (s) on/ |
| | 5 | minutes (min) off |

| | |
|---|---|
| | (range 7 s-120 s on/ |
| | 18 s-60 min off) |
| Laterality | Bilateral or alternating |
| | between left and right |

In some embodiments, an automatic detection-therapy system for neuropsychiatric disorders may include a neuromodulation unit that may have a component which is a supraorbital nerve stimulation unit. The supraorbital nerve is a branch of the ophthalmic division of the trigeminal nerve. There are two supraorbital nerves, one on each side of the forehead. (Some non-invasive transcutaneous supraorbital nerve stimulators, such as the CEFALY device, are placed on mid-forehead and are designed to deliver electrical stimulation to the supraorbital nerves on both sides of the forehead simultaneously.) The supraorbital nerve stimulation unit includes a supraorbital nerve stimulating electrode configured to contact the supraorbital nerve innervated area of the wearer's forehead. A processing unit is in electronic communication with the auricular EEG monitoring system and the supraorbital nerve stimulation unit. The processing unit is configured to analyze the EEG data recorded by the auricular EEG monitoring system to detect the presence or cessation of EEG signals suggestive of at least one neuropsychiatric disorder. When the presence of EEG signals suggestive of at least one neuropsychiatric disorder is detected by the processing unit, the processing unit is configured to immediately send signals to the supraorbital nerve stimulation unit to automatically start sending pre-determined electric stimulation to the wearer's supraorbital nerves. When cessation of EEG signals suggestive of at least one neuropsychiatric disorder is detected by the processing unit, the processing unit is further configured to immediately send signals to the supraorbital nerve stimulation unit to automatically stop sending pre-determined electric stimulation to the wearer's supraorbital nerves. The processing unit is further configured to analyze the EEG data recorded by the EEG monitoring system to detect presence or cessation of EEG signals suggestive of impending neuropsychiatric disorders. When the presence of EEG signals suggestive of at least one impending neuropsychiatric disorder is detected by the processing unit, the processing unit is configured to immediately send signals to the supraorbital nerve stimulation unit to automatically start sending predetermined electric stimulation to the wearer's supraorbital nerves. When cessation of EEG signals suggestive of the at least one impending neuropsychiatric disorder is detected by the processing unit, the processing unit is further configured to immediately send signals to the supraorbital nerve stimulation unit to automatically stop sending pre-determined electric stimulation to the wearer's supraorbital nerves. The setups and functions of an infraorbital nerve stimulation unit is similar to the aforementioned description for the supraorbital nerve stimulation unit.

In some embodiments, an automatic detection-therapy system for neuropsychiatric disorders may include a neuromodulation unit that may have a component which is an auriculotemporal nerve (ATN) stimulation unit. The auriculotemporal nerve (ATN) is a branch of the mandibular division of the trigeminal nerve. The ATN stimulation unit includes an ATN stimulating electrode configured to contact the ATN innervated area of the wearer's ear. The ATN innervated area includes the anterior-outer part of tragus, upper anterior part of the helix, anterior portion of of cavum concha, anterior and superior walls of external ear canal, outer surface of the tympanic membrane and area immediately in front of the tragus. The auriculotemporal nerve (ATN) innervated area is anatomically adjacent to or overlapping with the vagus-innervated auricular skin. (For comparison: The vagus-innervated auricular skin includes: inner posterior portion of tragus, cymba-concha, cavum-concha, posterior and inferior walls of the external ear canal and small adjacent regions of the external ear. The ATN innervated auricular skin includes: anterior outer part of tragus, the anterior-superior part of cavum concha, anterior and superior walls of the external ear canal and, anterior and superior part of pinna including anterior-superior helix.) Because of this, it is feasible for the ATN stimulation unit and the taVNS unit to share a housing device or even share a stimulating electrode. However, it is usually preferred to have separate devices (could be integrated into one housing) with separate stimulating electrodes and separate stimulating parameters because their optimal distinct stimulating parameters are often different. A processing unit may be in electronic communication with the auricular EEG recording module and the auriculotemporal nerve stimulation unit. The processing unit may be configured to analyze the EEG data recorded by the EEG monitoring system to detect the presence or cessation of EEG signals suggestive of at least one neuropsychiatric disorder. When the presence of EEG signals suggestive of at least one neuropsychiatric disorder is detected by the processing unit, the processing unit may be configured to immediately send signals to the auriculotemporal nerve (ATN) stimulation unit to automatically start sending pre-determined electric stimuli to the wearer's ATN. When cessation of EEG signals suggestive of at least one neuropsychiatric disorder is detected by the processing unit, the processing unit may be configured to immediately send signals to the ATN stimulation unit to automatically stop sending pre-determined electric stimuli to the wearer's ATN. The processing unit may be further configured to analyze the EEG data recorded by the EEG monitoring system to detect presence or cessation of EEG signals suggestive of impending neuropsychiatric disorders. When the presence of EEG signals suggestive of at least one impending neuropsychiatric disorder is detected by the processing unit, the processing unit may be configured to immediately send signals to the auriculotemporal nerve (ATN) stimulation unit to automatically start sending predetermined electric stimulation to the wearer's ATN. When cessation of EEG signals suggestive of at least one impending neuropsychiatric disorder is detected by the processing unit, the processing unit may be further configured to immediately send signals to the ATN stimulation unit to automatically stop sending pre-determined electric stimulation to the wearer's auriculotemporal nerve.

In some embodiments, an automatic detection-therapy system for neuropsychiatric disorders may include a neuromodulation unit which may have a component that is an occipital nerve stimulation unit. An occipital nerve stimulation unit may deliver stimulation to unilateral occipital nerve or bilateral occipital nerves, depending on user's condition. (An occipital nerve stimulator placed at mid-occipital area could be used to reach bilateral occipital nerves.) (Traditionally, an occipital nerve stimulator is placed by surgery under the skin in occipital region. Nowadays, transcutaneous occipital nerve stimulator is available.) The occipital nerve stimulation unit includes one or two stimulating electrodes configured to contact the occipital nerves innervated areas of the wearer's occipital region of the head. A processing unit is in electronic communication with the auricular EEG monitoring system and the occipital nerve stimulation unit. The processing unit is configured to analyze the EEG data recorded by the auricular EEG monitoring system to detect the presence or cessation of EEG signals suggestive of neuropsychiatric disorders. When the presence of EEG signals suggestive of at least one neuropsychiatric disorder is detected by the processing unit, the processing unit is configured to immediately send signals to the occipital nerve stimulation unit to automatically start sending pre-determined electric stimulation to the wearer's occipital nerve(s). When cessation of EEG signals suggestive of the at least one neuropsychiatric disorder is detected by the processing unit, the processing unit is further configured to immediately send signals to the occipital nerve stimulation unit to automatically stop sending pre-determined electric stimulation to the wearer's occipital nerve(s). The processing unit is further configured to analyze the EEG data recorded by the auricular EEG monitoring system to detect presence or cessation of EEG signals suggestive of impending neuropsychiatric disorders. When the presence of EEG signals suggestive of at least one impending neuropsychiatric disorder is detected by the processing unit, the processing unit is configured to immediately send signals to the occipital nerve stimulation unit to automatically start sending predetermined electric stimulation to the wearer's occipital nerve(s). When cessation of EEG signals suggestive of the at least one impending neuropsychiatric disorder is detected by the processing unit, the processing unit is further configured to immediately send signals to the occipital nerve stimulation unit to automatically stop sending pre-determined electric stimuli to the wearer's occipital nerve(s).

In some embodiments, an automatic detection-therapy system for neuropsychiatric disorders may include a neuromodulation unit that may have a component which is a greater auricular nerve (GAN) stimulation unit. The greater auricular nerve (GAN) stimulation unit includes a stimulating electrode configured to contact the GAN innervated area of the wearer's auricular skin (for example the skin of the wearer's cavum concha). A processing unit is in electronic communication with the auricular EEG monitoring system and the GAN stimulation unit. The processing unit is configured to analyze the EEG data recorded by the auricular EEG monitoring system to detect the presence or cessation of EEG signals suggestive of neuropsychiatric disorders. When the presence of EEG signals suggestive of at least one neuropsychiatric disorder is detected by the processing unit, the processing unit is configured to immediately send signals to the greater auricular nerve (GAN) stimulation unit to automatically start sending pre-determined electric stimulation to the wearer's GAN innervated auricular skin. When cessation of EEG signals suggestive of the at least one neuropsychiatric disorder is detected by the processing unit, the processing unit is further configured to immediately send signals to the GAN stimulation unit to automatically stop sending pre-determined electric stimuli to the wearer's GAN innervated auricular skin. The processing unit is further configured to analyze the EEG data recorded by the auricular EEG monitoring system to detect presence or cessation of EEG signals suggestive of impending neuropsychiatric disorders. When the presence of EEG signals suggestive of at least one impending neuropsychiatric disorder is detected by the processing unit, the processing unit is configured to immediately send signals to the GAN stimulation unit to automatically start sending predetermined electric stimulation to the wearer's GAN innervated auricular skin. When cessation of EEG signals suggestive of the at least one impending neuropsychiatric disorder is detected by the processing unit, the processing unit is further configured to immediately send signals to the GAN stimulation unit to automatically stop sending pre-determined electric stimulation to the wearer's GAN innervated auricular skin.

In modified embodiments, the automatic detection-therapy system for neuropsychiatric disorders may have a neuromodulation unit that comprises more than one component. For example, the neuromodulation unit may comprise a taVNS unit and an auriculotemporal nerve (ATN) stimulation unit. When the processing unit detects presence of EEG signals suggestive of at least one neuropsychiatric disorder, the processing unit may be configured to send signals to both the taVNS unit and the ATN stimulation unit to prompt the ta VNS unit to send pre-determined electric stimuli to the wearer's vagus-innervated auricular skin and also to prompt the ATN stimulation unit to send pre-determined electric stimuli to the wearer's ATN innervated auricular skin. When the processing unit detects presence of EEG signals suggestive of at least one impending neuropsychiatric disorder, the processing unit may be configured to send signals to both the taVNS unit and the auriculotemporal nerve (ATN) stimulation unit to prompt the ta VNS unit to send pre-determined electric stimuli to the wearer's vagus-innervated auricular skin and also to prompt the ATN stimulation unit to send pre-determined electric stimuli to the wearer's ATN innervated auricular skin. When the processing unit detects cessation of EEG signals suggestive of at least one neuropsychiatric disorder, the processing unit may be configured to send signals to both the taVNS unit and the ATN stimulation unit to prompt the taVNS unit to stop sending pre-determined electric stimuli to the wearer's vagus-innervated auricular skin and also to prompt the ATN stimulation unit to stop sending pre-determined electric stimulation to the wearer's ATN innervated auricular skin. When the processing unit detects cessation of EEG signals suggestive of at least one impending neuropsychiatric disorder, the processing unit may be further configured to send signals to both the taVNS unit and the auriculotemporal nerve (ATN) stimulation unit to prompt the taVNS unit to stop sending pre-determined electric stimulation to the wearer's vagus-innervated auricular skin and also to prompt the ATN stimulation unit to stop sending pre-determined electric stimuli to the wearer's ATN innervated auricular skin.

In another modified embodiment, the neuromodulation unit for the automatic detection-therapy system for neuropsychiatric disorders may include two components: a taVNS unit and an occipital nerve stimulation unit. When the processing unit detects presence of EEG signals suggestive of at least one neuropsychiatric disorder, the processing unit may be configured to send signals to both the taVNS unit and the occipital nerve stimulation unit to prompt the taVNS unit to send pre-determined electric stimuli to the wearer's vagus-innervated auricular skin and also to prompt the occipital nerve stimulation unit to send pre-determined electric stimuli to the wearer's occipital nerve innervated region. When the processing unit detects presence of EEG signals suggestive of at least one impending neuropsychiatric disorder, the processing unit may be configured to send signals to both the taVNS unit and the occipital nerve stimulation unit to prompt the taVNS unit to send pre-determined electric stimulation to the wearer's vagus-innervated auricular skin and also to prompt the occipital nerve stimulation unit to send pre-determined electric stimuli to the wearer's occipital nerve innervated region. When the processing unit detects cessation of EEG signals suggestive of at least one neuropsychiatric disorder, the processing unit may be configured to send signals to both the taVNS unit and the occipital nerve stimulation unit to prompt the taVNS unit to stop sending pre-determined electric stimulation to the wearer's vagus-innervated auricular skin and also to prompt the occipital nerve stimulation unit to stop sending pre-determined electric stimulation to the wearer's occipital nerve innervated region. When the processing unit detects cessation of EEG signals suggestive of at least one impending neuropsychiatric disorder, the processing unit may be further configured to send signals to both the taVNS unit and the occipital nerve stimulation unit to prompt the taVNS unit to stop sending pre-determined electric stimulation to the wearer's vagus-innervated auricular skin and also to prompt the occipital nerve stimulation unit to stop sending pre-determined electric stimulation to the wearer's occipital nerve innervated region.

In yet another modified embodiment, the neuromodulation unit for the automatic detection-therapy system for neuropsychiatric disorders may include the following two components: a taVNS unit and a supraorbital nerve stimulation unit. When the processing unit detects presence of EEG signals suggestive of at least one neuropsychiatric disorder, the processing unit may be configured to send signals to both the taVNS unit and the supraorbital nerve stimulation unit to prompt the taVNS unit to send pre-determined electric stimulation to the wearer's vagus-innervated auricular skin and also to prompt the supraorbital nerve stimulation unit to send pre-determined electric stimuli to the wearer's supraorbital nerve innervated forehead skin. When the processing unit detects presence of EEG signals suggestive of impending at least one neuropsychiatric disorder, the processing unit may be configured to send signals to both the taVNS unit and the supraorbital nerve stimulation unit to prompt the ta VNS unit to send pre-determined electric stimulation to the wearer's vagus-innervated auricular skin and also to prompt the supraorbital nerve stimulation unit to send pre-determined electric stimulation to the wearer's supraorbital nerve innervated forehead skin. When the processing unit detects cessation of EEG signals suggestive of at least one neuropsychiatric disorder, the processing unit may be configured to send signals to both the taVNS unit and the supraorbital nerve stimulation unit to prompt the taVNS unit to stop sending pre-determined electric stimulation to the wearer's vagus-innervated auricular skin and also to prompt the supraorbital nerve stimulation unit to stop sending pre-determined electric stimuli to the wearer's supraorbital nerve innervated forehead skin. When the processing unit detects cessation of EEG signals suggestive of at least one impending neuropsychiatric disorder, the processing unit may be further configured to send signals to both the taVNS unit and the supraorbital nerve stimulation unit to prompt the taVNS unit to stop sending pre-determined electric stimulation to the wearer's vagus-innervated auricular skin and also to prompt the supraorbital nerve stimulation unit to stop sending pre-determined electric stimulation to the wearer's supraorbital nerve innervated forehead skin.

In still another modified embodiment, the neuromodulation unit for the automatic detection-therapy system for neuropsychiatric disorders may include the following two components: an auriculotemporal nerve (ATN) stimulation unit and a supraorbital nerve stimulation unit. The supraorbital nerve and the auriculotemporal nerve are different branches of the trigeminal nerve. Studies have shown that simultaneous stimulation of both branches of the trigeminal nerve may provide more comprehensive effects. When the processing unit detects presence of EEG signal suggestive of at least one neuropsychiatric disorder, the processing unit may be configured to send signals to both the auriculotemporal nerve (ATN) stimulation unit and the supraorbital nerve stimulation unit to prompt the ATN stimulation unit to send pre-determined electric stimulation to the wearer's ATN innervated auricular skin and also to prompt the supraorbital nerve stimulation unit to send pre-determined electric stimulation to the wearer's supraorbital nerve innervated forehead skin. When the processing unit detects presence of EEG signals suggestive of at least one impending neuropsychiatric disorder, the processing unit may be configured to send signals to both the ATN stimulation unit and the supraorbital nerve stimulation unit to prompt the ATN stimulation unit to send pre-determined electric stimulation to the wearer's ATN innervated auricular skin and also to prompt the supraorbital nerve stimulation unit to send pre-determined electric stimulation to the wearer's supraorbital nerve innervated forehead skin. When the processing unit detects cessation of EEG signal suggestive of at least one neuropsychiatric disorder, the processing unit may be configured to send signals to both the auriculotemporal nerve (ATN) stimulation unit and the supraorbital nerve stimulation unit to prompt the ATN stimulation unit to stop sending pre-determined electric stimulation to the wearer's ATN innervated auricular skin and also to prompt the supraorbital nerve stimulation unit to stop sending pre-determined electric stimulation to the wearer's supraorbital nerve innervated forehead skin. When the processing unit detects cessation of EEG signals suggestive of at least one impending neuropsychiatric disorder, the processing unit may be further configured to send signals to both the auriculotemporal nerve (ATN) stimulation unit and the supraorbital nerve stimulation unit to prompt the ATN stimulation unit to stop sending pre-determined electric stimulation to the wearer's ATN innervated auricular skin and also to prompt the supraorbital nerve stimulation unit to stop sending pre-determined electric stimulation to the wearer's supraorbital nerve innervated forehead skin.

In still yet another modified embodiment, the neuromodulation unit for the automatic detection-therapy system for neuropsychiatric disorders may include the following two components: a taVNS stimulation unit and a greater auricular nerve (GAN) stimulation unit, similar to the aforementioned description. Other components of the neuromodulation unit may also be combined, similar to the aforementioned descriptions. In further modified embodiments, the neuromodulation unit for the automatic detection-therapy system for neuropsychiatric disorders may include a combination of 3 different components of the neuromodulation unit, for example, a combination of a taVNS unit, an auriculotemporal nerve stimulation unit and a supraorbital nerve stimulation unit, or a combination of a taVNS unit, an auriculotemporal nerve stimulation unit and an occipital nerve stimulation unit. The setups and functions of combining 3 components of the neuromodulation unit are similar to the aforementioned descriptions. All of these various combinations are within the scope of this invention.

In some embodiments, an automatic detection-therapy system for neuropsychiatric disorders may include a second set of an auricular EEG recording module to be linked to the wearer's second ear. The bilateral EEG recording modules are in electronic communication with the processing unit. The bilateral EEG will enhance the capability of this system to detect neuropsychiatric disorders or impending neuropsychiatric disorders. For most patients with neuropsychiatric disorders, two auricular EEG recording modules, one on each side of the head 901 linked to each ear, will be preferred. This is especially true for patients with generalized seizures or focal seizures which become secondarily generalized. In rare situations, only one auricular EEG recording module may be utilized for patients with strictly localized seizures. In some embodiments, an automatic detection-therapy system for neuropsychiatric disorders may include a second neuromodulation unit that may include a second ta VNS unit. For some patients with neuropsychiatric disorders, two taVNS units may be utilized, with one taVNS unit on each side of the head linked to each ear. In rare situations if the patient is unable to tolerate a taVNS unit in the right ear, only one taVNS unit will be utilized and be placed in the left ear. In some embodiments, an automatic detection-therapy system for neuropsychiatric disorders may include two neuromodulation units. The second neuromodulation unit may include a second auriculotemporal nerve stimulation unit placed in the wearer's second ear. Similarly, the second neuromodulation unit may include a second greater auricular nerve stimulation unit placed in the wearer's second ear.

The aforementioned descriptions for neuropsychiatric disorders may be applied to each individual neuropsychiatric disorder, including seizure, migraine, cluster headache, major depressive disorder (MDD), bipolar disorder, schizophrenia, obsessive-compulsive disorder (OCD), attention deficit and hyperactivity disorder (ADHD), autism spectrum disorder (ASD), post-traumatic stress disorder (PTSD), anxiety disorder and panic disorder, etc., with similar or some modification of the stimulation parameters for each component of the neuromodulation unit.

In some embodiments, an automatic detection-therapy system for seizure may include an auricular EEG monitoring system, a neuromodulation unit and a processing unit. The auricular EEG monitoring system may be configured to detect seizure or impending seizure. When presence of EEG signals suggestive of seizure is detected, the processing unit is configured to send signals to at least one of the following: a transcutaneous auricular nerve stimulation unit (taVNS unit), a supraorbital nerve stimulation unit, an auriculotemporal nerve (ATN) stimulation unit, an occipital nerve stimulation unit, a greater auricular nerve (GAN) stimulation unit and an infraorbital nerve stimulation unit (or various combinations thereof) to start sending neuromodulating electric stimulation. Likewise, when presence of EEG signals suggestive of impending seizure is detected, the processing unit is configured to send signals to at least one of the following: the taVNS unit, the supraorbital nerve stimulation unit, the ATN stimulation unit, the occipital nerve stimulation unit, the GAN stimulation unit and the infraorbital nerve stimulation unit (or various combinations thereof) to start sending neuromodulating electric stimulation. When cessation of EEG signals suggestive of seizure or impending seizure is detected, the processing unit is configured to send signals to the neuromodulation unit to stop sending neuromodulating electric stimulation from any of the following: the taVNS unit, the supraorbital nerve stimulation unit, the ATN stimulation unit, the occipital nerve stimulation unit, the GAN stimulation unit and the infraorbital nerve stimulation unit.

In some embodiments, an automatic detection-therapy system for migraine may include an auricular EEG monitoring system, a neuromodulation unit and a processing unit. The auricular EEG monitoring system may be configured to detect migraine or impending migraine. When presence of EEG signals suggestive of migraine is detected, the processing unit is configured to send signals to at least one of the following: a transcutaneous auricular nerve stimulation unit (taVNS unit), a supraorbital nerve stimulation unit, an auriculotemporal nerve (ATN) stimulation unit, an occipital nerve stimulation unit, a greater auricular nerve (GAN) stimulation unit and an infraorbital nerve stimulation unit (or various combinations thereof) to start sending neuromodulating electric stimulation. Likewise, when presence of EEG signals suggestive of impending migraine is detected, the processing unit is configured to send signals to at least one of the following: the taVNS unit, the supraorbital nerve stimulation unit, the ATN stimulation unit, the occipital nerve stimulation unit, the GAN stimulation unit and the infraorbital nerve stimulation unit (or various combinations thereof) to start sending neuromodulating electric stimulation. When cessation of EEG signals suggestive of migraine or impending migraine is detected, the processing unit is configured to send signals to the neuromodulation unit to stop sending neuromodulating electric stimuli from any of the following: the taVNS unit, the supraorbital nerve stimulation unit, the ATN stimulation unit, the occipital nerve stimulation unit, the GAN stimulation unit and the infraorbital nerve stimulation unit.

In some embodiments, an automatic detection-therapy system for cluster headache may include an auricular EEG monitoring system, a neuromodulation unit and a processing unit. The auricular EEG monitoring system may be configured to detect cluster headache or impending cluster headache. When presence of EEG signals suggestive of cluster headache is detected, the processing unit is configured to send signals to at least one of the following: a transcutaneous auricular nerve stimulation unit (ta VNS unit), a supraorbital nerve stimulation unit, an auriculotemporal nerve (ATN) stimulation unit, an occipital nerve stimulation unit, a greater auricular nerve (GAN) stimulation unit and an infraorbital nerve stimulation unit (or various combinations thereof) to start sending neuromodulating electric stimulation. Likewise, when presence of EEG signals suggestive of impending cluster headache is detected, the processing unit is configured to send signals to at least one of the following: the taVNS unit, the supraorbital nerve stimulation unit, the ATN stimulation unit, the occipital nerve stimulation unit, the GAN stimulation unit and the infraorbital nerve stimulation unit (or various combinations thereof) to start sending neuromodulating electric stimulation. When cessation of EEG signals suggestive of cluster headache or impending cluster headache is detected, the processing unit is configured to send signals to the neuromodulation unit to stop sending neuromodulating electric stimuli from any of the following: the taVNS unit, the supraorbital nerve stimulation unit, the ATN stimulation unit, the occipital nerve stimulation unit, the GAN stimulation unit and the infraorbital nerve stimulation unit.

Extended EEG monitoring with auricular EEG monitoring system has also been found to be very useful in detecting several psychiatric disorders and impending psychiatric disorders, including major depressive disorder (MDD), bipolar disorder, schizophrenia, obsessive-compulsive disorder (OCD), attention deficit and hyperactivity disorder (ADHD), autism spectrum disorder (ASD), post-traumatic stress disorder (PTSD), anxiety disorder and panic disorder. Studies have also shown that vagus nerve stimulation (through taVNS), supraorbital nerve stimulation, auriculotemporal nerve (ATN) stimulation, occipital nerve stimulation, greater auricular nerve (GAN) stimulation, infraorbital nerve stimulation and various combinations thereof are very helpful for these psychiatric disorders. Trigeminal nerve stimulation can be given via the supraorbital nerve or the auriculotemporal nerve or the infraorbital nerve.

In some embodiments, an automatic detection-therapy system for psychiatric disorders (including MDD, bipolar disorder, schizophrenia, OCD, ADHD, ASD, PTSD, anxiety disorder and panic disorder) may be configured in a way similar to the aforementioned descriptions for the automatic detection-therapy systems for neuropsychiatric disorders. The aforementioned embodiments for treating neuropsychiatric disorders can be modified to treat each psychiatric disorder. There are several psychiatric disorders that can be treated with the automatic detection-therapy system of this invention. For example, in some embodiments, an automatic detection-therapy system for schizophrenia may include an auricular EEG monitoring system, a neuromodulation unit and a processing unit. The auricular EEG monitoring system may be configured to detect schizophrenia or impending schizophrenia. When presence of EEG signals suggestive of schizophrenia is detected, the processing unit is configured to send signals to at least one of the following: a transcutaneous auricular nerve stimulation unit (taVNS unit), a supraorbital nerve stimulation unit, an auriculotemporal nerve (ATN) stimulation unit, an occipital nerve stimulation unit, a greater auricular nerve (GAN) stimulation unit and an infraorbital nerve stimulation unit (or various combinations thereof) to start sending neuromodulating electric stimuli. Likewise, when presence of EEG signals suggestive of impending schizophrenia is detected, the processing unit is configured to send signals to at least one of the following: the taVNS unit, the supraorbital nerve stimulation unit, the ATN stimulation unit, the occipital nerve stimulation unit, the GAN stimulation unit and the infraorbital nerve stimulation unit (or various combinations thereof) to start sending neuromodulating electric stimulation. When cessation of EEG signals suggestive of schizophrenia or impending schizophrenia is detected, the processing unit is configured to send signals to the neuromodulation unit to stop sending neuromodulating electric stimulation from any of the following: the taVNS unit, the supraorbital nerve stimulation unit, the ATN stimulation unit, the occipital nerve stimulation unit, the GAN stimulation unit and the infraorbital nerve stimulating electrode.

In some embodiments, an automatic detection-therapy system for major depressive disorder (MDD) may include an auricular EEG monitoring system, a neuromodulation unit and a processing unit. The auricular EEG monitoring system may be configured to detect MDD or impending MDD. When presence of EEG signals suggestive of MDD is detected, the processing unit is configured to send signals to at least one of the following: a transcutaneous auricular nerve stimulation unit (taVNS unit), a supraorbital nerve stimulation unit, an auriculotemporal nerve (ATN) stimulation unit, an occipital nerve stimulation unit, a greater auricular nerve (GAN) stimulation unit and an infraorbital nerve stimulation unit (or various combinations thereof) to start sending neuromodulating electric stimulation. Likewise, when presence of EEG signals suggestive of impending MDD is detected, the processing unit is configured to send signals to at least one of the following: the ta VNS unit, the supraorbital nerve stimulation unit, the ATN stimulation unit, the occipital nerve stimulation unit, the GAN stimulation unit and the infraorbital nerve stimulation unit (or various combinations thereof) to start sending neuromodulating electric stimulation. When cessation of EEG signals suggestive of MDD or impending MDD is detected, the processing unit is configured to send signals to the neuromodulation unit to stop sending neuromodulating electric stimuli from any of the following: the taVNS unit, the supraorbital nerve stimulation unit, the ATN stimulation unit, the occipital nerve stimulation unit, the GAN stimulation unit and the infraorbital nerve stimulation unit.

In some embodiments, an automatic detection-therapy system for bipolar disorder may include an auricular EEG monitoring system, a neuromodulation unit and a processing unit. The auricular EEG monitoring system may be configured to detect bipolar disorder or impending bipolar disorder. When presence of EEG signals suggestive of bipolar disorder is detected, the processing unit is configured to send signals to at least one of the following: a transcutaneous auricular nerve stimulation unit (taVNS unit), a supraorbital nerve stimulation unit, an auriculotemporal nerve (ATN) stimulation unit, an occipital nerve stimulation unit, a greater auricular nerve (GAN) stimulation unit and an infraorbital nerve stimulation unit (or various combinations thereof) to start sending neuromodulating electric stimuli. Likewise, when presence of EEG signals suggestive of impending bipolar disorder is detected, the processing unit is configured to send signals to at least one of the following: the taVNS unit, the supraorbital nerve stimulation unit, the ATN stimulation unit, the occipital nerve stimulation unit, the GAN stimulation unit and the infraorbital nerve stimulation unit (or various combinations thereof) to start sending neuromodulating electric stimuli. When cessation of EEG signals suggestive of bipolar disorder or impending bipolar disorder is detected, the processing unit is configured to send signals to the neuromodulation unit to stop sending neuromodulating electric stimuli from any of the following: the taVNS unit, the supraorbital nerve stimulation unit, the ATN stimulation unit, the occipital nerve stimulation unit, the GAN stimulation unit and the infraorbital nerve stimulation unit.

An auricular EEG monitoring system can be used to detect attention deficit hyperactivity disorder (ADHD) or impending ADHD. In some embodiments, an automatic detection-therapy system for ADHD may include an auricular EEG monitoring system, a neuromodulation unit and a processing unit. The auricular EEG monitoring system may be configured to detect ADHD or impending ADHD. When presence of EEG signals suggestive of ADHD is detected, the processing unit is configured to send signals to at least one of the following: a transcutaneous auricular nerve stimulation unit (taVNS unit), a supraorbital nerve stimulation unit, an auriculotemporal nerve (ATN) stimulation unit, an occipital nerve stimulation unit, a greater auricular nerve (GAN) stimulation unit and an infraorbital nerve stimulation unit (or various combinations thereof) to start sending neuromodulating electric stimuli. Likewise, when presence of EEG signals suggestive of impending ADHD is detected, the processing unit is configured to send signals to at least one of the following: the taVNS unit, the supraorbital nerve stimulation unit, the ATN stimulation unit, the occipital nerve stimulation unit, the GAN stimulation unit and the infraorbital nerve stimulation unit (or various combinations thereof) to start sending neuromodulating electric stimuli. When cessation of EEG signals suggestive of ADHD or impending ADHD is detected, the processing unit is configured to send signals to the neuromodulation unit to stop sending neuromodulating electric stimuli from any of the following: the taVNS unit, the supraorbital nerve stimulation unit, the ATN stimulation unit, the occipital nerve stimulation unit, the GAN stimulation unit and the infraorbital nerve stimulation unit.

An auricular EEG monitoring system can be used to detect obsessive compulsive disorder (OCD) or impending OCD. In some embodiments, an automatic detection-therapy system for OCD may include an auricular EEG monitoring system, a neuromodulation unit and a processing unit. The auricular EEG monitoring system may be configured to detect OCD or impending OCD. When presence of EEG signals suggestive of OCD is detected, the processing unit is configured to send signals to at least one of the following: a transcutaneous auricular nerve stimulation unit (taVNS unit), a supraorbital nerve stimulation unit, an auriculotemporal nerve (ATN) stimulation unit, an occipital nerve stimulation unit, a greater auricular nerve (GAN) stimulation unit and an infraorbital nerve stimulation unit (or various combinations thereof) to start sending neuromodulating electric stimuli. Likewise, when presence of EEG signals suggestive of impending OCD is detected, the processing unit is configured to send signals to at least one of the following: the taVNS unit, the supraorbital nerve stimulation unit, the ATN stimulation unit, the occipital nerve stimulation unit, the GAN stimulation unit and the infraorbital nerve stimulation unit (or various combinations thereof) to start sending neuromodulating electric stimuli. When cessation of EEG signals suggestive of OCD or impending OCD is detected, the processing unit is configured to send signals to the neuromodulation unit to stop sending neuromodulating electric stimuli from any of the following: the taVNS unit, the supraorbital nerve stimulation unit, the ATN stimulation unit, the occipital nerve stimulation unit, the GAN stimulation unit and the infraorbital nerve stimulation unit.

An auricular EEG monitoring system can be used to detect autism spectrum disorder (ASD) or impending ASD. In some embodiments, an automatic detection-therapy system for ASD may include an auricular EEG monitoring system, a neuromodulation unit and a processing unit. The auricular EEG monitoring system may be configured to detect ASD or impending ASD. When presence of EEG signals suggestive of ASD is detected, the processing unit is configured to send signals to at least one of the following: a transcutaneous auricular nerve stimulation unit (taVNS unit), a supraorbital nerve stimulation unit, an auriculotemporal nerve (ATN) stimulation unit, an occipital nerve stimulation unit, a greater auricular nerve (GAN) stimulation unit and an infraorbital nerve stimulation unit (or various combinations thereof) to start sending neuromodulating electric stimuli. Likewise, when presence of EEG signals suggestive of impending ASD is detected, the processing unit is configured to send signals to at least one of the following: the taVNS unit, the supraorbital nerve stimulation unit, the ATN stimulation unit, the occipital nerve stimulation unit, the GAN stimulation unit and the infraorbital nerve stimulation unit (or various combinations thereof) to start sending neuromodulating electric stimuli. When cessation of EEG signals suggestive of ASD or impending ASD is detected, the processing unit is configured to send signals to the neuromodulation unit to stop sending neuromodulating electric stimuli from any of the following: the taVNS unit, the supraorbital nerve stimulation unit, the ATN stimulation unit, the occipital nerve stimulation unit, the GAN stimulation unit and the infraorbital nerve stimulation unit.

An auricular EEG monitoring system can be used to detect post-traumatic stress disorder (PTSD) or impending PTSD. In some embodiments, an automatic detection-therapy system for PTSD may include an auricular EEG monitoring system, a neuromodulation unit and a processing unit. The auricular EEG monitoring system may be configured to detect PTSD or impending PTSD. When presence of EEG signals suggestive of PTSD is detected, the processing unit is configured to send signals to at least one of the following: a transcutaneous auricular nerve stimulation unit (taVNS unit), a supraorbital nerve stimulation unit, an auriculotemporal nerve (ATN) stimulation unit, an occipital nerve stimulation unit, a greater auricular nerve (GAN) stimulation unit and an infraorbital nerve stimulation unit (or various combinations thereof) to start sending neuromodulating electric stimuli. Likewise, when presence of EEG signals suggestive of impending PTSD is detected, the processing unit is configured to send signals to at least one of the following: the taVNS unit, the supraorbital nerve stimulation unit, the ATN stimulation unit, the occipital nerve stimulation unit, the GAN stimulation unit and the infraorbital nerve stimulation unit (or various combinations thereof) to start sending neuromodulating electric stimuli. When cessation of EEG signals suggestive of PTSD or impending PTSD is detected, the processing unit is configured to send signals to the neuromodulation unit to stop sending neuromodulating electric stimuli from any of the following: the taVNS unit, the supraorbital nerve stimulation unit, the ATN stimulation unit, the occipital nerve stimulation unit, the GAN stimulation unit and the infraorbital nerve stimulation unit.

In some embodiments, an automatic detection-therapy system for anxiety disorder may include an auricular EEG monitoring system, a neuromodulation unit and a processing unit. The auricular EEG monitoring system may be configured to detect anxiety disorder or impending anxiety disorder. When presence of EEG signals suggestive of anxiety disorder is detected, the processing unit is configured to send signals to at least one of the following: a transcutaneous auricular nerve stimulation unit (taVNS unit), a supraorbital nerve stimulation unit, an auriculotemporal nerve (ATN) stimulation unit, an occipital nerve stimulation unit, a greater auricular nerve (GAN) stimulation unit and an infraorbital nerve stimulation unit (or various combinations thereof) to start sending neuromodulating electric stimuli. Likewise, when presence of EEG signals suggestive of impending anxiety disorder is detected, the processing unit is configured to send signals to at least one of the following: the ta VNS unit, the supraorbital nerve stimulation unit, the ATN stimulation unit, the occipital nerve stimulation unit, the GAN stimulation unit and the infraorbital nerve stimulation unit (or various combinations thereof) to start sending neuromodulating electric stimuli. When cessation of EEG signals suggestive of anxiety disorder or impending anxiety disorder is detected, the processing unit is configured to send signals to the neuromodulation unit to stop sending neuromodulating electric stimuli from any of the following: the taVNS unit, the supraorbital nerve stimulation unit, the ATN stimulation unit, the occipital nerve stimulation unit, the GAN stimulation unit and the infraorbital nerve stimulation unit.

In some embodiments, an automatic detection-therapy system for panic disorder may include an auricular EEG monitoring system, a neuromodulation unit and a processing unit. The auricular EEG monitoring system may be configured to detect panic disorder or impending panic disorder. When presence of EEG signals suggestive of panic disorder is detected, the processing unit is configured to send signals to at least one of the following: a transcutaneous auricular nerve stimulation unit (taVNS unit), a supraorbital nerve stimulation unit, an auriculotemporal nerve (ATN) stimulation unit, an occipital nerve stimulation unit, a greater auricular nerve (GAN) stimulation unit and an infraorbital nerve stimulation unit (or various combinations thereof) to start sending neuromodulating electric stimuli. Likewise, when presence of EEG signals suggestive of impending panic disorder is detected, the processing unit is configured to send signals to at least one of the following: the taVNS unit, the supraorbital nerve stimulation unit, the ATN stimulation unit, the occipital nerve stimulation unit, the GAN stimulation unit and the infraorbital nerve stimulation unit (or various combinations thereof) to start sending neuromodulating electric stimuli. When cessation of EEG signals suggestive of panic disorder or impending panic disorder is detected, the processing unit is configured to send signals to the neuromodulation unit to stop sending neuromodulating electric stimuli from any of the following: the taVNS unit, the supraorbital nerve stimulation unit, the ATN stimulation unit, the occipital nerve stimulation unit, the GAN stimulation unit and the infraorbital nerve stimulation unit.

### BRIEF DESCRIPTION OF THE DRAWINGS

Some embodiments of the present invention are illustrated as an example and are not limited by the figures of the accompanying drawings, in which like references may indicate similar elements and in which:

FIG. 1-FIG. 1 depicts a diagram of an example of an auricular electroencephalogram (EEG) monitoring system in a modified earbud housing, according to various embodiments described herein.

FIG. 2-FIG. 2 illustrates a diagram of an example of an automatic detection-therapy system in a modified earbud housing, according to various embodiments described herein.

FIG. 3-FIG. 3 shows a diagram of a further example of an automatic detection-therapy system having a modified in-the-ear housing that is placed inside the tragus-concha bowl of the wearer's ear, according to various embodiments described herein.

FIG. 4-FIG. 4 illustrates an elevation view of the anatomy of a human external ear, including the locations of tragus, cymba-concha, cavum-concha, triangular fossa, helix, lobule, external ear canal, etc.

FIG. 5-FIG. 5 shows a diagram of a further example of an automatic detection-therapy system in a behind-the-ear-hearing-aid-style housing, according to various embodiments described herein.

FIG. 6-FIG. 6 depicts a diagram of yet a further example of an automatic detection-therapy system in a modified in-the-ear housing, according to various embodiments described herein.

FIG. 7-FIG. 7 depicts a diagram of still another example of an automatic detection-therapy system having a modified earbud housing engaged to an ear of a wearer according to various embodiments described herein.

FIG. 8-FIG. 8 shows a diagram of an example of an auricular electroencephalogram (EEG) monitoring system having a modified earbud housing engaged or coupled to an ear of a wearer according to various embodiments described herein.

FIG. 9-FIG. 9 depicts a diagram of an example of an auricular electroencephalogram (EEG) monitoring system having a tubular-shaped structure engaged or coupled to the ear of a wearer according to various embodiments described herein.

FIG. 10-FIG. 10 illustrates a diagram of an example of an auricular electroencephalogram (EEG) monitoring system having a behind-the-ear-hearing-aid-style housing engaged or coupled to an ear of a wearer according to various embodiments described herein.

FIG. 11-FIG. 11 shows a diagram of an example of an automatic detection-therapy system having a modified earbud housing engaged or coupled to an ear of a wearer according to various embodiments described herein.

FIG. 12-FIG. 12 illustrates a diagram of an example of an automatic detection-therapy system having a modified in-the-ear housing engaged or coupled to an ear of a wearer according to various embodiments described herein

FIG. 13-FIG. 13 depicts a diagram of an example of an automatic detection-therapy system having a behind-the-ear-hearing-aid-style housing engaged or coupled to an ear of a wearer according to various embodiments described herein

FIG. 14-FIG. 14 illustrates a block diagram showing some exemplary elements of an example of a processing unit for an EEG monitoring system or an automatic detection-therapy system according to various embodiments described herein.

FIG. 15-FIG. 15 shows a block diagram showing some exemplary elements of an example of an auricular EEG monitoring system according to various embodiments described herein.

FIG. 16-FIG. 16 depicts a block diagram illustrating some exemplary elements of an example of a transcutaneous auricular vagus nerve stimulation (taVNS) unit according to various embodiments described herein.

FIG. 17-FIG. 17 shows a block diagram illustrating some exemplary elements of an example of a client device according to various embodiments described herein.

FIG. 18-FIG. 18 illustrates a schematic diagram illustrating some exemplary components of auricular EEG monitoring system and some exemplary components of an automatic detection-therapy system according to various embodiments described herein.

FIG. 19-FIG. 19 illustrates a perspective diagram of an example of a tubular-shaped structure for housing of the EEG sensor electrodes and some exemplary locations for the EEG sensor electrodes according to various embodiments described herein.

FIG. 20-FIG. 20 illustrates a side elevation diagram of an example of a tubular-shaped structure for housing of the EEG sensor electrodes and some exemplary locations for the EEG sensor electrodes according to various embodiments described herein.

FIG. 21-FIG. 21 shows a sectional, through line 21-21 shown in FIG. 20, elevation view of an example of a tubular-shaped structure for housing of the EEG sensor electrodes and some exemplary locations for the EEG sensor electrodes according to various embodiments described herein.

FIG. 22-FIG. 22 depicts a sectional, through line 22-22 shown in FIG. 20, elevation view of an example of a tubular-shaped structure for housing of the EEG sensor electrodes and some exemplary locations for the EEG sensor electrodes according to various embodiments described herein.

FIG. 23-FIG. 23 shows a sectional, through line 23-23 shown in FIG. 20, elevation view of an example of a tubular-shaped structure for housing of the EEG sensor electrodes and some exemplary locations for the EEG sensor electrodes according to various embodiments described herein.

FIG. 24-FIG. 24 illustrates a sectional, through line 24-24 shown in FIG. 20, elevation view of an example of a tubular-shaped structure for housing of the EEG sensor electrodes and some exemplary locations for the EEG sensor electrodes according to various embodiments described herein.

FIG. 25-FIG. 25 depicts a sectional, through line 25-25 shown in FIG. 20, elevation view of an example of a tubular-shaped structure for housing of the EEG sensor electrodes and some exemplary locations for the EEG sensor electrodes according to various embodiments described herein.

FIG. 26-FIG. 26 shows a sectional, through line 26-26 shown in FIG. 20, elevation view of an example of a tubular-shaped structure for housing of the EEG sensor electrodes and some exemplary locations for the EEG sensor electrodes according to various embodiments described herein.

FIG. 27-FIG. 27 depicts a diagram of an example of a combined neuromodulation system placed in the tragus-concha bowl of a wearer's ear, according to various embodiments described herein.

FIG. 28-FIG. 28 shows a diagram of still a further example of an automatic detection-therapy system having a behind-the-ear-hearing-aid-style housing according to various embodiments described herein.

FIG. 29-FIG. 29 shows a diagram of a supraorbital nerve stimulation unit.

FIG. 30-FIG. 30 shows a diagram of an occipital nerve stimulation unit with a transparent view of the occipital nerves.

FIG. 31-FIG. 31 shows a block diagram illustrating some exemplary elements of an example of a trigeminal nerve stimulation unit. Examples of a trigeminal nerve stimulation unit includes a supraorbital nerve stimulation unit, an auriculotemporal nerve stimulation unit and an infraorbital nerve stimulation unit.

FIG. 32-FIG. 32 shows a block diagram illustrating some exemplary elements of an example of an occipital nerve stimulation unit.

FIG. 33-FIG. 33 shows a block diagram illustrating some exemplary elements of an example of a greater auricular nerve stimulation unit.

### DETAILED DESCRIPTION OF THE INVENTION

The terminology used herein is for the purpose of describing particular embodiments only and is not intended to be limiting of the invention. As used herein, the term “and/or” includes any and all combinations of one or more of the associated listed items. As used herein, the singular forms “a,” “an,” and “the” are intended to include the plural forms as well as the singular forms, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

Unless otherwise defined, all terms (including technical and scientific terms) used herein have the same meaning as commonly understood by one having ordinary skill in the art to which this invention belongs. It will be further understood that terms, such as those defined in commonly used dictionaries, may be interpreted as having a meaning that is consistent with their meaning in the context of the relevant art and the present disclosure and will not be interpreted in an idealized or overly formal sense unless expressly so defined herein.

In describing the invention, it will be understood that a number of techniques and steps are disclosed. Each of these has individual benefit and each can also be used in conjunction with one or more, or in some cases all, of the other disclosed techniques. Accordingly, for the sake of clarity, this description will refrain from repeating every possible combination of the individual steps in an unnecessary fashion. Nevertheless, the specification and claims may be read with the understanding that such combinations are entirely within the scope of the invention and the claims.

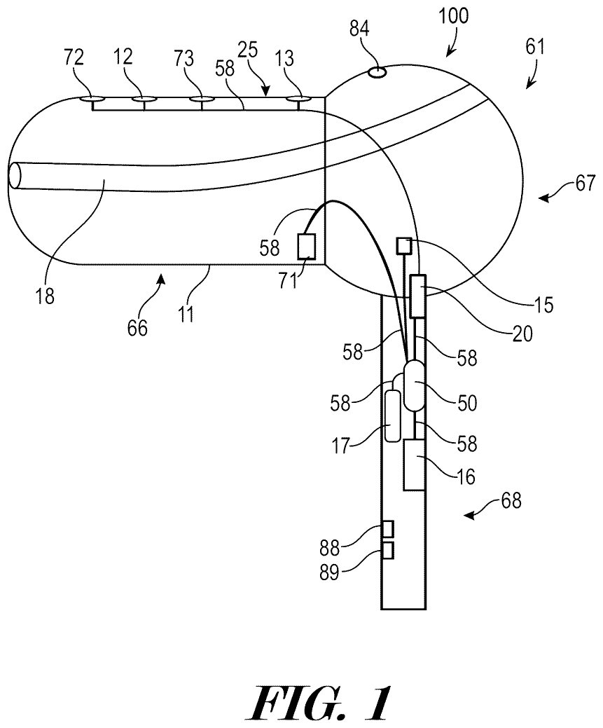

For purposes of description herein, the terms “upper”, “lower”, “left”, “right”, “rear”, “front”, “side”, “vertical”, “horizontal”, and derivatives thereof shall relate to the invention as oriented in FIG. 1. However, one will understand that the invention may assume various alternative orientations and step sequences, except where expressly specified to the contrary. Therefore, the specific devices and processes illustrated in the attached drawings, and described in the following specification, are simply exemplary embodiments of the inventive concepts defined in the appended claims. Hence, specific dimensions and other physical characteristics relating to the embodiments disclosed herein are not to be considered as limiting, unless the claims expressly state otherwise.

The term “client device” as used herein is a type of computer or computing device comprising circuitry and configured to generally perform functions such as recording audio, photos, and videos; displaying or reproducing audio, photos, and videos; storing, retrieving, or manipulation of electronic data; providing electrical communications and network connectivity; or any other similar function. Non-limiting examples of client devices include: personal computers (PCs), workstations, servers, laptops, tablet PCs including the iPad, cell phones including iOS phones made by Apple Inc., Android OS phones, Microsoft OS phones, Blackberry phones, Apple iPads, Anota digital pens, smart watches (e.g., Apple Watch, Samsung Galaxy Watch, etc.), digital music players, or any electronic device capable of running computer software and displaying information to a user, memory cards, other memory storage devices, digital cameras, external battery packs, external charging devices, and the like. Certain types of electronic devices which are portable and easily carried by a person from one location to another may sometimes be referred to as a “portable electronic device” or “portable device”. Some nonlimiting examples of portable devices include: cell phones, smartphones, tablet computers, laptop computers, tablets, digital pens, wearable computers such as Apple Watch, other smartwatches, Fitbit, other wearable fitness trackers, Google Glasses, and the like.

As used herein the term “data network” or “network” shall mean an infrastructure capable of connecting two or more computers such as client devices either using wires or wirelessly allowing them to transmit and receive data. Non-limiting examples of data networks may include the internet or wireless networks or (i.e., a “wireless network”) which may include BLE (Bluetooth), LoRa and LoRaWAN (and other low-power, wide-area (LPWA) networking protocols), Wi-Fi, and cellular networks. For example, a network may include a local area network (LAN), a wide area network (WAN) (e.g., the Internet), a mobile relay network, a metropolitan area network (MAN), an ad hoc network, a telephone network (e.g., a Public Switched Telephone Network (PSTN)), a cellular network, a Zigbee network, or a voice-over-IP (VOIP) network.

Although the terms “first”, “second”, etc. are used herein to describe various elements, these elements may not be limited by these terms. These terms are only used to distinguish one element from another element. For example, the first element may be designated as the second element, and the second element may be likewise designated as the first element without departing from the scope of the invention.

As used in this application, the term “about” or “approximately” refers to a range of values within plus or minus 15% of the specified number. Additionally, as used in this application, the term “substantially” means that the actual value is within about 10% of the actual desired value, particularly within about 5% of the actual desired value and especially within about 1% of the actual desired value of any variable, element or limit set forth herein.

A new auricular electroencephalogram (EEG) monitoring system and an automatic detection-therapy system are discussed herein. In the following description, for purposes of explanation, numerous specific details are set forth in order to provide a thorough understanding of the present invention. It will be evident, however, to one skilled in the art that the present invention may be practiced without these specific details.

The present disclosure is to be considered as an exemplification of the invention and is not intended to limit the invention to the specific embodiments illustrated by the figures or description below.

The present invention will now be described by example and through referencing the appended figures representing preferred and alternative embodiments.

According to one embodiment consistent with the principles of the present invention, an auricular electroencephalogram (EEG) monitoring system (“the auricular EEG monitoring system”) 100 is disclosed (FIGS. 1, 8-10, and 18). In some embodiments, the auricular EEG monitoring system 100 may comprise one or more auricular EEG recording modules 20 which may have a plurality of (at least two, but preferably more than two) wired or wireless EEG sensor electrodes 12, 13, 72, 73, 82, 83. Each auricular EEG recording module 20 may have a plurality of EEG sensor electrodes 12, 13, 72, 73, 82, 83. Optionally, the EEG recording module 20 may further comprises an optional wired or wireless reference electrode 84, to act as a baseline voltage reference for other active EEG sensor electrodes 12, 13, 72, 73, 82, 83. (The optional reference electrode 84 would be desirable but is not always needed. Instead, average of all of the EEG sensor electrodes 12, 13, 72, 73, 82, 83, can be used as a reference, i.e. common average reference). The EEG sensor electrodes 12, 13, 72, 73, 82, 83, and the optional reference electrode 84 are configured to contact separate areas of the wearer's ear 902 or peri-auricular area 903. The areas that the EEG sensor electrodes 12, 13, 72, 73, 82, 83, and the optional reference electrode 84 are configured to contact may be selected from at least one of the following: the external ear 902, external ear canal 904, and peri-auricular area 903. Preferably, all of the EEG sensor electrodes 12, 13, 72, 73, 82, 83, may be configured to contact the skin of the wearer's external ear canal 904, while the optional reference electrode 84 may be configured to contact the skin of the tragus-concha bowl 957 or the skin of the mastoid in the peri-auricular area 903. The peri-auricular area 903 refers to the portion of the head around the auricle. The peri-auricular area 903 is typically hairless. The peri-auricular area 903 includes a portion of the head in front of the auricle (preauricular area) and a portion of the head above and behind the auricle (post-auricular area). The pre-auricular area is small, about one inch wide and about two inches long and curved along the anterior edge of the auricle. The post-auricular area is also small and is about one inch wide and about three inches long and curved along the superior and posterior edges of the auricle. The post-auricular area is where a behind-the-ear hearing aid is usually attached to. The preauricular area and the post-auricular area together is called “peri-auricular area” herein. (Anterior, posterior, superior, in front of and behind etc. all refer to the directions relative to the wearer's head when the wearer is in an upright position.) The auricular EEG recording module 20 may be configured to record EEG data of the wearer 900 via electrical activities detected by the EEG sensor electrodes 12, 13 72, 73, 82, 83, and the optional reference electrode 84 (if incorporated). A network interface 53, 406, may be configured to generate a notification to a client device 400. A processing unit 50, 401, may be in electronic communication with the first auricular EEG recording module 20 and the network interface 53, 406. Preferably, the processing unit 50, 401, may be configured to analyze the EEG data recorded by the auricular EEG recording module 20 to detect presence or cessation of EEG signals suggestive of neuropsychiatric disorders. When the presence or cessation of EEG signals suggestive of at least one neuropsychiatric disorder is detected, the processing unit 50, 401, may be further configured to immediately send signals to the network interface 53, 406, to generate a notification to the client device 400, such as to a client device 400 of a wearer 900 or a client device 400 of the wearer's healthcare provider 950.

The auricular EEG recording module 20 may be configured to record EEG data of the wearer 900 via miniature wired or wireless dry EEG sensor electrodes 12, 13, 72, 73, 82, 83, and the optional wired or wireless dry reference electrode 84 (wired and wireless miniature dry EEG electrodes as known in the art). The recorded EEG data may be transmitted or otherwise electronically communicated wired or wirelessly to a processing unit 50, 401. With the help of various advanced EEG analysis algorithms together with machine learning, deep learning and artificial intelligence (as known in the art), the processing unit 50, 401, may be configured to analyze the EEG data to detect presence or cessation of EEG signals suggestive of neuropsychiatric disorders. The processing unit 50, 401, may also be configured to analyze the EEG data to detect presence or cessation of EEG signals suggestive of impending neuropsychiatric disorders. These neuropsychiatric disorders include seizure, migraine, cluster headache, major depressive disorder, bipolar disorder, schizophrenia, obsessive compulsive disorder, attention deficit and hyperactivity disorder, autism spectrum disorder, post-traumatic stress disorder, anxiety disorder and panic disorder, etc.

In some embodiments, a processing unit 50, 401, of the auricular EEG monitoring system 100 may be configured to analyze the EEG data recorded by the auricular EEG recording module 20 to detect presence or cessation of EEG signals suggestive of neuropsychiatric disorders. When presence of EEG signals suggestive of at least one neuropsychiatric disorder is detected, the processing unit 50, 401, may be further configured to immediately send signals to the network interface 53 to generate a notification to a client device 400, such as to a client device 400 of a wearer 900 or a client device 400 of the wearer's healthcare provider 950. In further embodiments, the processing unit 50, 401, may be configured to analyze the EEG data recorded by the auricular EEG recording module 20 to detect presence or cessation of EEG signals suggestive of impending neuropsychiatric disorders. When the presence of EEG signals suggestive of impending neuropsychiatric disorder is detected, the processing unit 50, 401, may be further configured to immediately send signals to the network interface 53 to generate a notification to the client device 400, such as to a client device 400 of a wearer 900 or a client device 400 of the wearer's healthcare provider 950.

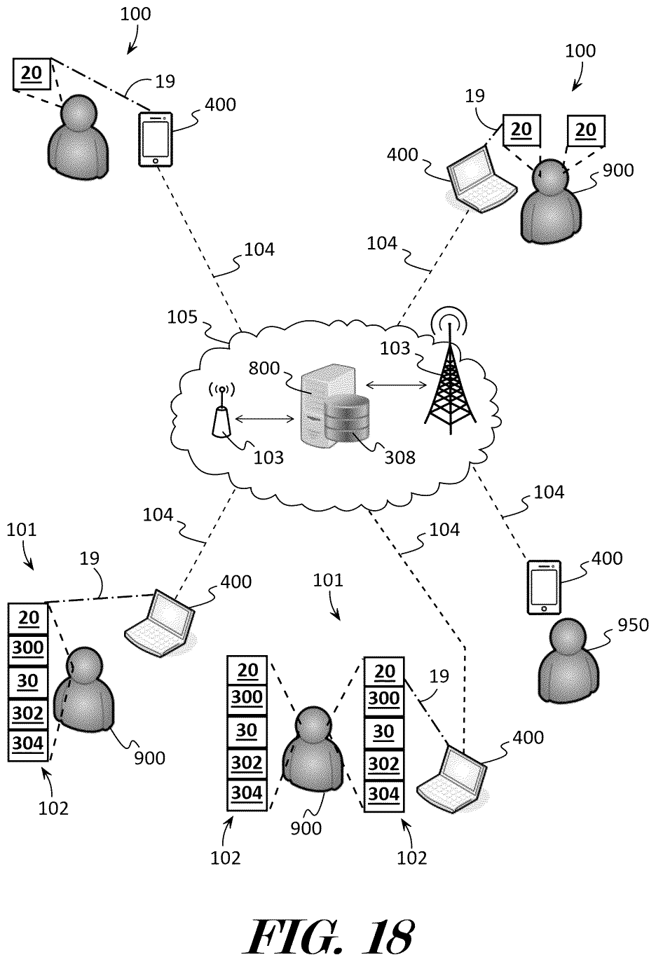

It should be understood that the auricular EEG monitoring system 100 may comprise one or more auricular EEG recording modules 20 as shown in FIG. 18. For example, the auricular EEG monitoring system 100 may comprise a first auricular EEG recording module 20 with its EEG sensor electrodes 12, 13, 72, 73, 82, 83, and a first optional reference electrode 84, attached to the wearer's 900 first ear 902 or a peri-auricular area 903 around the wearer's first ear and a second auricular EEG recording module 20 with its EEG sensor electrodes 12, 13, 72, 73, 82, 83, and a second optional reference electrode 84 attached to the wearer's 900 second ear 902 or a peri-auricular area 903 around the wearer's second ear. The first auricular EEG recording module 20 may include a plurality of (at least two, but preferably more than two) EEG sensor electrodes, 12, 13, 72, 73, 82, 83, and the first optional reference electrode 84 and all of these electrodes are configured to contact separate areas of the wearer's first ear 902 or peri-auricular area 903 around the wearer's first ear. The areas that the EEG sensor electrodes 12, 13, 72, 73, 82, 83, and the optional first reference electrode 84 may be configured to contact are selected from at least one of the following: the first external ear 902, first external ear canal 904, and first peri-auricular area 903. Preferably, all of the EEG sensor electrodes 12, 13, 72, 73, 82, 83, of the first auricular EEG recording module 20 may be configured to contact the skin of the external ear canal 904 of the wearer's first ear 902, while the first optional reference electrode 84 may be configured to contact the skin of the tragus-concha bowl 957 of the wearer's first ear 902 or the mastoid of the first peri-auricular area 903. Likewise, the second auricular EEG recording module 20 may include a plurality of (at least two, but preferably more than two) EEG sensor electrodes 12, 13, 72, 73, 82, 83, and a second optional reference electrode 84. These electrodes 12, 13, 72, 73, 82, 83, 84, may be configured to contact separate areas of the wearer's second ear 902 or peri-auricular area 903 round the wearer's second ear. Preferably, all of the EEG sensor electrodes 12, 13, 72, 73, 82, 83, of the second auricular EEG recording module 20 may be configured to contact the skin of the external ear canal 904 of the wearer's second ear 902, while the second optional reference electrode 84 may be configured to contact the skin of the tragus-concha bowl 957 of the wearer's second ear or the mastoid of the wearer's second peri-auricular area 903. The second auricular EEG recording module 20 may also be configured to record EEG data of the wearer 900, and the second EEG recording module 20 may also be in electronic communication with the processing unit 50 so that the processing unit 50 may use EEG data from the first auricular EEG recording module 20 and the second auricular EEG recording module 20 to detect the presence or cessation of EEG signals suggestive neuropsychiatric disorders and/or to detect the presence or cessation of EEG signals suggestive of impending neuropsychiatric disorders.

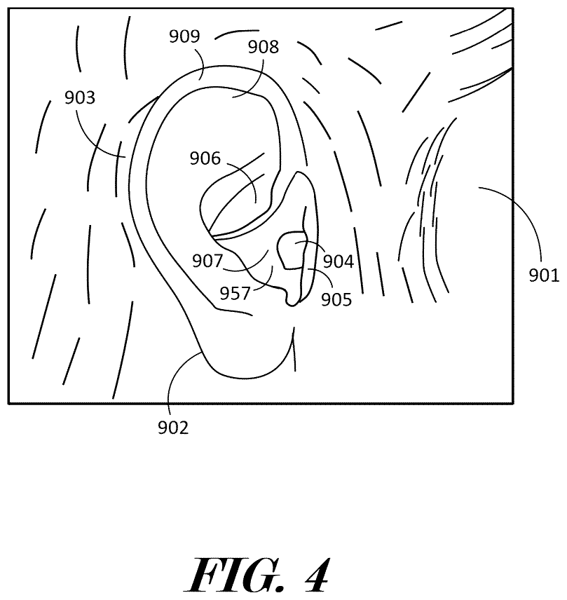

In preferred embodiments, the auricular EEG monitoring system 100 may comprise a miniature auricular EEG recording module 20 that may include a plurality of miniature EEG sensor electrodes 12, 13. 72, 73, 82, 83, and an optional reference electrode 84. All of the EEG sensor electrodes 12, 13, 72, 73, 82, 83, and the optional reference electrode 84 may be linked to an ear 902 of the wearer, such as be housed or contained in an auricular housing 11 (housing refers to protective covers or protective structures). The auricular housing 11 may be attached to the head 901, preferably to the external ear canal 904 or external ear 902 or the peri-auricular area 903. (As referred to herein, the external ear canal 904 refers to the part of the ear 902 that connects the visible outer ear (pinna) to the middle ear, essentially the tube that carries sound waves to the eardrum). The anatomy of human external ear 902 and head 901 proximate to the ear 902 is shown in FIG. 4. As used herein, the term “tragus-concha bowl” 957 refers to an area surrounding the opening of the external ear canal 904 and this area is bounded by inner tragus 905 and concha 906, 907, forming a bowl-shaped cavity to funnel sound into the external ear canal 904. This bowl-shaped cavity is called tragus-concha bowl 957 herein (official name is concha auriculae and is sometimes referred to simply as the concha bowl).

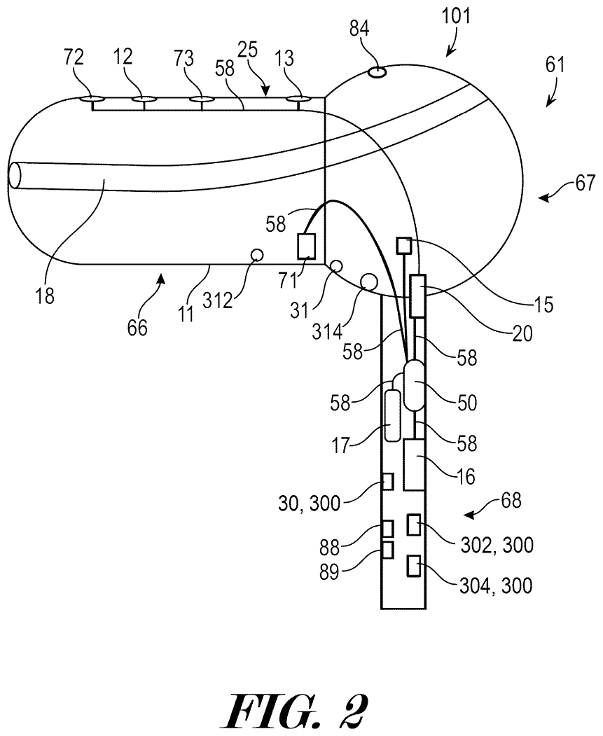

In some embodiments, an auricular housing 11 of an auricular EEG monitoring system 100 and/or an automatic detection-therapy system 101 may be configured in any size and shape that may be suitable for being attached to the external ear 902, the external ear canal 904, the tragus-concha bowl 957 or the peri-auricular area 903. In some embodiments, an auricular housing 11 may comprise a sound conduit 18 which may extend through the auricular housing 11 (such as shown in FIGS. 1 and 2) and which may facilitate the ability of sound to enter the ear 902. A sound conduit 18 may comprise an opening, channel, conduit, etc., which may extend through a portion of the auricular housing 11 so that sound waves may pass through the sound conduit 18 to facilitate or enable the wearer 900 to hear sounds in the environment. The sound conduit 18 may also help to equalize air pressure between the external ear canal 904 and the outside environment, reducing pressure buildup, improving comfort, and enhancing sound fidelity.

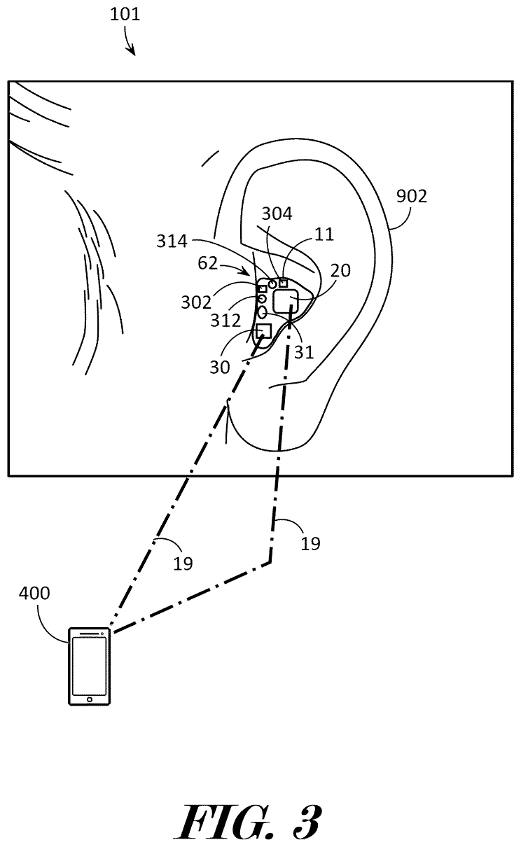

In some embodiments, an auricular housing 11 of an auricular EEG monitoring system 100 and/or an automatic detection-therapy system 101 may be shaped or configured as a modified in-the-ear housing 62. (A modified in-the-ear housing 62 may be modified from an in-the-ear (ITE) hearing aid to include a tubular-shaped structure 66 and a body-structure 67. The tubular-shaped structure 66 has an elongated tubular part that is inserted into a wearer's external ear canal 904 when in use. The body-structure 67 is located at the opening of the external ear canal 904 and sits or be placed inside the tragus-concha bowl 957 when in use.) All or a majority of the auricular housing 11 may be inserted into a portion of the external ear canal 904 and the tragus-concha bowl 957, such as shown in FIGS. 3, 12. Example of a modified in-the-ear housing 62 is modified from a combination of an in-the-ear (ITE) hearing aid and an in-the-canal (ITC) hearing aid.

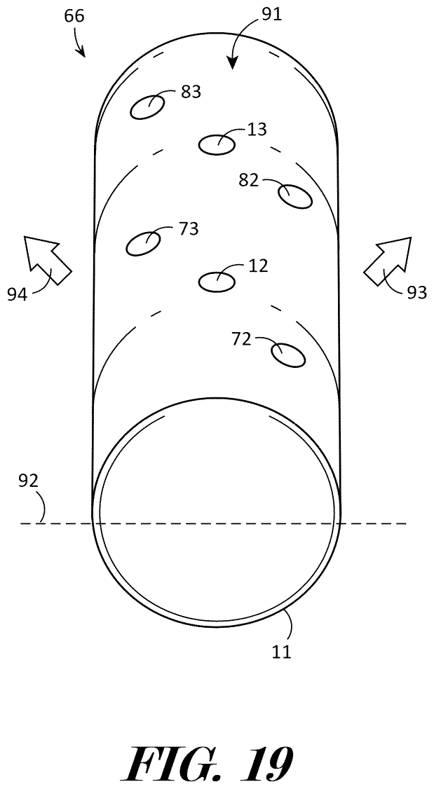

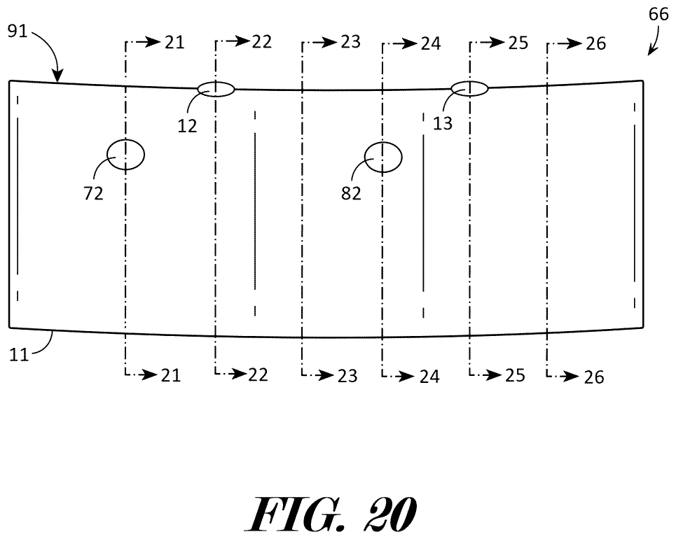

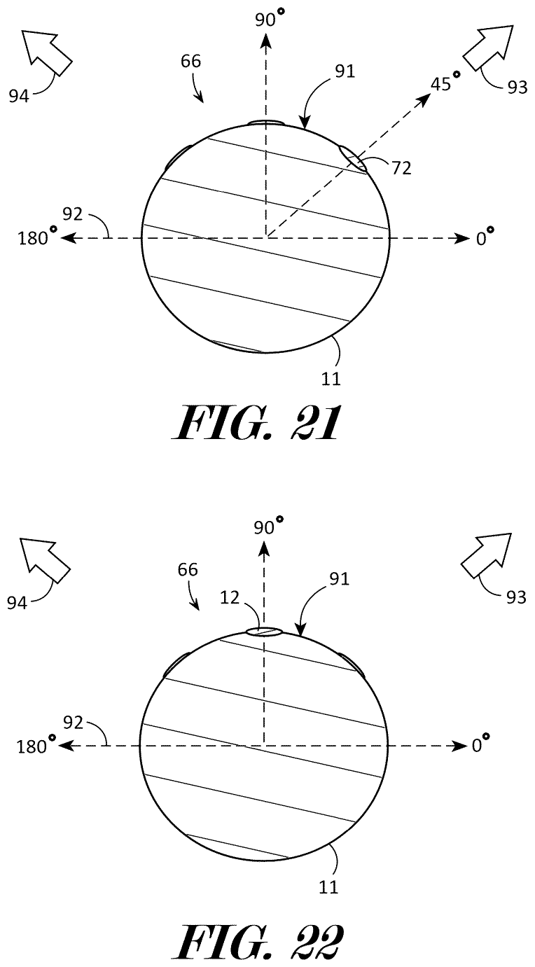

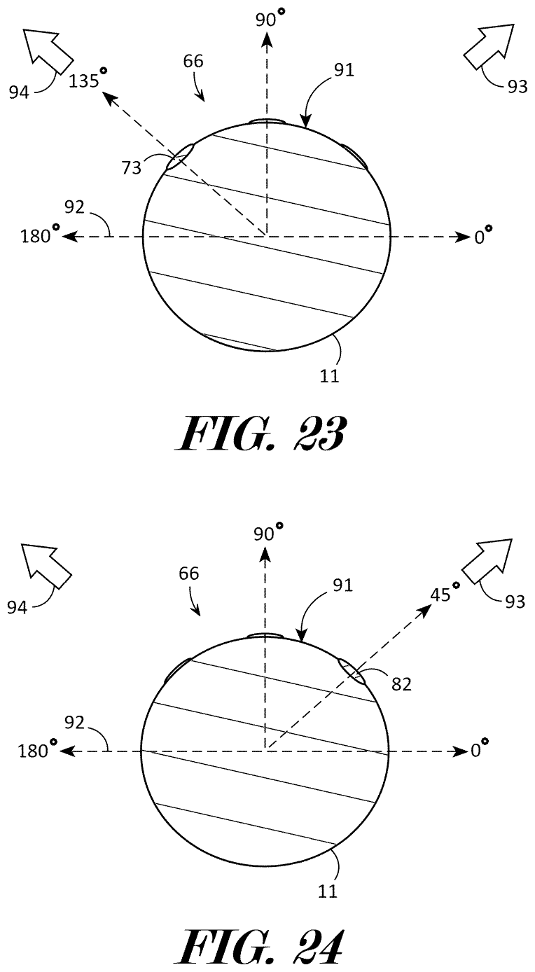

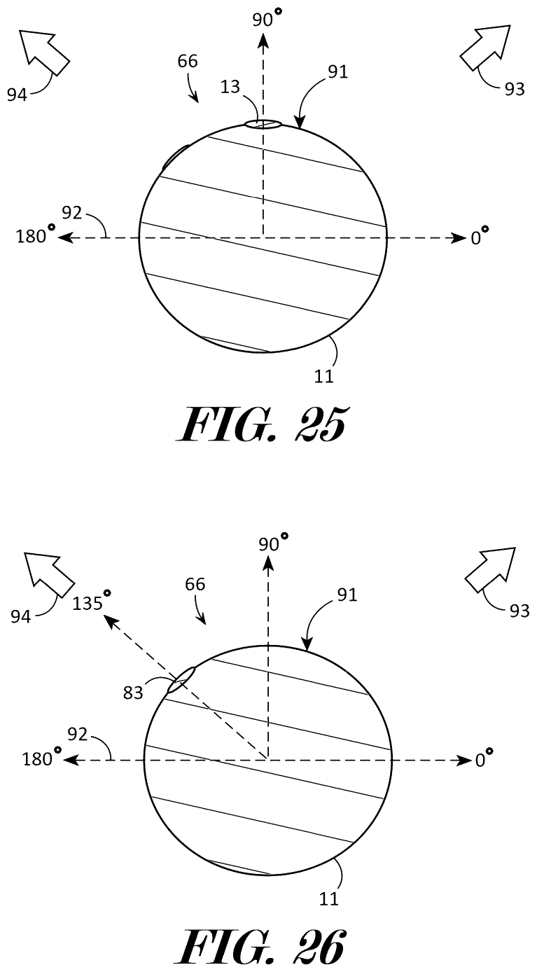

In some embodiments, an auricular housing 11 of an auricular EEG monitoring system 100 and/or an automatic detection-therapy system 101 may be configured to include a tubular-shaped structure 66. All of the EEG sensor electrodes 12, 13, 72, 73, 82, 83 are configured to be located at the surface 91 of the tubular-shaped structure 66 and so that all of the EEG sensor electrodes 12, 13, 72, 73, 82, 83 are housed in the tubular-shaped structure 66. Optionally, the other components for system 100 and/or system 101 may be configured to be located or housed in other portions of the auricular housing 11, to be described hereinafter. Alternatively, the tubular-shaped structure 66 may be configured as a standalone tubular-shaped structure 66 for housing of all of the EEG sensor electrodes 12, 13, 72, 73, 82, 83 and most of the other components of system 100 and/or system 101. The tubular-shaped structure 66 is configured to be inserted into a wearer's external ear canal 904 when in use. In preferred embodiments, one or more of the EEG sensor electrodes 12, 13, 72, 73, 82, 83 is/are configured to be located at the upper surface 91 (upper surface at approximately 90 degrees above horizontal level 92, as shown by electrodes 12, 13, in FIGS. 19, 22, 25) of the tubular-shaped structure 66. In preferred embodiments, one or more of the EEG sensor electrodes is/are configured to be located at between 0 and 90 degrees, and more preferably at approximately 45 degrees (plus or minus fifteen degrees, i.e. 30-60 degrees) above horizontal level 92 of the tubular-shaped structure 66 and is/are configured to face forward-upward direction 93 (e.g., as shown by electrodes 72, 82, in FIGS. 19, 21, 24). In preferred embodiments, one or more of the EEG sensor electrodes is/are configured to be located at between 90 and 180 degrees, and more preferably at approximately 135 degrees (plus or minus fifteen degrees, i.e. 120-150 degrees) above the horizontal level 92 of the tubular-shaped structure 66 and is/are configured to face backward-upward direction 94 (e.g., as shown by electrodes 73, 83, in FIGS. 19, 23, 26). (Upper surface, horizontal level, forward, backward and upward all refer to directions relative to the head 901 of the wearer 900 with the wearer in upright position after the tubular-shaped structure 66 has been inserted into the wearer's external ear canal 904.) These arrangements will enable the EEG sensor electrodes 12, 13, 72, 73, 82, 83, to have one of the best locations and directions from the external ear canal 904 to record the wearer's EEG activities.

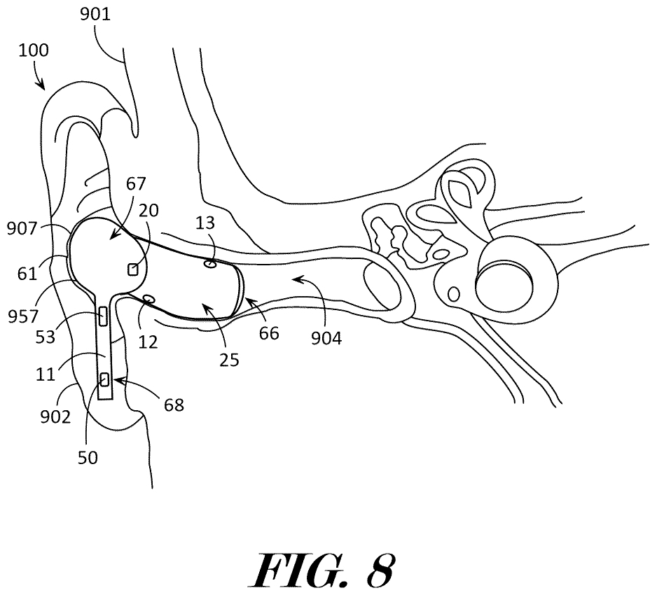

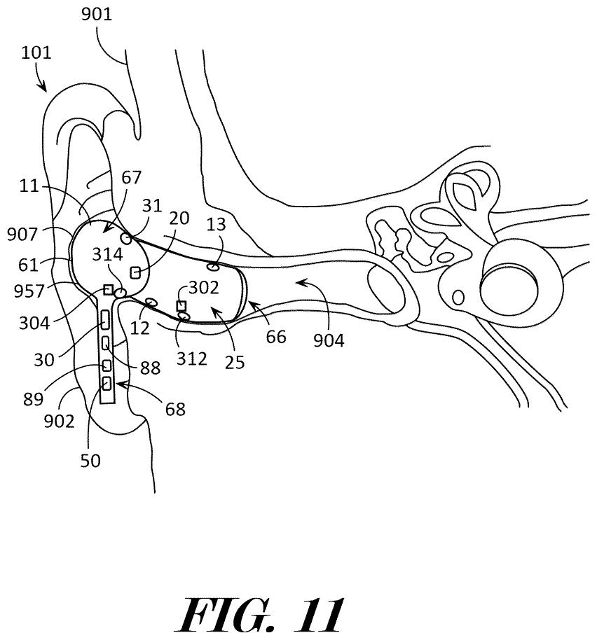

In some embodiments, an auricular housing 11 of an auricular EEG monitoring system 100 and/or an automatic detection-therapy system 101 may be shaped or configured as a modified earbud housing 61. (housing refers to protective covers or protective structures). The modified earbud housing 61 includes a tubular-body portion 25 and a stem portion (“stem” or “stalk” portion) 68. The tubular-body portion 25 comprises a tubular-shaped structure 66 and a body-structure 67. The tubular-shaped structure 66 is essentially an elongated version of an “ear-tip” and a “nozzle” of an earbud and the tubular-shaped structure 66 can be inserted into a wearer's external ear canal 904 when in use. The body-structure 67 is essentially similar to a “body” (or a “shell”) of an earbud. The body-structure 67 is placed at the opening of a wearer's external ear canal 904 and sits (or be placed) inside a tragus-concha bowl 957 when in use. The modified earbud housing 61 may be shaped and sized so that when the tubular-body portion 25 is placed in a wearer's external ear 902 when in use, the tubular-shaped structure 66 is inserted into the wearer's external ear canal 904 and the body-structure 67 is sitting inside the tragus-concha bowl 957 which is immediately outside the opening of the external ear canal 904, such as shown in FIGS. 8 and 11. Examples that a modified earbud housing 61 is modified from include: earbuds, ear phones, Apple AirPods®, in-ear monitors (IEM) and the like. As used herein, the term “tragus-concha bowl” 957 refers to an area surrounding the opening of the external ear canal 904 and this area is bounded by inner tragus 905 and concha 906, 907, forming a bowl-shaped cavity to funnel sound into the external ear canal 904. This bowl-shaped cavity is called tragus-concha bowl 957 herein (official name is concha auriculae and is sometimes referred to simply as the concha bowl).

In some embodiments of an auricular EEG monitoring system 100, all or portion of the following: an EEG recording module 20 and its EEG sensor electrodes 12, 13, 72, 73, 82, 83, a processing unit 50, and a network interface 53 may be housed or contained in a modified earbud housing 61 (FIGS. 1 and 8). The modified earbud housing 61 includes a tubular-body portion 25 and a stem portion 68. The tubular-body portion 25 includes a tubular-shaped structure 66 and a body-structure 67. All of the EEG sensor electrodes 12, 13, 72, 73, 82, 83, may be located on a surface and partially embedded in the surface with slight protrusion at the surface of the tubular-shaped structure 66. An optional reference electrode 84 may be located on a surface and partially embedded in the surface with slight protrusion at the surface of the body-structure 67. Preferably, the tubular-body portion 25 (including the tubular-shaped structure 66 and the body-structure 67) may comprise or may be made from an elastic flexible and adaptable material. The material of the tubular-body portion 25 of the modified earbud housing 61 is configured to have appropriate elasticity flexibility and adaptability so that all of the EEG sensor electrodes 12, 13, 72, 73, 82, 83, of the EEG recording module 20 are naturally in close contact (provided by the elastic flexible, and adaptable characteristics of the material so that it can conform to the contours of the external ear canal 904 and the tragus-concha bowl 957 and snugly fill the interior of the external ear canal 904 and the tragus-concha bowl 957) with the skin of the external ear canal 904 of the wearer's ear 902 and so that the optional reference electrode 84 is naturally in close contact with the skin of the tragus-concha bowl 957 of the wearer's ear when the tubular-body portion 25 of the modified earbud housing 61 is placed in the wearer's ear 902.

Similarly, in some embodiments of an automatic detection-therapy system 101, all or portion of the following: an EEG recording module 20 with its EEG sensor electrodes 12, 13, 72, 73, 82, 83, a processing unit 50, a neuromodulation unit 300 and a network interface 53 may be housed or contained in a modified earbud housing 61. The neuromodulation unit 300 may include at least one of the following components: a taVNS unit 30, an auriculotemporal nerve (ATN) stimulation unit 302, a supraorbital nerve stimulation unit 301, an occipital nerve stimulation unit 303, a greater auricular nerve (GAN) stimulation unit 304 and an infraorbital nerve stimulation unit 305. In some embodiments, a neuromodulation unit 300 may have 2 components, for example a taVNS unit 30, and an auriculotemporal nerve (ATN) stimulation unit 302. (Other examples of a neuromodulation unit 300 having only one component or combination of two different components would be similar to the following description). Combination of the taVNS unit 30 and the ATN stimulation unit 302 into a single auricular housing 11, such as a modified earbud housing 61, for automatic detection-therapy system 101 would be preferred due to the proximity and overlapping of their target skin areas and their synergistic effect. The taVNS unit 30 comprises a taVNS stimulating electrode 31 that is configured to give pre-determined neuromodulating electric stimuli to vagus innervated auricular skin of the wearer 900 when activated or prompted. The auriculotemporal nerve (ATN) stimulation unit 302 comprises an ATN stimulation electrode 312 that is configured to give pre-determined neuromodulating electric stimulation to the wearer's ATN innervated auricular skin when activated or prompted. Optionally, the auricular EEG recording module 20 may contain an optional reference electrode 84 (to act as a baseline voltage reference for other active EEG sensor electrodes 12, 13, 72, 73, 82, 83). All of the EEG sensor electrodes 12, 13, 72, 73, 82, 83, the optional reference electrode 84, the taVNS stimulating electrode 31 and the ATN stimulating electrode 312 are configured to be housed or contained in the modified earbud housing 61 (FIGS. 2, 7, and 11). The modified earbud housing 61 includes a tubular-body portion 25 and a stem portion 68. The tubular-body portion 25 includes a tubular-shaped structure 66 (to be inserted into a wearer's external ear canal 904 when in use) and a body-structure 67 (to be placed at the opening of the wearer's external ear canal 904 and to sit or be placed inside the wearer's tragus-concha bowl 957 when in use). The tubular-shaped structure 66 is equivalent to an elongated version of a “nozzle” and an “ear-tip” of an earbud. The body-structure 67 is equivalent to a “body” (or “shell”) of an earbud. All of the EEG sensor electrodes 12, 13, 72, 73, 82, 83, the optional reference electrode 84, the taVNS stimulating electrode 31 and the auriculotemporal nerve (ATN) stimulating electrode 312 may be located on a surface of the tubular-body portion 25 of the modified earbud housing 61. All of the EEG sensor electrodes 12, 13, 72, 73, 82, 83, the optional reference electrode 84, the taVNS stimulating electrode 31 and the ATN stimulating electrode 312 may be configured to be partially embedded in the surface with slight protrusion at the surface of the tubular-body portion 25 of the modified earbud housing 61. Preferably all of the EEG sensor electrodes 12, 13, 72, 73, 82, 83, may be located on the surface 91 (and partially embedded in the surface with protrusion at the surface) of the tubular-shaped structure 66. Preferably the ta VNS stimulating electrode 31 and the auriculotemporal nerve (ATN) stimulating electrode 312 may be located on the surface (and partially embedded in the surface with protrusion at the surface) of one of; the body-structure 67 and the tubular-shaped structure 66. Preferably, the optional reference electrode 84 may be configured to be located on a surface (and partially embedded in the surface with slight protrusion at the surface) of the body-structure 67. The tubular-body portion 25 may comprise or may be made from an elastic flexible and adaptable material. The material for the tubular-body portion 25 of the modified earbud housing 61 is configured to have appropriate elasticity flexibility and adaptability so that the tubular-shaped structure 66 will naturally adapt to the contour and fill the interior of the wearer's external ear canal 904 when the tubular-shaped structure 66 is inserted into the wearer's external ear canal 904, and, meanwhile, the body-structure 67 will naturally adapt to the contour of the wearer's tragus-concha bowl 957 and snugly fill the interior of the wearer' tragus-concha bowl 957 when the body-structure 67 is placed in the wearer's tragus-concha bowl 957; and so that all of the EEG sensor electrodes 12, 13, 72, 73, 82, 83, will be naturally in close contact with the skin of the wearer's external ear canal 904, and meanwhile the optional reference electrode 84 will be naturally in close contact with the skin of the wearer's tragus-concha bowl 957; and furthermore, at the same time, the taVNS stimulating electrode 31 and the ATN stimulating electrode 312 will be naturally in close contact with the skin or the wearer's external ear canal 904 or the skin of the wearer's tragus-concha bowl 957 when the tubular-body portion 25 is placed in the wearer's ear 902. (provided by the elastic, flexible, and adaptable characteristics of the material so that it can conform to the contour of the external ear canal 904 and the contour of the tragus-concha bowl 957 and snugly fill the interior of the external ear canal 904 and the interior of the tragus-concha bowl 957) (FIG. 11). Attaching and removing all of the EEG sensor electrodes 12, 13, 72, 73, 82, 83, the optional reference electrode 84, the taVNS stimulating electrode 31 and the auriculotemporal nerve stimulating electrode 312 will be as easy as inserting and removing the modified earbud housing 61 from the wearer's ear 902. Please note that the aforementioned description is for a neuromodulation unit 300 having two components (i.e. a taVNS unit 30 and an ATN stimulation unit 302). This description may be similarly applied to a neuromodulation unit 300 having only one component or having different combinations of 2 components. For a neuromodulation unit having 3 components (i.e. a taVNS unit 30, an ATN stimulation unit 302 and a GAN stimulation unit 304) will be described hereinafter.

(For comparison: The vagus-innervated auricular skin includes: inner posterior portion of tragus 905, cymba-concha 906, cavum-concha 907, posterior and inferior walls of the external ear canal 904 and small adjacent regions of the external ear. The auriculotemporal nerve innervated auricular skin includes: anterior outer part of tragus 905, anterior-superior part of cavum concha 907, anterior and superior walls of the external ear canal 904 and, anterior and superior part of pinna including anterior-superior helix 909. The GAN innervated auricular skin includes the cavum concha, specifically the lower or posterior portion of cavum concha.) From the above comparison, it is obvious that the tragus-concha bowl 957 of a wearer's ear 902 received mixed and overlapped innervation from the auricular branch of vagus nerve, the auriculotemporal nerve and the greater auricular nerve. In some embodiments, a neuromodulation unit 300 may have 3 components having a taVNS stimulating electrode 31, an auriculotemporal nerve stimulating electrode 312 and a greater auricular nerve stimulating electrode 314 and all of these electrodes (31, 312, 314) may be located on the body-structure 67. By carefully selecting the location for the taVNS stimulating electrode 31 on the body-structure 67 to match the aforementioned innervation locations of the vagus innervated skin on the tragus-concha bowl 957, the taVNS stimulating electrode 31 will automatically get in close contact with its target skin on the tragus-concha bowl 957 when the body-structure 67 is placed inside the tragus-concha bowl 957. By carefully selecting the location for the auriculotemporal nerve stimulating electrode 312 on the body-structure 67 to match the aforementioned innervation locations of the auriculotemporal nerve on the tragus-concha bowl 957, the auriculotemporal nerve stimulating electrode 312 will automatically get in close contact with its target skin on the tragus-concha bowl 957 when the body-structure 67 is placed in the tragus-concha bowl 957. Similarly, by carefully selecting the location for the greater auricular nerve stimulating electrode 314, on the body-structure 67 to match the aforementioned innervation locations of the greater auricular nerve innervated skin on the tragus-concha bowl 957, the greater auricular nerve stimulating electrode 314 will automatically get in close contact with its target skin on the tragus-concha bowl 957 when the body-structure 67 is placed in the tragus-concha bowl 957. Alternatively, the taVNS stimulating electrode 31 and the auriculotemporal nerve stimulating electrode 312 may be located on the tubular-shaped structure 66. By carefully selecting the location for the taVNS stimulating electrode 31 on the tubular-shaped structure 66 to match its target skin locations on the external ear canal 904, the taVNS stimulating electrode 31 will be automatically get in close contact with its target skin in the external ear canal 904. Similarly, by carefully selecting the location for the auriculotemporal nerve stimulating electrode 312 on the tubular-shaped structure 66 to match its target skin locations on the external ear canal 904, the auriculotemporal nerve stimulating electrode 312 will be automatically get in close contact with its target skin in the external ear canal 904.

In alternative embodiments of an automatic detection-therapy system 101, the auricular EEG recording module 20 may contain an optional reference electrode 84 (to act as a baseline voltage reference for other active EEG sensor electrodes 12, 13, 72, 73, 82, 83). The neuromodulation unit 300 may include at least one of the following components: a taVNS unit 30, an auriculotemporal nerve stimulation unit 302, a supraorbital nerve stimulation unit 301, an occipital nerve stimulation unit 303, a greater auricular nerve stimulation unit 304 and an infraorbital nerve stimulation unit 305. In some embodiments, an automatic detection-therapy system 101 may have a neuromodulation unit 300 that has 3 components, including a taVNS unit 30, an auriculotemporal nerve (ATN) stimulation unit 302 and a greater auricular nerve (GAN) stimulation unit 304 and these 3 components may be configured to be housed in a single auricular housing 11, such as a modified earbud housing 61. (This is feasible and could be preferred due to the proximity and overlapping of their target skin areas and their synergistic effect.) All or portion of the following: all of the EEG sensor electrodes 12, 13, 72, 73, 82, 83, an optional reference electrode 84, a taVNS stimulating electrode 31, an auriculotemporal nerve (ATN) stimulating electrode 312 and a greater auricular nerve (GAN) stimulating electrode 314 are configured to be housed or contained in a modified earbud housing 61 (FIGS. 2, 7, and 11). The modified earbud housing 61 includes a tubular-body portion 25 and a stem portion 68. The tubular-body portion 25 includes a tubular-shaped structure 66 (to be inserted into a wearer's external ear canal when in use) and a body-structure 67 (to be placed at the immediate opening of the wearer's external ear canal 904 and to sit or be placed in the wearer's tragus-concha bowl 957 when in use). The tubular-shaped structure 66 is equivalent to an elongated version of a “nozzle” and an “ear-tip” of an earbud. The body-structure 67 is equivalent to a “body” (or “shell”) of an earbud. All of the EEG sensor electrodes 12, 13, 72, 73, 82, 83, the optional reference electrode 84, the taVNS stimulating electrode 31, the auriculotemporal nerve (ATN) stimulating electrode 312 and the greater auricular nerve (GAN) stimulating electrode 314 are configured to be located on a surface of the tubular-body portion 25 of the modified earbud housing 61. All of the EEG sensor electrodes 12, 13, 72, 73, 82, 83, the optional reference electrode 84, the taVNS stimulating electrode 31, the ATN stimulating electrode 312 and the GAN stimulating electrode 314 may be configured to be partially embedded in the surface with slight protrusion at the surface of the tubular-body portion 25 of the modified earbud housing 61. Preferably all of the EEG sensor electrodes 12, 13, 72, 73, 82, 83, may be located on the surface 91 (and partially embedded in the surface with slight protrusion at the surface) of the tubular-shaped structure 66. Preferably the taVNS stimulating electrode 31 and the ATN stimulating electrode 312 may be located on the surface (and partially embedded in the surface with slight protrusion at the surface) of one of; the body-structure 67 and the tubular-shaped structure 66. Preferably, the optional reference electrode 84 and the GAN stimulating electrode 314 may be located on a surface (and partially embedded in the surface with slight protrusion at the surface) of the body-structure 67. The tubular-body portion 25 may comprise or may be made from an elastic flexible and adaptable material. The material for the tubular-body portion 25 of the modified earbud housing 61 is configured to have appropriate elasticity flexibility and adaptability so that when the tubular-body portion 25 is placed in the wearer's ear 902, all of the EEG sensor electrodes 12, 13, 72, 73, 82, 83 will be naturally and snugly in contact with the skin of the wearer's external ear canal 904; so that the greater auricular nerve (GAN) stimulating electrode 314 and the optional reference electrode 84 will be naturally snugly in contact with the skin of the wearer's tragus-concha bowl 957; and so that the ta VNS stimulating electrode 31 and the auriculotemporal nerve (ATN) stimulating electrode 312 will be naturally snugly in close contact with the skin of wearer's tragus-concha bowl 957 or the skin of the wearer's external ear canal 904 (provided by the elastic, flexible, and adaptable characteristics of the material such that it can conform to the contour of the external ear canal 904 and the contour of the tragus-concha bowl 957 and snugly fill the interior of the external ear canal 904 and the interior of the tragus-concha bowl 957). At the same time, the taVNS stimulating electrode 31 will naturally contact vagus-innervated auricular skin, the auriculotemporal nerve (ATN) stimulating electrode 312 will naturally contact ATN innervated auricular skin and the greater auricular nerve (GAN) stimulating electrode 314 will also naturally snugly contact the GAN innervated auricular skin when the tubular-body portion 25 of the modified earbud housing 61 is placed in the wearer's ear 902 to be in use. (FIGS. 2, 11). Attaching and removing all of the EEG sensor electrodes 12, 13, 72, 73, 82, 83, the optional reference electrode 84, the taVNS stimulating electrode 31, the auriculotemporal nerve stimulating electrode 312 and the greater auricular nerve stimulating electrode 314 will be as easy as inserting and removing the modified earbud housing 61 from the wearer's ear 902.

(For comparison: The vagus-innervated auricular skin includes: inner posterior portion of tragus 905, cymba-concha 906, cavum-concha 907, posterior and inferior walls of the external ear canal 904 and small adjacent regions of the external ear. The auriculotemporal nerve innervated auricular skin includes: anterior outer part of tragus 905, anterior-superior part of cavum concha 907, anterior and superior walls of the external ear canal 904 and, anterior and superior part of pinna including anterior-superior helix 909. The greater auricular nerve innervated auricular skin includes: cavum concha, lower two thirds of anterior and posterior pinna and the mastoid process.) From the above comparison, it is obvious that the tragus-concha 957 of a wearer's ear 902 received mixed and overlapped innervation from the auricular branch of vagus nerve, the auriculotemporal nerve (ATN) and the greater auricular nerve (GAN), while the external ear canal 904 received mixed and overlapped innervation from the auricular branch of vagus nerve and the ATN. When the taVNS stimulating electrode 31, the auriculotemporal nerve (ATN) stimulating electrode 312 and the greater auricular nerve (GAN) stimulating electrode 314 are located in the body-shaped structure 67, by carefully selecting the locations for the taVNS stimulating electrode 31, the ATN stimulating electrode 312 and the GAN stimulating electrode 314 on the body-structure 67 to match the aforementioned innervation locations of the innervated skin on the tragus-concha bowl 957, these stimulating electrodes 31, 312, 314, will automatically get in close contact with their target skin on the tragus-concha bowl 957 when the body-structure 67 is placed in the tragus-concha bowl 957. Alternatively, when the taVNS stimulating electrode 31 and the ATN stimulating electrode 312 are located in the tubular-shaped structure 66, by carefully selecting the locations for the taVNS stimulating electrode 31 and the ATN stimulating electrode 312 on the tubular-shaped structure 66 to match the aforementioned innervation locations of the innervated skin on the external ear canal 904, these stimulating electrodes 31, 312, will automatically get in close contact with their target skin on the external ear canal 904 when the tubular-shaped structure 66 is placed in the external ear canal 904.

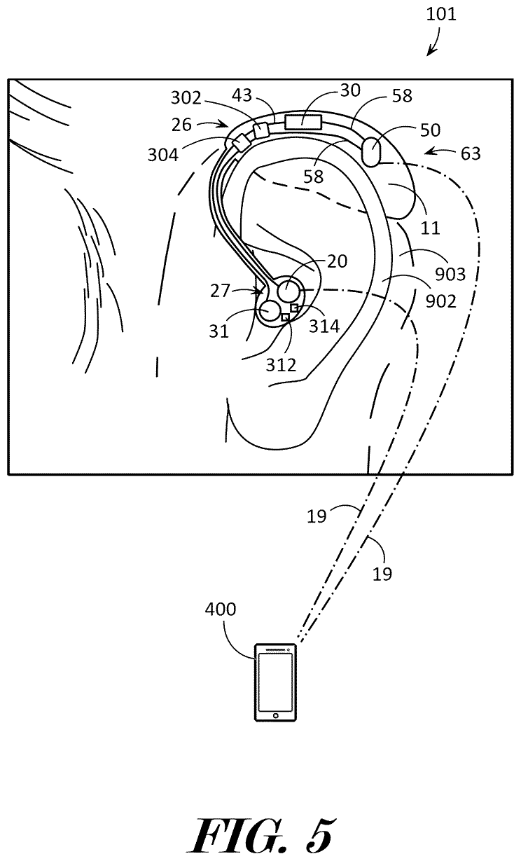

In some embodiments, an auricular housing 11 of an auricular EEG monitoring system 100 and/or an automatic detection-therapy system 101 may be shaped or configured as a behind-the-ear-hearing-aid-style housing 63 as shown in FIGS. 5, 10, and 13. The behind-the-ear-hearing-aid-style housing 63 may comprise a behind-the-ear portion 26 and an in-the-ear portion 27. Generally, a behind-the-ear-hearing-aid-style housing 63 may be shaped and sized so that all or a majority of the behind-the-ear portion 26 may be positioned behind the ear 902, such as to contact the post-auricular part of the peri-auricular area 903, as shown in FIGS. 5, 10, and 13. The in-the-ear portion 27 is essentially the same as the tubular-body portion 25 of a modified earbud housing 61. The in-the-ear portion 27 is also essentially the same as a modified in-the-ear housing 62, as aforementioned description. The in-the-ear portion 27 of the behind-the-ear-hearing-aid-style housing 63 may include a tubular-shaped structure 66 (to be placed inside the wearer's external ear canal 904 when in use) and a body-structure 67 (to be located immediately outside the opening of the wearer's external ear canal 904 and to sit or be placed inside the tragus-concha bowl 957). The in-the-ear portion 27 may be sized and shaped so that the tubular-shaped structure 66 can be inserted into the external ear canal 904 while the body-structure 67 will be located immediately at opening of the external ear canal 904 and sitting inside the tragus-concha bowl 957 when in use. Example of behind-the-ear-hearing-aid-style structures 63 include behind-the-ear (BTE), receiver-in-the-ear (RITE), CROS/BICROS (CROS stands for “Contralateral Routing of Signals” and BiCROS stands for “Bilateral Contralateral Routing of Signals”), and the like.

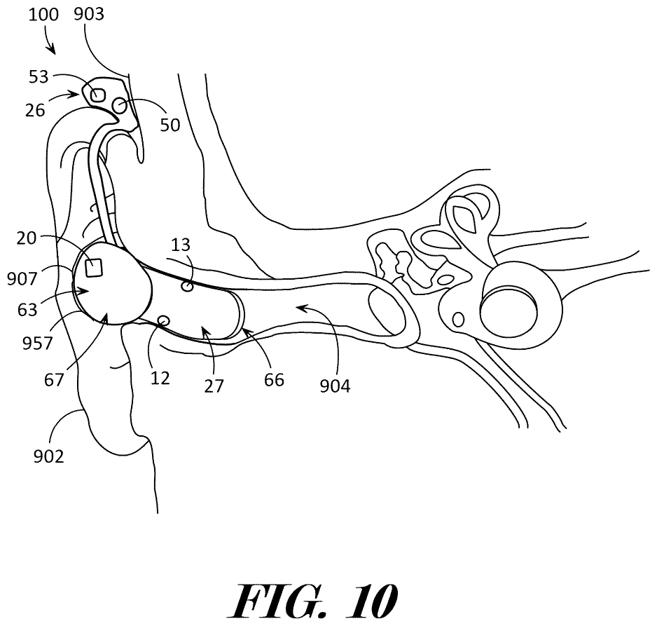

In some embodiments of an auricular EEG monitoring system 100, all or portion of the following: an EEG recording module 20 and its EEG sensor electrodes 12, 13, 72, 73, 82, 83, a processing unit 50, and a network interface 53 may be housed or contained in a behind-the-ear-hearing-aid-style housing 63. The auricular EEG monitoring system 100 may comprise an EEG recording module 20, a processing unit 50, and a network interface 53. The behind-the-ear-hearing-aid-style housing 63 includes an in-the-ear portion 27 that is essentially the same as the tubular-body portion 25 of a modified earbud housing 61. The in-the-ear portion 27 is also essentially the same as a modified in-the-ear housing 62, as aforementioned description. The in-the-ear portion 27 of the behind-the-ear-hearing-aid-style housing 63 may include a tubular-shaped structure 66 (to be placed inside the wearer's external ear canal 904 when in use) and a body-structure 67 (to be located immediately outside the opening of the wearer's external ear canal 904 and to sit inside the tragus-concha bowl 957). All of the EEG sensor electrodes 12, 13, 72, 73, 82, 83, may be located on a surface 91 of the tubular-shaped structure 66 of the in-the-ear portion 27 of the behind-the-ear-hearing-aid-style housing 63. All of the EEG sensor electrodes 12, 13, 72, 73, 82, 83, may be configured to be partially embedded in the surface 91 with slight protrusion at the surface 91 of the tubular-shaped structure 66. Optionally, the auricular EEG recording module 20 may contain an optional reference electrode 84 (to act as a baseline voltage reference for other active EEG sensor electrodes 12, 13, 72, 73, 82, 83). Preferably all of the EEG sensor electrodes 12, 13, 72, 73, 82, 83, may be located on the surface 91 of the tubular-shaped structure 66. Preferably the optional reference electrode 84 may be located on the surface and partially embedded in the surface with slight protrusion at the surface of the body-structure 67. Preferably, the in-the-ear portion 27 of the behind-the-ear-hearing-aid-style housing 63 may comprise or may be made from an elastic flexible and adaptable material. The material for the in-the-ear portion 27 is configured to have appropriate elasticity flexibility and adaptability so that all of the EEG sensor electrodes 12, 13, 72, 73, 82, 83, of the EEG recording module 20 are naturally in close contact (provided by the elastic, flexible, and adaptable characteristics of the resilient material so that it can conform to the contours of the external ear canal 904 and snugly fill the interior of the external ear canal 904) with the skin of the external ear canal 904 of the wearer's ear 901 when the in-the-ear portion 27 is placed in the wearer's ear 902. Meanwhile, the optional reference electrode 84 will be naturally in close contact with the skin of tragus-concha bowl 957 of the wearer's ear 902. (FIG. 10).

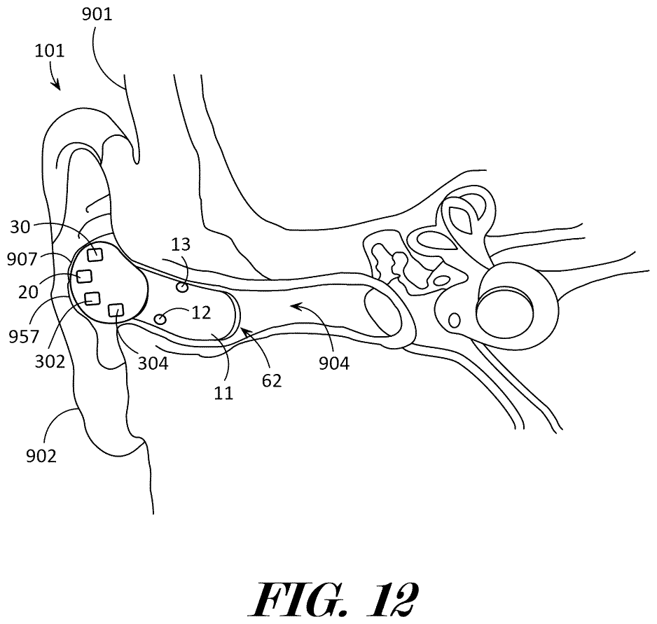

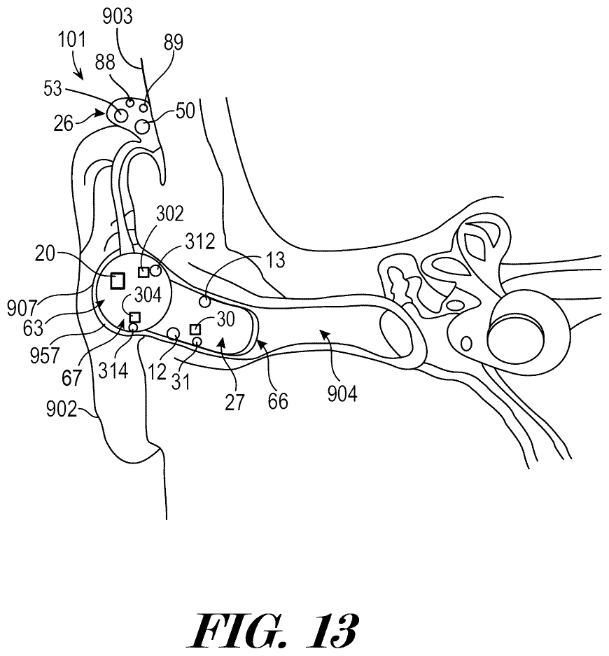

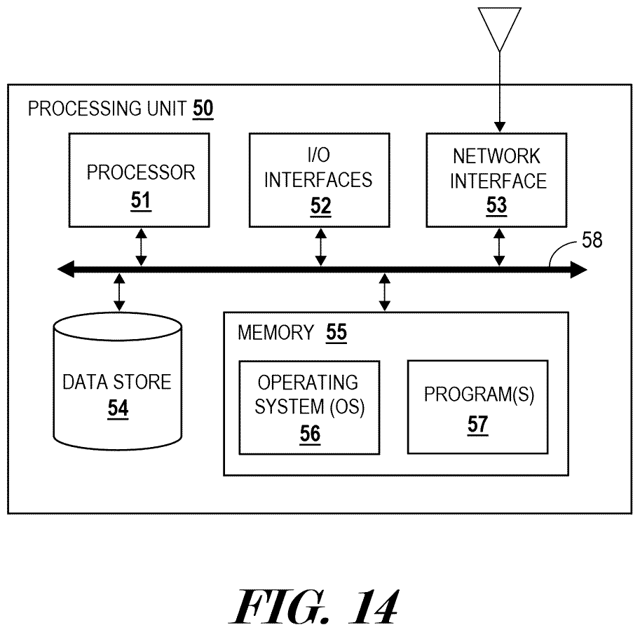

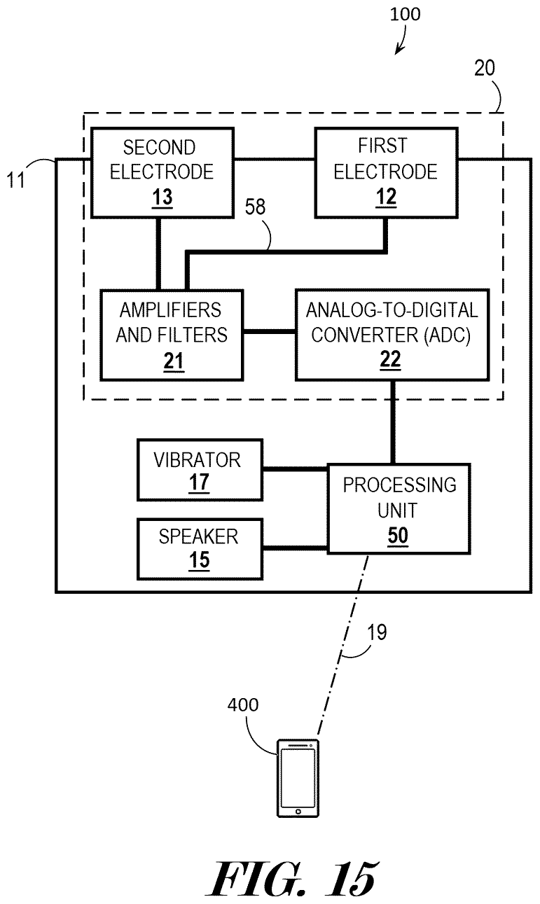

Similarly, in some embodiments of an automatic detection-therapy system 101, all or portion of the following: an EEG recording module 20 with its EEG sensor electrodes 12, 13, 72, 73, 82, 83, a processing unit 50, a neuromodulation unit 300 and a network interface 53 may be housed or contained in a behind-the-ear-hearing-aid-style housing 63. The automatic detection-therapy system 101 may comprise an EEG recording module 20, a processing unit 50, a neuromodulation unit 300 and a network interface 53. The neuromodulation unit 300 may include at least one of the following components: a taVNS unit 30, an auriculotemporal nerve stimulation unit 302, a supraorbital nerve stimulation unit 301, an occipital nerve stimulation unit 303, a greater auricular nerve stimulation unit 304 and an infraorbital nerve stimulation unit 305. In some embodiments, an automatic detection-therapy system 101 may have a neuromodulation unit that may have two components, for example: a taVNS unit 30 and an auriculotemporal nerve (ATN) stimulation unit 302. (Other examples of a neuromodulation unit 300 having only one component or combination of two different components would be similar to the following description). Combination of both taVNS unit 30 and ATN stimulation unit 302 into a single auricular housing 11, such as a behind-the-ear-hearing-aid-style housing 63, for automatic detection-therapy system 101 would be preferred due to the proximity of their target skin areas and their synergistic effect. The behind-the-ear-hearing-aid-style housing 63 comprises an in-the-ear portion 27 and a behind-the-ear portion 26. The in-the-ear portion 27 includes a tubular-shaped structure 66 and a body-structure 67. (The in-the-ear portion 27 is essentially the same as the tubular-body portion 25 of a modified earbud housing 61.) Preferably, all of the EEG sensor electrodes 12, 13, 72, 73, 82, 83, may be located on a surface 91 of the tubular-shaped structure 66 of the in-the-ear portion 27 of the behind-the-ear-hearing-aid-style housing 63. All of the EEG sensor electrodes 12, 13, 72, 73, 82, 83, may be configured to be partially embedded in the surface with slight protrusion at the surface of tubular-shaped structure 66. Preferably the taVNS stimulating electrode 31 and the auriculotemporal nerve (ATN) stimulating electrode 312 may be located on the surface and partially embedded in the surface with slight protrusion at the surface of one of; the body-structure 67 and the tubular-shaped structure 66. Optionally, the auricular EEG recording module 20 may contain an optional reference electrode 84 (to act as a baseline voltage reference for other active EEG sensor electrodes 12, 13, 72, 73, 82, 83). Preferably, the optional reference electrode 84 may be placed on a surface (and partially embedded in the surface with slight protrusion at the surface) of the body-structure 67. Preferably, the in-the-ear portion 27 may comprise or may be made from an elastic flexible and adaptable material. The material for the in-the-ear portion 27 is configured to have appropriate elasticity flexibility and adaptability so that when the in-the-ear portion 27 is placed in the wearer's external ear canal 904 and the tragus-concha bowl 957, the in-the-ear portion 27 will naturally adapt to the contour of the wearer's external ear canal 904 and the contour of the wearer's tragus-concha bowl 957 and snugly fill the interior of the wearer's external ear canal 904 and also fill the interior of the wearer's tragus-concha bowl 957; so that all of the EEG sensor electrodes 12, 13, 72, 73, 82, 83, will be naturally in close contact with the skin of the wearer's external ear canal 904, and meanwhile, the optional reference electrode 84 will be naturally in close contact with the skin of the wearer's tragus-concha bowl 957; and at the same time, the taVNS stimulating electrode 31 and the ATN stimulating electrode 312 will be naturally in close contact with the skin of the wearer's external ear canal 904 or the skin of the wearer's tragus-concha bowl 957. (provided by the elastic, flexible, and adaptable characteristics of the resilient material so that it can conform to the contours of the external ear canal 904 and snugly fill the interior of the external ear canal 904). (FIG. 13) Meanwhile, the taVNS stimulating electrode 31 will be naturally contacting vagus-innervated auricular skin of the wearer's ear 902; and the auriculotemporal nerve (ATN) stimulating electrode 312 will be naturally contacting ATN innervated auricular skin. This is feasible due to the proximity of the innervation patterns of the auricular branch of vagus nerve and the auriculotemporal nerve in the auricular area, and by careful selection of the locations for 31, 312, on the body-structure 67 or the tubular-shaped structure 66 to match the locations of the innervation patterns of their respective target skin on the wearer's ear 902 (including the tragus-concha bowl 957 or external ear canal 904), as described hereinbefore. Installing and removing all of the EEG sensor electrodes 12, 13, 72, 73, 82, 83, the optional reference electrode 84, the taVNS stimulating electrode 31 and the auriculotemporal nerve stimulating electrode 312 will be as easy as inserting and removing the in-the-ear portion 27 from the wearer's external ear 902. (FIGS. 5, 13, 28). This description may be similarly applied for a neuromodulation unit 300 having only one component or having different combinations of 2 components. For a neuromodulation unit having 3 components (i.e. a taVNS unit 30, an ATN stimulation unit 302 and a GAN stimulation unit 304) will be described hereinafter.

In alternative embodiments of an automatic detection-therapy system 101 may comprise an auricular EEG monitoring system 100 having an EEG recording module 20, a processing unit 50, a neuromodulation unit 300 and a network interface 53. The neuromodulation unit 300 may include at least one of the following components: a taVNS unit 30, an auriculotemporal nerve stimulation unit 302, a supraorbital nerve stimulation unit 301, an occipital nerve stimulation unit 303, a greater auricular nerve stimulation unit 304 and an infraorbital nerve stimulation unit 305. In some embodiments, an automatic detection-therapy system 101 may have a neuromodulation unit that may have three components, for example: a taVNS unit 30, an auriculotemporal nerve (ATN) stimulation unit 302 and a greater auricular nerve (GAN) stimulation unit 304. Combination of a taVNS unit 30, an ATN stimulation unit 302 and a GAN stimulation unit 304 into a single auricular housing 11, such as a behind-the-ear-hearing-aid-style housing 63 would be feasible and could be preferred due to the proximity and overlapping of their target skin areas and their synergistic effects. The behind-the-ear-hearing-aid-style housing 63 includes an in-the-ear portion 27 that comprises a tubular-shaped structure 66 (to be inserted into a wearer's external ear canal 904 when in use) and a body-structure 67 (to be placed at the opening of the wearer's external ear canal 904 and to sit or be placed inside the wearer's tragus-concha bowl 957 when in use). (The in-the-ear portion 27 is essentially the same as the tubular-body portion 25 of a modified earbud housing 61). Optionally, the auricular EEG recording module 20 may contain an optional reference electrode 84 (to act as a baseline voltage reference for other active EEG sensor electrodes 12, 13, 72, 73, 82, 83). Preferably all of the EEG sensor electrodes 12, 13, 72, 73, 82, 83, may be located on the surface 91 of the tubular-shaped structure 66 and partially embedded in the surface with slight protrusion at the surface of tubular-shaped structure 66 (of the behind-the-ear-hearing-aid-style housing 63). Preferably the optional reference electrode 84 and the greater auricular nerve (GAN) stimulating electrode 314 may be located on a surface (and partially embedded in the surface with slight protrusion at the surface) of the body-structure 67. Preferably the ta VNS stimulating electrode 31 and the auriculotemporal nerve (ATN) stimulating electrode 312 may be located on the surface and partially embedded in the surface with slight protrusion at the surface of one of, the body-structure 67 and the tubular-shaped structure 66. Preferably, the in-the-ear portion 27 may comprise or may be made from an elastic flexible and adaptable material. The material for the in-the-ear portion 27 is configured to have appropriate elasticity flexibility and adaptability so that when the tubular-shaped structure is inserted into the wearer's external ear canal 904 the tubular-shaped structure 66 will naturally adapt to the contour of the wearer's external ear canal 904 and snugly fill the interior of the wearer's external ear canal 904, so that all of the EEG sensor electrodes 12, 13, 72, 73, 82, 83, are naturally snugly in contact with the skin of the wearer's external ear canal; meanwhile, when the body-structure 67 is placed in the wearer's tragus-conchae bowl 957 the body-structure will naturally adapt to the contour of the wearer's tragus-concha bowl 957 and snugly fill the interior of the wearer's tragus-concha bowl 957 so that the optional reference electrode 84 and the GAN stimulating electrode 314 are naturally snugly in contact with the skin of the wearer's tragus-concha bowl 957. Furthermore, at the same time, the taVNS stimulating electrode 31 and the auriculotemporal nerve (ATN) stimulating electrode 312 are naturally snugly in close contact with skin of the wearer's external ear canal 904 or skin of the wearer's tragus-concha bowl 957 (provided by the elastic, flexible, and adaptable characteristics of the resilient material so that it can conform to the contours of the external ear canal 904 and contour of tragus-concha bowl 957 and snugly fill the interior of the external ear canal 904 and interior of tragus-concha bowl 957) when the in-the-ear portion 27 is placed in the wearer's ear 902. (FIG. 13). At the meantime, the taVNS stimulating electrode 31 will naturally contact the wearer's vagus innervated auricular skin, the auriculotemporal nerve (ATN) stimulating electrode 312 will naturally contact the wearer's ATN innervated auricular skin, and the greater auricular nerve (GAN) stimulating electrode 314 will naturally contact the GAN innervated auricular skin of the wearer 900 when the in-the-ear portion is placed in the wearer's ear 902. Installing and removing all of the EEG sensor electrodes 12, 13, 72, 73, 82, 83, the optional reference electrode 84, the taVNS stimulating electrode 31, the auriculotemporal nerve stimulating electrode 312 and the greater auricular nerve stimulating electrode 314 will be as easy as inserting and removing the in-the-ear portion 27 from the wearer's external ear 902. (FIGS. 5, 13, 28). This is feasible due to the proximity of the innervation patterns of the auricular branch of vagus nerve, the auriculotemporal nerve and the greater auricular nerve in the auricular area, and the anatomic features of the external ear canal 904 and the bowl-cavity characteristics of the tragus-concha bowl 957, as described hereinbefore.

(For comparison: The vagus-innervated auricular skin includes: inner posterior portion of tragus 905, cymba-concha 906, cavum-concha 907, posterior and inferior walls of the external ear canal 904 and small adjacent regions of the external ear. The auriculotemporal nerve innervated auricular skin includes: anterior outer part of tragus 905, anterior-superior part of cavum concha 907, anterior and superior walls of the external ear canal 904 and, anterior and superior part of pinna including anterior-superior helix 909. The greater auricular nerve innervated auricular skin includes: cavum concha, lower two thirds of anterior and posterior pinna and the mastoid process.) From the above comparison, it is obvious that the tragus-concha 957 of a wearer's ear 902 received mixed and overlapping innervation from the auricular branch of vagus nerve, the auriculotemporal nerve (ATN) and the greater auricular nerve (GAN), while the external ear canal 904 receives mixed and overlapping innervation from the auricular branch of vagus nerve and the ATN. When the taVNS stimulating electrode 31, the ATN stimulating electrode 312 and the GAN stimulating electrode 314 are located on the body-structure 67, by carefully selecting the locations for the taVNS stimulating electrode 31, the auriculotemporal nerve stimulating electrode 312 and the greater auricular nerve stimulating electrode 314 on the body-structure 67 to match the aforementioned innervation locations of the innervated skin on the tragus-concha bowl 957, these stimulating electrodes 31, 312, 314, will automatically get in close contact with their target skin on the tragus-concha bowl when the body-structure 67 is placed in the tragus-concha bowl 957. Alternatively, when the taVNS stimulating electrode 31 and the ATN stimulating electrode 312 are located in the tubular-shaped structure 66, by carefully selecting the locations for the taVNS stimulating electrode 31 and the auriculotemporal nerve (ATN) stimulating electrode 312 on the tubular-shaped structure 66 to match the aforementioned innervation locations of the innervated skin on the external ear canal 904, these stimulating electrodes 31, 312, will automatically get in close contact with their target skin on the external ear canal 904 when the tubular-shaped structure 66 is placed in the external ear canal 904. (This is feasible due to the proximity of the innervation patterns of the auricular branch of vagus nerve, the auriculotemporal nerve and the greater auricular nerve in the auricular area, and by careful selection of the locations for 31, 312, 314, to match the locations of their respective target skin on the wearer's ear, as described hereinbefore.)

Generally, an elastic flexible and adaptable material may be flexible to allow slight deformation and optionally resilient so as to return to its original shape after deformation. In preferred embodiments, all or portions of a modified earbud housing 61 (e.g., a tubular-body portion 25), all or portions of a modified in-the-ear housing 62, all or portions of a behind-the-ear-hearing-aid-style housing 63 (e.g., in-the-ear portion 27), all or portions of the tubular-shaped structure 66 may be made from or comprise an elastic flexible and adaptable material such as natural and/or synthetic rubber material such as latex rubber, silicone foam, silicone rubber or polysiloxanes, rubber foam, urethane foam, plastic foam, neoprene foam, latex foam rubber, polyurethane foam rubber, forms of the organic compound isoprene, Polyacrylate Rubber, Ethylene-acrylate Rubber, Polyester Urethane, flexible plastics, such as high-density polyethylene (HDPE), polyvinyl chloride (PVC), polypropylene (PP), Polystyrene (PS), Polycarbonate (PC), low density polyethylene (LDPE), or any other flexible material including combinations of materials.

In some embodiments, an auricular housing 11 may house one or more components, such as a processing unit 50, an EEG recording module 20, EEG sensor electrodes 12, 13, 72, 73, 82, 83, an optional reference electrode 84, a speaker 15, a power source 16, a vibrator 17, etc., which may be communicatively coupled via a local interface 58. The local interface 58 can be, for example but not limited to, one or more buses, circuit boards, wiring harnesses, or other wired connections or wireless connections, as is known in the art. The local interface 58 can have additional elements, which are omitted for simplicity, such as controllers, buffers (caches), drivers, repeaters, and receivers, among many others, to enable communications. Further, the local interface 58 may include address, control, and/or data connections to enable appropriate communications among the aforementioned components.

In some embodiments, an auricular EEG monitoring system 100 may comprise a processing unit 50 which may be contained in the auricular housing 11. A processing unit 50 may include a processor 51 that may comprise a hardware device for executing software instructions. (FIG. 14). The processor 51 can be any custom made or commercially available processor, a central processing unit (CPU), an auxiliary processor among several processors associated with the processing unit 50, a semiconductor-based microprocessor (in the form of a microchip or chip set), or generally any device for executing software instructions. Optionally, when the processing unit 50 is in operation, the processor 51 may be configured to execute software stored within a memory 55, to communicate data to and from the memory 55, and to generally control one or more operations of the auricular EEG monitoring system 100 pursuant to the software instructions and/or from instructions. In an exemplary embodiment, the processor 51 may include a mobile optimized processor, such as optimized for power consumption and mobile applications.

In some embodiments, an auricular EEG monitoring system 100 may comprise one or more I/O interfaces 52 which can be used to provide user input and display system output data, such as operational status, from the auricular EEG monitoring system 100. The I/O interfaces 52 can include, for example, buttons, knobs, switches, LED indicator lights, LED display, LCD display, a serial port, a parallel port, a small computer system interface (SCSI), an infrared (IR) interface, a radio frequency (RF) interface, a universal serial bus (USB) interface, and the like.

In some embodiments, an auricular EEG monitoring system 100 may comprise a network interface 53 which may be contained in the auricular housing 11 and which may enable wired and/or wireless communication between one or more components, such as EEG recording module 20, processing unit 50, etc., with one or more client devices 400. Preferably, a network interface 53 may comprise a radio that may operate via WiFi and/or Bluetooth communication standards. In further embodiments, a network interface 53 may comprise a radio that may operate on a cellular band and may communicate with or receive a Subscriber Identity Module (SIM) card or other wireless network identifier. Any number of suitable wireless data communication protocols, techniques, or methodologies can be supported by a network interface 53, including, without limitation: RF; IrDA (infrared); Bluetooth; ZigBee (and other variants of the IEEE 802.15 protocol); IEEE 802.11 (any variation); IEEE 802.16 (WiMAX or any other variation); Direct Sequence Spread Spectrum; Near-Field Communication (NFC); Frequency Hopping Spread Spectrum; Long Term Evolution (LTE); cellular/wireless/cordless telecommunication protocols (e.g. 3G/4G, etc.); wireless home network communication protocols; paging network protocols; magnetic induction; satellite data communication protocols; wireless hospital or health care facility network protocols such as those operating in the WMTS bands; GPRS; proprietary wireless data communication protocols such as variants of Wireless USB; and any other protocols for wireless communication. In further embodiments, a network interface 53 may enable wired network communication and may include, for example, an Ethernet card or adapter (e.g., 10BaseT, Fast Ethernet, Gigabit Ethernet, 10 GbE) or a wireless local area network (WLAN) card or adapter (e.g., 802.11a/b/g/n). The network interface 53 may include address, control, and/or data connections to enable appropriate communications on the network.

In some embodiments, a processing unit 50 may comprise a memory 55 that may include any volatile memory elements (e.g., random access memory (RAM, such as DRAM, SRAM, SDRAM, etc.)), nonvolatile memory elements (e.g., ROM, hard drive, etc.), and combinations thereof. Moreover, the memory 55 may incorporate electronic, magnetic, optical, and/or other types of storage media. Note that the memory 55 may have a distributed architecture, where various components are situated remotely from one another, but can be accessed by the processor 51. Optionally, memory 55 can include one or more software programs, each of which includes an ordered listing of executable instructions for implementing logical functions. Optionally, the software in the memory system 55 includes a suitable operating system (O/S) 56 and program(s) 57. The operating system 56 essentially controls the execution of input/output interface 52 and other element functions, and provides scheduling, input-output control, file and data management, memory management, and communication control and related services. The operating system 56 may be, for example, LINUX (or another UNIX variant), Android (available from Google), Symbian OS, Microsoft Windows CE, Microsoft Windows 7 Mobile, iOS (available from Apple, Inc.), webOS (available from Hewlett Packard), Blackberry OS (Available from Research in Motion), and the like. The programs 57 may include various applications, add-ons, etc. configured to provide end user functionality of the system 100.

Further, many embodiments are described in terms of sequences of actions to be performed by, for example, elements of a computing device. It will be recognized that various actions described herein can be performed by specific circuits (e.g., application specific integrated circuits (ASICs)), by program instructions being executed by one or more processors, or by a combination of both. Additionally, these sequences of actions described herein can be considered to be embodied entirely within any form of computer readable storage medium having stored therein a corresponding set of computer instructions that upon execution would cause an associated processor to perform the functionality described herein. Thus, the various aspects of the invention may be embodied in a number of different forms, all of which have been contemplated to be within the scope of the claimed subject matter. In addition, for each of the embodiments described herein, the corresponding form of any such embodiments may be described herein as, for example, “logic configured to” perform the described action.

The auricular EEG monitoring system 100 may comprise one or more auricular EEG recording modules 20. In some embodiments, an auricular EEG monitoring system 100 may comprise an auricular EEG recording module 20 that may be contained in an auricular housing 11. An auricular EEG recording module 20 may record the electrical activities of the brain (EEG signals) of the wearer 900 to generate EEG data. The EEG data may be analyzed by a processing unit 50, 401, to detect presence or cessation of EEG signals suggestive of neuropsychiatric disorders. The processing unit 50, 401 is further configured to analyze the EEG data to detect presence or cessation of EEG signals suggestive of impending neuropsychiatric disorders. These neuropsychiatric disorders include seizure, migraine, cluster headache, neurodegenerative diseases, major depressive disorder, bipolar disorder, schizophrenia, obsessive traumatic stress disorder, anxiety disorder and panic disorder, etc.

Generally, amplifiers and filters 21 of an auricular EEG recording module 20 may pick up the electrical activities of the wearer's 900 brain via the plurality of EEG sensor electrodes 12, 13, 72, 73, 82, 83, and the optional reference electrode 84. An amplifier of amplifiers and filters 21 is responsible for amplifying the weak electrical signals received from the electrodes 12, 13, 72, 73, 82, 83, 84. The brain's electrical signals are typically very faint, (often in the 5-30 microvolts range for in-ear electrodes and about 10-100 microvolts for scalp electrodes). The amplifier boosts these signals to a level that can be accurately recorded and displayed. Modern EEG machines use sophisticated amplifiers that minimize noise and ensure signal clarity. Filters of amplifiers and filters 21 are used to remove unwanted noise and interference from the electrical signals. Common sources of noise include muscle contractions, electrical interference from other devices, and movement artifacts. EEG machines use various filters, such as high-pass, low-pass, and notch filters, to clean the signals, ensuring that the resulting EEG trace is clear and interpretable. An analog-to-digital converter (ADC) 22 may transform the analog electrical signals from the brain into digital data. This digital conversion is essential for processing, storing, and displaying the EEG data on a screen or print out. The ADC 22 ensures that the data is accurately digitized, preserving the integrity of the original signals. The ADC 22 may be in communication with a processing unit 50.

According to another embodiment consistent with the principles of the present invention, an automatic detection-therapy system 101 is disclosed (FIGS. 2, 3, 5-7, 11-13, 18 and 28). In some embodiments, an automatic detection-therapy system 101 may comprise an auricular electroencephalogram (EEG) monitoring system 100 having one or more EEG recording modules 20. Each EEG recording module 20 preferably has a plurality of miniature wired or wireless EEG sensor electrodes 12, 13, 72, 73, 82, 83, and an optional wired or wireless reference electrode 84 that are configured to contact or be attached to separate areas selected from at least one of the following: the external ear canal 904, external ear 902, or peri-auricular area 903, that may be configured to record EEG data of the wearer 900, such as discussed above. In some embodiments, an automatic detection-therapy system 101 may include one or two transcutaneous auricular vagus nerve stimulation units (taVNS units) 30, such as a first taVNS unit 30 and a second taVNS unit 30, with each taVNS unit 30 having a ta VNS stimulating electrode 31 configured to contact vagus innervated auricular skin of one of the wearer's ears 902. The vagus innervated auricular skin includes external ear canal 904, tragus 905, cymba-concha 906, cavum-concha 907 and small adjacent areas. (More specifically, the vagus innervated auricular skin includes: posterior and inferior walls of external ear canal 904, inner/posterior portion of tragus 905, cymba-concha 906, and majority of cavum-concha 907, and part of the eardrum.) The vagus innervated auricular skin that the stimulating electrode 31 is configured to contact may be selected from at least one of the following: external ear canal 904, tragus 905, cymba-concha 906, and cavum-concha 907. The one or more EEG recording modules 20 and the one or two taVNS units 30 are in wired or wireless electronic communication with the processing unit 50, 401.

In some embodiments, an automatic detection-therapy system 101 may comprise a neuromodulation unit 300 that includes a transcutaneous auricular vagus nerve stimulation (taVNS) unit, such as a first taVNS unit 30. The first taVNS unit 30 includes a first taVNS stimulating electrode 31 configured to contact vagus innervated auricular skin of the wearer's first ear 902. When activated, the first taVNS unit 30 is configured to send neuromodulating electric stimulation to the vagus innervated auricular skin of the wearer's first ear. The vagus innervated auricular skin includes: posterior and inferior walls of external ear canal 904, inner/posterior portion of tragus 905, cymba-concha 906, cavum-concha 907 and small adjacent areas. The vagus innervated auricular skin that the stimulating electrode 31 is configured to contact may be selected from at least one of the following: external ear canal 904, tragus 905, cymba-concha 906, and cavum-concha 907 of the wearer's ear 902. A processing unit 50, 401, may be in electronic communication with the auricular electroencephalogram (EEG) monitoring system 100 and with the first taVNS unit 30. The processing unit 50, 401, may be configured to analyze EEG data recorded by the EEG monitoring system 100 to detect the presence or cessation of EEG signals suggestive of neuropsychiatric disorders. When the presence of EEG signals suggestive of a neuropsychiatric disorder is detected by the processing unit 50, 401, the processing unit 50, 401, may be configured to immediately send signals to the first ta VNS unit 30 to prompt the first ta VNS unit 30 to automatically start sending pre-determined electric stimulation to the vagus innervated auricular skin of the wearer's ear 902 to which the first taVNS stimulating electrode 31 is in contact with. When cessation of EEG signals suggestive of the neuropsychiatric disorder is detected by the processing unit 50, 401, the processing unit 50, 401, may be further configured to immediately send signals to the first taVNS unit 30 to prompt the first taVNS unit 30 to automatically stop sending pre-determined electric stimulation to the vagus innervated auricular skin of the wearer's first ear 902. The processing unit 50, 401, may be further configured to analyze the EEG data recorded by the EEG monitoring system 100 to detect the presence or cessation of EEG signals suggestive of impending neuropsychiatric disorder. When the presence of EEG signals suggestive of impending neuropsychiatric disorder is detected by the processing unit 50, 401, the processing unit 50, 401, may be configured to immediately send signals to the first taVNS unit 30 to prompt the first taVNS unit 30 to automatically start sending predetermined electric stimuli to the vagus innervated auricular skin of the wearer's first ear 902. When cessation of EEG signals suggestive of the impending neuropsychiatric disorder is detected by the processing unit 50, 401, the processing unit 50, 401, may be further configured to immediately send signals to the first taVNS unit 30 to prompt the first taVNS unit 30 to automatically stop sending electric stimulation to the vagus innervated auricular skin of the wearer's first ear 902.

In some embodiments, an automatic detection-therapy system 101 may comprise two transcutaneous auricular vagus nerve stimulation (taVNS) units 30 (“taVNS unit 30”) such as shown in FIG. 18. Preferably, an automatic detection-remedy system 101 may comprise a first taVNS unit (such as described hereinbefore) and a second taVNS unit 30. The second taVNS unit 30 may include a second taVNS stimulating electrode 31 configured to contact vagus innervated auricular skin of the wearer's second ear 902. A processing unit 50, 401, may be in electronic communication with the second taVNS unit 30. When the presence of EEG signals suggestive of a neuropsychiatric disorder is detected by the processing unit 50, 401, the processing unit 50, 401, may be configured to immediately send signals to the second taVNS unit 30 to prompt the second taVNS unit 30 to automatically start sending pre-determined electric stimulation to the vagus innervated auricular skin of the wearer's second ear 902. When cessation of EEG signals suggestive of the neuropsychiatric disorder is detected by the processing unit 50, 401, the processing unit 50, 401, may be further configured to immediately send signals to the second taVNS unit 30 to prompt the second taVNS unit 30 to automatically stop sending pre-determined electric stimulation to the vagus innervated auricular skin of the wearer's second ear 902. When presence of EEG signals suggestive of impending neuropsychiatric disorder is detected by the processing unit 50, 401, the processing unit 50, 401, may be further configured to immediately send signals to the second ta VNS unit 30 to prompt the second taVNS unit 30 to automatically start sending pre-determined electric stimulation to the vagus innervated auricular skin of the wearer's second ear 902. When cessation of EEG signals suggestive of the impending neuropsychiatric disorder is detected by the processing unit 50, 401, the processing unit 50, 401, may be further configured to send signals to the second taVNS unit 30 immediately to prompt the second taVNS unit 30 to automatically stop sending electric stimulation to the vagus innervated auricular skin of the wearer's second ear 902.

In some embodiments, an automatic detection-therapy system 101 may comprise a neuromodulation unit 300 that may include at least one of the following components: a transcutaneous auricular vagus nerve stimulation unit (taVNS unit) 30, a supraorbital nerve stimulation unit 301, an infraorbital nerve stimulation unit 305, an auriculotemporal nerve (ATN) stimulation unit 302, an occipital nerve stimulation unit 303 and a greater auricular nerve (GAN) stimulation unit 304. There are evidences showing the benefits of neuromodulating electric stimulation from taVNS unit 30. More recently, there are also evidences showing benefits of neuromodulating electric stimulation from non-vagus electric neuromodulation unit. As used herein, the term “non-vagus electric neuromodulation unit” refers to one of the following: a supraorbital nerve stimulation unit 301, an infraorbital nerve stimulation unit 305, an auriculotemporal nerve stimulation unit 302, an occipital nerve stimulation unit 303, a greater auricular nerve stimulation unit 304, and various combinations thereof. Non-vagus electric neuromodulation has special benefits for migraine, cluster headache, other pain and other neuropsychiatric disorders. In some embodiments, an automatic detection-therapy system 101 may comprise a neuromodulation unit 300 that may include a taVNS unit 30 and a non-vagus electric neuromodulation unit having at least one of the following components: a supraorbital nerve stimulation unit 301, an infraorbital nerve stimulation unit 305, an auriculotemporal nerve stimulation unit 302, an occipital nerve stimulation unit 303, a greater auricular nerve stimulation unit 304, and various combinations thereof. Simultaneous stimulation from a taVNS unit 30 and a non-vagus electric neuromodulation unit has synergistic effects. Each component of the neuromodulation unit 300 is in wired or wireless communication with a processing unit 50, 401. In some embodiments, a neuromodulation unit 300 may include a taVNS unit 30. The setups and the functions of the taVNS unit 30 are as described hereinbefore. The setups and functions of the supraorbital nerve stimulation unit 301, the auriculotemporal nerve (ATN) stimulation unit 302, the occipital nerve stimulation unit 303, the GAN stimulation unit 304 and the infraorbital nerve stimulation unit 305 are also similar to the aforementioned descriptions for taVNS unit 30.

In some embodiments, an automatic detection-therapy system 101 may comprise a neuromodulation unit 300 that includes a supraorbital nerve stimulation unit 301. When activated, the supraorbital nerve stimulation unit 301 is configured to send neuromodulating electric stimulation to the supraorbital nerve innervated skin of the wearer's forehead. When prompted by EEG findings as described hereinbefore, the supraorbital nerve stimulation unit 301 may be configured to start neuromodulating electric stimulation to supraorbital nerve innervated skin at the wearer's 900 forehead via a supraorbital nerve stimulating electrode 311. When the processing unit 50, 401, detects presence of EEG signals suggestive of neuropsychiatric disorder, the processing unit 50, 401, may be configured to send signals to the supraorbital nerve stimulation unit 301 to prompt the supraorbital nerve stimulation unit 301 to start sending neuromodulating electric stimulation to the supraorbital nerve innervated forehead skin of the wearer 900. When the processing unit 50, 401, detects presence of EEG signals suggestive of impending neuropsychiatric disorder, the processing unit 50, 401, may be configured to send signals to the supraorbital nerve stimulation unit 301 to prompt the supraorbital nerve stimulation unit 301 to start sending neuromodulating electric stimulation to the supraorbital nerve innervated forehead skin of the wearer 900. When the processing unit 50, 401 detects cessation of EEG signals suggestive of neuropsychiatric disorder, the processing unit 50, 401, may be configured to send signals to the supraorbital nerve stimulation unit 301 to stop the neuromodulating electric stimulation. When the processing unit 50, 401 detects cessation of EEG signals suggestive of impending neuropsychiatric disorder, the processing unit 50, 401, may be further configured to send signals to the supraorbital nerve stimulation unit 301 to stop the neuromodulating electric stimulation. In alternate embodiments, an infraorbital nerve stimulation unit may be included as another component of the neuromodulation unit 300. Infraorbital nerve is a branch from the second division of the trigeminal nerve. Infraorbital nerve stimulation has been found to be effective for conditions like trigeminal neuralgia, post-herpetic neuralgia, attention deficit hyperactivity disorder, depression, seizure etc. However, infraorbital nerve stimulation is more complex to set up and sometimes requires invasive procedure. The functions of the infraorbital nerve stimulation unit 305 is essentially the same as the aforementioned descriptions for the supraorbital nerve stimulation unit 301.

In some embodiments, an automatic detection-therapy system 101 may comprise a neuromodulation unit 300 that includes an auriculotemporal nerve (ATN) stimulation unit 302. When activated, the ATN stimulation unit 302 is configured to send neuromodulating electric stimulation to the ATN innervated auricular skin of the wearer's ear 902. When prompted by EEG findings as described hereinbefore, the auriculotemporal nerve stimulation unit 302 may be configured to start neuromodulating electric stimulation to the auriculotemporal nerve innervated auricular skin of the wearer 900 via an ATN stimulation electrode 312. When the processing unit 50, 401, detects presence of EEG signals suggestive of neuropsychiatric disorder, the processing unit 50, 401, may be configured to send signals to the ATN stimulation unit 302 to prompt the ATN stimulation unit 302 to start sending neuromodulating electric stimulation to the ATN innervated skin of the wearer's ear 902. When the processing unit 50, 401, detects presence of EEG signals suggestive of impending neuropsychiatric disorder, the processing unit 50, 401, may be configured to send signals to the auriculotemporal nerve (ATN) stimulation unit 302 to prompt the ATN stimulation unit 302 to start sending neuromodulating electric stimulation to the ATN innervated skin of the wearer's ear 902. When the processing unit 50, 401 detects cessation of EEG signals suggestive of neuropsychiatric disorder, the processing unit 50, 401, may be configured to send signals to the ATN stimulation unit 302 to stop the neuromodulating electric stimulation. When the processing unit 50, 401 detects cessation of EEG signals suggestive of impending neuropsychiatric disorder, the processing unit 50, 401, may be further configured to send signals to the ATN stimulation unit 302 to stop the neuromodulating electric stimulation. The ATN innervated auricular skin includes the anterior outer part of tragus 905, anterior superior portion of the cavum-concha 907, anterior-superior helix 909, anterior and superior walls of the external ear canal 904, anterior auricle and part of the outer tympanic membrane. Thus, the tragus-concha bowl 957 and the external ear canal 904 received mixed innervation from both the auricular branch of vagus nerve and the ATN. Alternatively, the ATN stimulation unit 302 may be configured to deliver electric stimulation via a clip electrode 319 for neuromodulating electric stimulation to ATN innervated anterior-superior helix 909 of the wearer's ear 902 when prompted.

In some embodiments, an automatic detection-therapy system 101 may comprise a neuromodulation unit 300 that includes an occipital nerve stimulation unit 303. When activated, the occipital nerve stimulation unit 303 is configured to send neuromodulating electric stimulation to the occipital nerve innervated occipital region of the wearer. When prompted (by EEG findings as described hereinbefore), the occipital nerve stimulation unit 303 may be configured to start neuromodulating electric stimulation to occipital nerve innervated skin at occipital region of the wearer 900 via an occipital nerve stimulating electrode 313. When the processing unit 50, 401, detects presence of EEG signals suggestive of neuropsychiatric disorder, the processing unit 50, 401, may be configured to send signals to the occipital nerve stimulation unit 303 to prompt the occipital nerve stimulation unit 303 to start sending neuromodulating electric stimulation to the occipital nerve innervated occipital region of the wearer 900. When the processing unit 50, 401, detects presence of EEG signals suggestive of impending neuropsychiatric disorder, the processing unit 50, 401, may be configured to send signals to the occipital nerve stimulation unit 303 to prompt the occipital nerve stimulation unit 303 to start sending neuromodulating electric stimulation to the occipital nerve innervated occipital region of the wearer 900. When the processing unit 50, 401 detects cessation of EEG signals suggestive of neuropsychiatric disorder, the processing unit 50, 401, may be configured to send signals to the occipital nerve stimulation unit 303 to stop the neuromodulating electric stimulation. When the processing unit 50, 401 detects cessation of EEG signals suggestive of impending neuropsychiatric disorder, the processing unit 50, 401, may be further configured to send signals to the occipital nerve stimulation unit 303 to stop the neuromodulating electric stimulation. In some embodiments, the supraorbital nerve stimulation unit 301, the occipital nerve stimulation unit 303 and the infraorbital nerve stimulation unit 305 may be installed manually by the wearer 900 when the wearer 900 received pertinent notification from the network interface 406. Alternatively, the supraorbital nerve stimulation unit 301, the occipital nerve stimulation unit 303 and the infraorbital nerve stimulation unit 305 may be pre-installed and may be configured to deliver neuromodulating electric stimulation automatically when prompted via wireless communication from the processing unit 50, 401. It should be noted that, in preferred embodiments, the taVNS stimulating electrode 31, the auriculotemporal nerve (ATN) stimulating electrode 312, the greater auricular nerve (GAN) stimulating electrode 314, all of the EEG sensor electrodes 12, 13, 72, 73, 82, 83 and the optional reference electrode 84 may be configured to be housed in a same auricular housing 11 and therefore will be automatically pre-installed when the wearer 900 places the auricular housing 11 into an ear 902 of the wearer 900.

In some embodiments, an automatic detection-therapy system 101 may comprise a neuromodulation unit 300 that includes a greater auricular nerve (GAN) stimulation unit 304. When activated, the GAN stimulation unit 304 is configured to send neuromodulating electric stimulation to the GAN innervated auricular skin of the wearer's ear 902. When prompted (by EEG findings as described hereinbefore), the greater auricular nerve (GAN) stimulation unit 304 may be configured to start neuromodulating electric stimulation to the GAN innervated auricular skin of the wearer 900 via a GAN stimulation electrode 314. When the processing unit 50, 401, detects presence of EEG signals suggestive of neuropsychiatric disorder, the processing unit 50, 401, may be configured to send signals to the GAN stimulation unit 304 to prompt the GAN stimulation unit 304 to start sending neuromodulating electric stimulation to the GAN innervated skin of the wearer's ear 902. When the processing unit 50, 401, detects presence of EEG signals suggestive of impending neuropsychiatric disorder, the processing unit 50, 401, may be configured to send signals to the GAN stimulation unit 304 to prompt the GAN stimulation unit 304 to start sending neuromodulating electric stimuli to the GAN innervated skin of the wearer's ear 902. When the processing unit 50, 401, detects cessation of EEG signals suggestive of neuropsychiatric disorder, the processing unit 50, 401, may be configured to send signals to the GAN stimulation unit 304 to stop the neuromodulating electric stimulation. When the processing unit 50, 401 detects cessation of EEG signals suggestive of impending neuropsychiatric disorder, the processing unit 50, 401, may be further configured to send signals to the GAN stimulation unit 304 to stop the neuromodulating electric stimulation. The greater auricular nerve (GAN) is a pure sensory nerve originated from the cervical spinal cord (C2, C3) and provides sensory innervation to the skin over the parotid gland, the mastoid process, and the lower two-thirds of the outer ear (pinna). The greater auricular nerve (GAN) innervated auricular skin includes skin on both surfaces of the pinna (including the lobule) and cavum concha (inferior/lower part of concha). Thus, the tragus-concha bowl 957 received mixed innervation from the auricular branch of vagus nerve, the auriculotemporal nerve (ATN) and the greater auricular nerve (GAN). Research indicates that neuromodulation techniques targeting the GAN may provide therapeutic relief for various neurological (including migraine, cluster headache, seizure) and psychiatric disorders.

In preferred embodiments, an automatic detection-therapy system 101 may comprise one or two neuromodulation units 300. A first neuromodulation unit 300 may be configured to include at least one of the following: a first transcutaneous auricular vagus nerve stimulation unit (taVNS unit) 30, a supraorbital nerve stimulation unit 301, a first auriculotemporal nerve (ATN) stimulation unit 302, an occipital nerve stimulation unit 303, a first greater auricular nerve (GAN) stimulation unit 304 and an infraorbital nerve stimulation unit 305, as aforementioned description. The first taVNS unit 30, the first ATN stimulation unit 302 and the first GAN stimulation unit 304 may be configured to be located at the wearer's first ear 902. The supraorbital nerve stimulation unit 301 may be located at midforehead and be configured to stimulate unilateral or bilateral supraorbital nerves. The occipital nerve stimulation unit 303 may be located at mid-occipital region and be configured to stimulate unilateral or bilateral occipital nerves. The infraorbital nerve stimulation unit 305 may be located at mid-face and be configured to stimulate unilateral or bilateral infraorbital nerves. Optionally, a second neuromodulation unit 300 may include a second taVNS unit 30, a second auriculotemporal nerve (ATN) stimulation unit 302 and a second greater auricular nerve (GAN) stimulation unit 304, configured to be located at the wearer's second ear 902. The setups and functions of the second ta VNS unit 30 are essentially the same as the first taVNS unit 30. The setups and functions of the second ATN stimulation unit 302 are essentially the same as the first ATN stimulation unit 302. Likewise, the setups and functions of the second GAN stimulation unit 304 are essentially the same as the first GAN stimulation unit 304. Studies have shown that bilateral neuromodulation is more effective than unilateral neuromodulation. Separately, studies have shown that simultaneous neuromodulation of two or three nerves on the same side is more effective than single nerve neuromodulation due to their synergistic effects. Double neuromodulation may consist of simultaneous neuromodulation of two nerves, such as simultaneous stimulation of auricular branch of vagus nerve and auriculotemporal nerve (ATN), or simultaneous neuromodulation of vagus nerve and greater auricular nerve (GAN) or simultaneous neuromodulation of vagus nerve and occipital nerve, etc. Triple neuromodulation may consist of simultaneous neuromodulation of three nerves, such as simultaneous neuromodulating electric stimulation to auricular branch of vagus nerve, auriculotemporal nerve (ATN) and greater occipital nerve (GAN), etc.

In some embodiments, an automatic detection-therapy system 101 may comprise an auricular electroencephalogram (EEG) monitoring system 100 having one or more EEG recording modules 20. In some embodiments, an automatic detection-therapy system 101 may have an auricular EEG monitoring system 100 having two EEG recording module 20 with each EEG recording module 20 linked to each ear. Each EEG recording module 20 preferably has a plurality of miniature wired or wireless EEG sensor electrodes 12, 13, 72, 73, 82, 83, and an optional wired or wireless reference electrode 84 that are configured to contact or be attached to separate areas selected from at least one of the following: the external ear canal 904, external ear 902, or peri-auricular area 903, that may be configured to record EEG data of the wearer 900, such as discussed above. Preferably, the wired or wireless EEG sensor electrodes 12, 13, 72, 73, 82, 83, are configured to be located in the wearer's external ear canal while the optional reference electrode 84 may be configured to be located at the wearer's tragus-concha bowl 957 or be attached to the mastoid of the wearer's peri-auricular area 903. In some embodiments for an automatic detection-therapy system 101, each auricular EEG recording module 20 may be configured to record EEG data of the wearer 900 via a plurality of EEG sensor electrodes 12,13, 72, 73, 82, 83, and an optional reference electrode 84. The recorded EEG data by the two EEG recording modules 20 may be transmitted or otherwise electronically communicated wired or wirelessly to a processing unit 50, 401. With the help of various advanced EEG analysis algorithms together with machine learning, deep learning and artificial intelligence (as known in the art), the processing unit 50, 401, may be configured to analyze the EEG data recorded by the two EEG recording modules 20 to detect presence or cessation of EEG signals suggestive of neuropsychiatric disorder. The processing unit 50, 401, may be also configured to analyze the EEG data to detect presence or cessation of EEG signals suggestive of impending neuropsychiatric disorder. The neuropsychiatric disorders that the two EEG recording modules 20 may be configured to detect may include seizure, neurodegenerative diseases, migraine, cluster headache, major depressive disorder (MDD), bipolar disorder, schizophrenia, obsessive compulsive disorder (OCD), attention deficit hyperactivity disorder (ADHD), autism spectrum disorder (ASD), post-traumatic stress disorder (PTSD), anxiety disorder and panic disorder, etc.

In some embodiments, an automatic detection-therapy system 101 may include one or two neuromodulation units, for example, two transcutaneous auricular vagus nerve stimulation units (taVNS units) 30, (such as a first taVNS unit 30 and a second taVNS unit 30), with each taVNS unit 30 having a taVNS stimulating electrode 31 configured to contact vagus innervated auricular skin of one of the wearer's ears 902. The vagus innervated auricular skin that the stimulating electrode 31 is configured to contact may be selected from at least one of the following: external ear canal 904, tragus 905, cymba-concha 906, and cavum-concha 907. The one or more EEG recording modules 20 and the one or two taVNS units 30 are in wired or wireless electronic communication with the processing unit 50, 401. In some embodiments, an automatic detection-therapy system 101 may include two neuromodulation units 300, such as, two auriculotemporal nerve stimulation units 302 and/or two greater auricular nerve stimulation units 304 and/or two taVNS units 30, similar to the aforementioned descriptions. The setups and functions of the second neuromodulation unit 300 is similar to those of the first neuromodulation unit 300 as described hereinbefore.

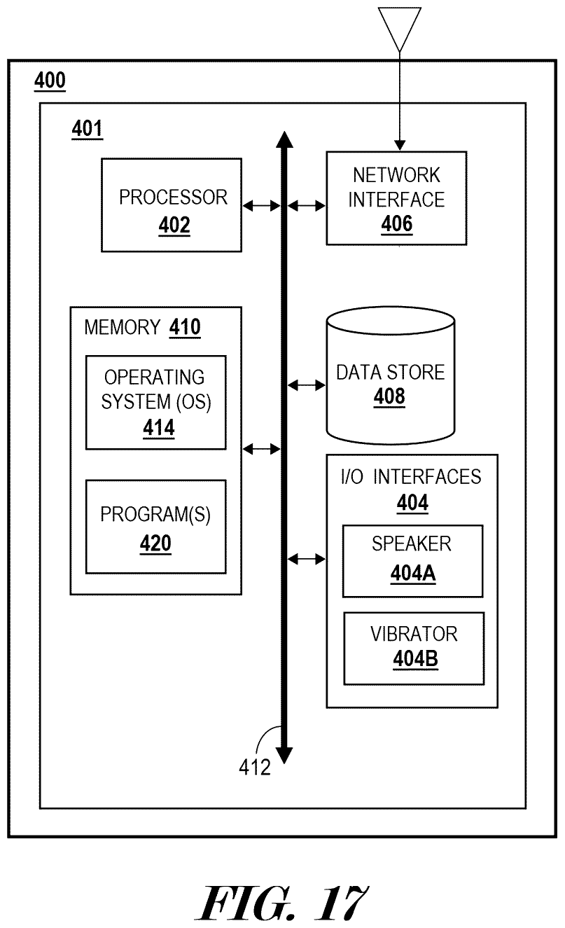

In some embodiments, an auricular EEG monitoring system 100 and/or an automatic detection-therapy system 101 may be in electronic communication with one or more client devices 400. In some embodiments, the auricular EEG monitoring system 100 may comprise one or more client devices 400. Referring to FIG. 17, in an exemplary embodiment, a block diagram illustrates a client device 400 of which may be a type of computing platform. A client device 400 can be a digital device that, in terms of hardware architecture, generally includes a processor 402, input/output (I/O) interfaces 404, a network interface 406, a data store 408, and memory 410. It may be appreciated by those of ordinary skill in the art that FIG. 17 depicts the client device 400 in an oversimplified manner, and a practical embodiment may include additional components and suitably configured processing logic to support known or conventional operating features that are not described in detail herein. The components (402, 404, 406, 408, and 410) are communicatively coupled via a local interface 412. The local interface 412 can be, for example but not limited to, one or more buses or other wired or wireless connections, as is known in the art. The local interface 412 can have additional elements, which are omitted for simplicity, such as controllers, buffers (caches), drivers, repeaters, and receivers, among many others, to enable communications.

In some embodiments, an auricular EEG monitoring system 100 and/or an automatic detection-therapy system 101 may comprise a processor 402. The processor 402 is a hardware device for executing software instructions. The processor 402 can be any custom made or commercially available processor, a central processing unit (CPU), an auxiliary processor among several processors associated with the client device 400, a semiconductor-based microprocessor (in the form of a microchip or chip set), or generally any device for executing software instructions. When the client device 400 is in operation, the processor 402 is configured to execute software stored within the memory 410, to communicate data to and from the memory 410, and to generally control operations of the client device 400 pursuant to the software instructions. In an exemplary embodiment, the processor 402 may include a mobile optimized processor such as optimized for power consumption and mobile applications.

In some embodiments, an auricular EEG monitoring system 100 and/or an automatic detection-therapy system 101 may comprise an I/O interface 404. The I/O interfaces 404 can be used to receive data and user input and/or for providing system output. User input can be provided via a plurality of I/O interfaces 404, such as a keypad, a touch screen, speaker 404A, a camera, a microphone, a scroll ball, a scroll bar, buttons, barcode scanner, voice recognition, eye gesture, and the like. System output can be provided via a display screen such as a liquid crystal display (LCD), touch screen, and the like. The I/O interfaces 404 can also include, for example, a global positioning service (GPS) radio, a serial port, a parallel port, a small computer system interface (SCSI), an infrared (IR) interface, a radio frequency (RF) interface, a universal serial bus (USB) interface, and the like. The I/O interfaces 404 can include a graphical user interface (GUI) that enables a user to interact with the client device 400. Additionally, the I/O interfaces 404 may be used to output notifications to a user and can include a speaker 404A or other sound emitting device configured to emit audio notifications, a vibrational device or vibrator 404B configured to vibrate, shake, or produce any other series of rapid and repeated movements to produce haptic notifications, and/or a light emitting diode (LED) or other light emitting element which may be configured to illuminate to provide a visual notification.

In some embodiments, an auricular EEG monitoring system 100 and/or an automatic detection-therapy system 101 may comprise a network interface 406. The network interface 406 enables wireless communication to an external access device or network. Any number of suitable wireless data communication protocols, techniques, or methodologies can be supported by the network interface 406, including, without limitation: RF; IrDA (infrared); Bluetooth; ZigBee (and other variants of the IEEE 802.15 protocol); IEEE 802.11 (any variation); IEEE 802.16 (WiMAX or any other variation); Direct Sequence Spread Spectrum; Frequency Hopping Spread Spectrum; Long Term Evolution (LTE); cellular/wireless/cordless telecommunication protocols (e.g. 3G/4G, etc.); wireless home network communication protocols; paging network protocols; magnetic induction; satellite data communication protocols; wireless hospital or health care facility network protocols such as those operating in the WMTS bands; GPRS; proprietary wireless data communication protocols such as variants of Wireless USB; and any other protocols for wireless communication.

In some embodiments, an auricular EEG monitoring system 100 and/or an automatic detection-therapy system 101 may comprise a data store 408. The data store 408 may be used to store data and is therefore a type of memory. The data store 408 may include any of volatile memory elements (e.g., random access memory (RAM, such as DRAM, SRAM, SDRAM, and the like)), nonvolatile memory elements (e.g., ROM, hard drive, tape, CDROM, and the like) and combinations thereof. Moreover, the data store 408 may incorporate electronic, magnetic, optical, and/or other types of storage media. In some embodiments, an auricular EEG monitoring system 100 and/or an automatic detection-therapy system 101 may comprise a memory 410. The memory 410 may include any of volatile memory elements (e.g., random access memory (RAM, such as DRAM, SRAM, SDRAM, etc.)), nonvolatile memory elements (e.g., ROM, hard drive, etc.), and combinations thereof. Moreover, the memory 410 may incorporate electronic, magnetic, optical, and/or other types of storage media. Note that the memory 410 may have a distributed architecture, where various components are situated remotely from one another, but can be accessed by the processor 402. The software in memory 410 can include one or more software programs 420, each of which includes an ordered listing of executable instructions for implementing logical functions. In the example of FIG. 17, the software in the memory system 410 includes an operating system (O/S) 414 and programs 420.

In some embodiments, an auricular EEG monitoring system 100 and/or an automatic detection-therapy system 101 may comprise an operating system 414. The operating system 414 essentially controls the execution of other computer programs, and provides scheduling, input-output control, file and data management, memory management, and communication control and related services. The operating system 414 may be, for example, LINUX (or another UNIX variant), Android (available from Google), Symbian OS, Microsoft Windows CE, Microsoft Windows 7 Mobile, Microsoft Windows 10, iOS (available from Apple, Inc.), webOS (available from Hewlett Packard), Blackberry OS (Available from Research in Motion), and the like.

In some embodiments, an auricular EEG monitoring system 100 and/or an automatic detection-therapy system 101 may comprise programs 420. The programs 420 may include various applications, add-ons, etc. configured to provide end user functionality with the client device 400. For example, exemplary programs 420 may include, but not limited to, a web browser, social networking applications, streaming media applications, games, mapping and location applications, electronic mail applications, financial applications, and the like.

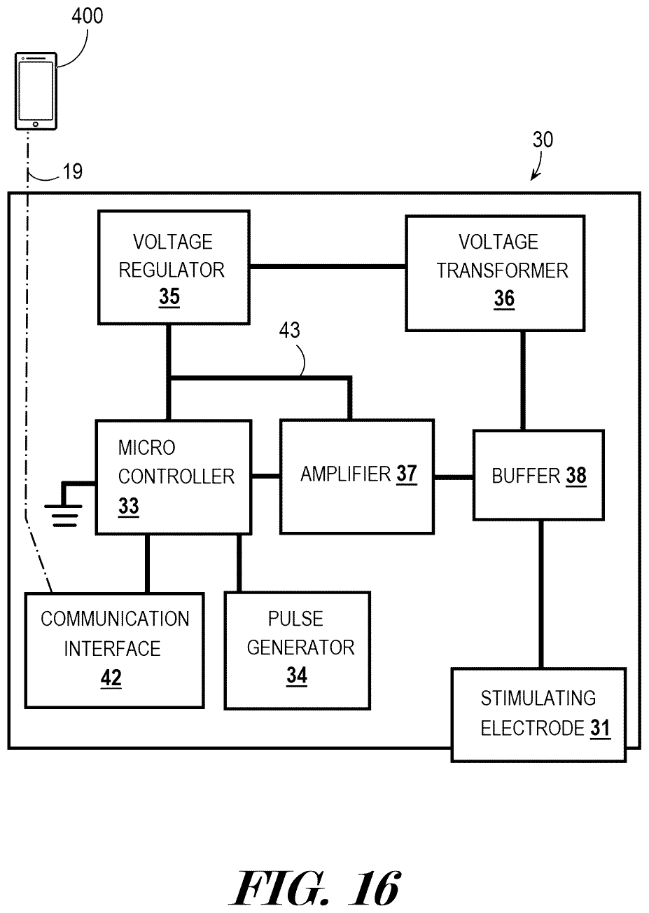

In some embodiments, an automatic detection-therapy system 101 may comprise a transcutaneous auricular vagus nerve stimulation unit (taVNS unit). The taVNS unit 30 may comprise any device that is able to provide transcutaneous auricular vagus nerve stimulation to a user's body. As an example, and referring to FIG. 16, a taVNS unit 30 may comprise a microcontroller 33 that may be in communication with a pulse generator 34, voltage regulator 35, voltage transformer 36, amplifier 37, and buffer 38, and that may be configured to generate taVNS stimuli that may be transmitted to vagus nerve innervated auricular skin via a taVNS stimulating electrode 31.

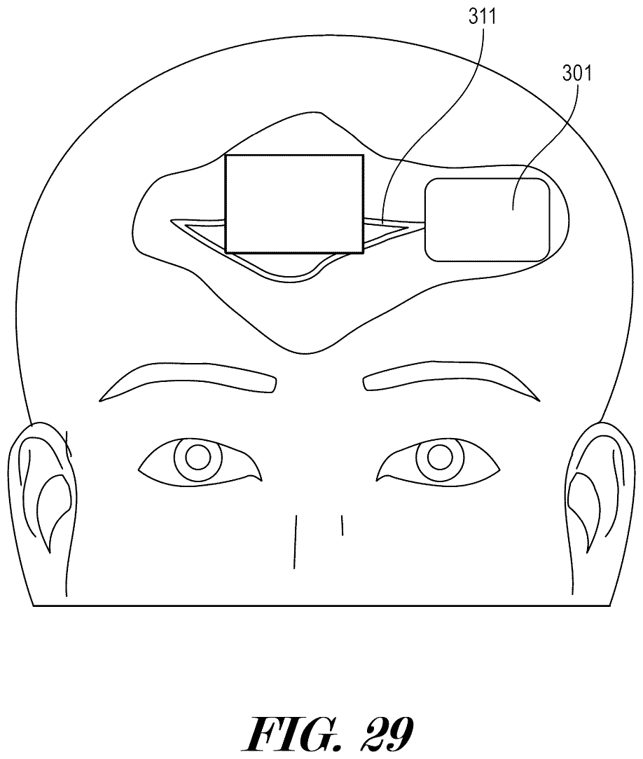

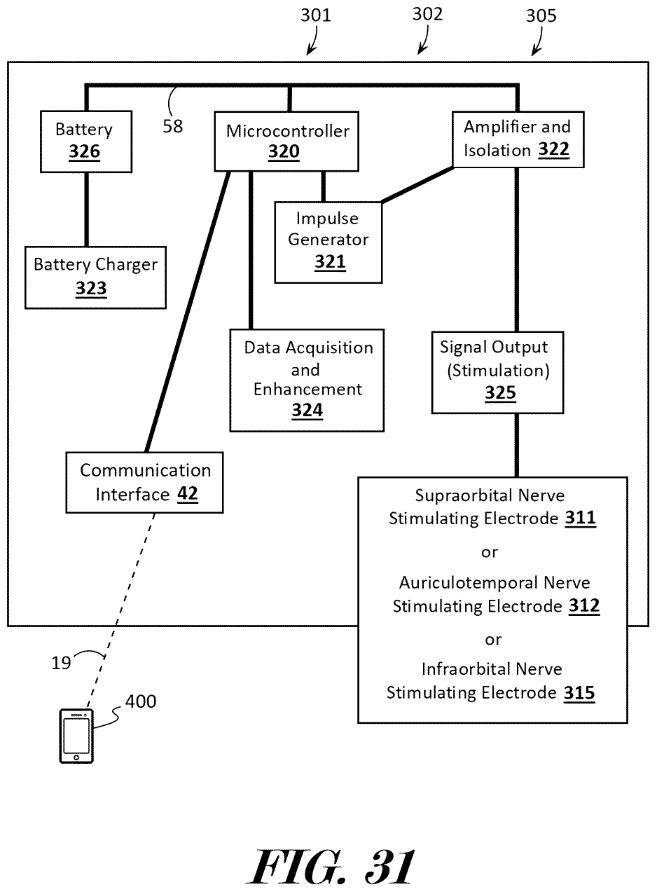

In some embodiments, an automatic detection-therapy system 101 may comprise a supraorbital nerve stimulation unit 301. The supraorbital nerve stimulation unit 301 may comprise any device that is able to provide transcutaneous supraorbital nerve stimulation to a user's body. As an example, and referring to FIGS. 29, 31, a supraorbital nerve stimulation unit 301 may comprise a microcontroller 320 that may be in communication with an impulse generator 321, amplifier and isolation 322, data acquisition and enhancement 324, signal output (stimuli) 325, and supraorbital nerve stimulating electrode 311 to supraorbital nerve(s). The supraorbital nerve stimulation unit 301 further comprises a battery 326, a battery charger 323 and a communication interface 42 for communication to a client device 400. The supraorbital nerve stimulation unit 301 may be configured to generate electric stimulation that may be transmitted to supraorbital nerve innervated forehead skin via the supraorbital nerve stimulating electrode 311. In some embodiments, an automatic detection-therapy system 101 may comprise an infraorbital nerve stimulation unit 305 having an infraorbital nerve stimulating electrode 315 configured to contact infraorbital nerve innervated midface region of the wearer 900. An infraorbital nerve stimulation unit 305 may comprise any device that is able to provide transcutaneous or non-transcutaneous supraorbital nerve stimulation to a user's body. The function of the infraorbital nerve stimulation unit 305 is essentially similar to the supraorbital nerve stimulation unit 301.

In some embodiments, an automatic detection-therapy system 101 may comprise an auriculotemporal nerve (ATN) stimulation unit 302. The ATN stimulation unit 302 may comprise any device that is able to provide transcutaneous auriculotemporal nerve stimulation to a user's body. As an example, and referring to FIG. 31, an ATN stimulation unit 302 may comprise a microcontroller 320 that may be in communication with an impulse generator 321, amplifier and isolation 322, data acquisition and enhancement 324, signal output (stimuli) 325, and auriculotemporal nerve (ATN) stimulating electrode 312, 319, to auriculotemporal nerve. The ATN stimulation unit 302 further comprises a battery 326, a battery charger 323 and a communication interface 42 for communication to a client device 400. The ATN stimulation unit 302 may be configured to generate electric stimuli that may be transmitted to ATN innervated auricular skin (such as external ear canal 904 or tragus-concha bowl 957) via the ATN stimulating electrode 312. Alternatively, the ATN stimulation unit 302 may be configured to generate electric stimulation that may be transmitted to ATN innervated auricular skin at anterior-superior helix 909 of the wearer's ear 902 via a clip electrode 319 for ATN stimulation.

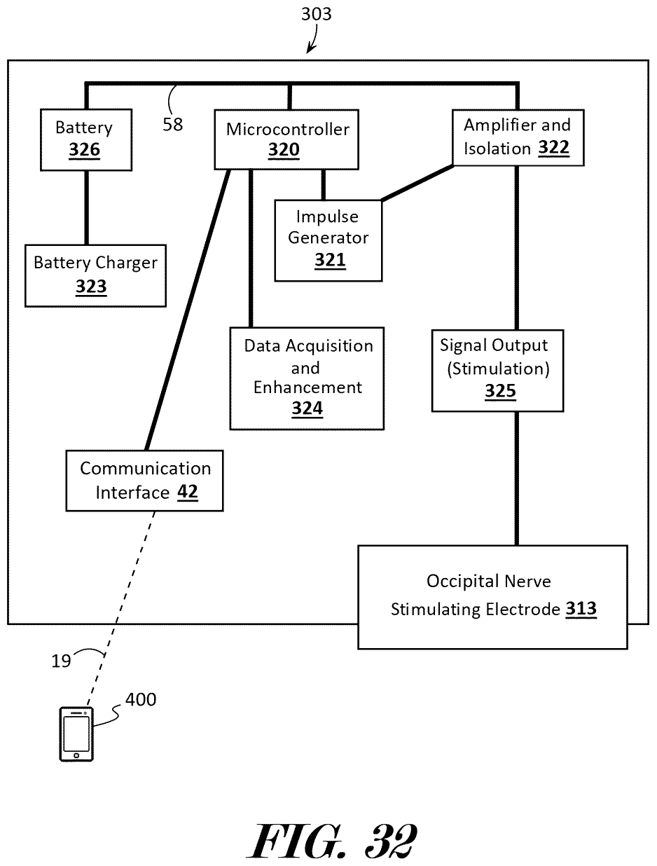

In some embodiments, an automatic detection-therapy system 101 may comprise an occipital nerve stimulation unit 303. The occipital nerve stimulation unit 303 may comprise any device that is able to provide occipital nerve stimulation to a user's body. As an example, and referring to FIG. 32, an occipital nerve stimulation unit 303 may comprise a microcontroller 320 that may be in communication with an impulse generator 321, amplifier and isolation 322, data acquisition and enhancement 324, signal output (stimuli) 325, and occipital nerve stimulating electrode 313. The occipital nerve stimulation unit 303 further comprises a battery 326, a battery charger 323 and a communication interface 42 for communication with a client device 400. The occipital nerve stimulation unit 303 may be configured to generate electric stimulation that may be transmitted to occipital nerve innervated occipital region via the occipital nerve stimulating electrode 313.

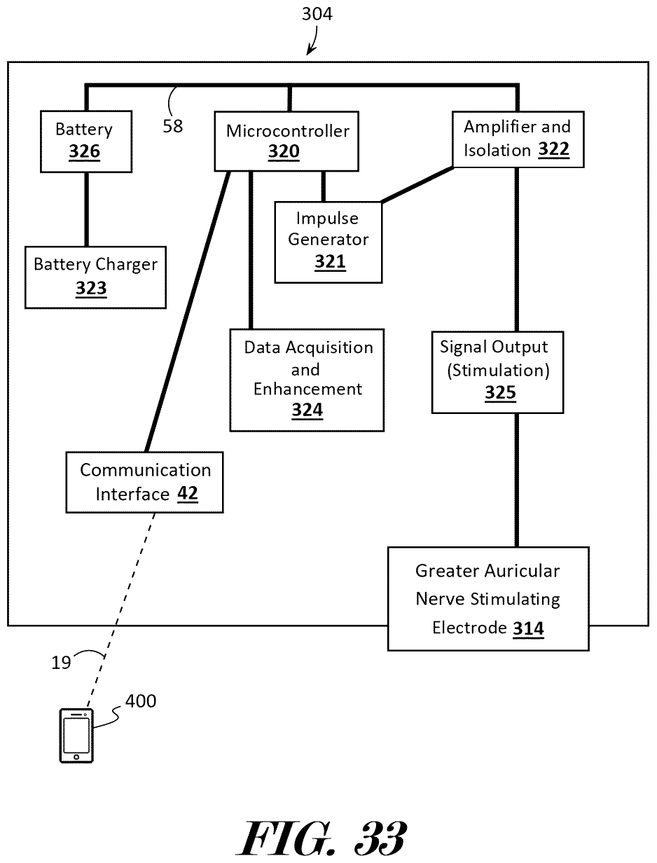

In some embodiments, an automatic detection-therapy system 101 may comprise a greater auricular nerve (GAN) stimulation unit 304. The GAN stimulation unit 304 may comprise any device that is able to provide greater auricular nerve stimulation to a user's body. As an example, and referring to FIG. 33, a GAN stimulation unit 304 may comprise a microcontroller 320 that may be in communication with an impulse generator 321, amplifier and isolation 322, data acquisition and enhancement 324, signal output (stimuli) 325, and GAN stimulating electrode 314. The GAN stimulation unit 304 further comprises a battery 326, a battery charger 323 and a communication interface 42 for communication with a client device 400. The GAN stimulation unit 304 may be configured to generate electric stimulation that may be transmitted to the GAN innervated auricular skin via the GAN stimulating electrode 314.

In some embodiments, each component of a neuromodulation unit 300 of an automatic detection-therapy system 101 may comprise a communication interface 42. Optionally, a taVNS unit 30 may comprise a communication interface 42 which may enable electronic communication 19 (e.g., wired and/or wireless communication) between the taVNS unit 30 and another electronic device, such as a client device 400, an auricular EEG monitoring system 100, etc. Optionally, a supraorbital nerve stimulation unit 301 may comprise a communication interface 42 which may enable electronic communication 19 (e.g., wired and/or wireless communication) with another electronic device, such as a client device 400, an auricular EEG monitoring system 100, etc. Optionally, an auriculotemporal nerve stimulation unit 302 may comprise a communication interface 42 which may enable electronic communication 19 (e.g., wired and/or wireless communication) with another electronic device, such as a client device 400, an auricular EEG monitoring system 100, etc. Likewise, optionally, an occipital nerve stimulation unit 303 may comprise a communication interface 42 which may enable electronic communication 19 (e.g., wired and/or wireless communication) with another electronic device, such as a client device 400, an auricular EEG monitoring system 100, etc. Optionally, an infraorbital nerve stimulation unit 305 may comprise a communication interface 42 which may enable electronic communication 19 (e.g., wired and/or wireless communication) with another electronic device, such as a client device 400, an auricular EEG monitoring system 100, etc. Similarly, a greater auricular nerve stimulation unit 304 may comprise a communication interface 42 which may enable electronic communication 19 (e.g., wired and/or wireless communication) with another electronic device, such as a client device 400, an auricular EEG monitoring system 100, etc. Preferably, a communication interface 42 may comprise a radio that may operate via WiFi and/or Bluetooth communication standards. In further embodiments, a communication interface 42 may be configured as a network interface 53 described above so that it may operate on any wireless and/or wired electronic communication 19 protocol that a network interface 53 may use.

In some embodiments, an automatic detection-therapy system 101 may comprise a taVNS unit 30. One or more components (31, 33, 34, 35, 36, 37, 38, 42) of a taVNS unit 30 may be contained in a housing and may be in electronic communication via a local interface 43. Preferably, an auricular housing 11 may be utilized for the housing purpose for the ta VNS unit 30. The auricular housing 11 may be configured in any size and shape, and may be made from or comprise plastic, elastomer, silicone or any other material used in the field of personal medical devices. In some embodiments, an auricular housing 11 may be configured as a modified earbud housing 61, a modified in-the-ear housing 62, a behind-the-ear-hearing-aid-style housing 63 or a tubular-shaped structure 66 (standalone tubular-shaped structure 66). Optionally, all of the EEG sensor electrodes 12, 13, 72, 73, 82, 83, the optional reference electrode 84 of an auricular EEG monitoring system 100 and the taVNS stimulating electrode 31 may be configured to be all housed in a single auricular housing 11. (Thus, the housing for taVNS stimulating electrode 31 and housing for all electrodes in system 100 may be integrally formed or molded together as a single unit). Optionally, a taVNS unit 30 may be housed in a standalone housing that may be separated from the auricular housing 11. In some embodiments, the taVNS unit 30 may include a taVNS stimulating electrode 31 which may be built within the auricular housing 11 for the taVNS unit 30 (so that the taVNS housing and taVNS stimulating electrode 31 may be located within a single unit). In further embodiments, a taVNS stimulating electrode 31 may be connected with the taVNS unit 30 through a wire (so that the taVNS stimulating electrode 31 may be remote from the taVNS unit 30).

In some embodiments, a neuromodulation unit 300 (of an automatic detection-therapy system 101) may comprise a taVNS unit 30 and an auriculotemporal nerve (ATN) stimulation unit 302. The ATN stimulation unit 302 may have a housing structure similar to the aforementioned housing for taVNS. In preferred embodiments, the taVNS unit 30 and the ATN stimulation unit 302 may share a same housing structure (an auricular housing 11). The taVNS unit 30 and the ATN stimulation unit 302 may be intergraded into a single unit and they might share a common stimulating electrode. However, in preferred embodiments, the ta VNS unit 30 and the auriculotemporal nerve (ATN) stimulation unit 302 should be separate units with separate stimulating electrodes (a taVNS stimulating electrode 31 and an ATN stimulating electrode 312) because it is preferred that the taVNS unit 30 and the ATN stimulation unit 302 use their own optimal distinct stimulation parameters. They may share a same housing structure (an auricular housing 11), such as one of: a modified earbud housing 61, a modified in-the-ear housing and a behind-the-ear-hearing-aid-style housing 63. In preferred embodiments, all of the EEG sensor electrodes 12, 13, 72, 73, 82, 83, the optional reference electrode 84, the taVNS stimulating electrode 31 and the ATN stimulating electrode 312 may be configured to be all sharing a same housing structure (an auricular housing 11), selected from one of: a modified earbud housing 61, a modified in-the-ear housing 62, and a behind-the-ear-hearing-aid-style housing 63. The setups and the locations of the electrodes 12, 13, 72, 73, 82, 83, 84, 31, 312 and the material of the auricular housing 11 will provide huge convenience and advantages for the wearer (user) as described hereinbefore. Vagus nerve innervated auricular skin that the taVNS stimulating electrode 31 may be attached to may be selected from at least one of the following: inner/posterior portion of tragus 905, cymba-concha 906, cavum-concha 907, (i.e. tragus-concha bowl 957) and posterior and inferior walls of external ear canal 904. These areas are innervated by the auricular branch of the vagus nerve. The external ear canal 904 and the tragus-concha bowl 957 are also suitable for attachment of the auriculotemporal nerve (ATN) stimulation electrode 312. Optionally, the taVNS unit 30 and the ATN stimulation unit 302 may also be attached to the tragus-concha bowl 957 or the external ear canal 904. The tragus-concha bowl 957 and the external ear canal 904 are inherently stable for attachment of a taVNS unit 30 and an ATN stimulation unit 302 on a long-term basis.

In some embodiments, a neuromodulation unit 300 (of an automatic detection-therapy system 101) may comprise a taVNS unit 30, an auriculotemporal nerve (ATN) stimulation unit 302 and a greater auricular nerve (GAN) stimulation unit 304. The ATN stimulation unit 302 and the GAN stimulation unit 304 may have a housing structure similar to the aforementioned housing for taVNS. Alternatively, in preferred embodiments, the taVNS unit 30, the ATN stimulation unit 302 and the GAN stimulation unit 304 may share a same housing structure (an auricular housing 11). The taVNS unit 30, the ATN stimulation unit 302 and the GAN stimulation unit 304 may be intergraded into a single unit and some of them might even share a common stimulating electrode. However, in preferred embodiments, the ta VNS unit 30, the ATN stimulation unit 302 and the GAN stimulation unit 304 should be separate units with separate stimulating electrodes (a taVNS stimulating electrode 31, an ATN stimulating electrode 312 and a GAN stimulating electrode 314) because it is preferred that the taVNS unit 30, the ATN nerve stimulation unit 302 and the GAN stimulation unit 304 use their own optimal distinct stimulation parameters. They may share a same housing structure (e.g. an auricular housing 11), such as one of: a modified earbud housing 61, a modified in-the-ear housing and a behind-the-ear-hearing-aid-style housing 63. In preferred embodiments, all of the EEG sensor electrodes 12, 13, 72, 73, 82, 83, the optional reference electrode 84, the taVNS stimulating electrode 31, the auriculotemporal nerve (ATN) stimulating electrode 312 and the greater auricular nerve (GAN) stimulating electrode 314 may be configured to be all sharing a same housing structure (an auricular housing 11), selected from one of: a modified earbud housing 61, a modified in-the-ear housing 62, and a behind-the-ear-hearing-aid-style housing 63. The setups and the locations of the electrodes 12, 13, 72, 73, 82, 83, 84, 31, 312, 314 and the material of the housing structure 11 will provide huge convenience and advantages for the wearer (user) as described hereinbefore. Vagus nerve innervated auricular skin that the taVNS stimulating electrode 31 may be attached to may be selected from at least one of the following: inner/posterior portion of tragus 905, cymba-concha 906, cavum-concha 907 (i.e. tragus-concha bowl 957), and posterior and inferior walls of external ear canal 904. The external ear canal 904 and the tragus-concha bowl 957 are also suitable for attachment of the auriculotemporal nerve (ATN) stimulation electrode 312. The cavum concha 907 is suitable for attachment of the greater auricular nerve (GAN) stimulating electrode 314. Optionally, the taVNS unit 30 and the ATN stimulation unit 302 may be attached to the tragus-concha bowl 957 or the external ear canal 904, while the GAN stimulating unit 304 may be attached to the tragus-concha bowl 957. The tragus-concha bowl 957 and external ear canal 904 are inherently stable for attachment of the ta VNS unit 30, the ATN stimulation unit 302 and the GAN stimulation unit 304 on a long-term basis.

In preferred embodiments of an automatic detection-therapy system 101, when presence of EEG signals suggestive of neuropsychiatric disorders is detected by the processing unit 50, 401, the processing unit 50, 401, may be configured to automatically send signals to the neuromodulation unit 300 to prompt at least one of the following: a taVNS unit 30, a supraorbital nerve stimulation unit 301, an auriculotemporal nerve stimulation unit 302, an occipital nerve stimulation unit 301, a greater auricular nerve stimulation unit 304 and an infraorbital nerve stimulation unit 305 to immediately start sending neuromodulating electric simulation, For example, when prompted, the taVNS unit 30 may be configured to start sending neuromodulating electric stimulation to the auricular branch of vagus nerve via the ta VNS stimulating electrode 31, utilizing pre-determined stimulation parameters, such as shown in Table 1 (for neuropsychiatric disorders) and Table 2 (for impending neuropsychiatric disorders).

TABLE 1
Example of taVNS unit 30 electric stimuli output
parameters for neuropsychiatric disorders.

| Output | Parameter | |
|---|---|---|
| Power supply | Direct current | |
| | 3-9 | volts |
| Pulse width | 0.05-1.0 | milliseconds (ms) |
| Frequency | 0.5-200 | Hz |
| Modes | Continuous wave or | |
| | sparse-dense wave | |
| Intensity | 0.1-15 | milliamperes (mA) |

TABLE 2
Example of taVNS unit 30 electric stimuli
output parameters for impending
neuropsychiatric disorders.

| Output | Parameter | |
|---|---|---|
| Power supply | Direct current | |
| | 3-9 | volts |
| Pulse width | 0.05-0.9 | milliseconds (ms) |
| Frequency | 0.5-150 | Hz |
| Modes | Continuous wave or | |
| | sparse-dense wave | |
| Intensity | 0.1-10 | milliamperes (mA) |

In preferred embodiments of an automatic detection-therapy system 101, when prompted, the taVNS unit 30 may be configured to start sending electric stimulation to the auricular branch of vagus nerve via the taVNS stimulating electrode 31, utilizing pre-determined stimulation parameters. The stimulation parameters for each individual neuropsychiatric disorder may be similar to or may be somewhat different from the general taVNS stimulation parameters for neuropsychiatric disorders. Examples of the taVNS stimulation parameters for seizure are shown in Table 3 (for seizure) and Table 4 (for impending seizure).

TABLE 3
Example of taVNS unit 30 electric stimuli
output parameters for seizure (epilepsy).

| Output | Parameter | |
|---|---|---|
| Power supply | Direct current | |
| | 3-9 | volts |
| Pulse width | 0.25-0.5 | ms |
| | (range 0.13-1 | ms) |
| Frequency | 10-25 | Hz |
| Modes | Continuous wave or | |
| | sparse-dense wave | |
| Intensity | 0.25-1.75 | mA |
| On/off time | 30 | seconds (s) on/ |
| | 3 | minutes (min) off |

| | |
|---|---|
| | (range 7 s-120 s on/ |
| | 18 s-30 min off) |
| Laterality | Bilateral or alternating |
| | between left and right |

TABLE 4
Example of taVNS unit 30 electric stimuli output
parameters for impending seizure (epilepsy)

| Output | Parameter | |
|---|---|---|
| Power supply | Direct current | |
| | 3-9 | volts |
| Pulse width | 0.25-0.5 | ms |
| | (range 0.13-1 | ms) |
| Frequency | 10-25 | Hz |
| Modes | Continuous wave or | |
| | sparse-dense wave | |
| Intensity | 0.25-1.25 | mA |
| On/off time | 30 | seconds (s) on/ |
| | 5 | minutes (min) off |

| | |
|---|---|
| | (range 7 s-120 s on/ |
| | 18 s-60 min off) |
| Laterality | Bilateral or alternating |
| | between left and right |

In some embodiments for an automatic detection-therapy system 101, examples of the taVNS unit 30 stimulation parameters for migraine are shown in Table 5 (for migraine) and Table 6 (for impending migraine).

TABLE 5
Example of taVNS unit 30 electric stimuli
output parameters for migraine

| Output | Parameter | |
|---|---|---|
| Power supply | Direct current | |
| | 3-9 | volts |
| Pulse width | 0.05-0.25 | ms |
| Frequency | 1-25 | Hz |
| Modes | Bi-phasic waveform | |
| Intensity | 0.1-5.0 | mA |
| | (Start at 0.1 | mA, |
| | adjust at | |
| | 0.1 mA | increment |
| | till maximum | |
| | tolerable intensity) | |
| On/off time | 30 | sec on/ |
| | 30 | sec off |
| Sessions | 30-240 | min/session, |
| | daily or 3 | times/week, |
| | total duration | |
| | 4-12 | weeks |

TABLE 6
Example of taVNS unit 30 electric stimuli
output parameters for impending migraine

| Output | Parameter | |
|---|---|---|
| Power supply | Direct current | |
| | 3-9 | volts |
| Pulse width | 0.05-0.25 | ms |
| Frequency | 1-25 | Hz |
| Modes | Bi-phasic waveform | |
| Intensity | 0.1-3.0 | mA |
| | (Start at 0.1 | mA, |
| | adjust at 0.1 | mA |
| | increment till maximum | |
| | tolerable intensity) | |
| On/off time | 30 | sec on/ |
| | 30 | sec off |
| Sessions | 30-200 | min/session, |
| | daily or 3 | times/week, |
| | total duration | |
| | 4-8 | weeks |

In preferred embodiments of an automatic detection-therapy system 101, examples of the taVNS unit 30 stimulation parameters for cluster headache are shown in Table 7 (for cluster headache) and Table 8 (for impending cluster headache).

TABLE 7
Example of taVNS unit 30 electric stimuli
output parameters for cluster headache

| Output | Parameter | |
|---|---|---|
| Power supply | Direct current | |
| | 3-9 | volts |
| Pulse width | 0.05-0.45 | ms |
| Frequency | 10-30 | Hz |
| Modes | Continuous wave or | |
| | sparse-dense wave | |
| Intensity | 0.1-5 | mA |
| Sessions | 20-60 | min/session, |
| | Duration 4 | weeks |

TABLE 8
Example of taVNS unit 30 electric stimuli output
parameters for impending cluster headache

| Output | Parameter | |
|---|---|---|
| Power supply | Direct current | |
| | 3-9 | volts |
| Pulse width | 0.05-0.45 | ms |
| Frequency | 10-30 | Hz |
| Modes | Continuous wave or | |
| | sparse-dense wave | |
| Intensity | 0.1-4.0 | mA |
| Sessions | 20-60 | min/session, |
| | Duration 3 | weeks |

In In preferred embodiments of an automatic detection-therapy system 101, examples of the taVNS unit 30 stimulation parameters for major depressive disorder (MDD) are shown in Table 9 (for MDD) and Table 10 (for impending MDD).

TABLE 9
Example of taVNS unit 30 electric stimuli output
parameters for major depressive disorder

| Output | Parameter | |
|---|---|---|
| Power supply | Direct current | |
| | 3-9 | volts |
| Pulse width | 0.2-1.0 | ms |
| Frequency | 20-25 | Hz |
| Modes | Continuous wave or | |
| | sparse-dense wave | |
| Intensity | 0.5-6.0 | milliampere (mA) |
| On/off time | 30 | sec on/ |
| | 30 | sec off |
| Sessions | 60-240 | min/day |
| | 5-7 | days/week, |
| | Duration 4-12 | weeks |

TABLE 10
Example of taVNS unit 30 electric stimuli output
parameters for impending major depressive disorder

| Output | Parameter | |
|---|---|---|
| Power supply | Direct current | |
| | 3-9 | volts |
| Pulse width | 0.2-1.0 | ms |
| Frequency | 20-25 | Hz |
| Modes | Continuous wave or | |
| | sparse-dense wave | |
| Intensity | 0.5-4.0 | milliampere (mA) |
| On/off time | 30 | sec on/ |
| | 30 | sec off |
| Sessions | 60-220 | min/day, |
| | 5-7 | days/week, |
| | Duration 4-8 | weeks |

In preferred embodiments of an automatic detection-therapy system 101, examples of the taVNS unit 30 stimulation parameters for bipolar disorder are shown in Table 11 (for bipolar disorder) and Table 12 (for impending bipolar disorder).

TABLE 11
Example of taVNS unit 30 electric stimuli
output parameters for bipolar disorder

| Output | Parameter | |
|---|---|---|
| Power supply | Direct current | |
| | 3-9 | volts |
| Pulse width | 0.25-1.0 | ms |
| Frequency | 20-30 | Hz |
| Modes | Continuous wave or | |
| | sparse-dense wave | |
| Intensity | 0.13-6.0 | milliampere (mA) |
| On/off time | 30 | sec on/ |
| | 60 | sec off |
| Sessions | 30-180 | min/day, |
| | 5-7 | days/week, |
| | Duration 2-6 | months |

TABLE 12
Example of taVNS unit 30 electric stimuli output
parameters for impending bipolar disorder

| Output | Parameter | |
|---|---|---|
| Power supply | Direct current | |
| | 3-9 | volts |
| Pulse width | 0.25-1.0 | ms |
| Frequency | 20-30 | Hz |
| Modes | Continuous wave or | |
| | sparse-dense wave | |
| Intensity | 0.13-4.0 | milliampere (mA) |
| On/off time | 30 | sec on/ |
| | 60 | sec off |
| Sessions | 30-160 | min/day, |
| | 5-7 | days/week, |
| | Duration 2-4 | months |

In some embodiments for automatic detection-therapy system 101, the taVNS unit 30 stimulating parameters for other neuropsychiatric disorders (including schizophrenia, ADHD, OCD, ASD, PTSD, anxiety disorder and panic disorder, etc.) are similar to those for MDD and bipolar disorder. It should be noted that the aforementioned taVNS stimulating parameters are just for exemplary purpose. Various stimulating parameters may be utilized without departing from the scope of this invention.

In preferred embodiments of an automatic detection-therapy system 101, the neuromodulating unit 300 may comprise at least one of the following components: a taVNS unit 30, a supraorbital nerve stimulation unit 301, an auriculotemporal nerve stimulation unit 302, an occipital nerve stimulation unit 303, a greater auricular nerve stimulation unit 304 and an infraorbital nerve stimulation unit 305. Examples of the stimulation parameters for the supraorbital nerve stimulation unit 301, the auriculotemporal nerve stimulation unit 302, the occipital nerve stimulation unit 303, the greater auricular nerve stimulation unit 304 and the infraorbital nerve stimulation unit 305 for neuropsychiatric disorders and impending neuropsychiatric disorders are as follows:
(1). Intensity/Strength: 2-4 mA (with a range of 1-10 mA, maximum of 16 mA), adjusted for comfort to a mild tingling sensation without pain.
(2). Frequency: 60-120 Hz
(3). Pulse width (Duration): 200 to 250 microseconds.
(4). Stimulation cycle: commonly 30 seconds on/30 seconds off or continuous for 20-60 minutes depending on condition.
(5). Session duration: up to 7-9 hours (overnight) as needed, depending on condition.
(6). Waveform: usually biphasic pulses.

Other examples of stimulation parameters for neuropsychiatric disorders for the taVNS unit 30, the supraorbital nerve stimulation unit 301, the auriculotemporal nerve stimulation unit 302, the occipital nerve stimulation unit 303, the greater auricular nerve stimulation unit 304 and the infraorbital nerve stimulation unit 305 are similar to or may be modified from the aforementioned parameters, such as discussed in the Background section.

In preferred embodiments for an automatic detection-therapy system 101, when a processing unit 50, 401, detects presence of EEG signals suggestive of neuropsychiatric disorders or impending neuropsychiatric disorders, the processing unit 50, 401, may be configured to send signals to the neuromodulation unit 300 to prompt it to start the neuromodulating electric stimulation. The settings or parameters for electric stimulation by each component of the neuromodulating unit 300 (including: a taVNS unit 30, a supraorbital nerve stimulation unit, an auriculotemporal nerve stimulation unit 302, an occipital nerve stimulation unit 303, a greater auricular nerve stimulation unit 304 and an infraorbital nerve stimulation unit 305) are pre-determined to have the most effective parameter for each neuropsychiatric disorder and for each impending neuropsychiatric disorder regarding each component of the neuromodulation unit 300. Similar or different stimulation parameters may be utilized for each neuropsychiatric disorder and for each impending neuropsychiatric disorder. As disclosed herein, an automatic detection-therapy system 101 having the novel integration of an auricular EEG recording module 20 and a neuromodulation unit 300 achieves very important dual functions, namely long-term EEG monitoring from the ear 902 and automatic instant therapeutic intervention by at least one component of the neuromodulation unit 300 in response to specific EEG findings.

In some embodiments for automatic detection-therapy system 101, for most patients or wearers 900 with neuropsychiatric disorders, two auricular EEG recording modules 20, one linked to each side of the external ear 902 or the peri-auricular area 903, may be preferred. In some situations, only one auricular EEG recording module 20 may be enough if one-sided partial EEG can adequately detect a neuropsychiatric disorder. In some embodiments, for patients with neuropsychiatric disorders, one neuromodulation unit 300 will be utilized. In some other embodiments, a second neuromodulation unit with at least one of: a second taVNS unit 30, a second auriculotemporal nerve stimulation unit 302 and a second greater auricular nerve stimulation unit 304 will be utilized. In some embodiments, an automatic detection-therapy system 101 may comprise an auricular EEG monitoring system 100 having two EEG recording module 20, with one EEG recording module 20 linked to each ear 902. The automatic detection-therapy system 101 may include two neuromodulation units 300 with a first neuromodulation unit 300 and a second neuromodulation unit 300. The first neuromodulation unit 300 comprises at least two of the following components: a first taVNS unit 30 (linked to a first ear 902 of the wearer), a supraorbital nerve stimulation unit 301, a first auriculotemporal nerve stimulation unit 302 (linked to the wearer's first ear 902), an occipital nerve stimulation unit 303, a first greater auricular nerve stimulation unit 304 (linked to the wearer's first ear 902) and an infraorbital nerve stimulation unit 305, (units 30, 301, 302, 303, 304, 305, as described hereinbefore). The second neuromodulation unit 300 may be linked to the wearer's second ear 902 and may comprise at least one of the following components: a second ta VNS unit 30, a second auriculotemporal nerve stimulation unit 302 and a second greater auricular nerve stimulation unit 304. The second taVNS unit 30 includes a second taVNS stimulating electrode 31 configured to contact vagus innervated auricular skin of the wearer's second ear 902. The second auriculotemporal nerve stimulation unit 302 includes a second auriculotemporal nerve stimulating electrode 312 configured to contact auriculotemporal nerve innervated auricular skin of the wearer's second ear 902. The second greater auricular nerve stimulation unit 304 includes a second greater auricular nerve stimulating electrode 314 configured to contact greater auricular nerve innervated auricular skin of the wearer's second ear 902. The processing unit 50, 401, may be configured to analyze the EEG data transmitted from the auricular EEG monitoring system 100, including EEG data recorded by the first EEG recording module 20 and EEG data recorded by the second EEG recording module 20, to detect presence or cessation of EEG signals suggestive of at least one neuropsychiatric disorder. The processing unit 50, 401, may be further configured to analyze the EEG data transmitted from the auricular EEG monitoring system 100, including EEG data recorded by the first EEG recording module 20 and EEG data recorded by the second EEG recording module 20, to detect presence or cessation of EEG signals suggestive of at least one impending neuropsychiatric disorder. When the processing unit 50, 401, detects at least one of the following: presence of EEG signals suggestive of at least one neuropsychiatric disorder by analyzing EEG data recorded by the first EEG recording module 20 and presence of EEG signals suggestive of at least one neuropsychiatric disorder by analyzing EEG data recorded by the second EEG recording module 20, the processing unit 50, 401, is configured to start sending predetermined neuromodulating electric stimulation from at least one of the following: at least two components of the first neuromodulation unit 300 and at least one component of the second neuromodulation unit 300. When the processing unit 50, 401, detects at least one of the following: presence of EEG signals suggestive of at least one impending neuropsychiatric disorder by analyzing EEG data recorded by the first EEG recording module 20 and presence of EEG signals suggestive of at least one impending neuropsychiatric disorder by analyzing EEG data recorded by the second EEG recording module 20, the processing unit 50, 401, is configured to start sending predetermined neuromodulating electric stimulation from at least one of the following: at least two components of the first neuromodulation unit 300 and at least one component of the second neuromodulation unit 300. When the processing unit 50, 401, detects all of the following: cessation of EEG signals suggestive of at least one neuropsychiatric disorder by analyzing EEG data recorded by the first EEG recording module 20, cessation of EEG signals suggestive of at least one impending neuropsychiatric disorder by analyzing EEG data recorded by the first EEG recording module 20, cessation of EEG signals suggestive of at least one neuropsychiatric disorder by analyzing EEG data recorded by the second EEG recording module 20 and cessation of EEG signals suggestive of at least one impending neuropsychiatric disorder by analyzing EEG data recorded by the second EEG recording module 20, the processing unit 50, 401, is configured to stop sending predetermined neuromodulating electric stimulation from any of the following: the first taVNS unit 30, the first auriculotemporal nerve stimulation unit 302, the first greater auricular nerve stimulation unit 304, the second taVNS unit 30, the second auriculotemporal nerve stimulation unit 302, the second greater auricular nerve stimulation unit 304, the supraorbital nerve stimulation unit 301, the infraorbital nerve stimulation unit 305 and the occipital nerve stimulation unit 303. In alternate embodiments, an automatic detection-therapy system 101 may comprise an auricular EEG monitoring system 100 having two EEG recording module 20, with one EEG recording module 20 linked to each ear 902. The automatic detection-therapy system 101 may include two neuromodulation units 300, including a first neuromodulation unit 300 and a second neuromodulation unit 300. The first neuromodulation unit 300 comprises at least one of the following components: a first ta VNS unit 30 (linked to a first ear 902 of the wearer 900), a supraorbital nerve stimulation unit 301, a first auriculotemporal nerve stimulation unit 302 (linked to the wearer's first ear 902), an occipital nerve stimulation unit 303, a first greater auricular nerve stimulation unit 304 (linked to the wearer's first ear 902) and an infraorbital nerve stimulation unit 305. The second neuromodulation unit 300 may be linked to a second ear 902 of the wearer 900 and may comprise at least one of the following components: a second taVNS unit 30, a second auriculotemporal nerve stimulation unit 302 and a second greater auricular nerve stimulation unit 304. The processing unit 50, 401, may be configured to analyze the EEG data transmitted from the auricular EEG monitoring system 100, including EEG data recorded by the first EEG recording module 20 and EEG data recorded by the second EEG recording module 20, to detect presence or cessation of EEG signals suggestive of at least one neuropsychiatric disorder. The processing unit 50, 401, is further configured to analyze the EEG data transmitted from the auricular EEG monitoring system 100, including EEG data recorded by the first EEG recording module 20 and EEG data recorded by the second EEG recording module 20, to detect presence or cessation of EEG signals suggestive of at least one impending neuropsychiatric disorder. When the processing unit 50, 401, detects at least one of the following: presence of EEG signals suggestive of at least one neuropsychiatric disorder by analyzing EEG data recorded by the first EEG recording module and presence of EEG signals suggestive of at least one neuropsychiatric disorder by analyzing EEG data recorded by the second EEG recording module, the processing unit 50, 401, is configured to start sending predetermined neuromodulating electric stimulation from at least one of the following: the first taVNS unit 30, the first auriculotemporal nerve stimulation unit 302, the first greater auricular nerve stimulation unit 304, the second taVNS unit 30, the second auriculotemporal nerve stimulation unit 302, the second greater auricular nerve stimulation unit 304, the supraorbital nerve stimulation unit 301, the infraorbital nerve stimulation unit 305 and the occipital nerve stimulation unit 303. When the processing unit 50, 401, detects at least one of the following: presence of EEG signals suggestive of at least one impending neuropsychiatric disorder by analyzing EEG data recorded by the first EEG recording module 20 and presence of EEG signals suggestive of at least one impending neuropsychiatric disorder by analyzing EEG data recorded by the second EEG recording module 20, the processing unit 50, 401, is configured to start sending predetermined neuromodulating electric stimulation from at least one of the following: the first taVNS unit 30, the first auriculotemporal nerve stimulation unit 302, the first greater auricular nerve stimulation unit 304, the second taVNS unit 30, the second auriculotemporal nerve stimulation unit 302, the second greater auricular nerve stimulation unit 304, the supraorbital nerve stimulation unit 301, the infraorbital nerve stimulation unit 305 and the occipital nerve stimulation unit 303. When the processing unit 50, 401, detects all of the following: cessation of EEG signals suggestive of at least one neuropsychiatric disorder by analyzing EEG data recorded by the first EEG recording module 20, cessation of EEG signals suggestive of at least one impending neuropsychiatric disorder by analyzing EEG data recorded by the first EEG recording module 20, cessation of EEG signals suggestive of at least one neuropsychiatric disorder by analyzing EEG data recorded by the second EEG recording module 20 and cessation of EEG signals suggestive of at least one impending neuropsychiatric disorder by analyzing EEG data recorded by the second EEG recording module 20, the processing unit 50, 401, is configured to stop sending predetermined neuromodulating electric stimulation from any of the following: the first taVNS unit 30, the first auriculotemporal nerve stimulation unit 302, the first greater auricular nerve stimulation unit 304, the second taVNS unit 30, the second auriculotemporal nerve stimulation unit 302, the second greater auricular nerve stimulation unit 304, the supraorbital nerve stimulation unit 301, the infraorbital nerve stimulation unit 305 and the occipital nerve stimulation unit 303.

In some embodiments, one or more elements of an automatic detection-therapy system 101 may be housed in a modified hearing aid style structure. The traditional hearing aids include in-the-ear-hearing-aid style and behind-the-ear-hearing-aid style and both styles have close contact with the skin of tragus 905, cavum-concha 907, and external ear canal 904. These areas are the optimal locations for attachment of the elements of the present invention. (FIG. 4 shows anatomy of external ear.) Combining elements of the present invention with a hearing aid will be a welcoming set-up for patients who need hearing aids and neuromodulation. For patients who do not need hearing aids, a modified earbud housing 61, a modified in-the-ear housing 62, or a behind-the-ear-hearing-aid-style housing 63 may be used for housing of an automatic detection-therapy system 101 of the present invention. These locations and structures provide inherently secure and stable attachment.

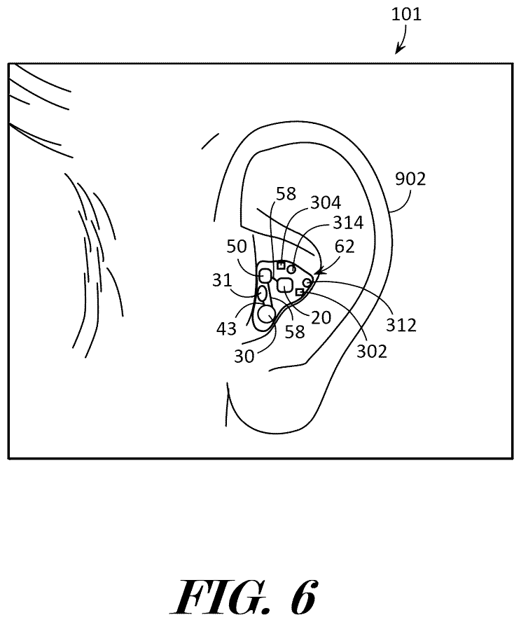

In some embodiments, a processing unit 50 can be incorporated within an auricular housing 11 of the automatic detection-therapy system 101 and housed in one of: a modified earbud housing 61, a modified in-the-ear housing 62, and a behind-the-ear-hearing-aid-style housing 63. (modified in-the-ear housing shown in FIGS. 3, 6). If the processing unit 50 is housed at a location very close to the auricular EEG recording module 20 and ta VNS unit 30, they can be connected through wire type local interface 58, instead of through wireless communication. For example, if the processing unit 50 is housed within a behind-the-ear-hearing-aid-style housing 63, the processing unit 50 can be connected with the EEG recording module 20 and the ta VNS 30 through wire type local interfaces 58. Alternatively, the processing unit 401 may use wireless communication with the EEG recording module 20, network interface 406 and the neuromodulation unit 300 (including one of the following components: a taVNS unit 30, a supraorbital nerve stimulation unit 301, an auriculotemporal nerve stimulation unit 302, an occipital nerve stimulation unit 303, a greater auricular nerve stimulation unit 304 and an infraorbital nerve stimulation unit 305) and the processing unit may be housed remotely as a component of a smart phone type of client device 400 or a smart watch or a health tracker with an app. The processing unit 50, 401, may also be an independent processing device, which is wearable or portable or handheld.

In some embodiments, an auricular EEG monitoring system 100 and/or an automatic detection-therapy system 101, may have an auricular housing 11 configured as one of: a modified earbud housing 61 (such as earphone, earbud or air-pod structures), a modified in-the-ear housing 62 (structure similar to an in-the ear hearing aid), a behind-the-ear-hearing-aid-style housing 63 (structure similar to a behind-the-ear hearing aid) and a tubular-shaped structure 66 (as part of the 61, 62 or 63 or as a standalone tubular-shaped structure 66). In some embodiments, an automatic detection-therapy system 101 may have an auricular housing 11. The auricular housing 11 may be configured to house all or portion of the following: all of the EEG sensor electrodes 12, 13, 72, 73, 82, 83, and an optional reference electrode 84 of an EEG recording module 20, a taVNS stimulating electrode 31, an auriculotemporal nerve stimulation unit 302 and a greater auricular nerve stimulation unit 304. These housing structures are inherently stable for secure attachment. They can be easily removed temporarily for power source 16 (battery) re-charging and can be put back in place easily. Nowadays, earphones, airpods and earbuds have become quite popular. They are nice looking and well accepted by most people. These housing structures enable easy and convenient long-term monitoring and automatic therapeutic intervention of various neuropsychiatric disorders.

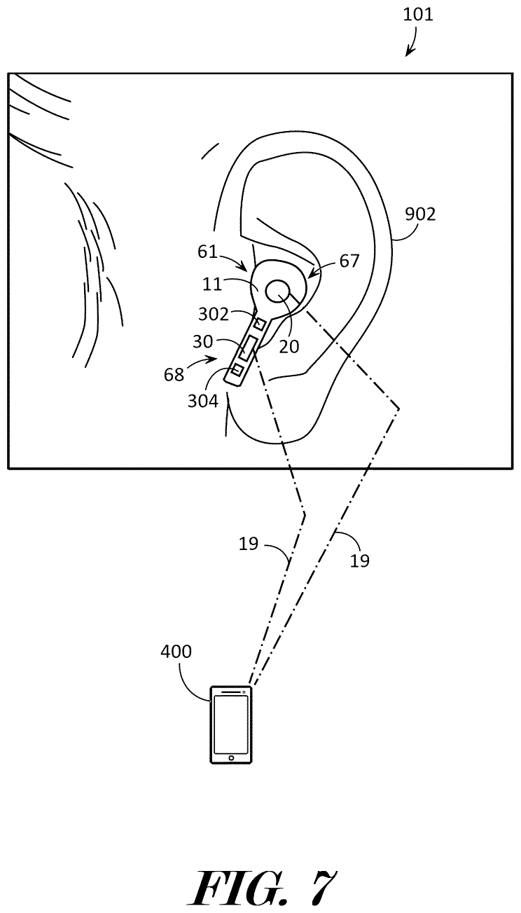

In preferred embodiments, an automatic detection-therapy system 101 may have an auricular housing 11 that may be configured as a modified earbud housing 61. The modified earbud housing 61 includes a tubular-body portion 25 and a stem portion 68. (FIGS. 1, 2). The stem portion 68 is equivalent to a “stem” (or a “stalk”) of an earbud. The tubular-body portion 25 includes a tubular-shaped structure 66 (to be placed inside a wearer's external ear canal 904 when in use) and a body-structure 67 (to be placed immediately at the opening of the wearer's external ear canal 904 and to sit or be placed inside the tragus-concha bowl 957 when in use). The tubular-shaped structure 66 is equivalent to a modified elongated version of an “ear-tip” and a “nozzle” of an earbud. The body-structure 67 is equivalent to a “body” (or a “shell”) of an earbud. The EEG recording module 20 may include an optional reference electrode 84. All of the EEG sensor electrodes 12, 13, 72, 73, 82, 83, may be configured to be placed on a surface 91 of the tubular-shaped structure 66. All of the EEG sensor electrodes 12, 13, 72, 73, 82, 83, are configured to be partially embedded in the surface 91 with slight protrusion at the surface 91 of the tubular-shaped structure 66. The taVNS stimulating electrode 31 and the auriculotemporal nerve (ATN) stimulating electrode 312 may be configured to be placed on a surface and partially embedded in the surface with slight protrusion at the surface of one of: the body-structure 67 and the tubular-shaped structure 66. The optional reference electrode 84 and the greater auricular nerve (GAN) stimulating electrode 314 may be configured to be placed on a surface (and partially embedded in the surface with protrusion at the surface) of the body-structure 67. Preferably the tubular-body portion 25 (including the tubular-shaped structure 66 and the body-structure 67) of the modified earbud housing 61 may be made with or may comprise an elastic flexible and adaptable material (such as silicone), in which the material for the tubular-body portion 25 is configured to have appropriate elasticity, flexibility and adaptability so that the tubular-shaped structure 66 will naturally adapt to the contour of the wearer's external ear canal 904 and naturally fill the interior of the wearer's external ear canal 904 when the tubular-shaped structure 66 is inserted into the wearer's external ear canal 904. Meanwhile, the body-structure 67 will naturally adapt to the contour of the wearer's tragus-concha bowl 957 and naturally fill the interior of the wearer's tragus-concha bowl 957 when the body-structure 67 is placed in the wearer's ear 902 (in the tragus-concha bowl 957). This set-up and the elasticity, flexibility and adaptability of the material will allow all of the EEG sensor electrodes 12, 13, 72, 73, 82, 83, to be naturally in close contact with the skin of the wearer's external ear canal 904 when the tubular-shaped structure 66 is inserted into the wearer's external ear canal 904; meanwhile, the taVNS stimulating electrode 31 and the auriculotemporal nerve (ATN) stimulating electrode 312 will be naturally in close contact with the skin of the wearer's external ear canal 904 or skin of the wearer's tragus-concha bowl 957, and at the same time, the optional reference electrode 84 and the GAN stimulating electrode 314 will be naturally snugly in contact with the skin of the wearer's tragus-concha bowl 957 when the tubular-body portion 25 is placed in the wearer's ear 902. At the same time, the taVNS stimulating electrode 31 will be naturally in close contact with its target skin of vagus innervated auricular skin since the external ear canal 904 and the tragus-concha bowl 957 are part of vagus innervated auricular skin, provided by carefully selecting a location for the taVNS stimulating electrode 31 on the tubular-shaped structure 66 or the body-structure 67 to match one of the innervation locations of the vagus innervated skin on the external ear canal 904 or the tragus-concha bowl 957. Besides that, at the same time, the auriculotemporal nerve (ATN) stimulating electrode 312 will be naturally in close contact with ATN innervated auricular skin since the external ear canal 904 and the tragus-concha bowl 957 are part of the ATN innervated auricular skin, provided by carefully selecting a location for the ATN stimulating electrode 312 on the tubular-shaped structure 66 or the body-structure 67 to match one of the innervation locations of the ATN innervated skin on the external ear canal 904 or the tragus-concha bowl 957. Furthermore, meanwhile, the GAN stimulating electrode 314 will be naturally in close contact with the GAN innervated auricular skin (i.e. the cavum concha 907), provided by carefully selecting the location for the GAN stimulating electrode 314 on the body-structure 67 to match one of the innervation locations of the GAN innervated skin on the tragus-concha bowl 957. (FIGS. 7, 11). For the wearer 900, attaching and removing these electrodes 12, 13, 72, 73, 82, 83, 84, 31, 312, 314, will be as easy as inserting and removing the tubular-body portion 25 of the modified earbud housing 61 from the wearer's external ear 902. There will be no need for a certified technologist to apply the electrodes. Applying adhesive material to secure these electrodes 12, 13, 72, 73, 82, 83, 84, 31, 312, 314, will also be unnecessary. This is feasible due to the unique anatomical features of the human external ear canal 904 and the cavity/bowl shape of the tragus-concha bowl 957 as illustrated in FIGS. 7 and 11. This will create huge convenience for the wearer. Thus, the automatic detection-therapy system 101, is wearable, user-installable, user-removable, ambulatory and very convenient for wearers (users). Please note that the above descriptions are for a neuromodulation unit 30 comprising 3 components, including a taVNS 30, an ATN stimulation unit 302 and a GAN stimulation unit 304. If the neuromodulation unit 300 includes only one or two of these components, the aforementioned convenience and advantages from the housing design can be similarly achieved. For a neuromodulation unit 300, having only one of the components (i.e. one of: a taVNS unit 30, an ATN simulation unit 302 and a GAN stimulation unit 304) or having two of these components in various combinations, the housing setups and the advantages will be similar to the above and will not be re-stated for simplicity purpose. These housing designs and housing setups will enable each of the following to be user-installable and user-removable: the EEG sensor electrodes 12, 13, 72, 73, 82, 83, the optional reference electrode 84, the ta VNS stimulating electrode 31, the auriculotemporal nerve stimulating electrode 312 and the greater auricular nerve stimulating electrode 314. Furthermore, these housing designs and housing setups will enable all of the following to user-installable simultaneously and user-removable simultaneously: the EEG sensor electrodes 12, 13, 72, 73, 82, 83, the optional reference electrode 84, the taVNS stimulating electrode 31, the auriculotemporal nerve stimulating electrode 312 and the greater auricular nerve stimulating electrode 314. These housing designs and housing setups enable any and all of the above electrodes 12, 13, 72, 73, 82, 83, 84, 31, 312, 314 to have automated installation feature and automated removal feature and this will provide huge convenience for the wearer (user) 900.

These advantages can be similarly achieved when the automatic detection-therapy system 101 comprises a behind-the-ear-hearing-aid-style housing 63. In some embodiments, for example, an automatic detection-therapy system 101 may have an auricular housing 11 that may be configured as a behind-the-ear-hearing-aid-style housing 63. The behind-the-ear-hearing-aid-style housing 63 includes an in-the-ear portion 27 and a behind-the-ear portion 26. The in-the-ear portion 27 is essentially the same as the tubular-body portion 25 of a modified earbud housing 61. The in-the-ear portion 27 includes a tubular-shaped structure 66 (configured to be placed inside the wearer's external ear canal 904 when in use) and a body-structure 67 (configured to be placed at the immediate opening of the wearer's external ear canal 904 and to sit or be placed inside the tragus-concha bowl 957 when in use). The in-the-ear portion 27 may be configured to be made with elastic flexible and adaptable material (such as silicone), in which the material for the in-the-ear portion 27 is configured to have appropriate elasticity, flexibility and adaptability so that the tubular-shaped structure 66 will naturally adapt to the contour of the wearer's external ear canal 904 and snugly fill the interior of the external ear canal 904 when the tubular-shaped structure 66 is inserted into the wearer's external ear canal 904; meanwhile, the body-structure 67 will naturally adapt to the contour of the wearer's tragus-concha bowl 957 and snugly fill the interior of the tragus-concha bowl 957 when the body-structure 67 is placed in the wearer's tragus-concha bowl 957. All of the EEG sensor electrodes 12, 13, 72, 73, 82, 83, may be placed on a surface 91 of the tubular-shaped structure 66 of the in-the-ear portion 27. All of the EEG sensor electrodes 12, 13, 72, 73, 82, 83 may be partially embedded in the surface 91 with slight protrusion at the surface 91 of the tubular-shaped structure 66 (of the in-the-ear portion 27) so that all of the EEG sensor electrodes 12, 13, 72, 73, 82, 83, will be naturally in close contact with the skin of the wearer's external ear canal 904 when the tubular-shaped structure 66 is inserted into the wearer's external ear canal 904. The EEG recording module 20 may further include an optional reference electrode 84. The optional reference electrode 84 and the greater auricular nerve (GAN) stimulating electrode 314 may be configured to be placed on a surface (and partially embedded in the surface with slight protrusion at the surface) of the body-structure 67. The taVNS stimulating electrode 31 and the auriculotemporal nerve (ATN) stimulating electrode 312 may be configured to be placed on a surface (and partially embedded in the surface with slight protrusion at the surface) of the in-the-ear portion 27 (in either the tubular-shaped structure 66 or the body-structure 67) so that when the in-the-ear portion 27 is placed in the wearer's external ear canal 904 and the tragus-concha bowl 957, the taVNS stimulating electrode 31 and the auriculotemporal nerve stimulating electrode 312 will be naturally in close contact with the skin of the wearer's external ear canal 904 or the skin of the wearer's tragus-concha bowl 957, and meanwhile the GAN stimulating electrode 314 and the optional reference electrode 84 will be naturally in close contact with the wearer's tragus-concha bowl 957. At the same time, the taVNS stimulating electrode 31 will be naturally in close contact with vagus innervated auricular skin since external ear canal 904 and the tragus-concha bowl 957 are part of vagus innervated auricular skin. Furthermore, the ATN stimulating electrode 312 will also be naturally in close contact with ATN innervated auricular skin since external ear canal 904 and the tragus-concha bowl 957 are also part of ATN innervated auricular skin. Besides that, the GAN stimulating electrode 314 will be naturally contacting the GAN innervated auricular skin (i.e. the cavum concha 907). Thus, the taVNS stimulating electrode 31 will be naturally in close contact with the vagus innervated auricular skin, provided by carefully selecting a location for the taVNS stimulating electrode 31 on the tubular-shaped structure 66 or the body-structure 67 to match one of the innervation locations of the vagus innervated skin on the external ear canal 904 or the tragus-concha bowl 957, and, at the same time, the auriculotemporal nerves (ATN) stimulating electrode 312 will be naturally in close contact with ATN innervated auricular skin, provided by carefully selecting a location for the ATN stimulating electrode 312 on the tubular-shaped structure 66 or the body-structure 67 to match one of the innervation locations of the ATN innervated skin on the external ear canal 904 or the tragus-concha bowl 957, and, at the same time, the greater auricular nerve (GAN) stimulating electrode 314 will be naturally in close contact with GAN innervated auricular skin, provided by carefully selecting a location for the GAN stimulating electrode 314 on the body-structure 67 to match one of the innervation locations of the GAN innervated skin on tragus-concha bowl 957. For a wearer 900, installing and removing all of these electrodes 12, 13, 72, 73, 82, 83, 84, 31, 312, 314, will be as easy as placing and removing the in-the-ear portion 26 (of the behind-the-ear-hearing-aid-style housing 63) from the wearer's ear 902. There will be no need for a certified technologist to install all of these electrodes 12, 13, 72, 73, 82, 83, 84, 31. 312, 314. Applying adhesive material to secure these electrodes 12, 13, 72, 73, 82, 83, 31, 312, 314, will also be unneeded. Thus, this automatic detection-therapy system 101 is fully wearable, user-installable (self-installable), user-removable (self-removable), freely ambulatory and very convenient for wearers (users). (For comparison: The vagus-innervated auricular skin includes: inner posterior portion of tragus 905, cymba-concha 906, cavum-concha 907, posterior and inferior walls of the external ear canal 904 and small adjacent regions of the external ear. The auriculotemporal nerve (ATN) innervated auricular skin includes: anterior outer part of tragus 905, anterior-superior part of cavum concha 907, anterior and superior walls of the external ear canal 904 and, anterior and superior part of pinna including anterior-superior helix 909. The greater auricular nerve (GAN) innervated auricular skin includes lower concha or cavum concha 907, lobule, posterior pinna and mastoid.) Please note that the above descriptions are for a neuromodulation unit 30 comprising 3 components, including a taVNS 30, an ATN stimulation unit 302 and a GAN stimulation unit 304. If the neuromodulation unit 300 includes only one or two of these components, the aforementioned convenience and advantages from the housing design can be similarly achieved. For a neuromodulation unit 300, having only one of the components (i.e. one of: a taVNS unit 30, an ATN simulation unit 302 and a GAN stimulation unit 304) or having two of these components in various combinations, the housing setups and the advantages will be similar to the above and will not be re-stated for simplicity purpose. These housing designs and housing setups will enable each of the following to be user-installable and user-removable: the EEG sensor electrodes 12, 13, 72, 73, 82, 83, the optional reference electrode 84, the taVNS stimulating electrode 31, the auriculotemporal nerve stimulating electrode 312 and the greater auricular nerve stimulating electrode 314. Furthermore, these housing designs and housing setups will enable all of the following to user-installable simultaneously and user-removable simultaneously: the EEG sensor electrodes 12, 13, 72, 73, 82, 83, the optional reference electrode 84, the taVNS stimulating electrode 31, the auriculotemporal nerve stimulating electrode 312 and the greater auricular nerve stimulating electrode 314. These housing designs and housing setups enable any and all of the above electrodes 12, 13, 72, 73, 82, 83, 84, 31, 312, 314 to have automated installation feature and automated removal feature and this will provide huge convenience for the wearer (user) 900.

In some embodiments, the aforementioned advantages can also be similarly achieved when an auricular housing 11 of an automatic detection-therapy system 101 may be shaped or configured as a modified in-the-ear housing 62. The modified in-the-ear housing 62 is essentially similar to the in-the-ear portion 27 of a behind-the-ear-hearing-aid-style housing 63. The modified in-the-ear housing 62 is also essentially the same as a tubular-body portion 25 of a modified earbud housing 61. The modified in-the-ear housing 62 also includes a tubular-shaped structure 66 and a body-structure 67. The tubular-shaped structure 66 will be placed in a wearer's external ear canal 904 when in use. The body-structure 67 will be placed at the opening of the wearer's external ear canal 904 and sit (or be placed) inside a tragus-concha bowl 957 of a wearer's ear 902 when in use. Preferably, all of the EEG sensor electrodes 12, 13, 72, 73, 82, 83, may be configured to be located at a surface 91 and partially embedded in the surface 91 with slight protrusion at the surface 91 of the tubular-shaped structure 66. Preferably, a ta VNS stimulating electrode 31 and an auriculotemporal nerve stimulating electrode 312 may be configured to be located at a surface and partially embedded in the surface with slight protrusion at the surface of one of: the body-structure 67 and the tubular-shaped structure 66. Preferably, the greater auricular nerve stimulating electrode 314 and the optional reference electrode 84 may be located on a surface (and partially embedded in the surface with slight protrusion at the surface) of the body-structure 67. Preferably, the modified in-the-ear housing 62 may be made with or may comprise elastic flexible and adaptable material (such as silicone), in which the material for the modified in-the-ear housing 62 is configured to have appropriate elasticity flexibility and adaptability so that when the modified in-the-ear housing 62 is placed in a wearer's ear 902, the tubular-shaped structure 66 will naturally adapt to the contour of wearer's external ear canal 904 and will snugly fill the interior of the wearer's external ear canal 904 and, meanwhile, the body-structure 67 will naturally adapt to the contour of the wearer's tragus-concha bowl 957 and fill the interior of the tragus-concha bowl 957. This set-up and the elasticity flexibility and adaptability of the material will enable all of these electrodes 12, 13, 72, 73, 82, 83, 31, 312, 314, to be naturally in close contact with the skin of the wearer's external ear canal 904 or skin of the tragus-concha bowl 957 when the modified in-the-ear housing 62 is placed in the wearer's ear 902. At the same time, the taVNS stimulating electrode 31 will be naturally in close contact with the wearer's vagus innervated auricular skin and the auriculotemporal nerve (ATN) stimulating electrode 312 will be naturally in close contact with the wearer's ATN innervated auricular skin since the skin of the external ear canal 904 and skin of the tragus-concha bowl 957 received mixed innervation from the auricular branch of vagus nerve and the auriculotemporal nerve. Furthermore, the GAN stimulating electrode 314 will also be naturally in close contact with the GAN innervated skin of the wearer's cavum concha 907. Thus, this system 101 is wearable, self-installable, self-removable, fully ambulatory and very convenient for wearers (users). Please note that the above descriptions are for a neuromodulation unit 30 comprising 3 components, including a taVNS 30, an ATN stimulation unit 302 and a GAN stimulation unit 304. If the neuromodulation unit 300 includes only one or two of these components, the aforementioned convenience and advantages from the housing design can be similarly achieved. For a neuromodulation unit 300, having only one of the components (i.e. one of: a taVNS unit 30, an ATN simulation unit 302 and a GAN stimulation unit 304) or having two of these components in various combinations, the housing setups and the advantages will be similar to the above and will not be re-stated for simplicity purpose. These housing designs and housing setups will enable each of the following to be user-installable and user-removable: the EEG sensor electrodes 12, 13, 72, 73, 82, 83, the optional reference electrode 84, the taVNS stimulating electrode 31, the auriculotemporal nerve stimulating electrode 312 and the greater auricular nerve stimulating electrode 314. Furthermore, these housing designs and housing setups will enable all of the following to user-installable simultaneously and user-removable simultaneously: the EEG sensor electrodes 12, 13, 72, 73, 82, 83, the optional reference electrode 84, the taVNS stimulating electrode 31, the auriculotemporal nerve stimulating electrode 312 and the greater auricular nerve stimulating electrode 314. These housing designs and housing setups enable any and all of the above electrodes 12, 13, 72, 73, 82, 83, 84, 31, 312, 314 to have automated installation feature and automated removal feature and this will provide huge convenience for the wearer (user) 900.

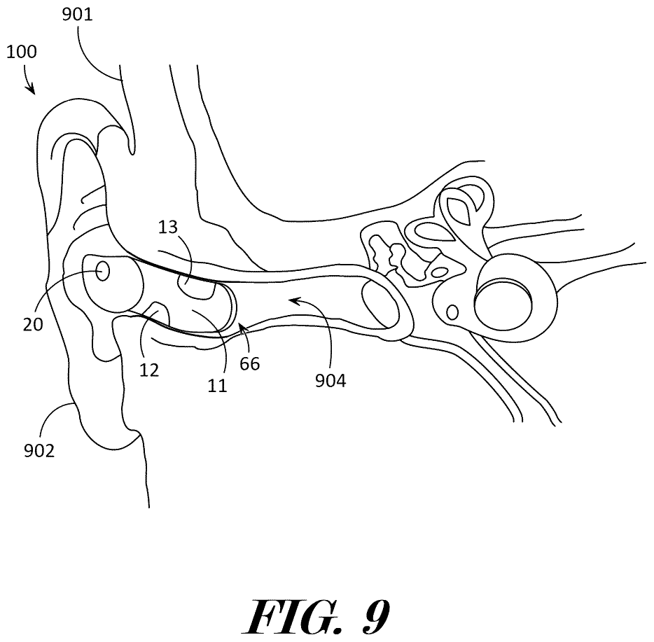

In some embodiments, a standalone tubular-shaped structure 66 may be configured as a housing choice for an auricular EEG monitoring system 100 and/or an automatic detection-therapy system 101. (FIG. 9). Obviously, the aforementioned modified earbud housing 61, modified in-the-ear housing 62 and behind-the-ear-hearing-aid-style housing 63 all comprise a tubular-shaped structure 66. Thus, all of the aforementioned descriptions for the tubular-shaped structure 66 can be applied to a standalone tubular-shaped structure 66 as a housing choice for system 100 and system 101. For an auricular EEG monitoring system 100, all of the EEG sensor electrodes 12, 13, 72, 73, 82, 83, may be configured to be located on a surface and partially embedded in the surface with slight protrusion at the surface of the tubular-shaped structure 66. For an automatic detection-therapy system 101, all of the EEG sensor electrodes 12, 13, 72, 73, 82, 83, a taVNS stimulating electrode 31 and an auriculotemporal nerve stimulating electrode 312 may be configured to be located on a surface and partially embedded in the surface with slight protrusion at the surface of the tubular-shaped structure 66, with the setups, functions and advantages similar to the aforementioned descriptions. There will be no need to have an optional reference electrode 84 when a standalone tubular-shaped structure 66 is used for housing for system 100 or system 101 because the external ear canal 904 may not be an ideal location for a reference electrode 84 (although it may be feasible to place a reference electrode 84 at an inferior surface of the tubular-shaped structure 66.) Instead, average of all of the EEG sensor electrodes 12, 13, 72, 73, 82, 83, can be used as a reference (common average reference). A greater auricular nerve (GAN) stimulating electrode 314 will not be placed in a standalone tubular-shaped structure 66 since the external ear canal 904 does not receive GAN innervation. These housing designs and housing setups will enable each of the following to be user-installable and user-removable: the EEG sensor electrodes 12, 13, 72, 73, 82, 83, the taVNS stimulating electrode 31 and the auriculotemporal nerve stimulating electrode 312. Furthermore, these housing designs and housing setups will enable all of the following to user-installable simultaneously and user-removable simultaneously: the EEG sensor electrodes 12, 13, 72, 73, 82, 83, the taVNS stimulating electrode 31 and the auriculotemporal nerve stimulating electrode 312. These housing designs and housing setups enable any and all of the above electrodes 12, 13, 72, 73, 82, 83, 31, 312, to have automated installation feature and automated removal feature and this will provide huge convenience for the wearer (user) 900.

In some embodiments, a separate client device 400 may be used for housing of one or more of the components of an auricular EEG monitoring system 100 and/or an automatic detection-therapy system 101. For example, an EEG recording module 20 and the processing unit 401 may be housed remotely in a wearable client device 400, such as a smart watch type device or a smart phone type device. The EEG sensor electrodes 12, 13, 72, 73, 82, 83, the optional reference electrode 84, the taVNS stimulation unit 30, the auriculotemporal (ATN) nerve stimulation unit 302 and the greater auricular nerve (GAN) stimulating electrode 314 may be housed in one of: a modified earbud housing 61, a modified in-the-ear housing 26 and in an in-the-ear portion 27 of a behind-the-ear-hearing-aid-style housing 63. Wireless EEG sensor electrodes may be used for all of the EEG sensor electrodes 12, 13, 72, 73, 82, 83 and the optional reference electrode 84. (Wireless dry electrodes for in-ear EEG such as that described by Ryan Kaveh et al. in Nature Communications on Aug. 2, 2024. Wireless dry EEG electrodes are also available from Zeto, Inc. headquarter in Santa Clara, California.) There are well-known wired or wireless EEG amplifiers available, such as EEG Electroencephalogram Smart Amplifier (Part #: EEG100D), or preferably BioNomadix 2Ch Wireless EEG Amplifier (Part #: BN-EEG2), both being made by the same company BIOPAC Systems, Inc. (Goleta, California). By using wireless EEG electrodes 12, 13, 72, 73, 82, 83, wireless optional reference electrode 84 and wireless EEG amplifier 21, the EEG recording module 20, together with the processing unit 401, may be housed remotely in a wearable client device 400 (such as a watch-type device or a smart phone type device) and communicate wirelessly with all of the EEG sensor electrodes 12, 13, 72, 73, 82, 83 and the optional reference electrode 84.

In some embodiments, an auricular EEG monitoring system 100 and/or an automatic detection-therapy system 101 may comprise a network interface 53. The network interface 53 is in electronic communication with a speaker 15, 404A, and/or a vibrator 17, 404B, which may be configured to generate an audible and/or tactile notification to a wearer 900, or the wearer's healthcare provider 950, or other individual.

A speaker 15, 404A, may comprise a sound emitting device which can provide audible notification function. A speaker of a speaker 15, 404A, may comprise a buzzer, a piezoelectric sound producing device, a dielectric elastomer sound producing device, a buzzer, a moving coil loudspeaker, an electrostatic loudspeaker, an isodynamic loudspeaker, a piezoelectric loudspeaker, or any other device capable of producing one or more sounds. In preferred embodiments, an auricular EEG monitoring system 100 and/or an automatic detection-therapy system 101 may comprise a speaker 15, 404A, that may be in electronic communication with a network interface 53, 406, and/or a processing unit 50, 401. The speaker 15, 404A, may be configured to generate an audible notification when the processing unit 50, 401, detects one or more of: the presence of EEG signals of the wearer 900 suggestive of neuropsychiatric disorder; the presence of EEG signals of the wearer 900 suggestive of impending neuropsychiatric disorder, the cessation of EEG signals of the wearer 900 suggestive of neuropsychiatric disorder; and the cessation of EEG signals of the wearer 900 suggestive of the impending neuropsychiatric disorder.

A vibrator 17, 404B, may comprise a weight that may be rapidly moved by a long life brushless (BLDC) vibration motor, a coin or pancake vibration motor, an encapsulated vibration motor, an enclosed vibration motor, a pager motor, an eccentric rotating mass (ERM) motor, a linear resonant actuator (LRA), a printed circuit board (PCB) mounted vibration motor, or any other electrical device capable of producing a series of rapid and repeated movements. In preferred embodiments, an auricular EEG monitoring system 100 and/or an automatic detection-therapy system 101 may comprise a vibrator 17, 404B, that may be in electronic communication with a network interface 53, 406, and/or a processing unit 50, 401. The vibrator 17, 404B, may be configured to generate a tactile notification when the processing unit 50, 401, detects one or more of: the presence of EEG signals of the wearer 900 suggestive of neuropsychiatric disorder; the presence of EEG signals of the wearer 900 suggestive of an impending neuropsychiatric disorder, the cessation of EEG signals of the wearer 900 suggestive of the neuropsychiatric disorder; and the cessation of EEG signals of the wearer 900 suggestive of the impending neuropsychiatric disorder.

In some embodiments, an auricular EEG monitoring system 100 and an automatic detection-therapy system 101 may comprise a switch 88 and a timer 89 to enable the wearer 900 to turn on or off the system 100 or system 101 manually and setting a duration, an interval or a schedule etc. Preferably, the switch 88 may be configured as a multi-mode switch 88 and the timer 89 may be configured as a multi-mode timer 89. In some embodiments, a multi-mode switch 88 may be configured to enable the wearer to set the system 100 and system 101 at an automatic mode or a manual mode. The functions and setups of the automatic mode for system 101 are as described hereinbefore. In some embodiments for an automatic detection-therapy system 101, the multi-mode switch 88 may be configured to allow the wearer 900 to choose at least one of the following switch selections: turning on or off an auricular EEG monitoring system 100, turning on or off a taVNS unit 30, turning on or off a supraorbital nerve stimulation unit 301, turning on or off an auriculotemporal nerve stimulation unit 302, turning on or off an occipital nerve stimulation unit 303, turning on or off a greater auricular nerve stimulation unit 304, turning on or off an infraorbital nerve stimulation unit 305, including various combinations thereof, and selecting from various stimulation modes. The components of the neuromodulation unit 300 is configured to be selectable by the wearer 900 via the multi-mode switch 88, and the wearer 900 can switch back and forth among different components of the neuromodulation unit 300 via the multi-mode switch 88. The multi-mode timer 89 may be configured to function together with the multi-mode switch 88 to set each switch selection at a desirable timing course such as duration, interval, countdown, daily/weekly schedule, 10 minute/30 minutes/60 minutes (or other duration) selection, continuous operation, manual turning on and manual shutdown etc. Furthermore, the multi-mode switch 88 and multi-mode timer 89 may be configured as programmable. Furthermore, the multi-mode switch 88 and multi-mode timer 89 may be configured as a multifunctional switch-timer or as a programmable multifunctional timer-switch. In some embodiments, the programmable multifunctional timer-switch may be incorporated in a client device 400 of a wearer 900 to allow the wearer flexibility and ease in choosing manual or automatic controls and choosing various time courses and various switch selections (selection of various combinations of the components of the neuromodulation unit, including the ta VNS unit 30, the supraorbital nerve stimulation unit 301, the auriculotemporal nerve stimulation unit 302, the occipital nerve stimulation unit 303, the greater auricular nerve stimulation unit 304 and the infraorbital nerve stimulation unit 305.) In some embodiments, the multi-mode switch 88 function and the multi-mode timer 89 function may be configured to be incorporated into a wearer's client device 400 and can be easily set up as programmable. In the manual mode, the wearer 900 can use at least one of: the taVNS unit 30, the supraorbital nerve stimulation unit 301, the auriculotemporal nerve stimulation unit 302, the occipital nerve stimulation unit 303, the greater auricular nerve stimulation unit 304 and the infraorbital nerve stimulation unit 305, or various combinations thereof for prophylactic purpose against neuropsychiatric disorders or for health maintenance purpose. Studies have shown that the taVNS unit 30 is effective not only for therapy, but also for prophylaxis, for various neuropsychiatric disorders. Studies have also shown that the ta VNS unit 30, the supraorbital nerve stimulation unit 301, the auriculotemporal nerve stimulation unit 302, the occipital nerve stimulation unit 303, the greater auricular nerve stimulation unit 304 and the infraorbital nerve stimulation unit 305 (and various combinations thereof) are effective not only for therapy, but also for prophylaxis, for various neuropsychiatric disorders. The aforementioned programmable set up for the system 101 will enable the system 101 to be used for prophylactic purpose and health maintenance purpose. The neuromodulation unit 300 comprises 6 neuromodulation components, including a taVNS stimulation unit 30, a supraorbital nerve stimulation unit 301, an auriculotemporal nerve stimulation unit 302, an occipital nerve stimulation unit 303, a greater auricular nerve stimulation unit 304 and an infraorbital nerve stimulation unit 305. The multi-mode switch 88, multi-mode timer 89 and the programmable design may enable the wearer the option to choose from one of the following: neuromodulation from one neuromodulation component, simultaneous neuromodulation from two neuromodulation components and simultaneous neuromodulation from three neuromodulation components. Thus, when prompted, the neuromodulation unit 300 is configured to generate neuromodulating electric stimulation to a wearer 900 according to the choice of the stimulation mode selected by the wearer 900. The choices of the stimulation mode include the following: single neuromodulation by one component of the neuromodulation unit 300, double neuromodulation by various combinations of two components of the neuromodulation unit 300, triple neuromodulation by various combinations of three components of the neuromodulation unit 300, etc. In some embodiments, the neuromodulation unit 300 is configured to be controlled by the multi-mode switch 88 and multi-mode-timer 89 to allow the wearer 900 to switch back and forth among different components of the neuromodulating unit 300 via the multi-mode switch 88 and to select different time courses via the multi-mode-timer 89 (or an integrated programmable multifunctional switch-timer). In alternate embodiments, an automatic detection-therapy system 101 for neuropsychiatric disorders may comprise a first and second EEG recording modules 20 and a first and a second neuromodulation units 300 (as described hereinbefore) and the system 101 may further comprise a multi-mode switch 88 and a multi-mode timer 89 (or a programmable multi-functional switch-timer) that are configured to be linked to both the first and the second EEG recording modules 20 and the first and the second neuromodulation units 300, and are configured to enable the wearer 900 to select stimulation components from both the first and the second neuromodulation units 300 and to set the time courses involving stimulation components from both the first and the second neuromodulation units 300.

In still another aspect consistent with the principles of this invention, a combined neuromodulation system 102 is disclosed. The combined neuromodulation system 102 comprises at least two neuromodulating components. In some embodiments, a combined neuromodulation system 102 comprises two neuromodulating components, including a transcutaneous auricular vagus nerve stimulation unit (taVNS unit) 30 and an auriculotemporal nerve (ATN) stimulation unit 302. In other embodiments, a combined neuromodulation system 102 comprises two neuromodulating components, including a transcutaneous auricular vagus nerve stimulation unit (taVNS unit) 30 and a greater auricular nerve (GAN) stimulation unit 304. In still other embodiments, a combined neuromodulation system 102 comprises two neuromodulating components, including an auriculotemporal nerve (ATN) stimulation unit 302 and a GAN stimulation unit 304. In modified embodiments, a combined neuromodulation system 102 comprises 3 neuromodulating components, including a taVNS unit 30, an ATN stimulation unit 302 and a GAN stimulation unit 304. The setups and functions of each neuromodulating component (i.e. the ta VNS unit 30, the ATN stimulation unit 302, the AGN stimulation unit 304) are as described hereinbefore. The combined neuromodulation system 102 further comprises a multi-mode switch 88 and a multi-mode timer 89 (or an integrated multifunctional switch-timer). The multi-mode switch 88 and the multi-mode timer 89 are in electronic communication with each neuromodulating component of the combined neuromodulation system 102. The multi-mode switch is configured to enable a wearer (user) 900 to select among various stimulation modes, including the following: single neuromodulation by one neuromodulating component of the neuromodulation system 102, double neuromodulation by various combinations of two neuromodulating components of the neuromodulation system 102, triple neuromodulation by all three neuromodulating components of the neuromodulation system 102. The multi-mode timer 89 is configured to enable the wearer 900 to select among various stimulation time courses. The multi-mode timer 89 may be configured to function together with the multi-mode switch 88 to allow the wearer 900 to set each switch selection of stimulation mode at a desirable timing course such as duration, interval, countdown, daily/weekly schedule, 10 minute/30 minutes/60 minutes (or other duration) selection, continuous operation, manual turning on and manual shutdown etc. In some embodiments, the multi-mode switch 88 function and the multi-mode timer 89 function may be configured to be incorporated into a wearer's client device 400 and can be easily set up as programmable. The wearer (user) 900 may select among various stimulation modes and select among various timing courses from the client device 400. The combined neuromodulation system 102 may be configured to be housed in an auricular housing 11 (such as a modified in-the-ear housing 62 or a modified earbud housing 61) that includes a body-structure 67. The body-structure 67 is configured to be placed in the wearer's tragus-concha bowl 957 when in use. All of the stimulating electrodes (including the ta VNS stimulating electrode 31, the ATN stimulating electrode 312 and the GAN stimulating electrode 314) may be configured to be located on a surface of the body-structure 67 and are partially embedded in the surface with slight protrusion at the surface of the body-structure 67. The body-structure 67 is configured to be made of elastic flexible and adaptable material. The material of the body-structure 67 is configured to have appropriate elasticity, flexibility and adaptability so that the body-structure 67 will naturally adapt to the contour of the wearer's tragus-concha bowl 957 and snugly fill the interior of the wearer's tragus-concha bowl 957 when the body-structure 67 is placed in the wearer's tragus-concha bowl 67; and so that the taVNS stimulating electrode 31, the ATN stimulating electrode 312 and the GAN stimulating electrode 314 will be naturally in close contact with the skin of the wearer's tragus-concha bowl 957 when the body-structure 67 is placed in the wearer's tragus-concha bowl 957. It should be noted that the tragus-concha bowl 945 received mixed innervation from the auricular branch of vagus nerve, the auriculotemporal nerve and the greater auricular nerve. With careful selection of the locations on the body-structure 67 for the taVNS stimulating electrode 31, ATN stimulating electrode 312 and GAN stimulating electrode 314 to match their innervation patterns and innervation locations on the tragus-concha bowl 957, these stimulating electrodes 31, 312, 314, will be naturally in close contact with their target skin on the tragus-concha bowl 957 when the body-structure 67 is placed in the wearer's tragus-concha bowl 957. In alternative embodiments, a combined neuromodulation system 102 may be configured to be housed in an auricular housing 11, and the auricular housing 11 may comprise a tubular-shaped structure 66 (to be inserted into a wearer's external ear canal 904 when in use) and a body-structure 67 (to be placed at immediate opening of the wearer's external ear canal 904 and to be placed in the wearer's tragus-concha bowl 957 when in use). The GAN stimulating electrode 314 may be configured to be located on a surface (and partially embedded in the surface with slight protrusion at the surface) of the body-structure 67 while the taVNS stimulating electrode 31 and the ATN stimulating electrode 312 may be configured to be located on a surface (and partially embedded in the surface with slight protrusion at the surface) of the tubular-shaped structure 66. The tubular-shaped structure 66 and the body-structure 67 are configured to be made of elastic flexible and adaptable material and the material is configured to be of appropriate elasticity flexibility and adaptability so that the GAN stimulating electrode 314 will be naturally in close contact with the skin of the tragus-concha bowl 957 when the body-structure 67 is placed in the tragus-concha bowl 957, while the ta VNS stimulating electrode 31 and the ATN stimulating electrode 312 will be naturally in close contact with the wearer's external ear canal 904 when the tubular-shaped structure 66 is inserted into the wearer's external ear canal 904 (similar to descriptions hereinbefore). By carefully selecting the locations for the taVNS stimulating electrode 31 and ATN stimulating electrode 312 on the tubular-shaped structure 66 to match their innervation patterns and innervation locations on the external ear canal 904, these stimulating electrodes 31, 312, will be naturally in close contact with their target skin on the external ear canal 904 when the tubular-shaped structure 66 is inserted into the wearer's external ear canal 904. Similarly, by carefully selecting the location of the GAN stimulating electrode 314 on the body-structure 67 to match one of the GAN innervation locations on the tragus-concha bowl 957, the GAN stimulating electrode 314 will be naturally in close contact with its target skin in the wearer's tragus-concha bowl 957 when the body-structure 67 is placed in the wearer's tragus-concha bowl 957 (similar to descriptions hereinbefore). In some embodiments, a combined neuromodulation system 102 is configured to be controlled by the multi-mode switch 88 and multi-mode-timer 89 to allow the wearer 900 to switch back and forth among different neuromodulating components of the combined neuromodulation system 102 via the multi-mode switch 88 and to set different time courses via the multi-mode-timer 89 (or multifunctional switch-timer). This combined neuromodulation system 102 will be very convenient and useful for the wearer for health maintenance purpose and for prophylaxis purpose against many neuropsychiatric disorders. There are evidences showing that bilateral neuromodulation of auricular branch of vagus nerve, (or ATN or GAN) is more effective than unilateral neuromodulation. This combined neuromodulation system 102 may utilize two combined neuromodulation systems 102 with a first system 102 attached to right ear 902 of the wearer and a second system 102 attached to the left ear 902 of the wearer 900.

As perhaps best shown by FIG. 18, an illustrative example of some of the physical components which may be used with an auricular EEG monitoring system 100 and/or an automatic detection-therapy system 101, according to some embodiments are presented. An auricular EEG monitoring system 100 and an automatic detection-therapy system 101 may be configured to facilitate the transfer of data and information between one or more access points 103, client devices 400, and servers 800 over a data network 105. Client devices 400 and servers 800 may send data to and receive data from the data network 105 through a network connection 104 with an access point 103. A data store 308 accessible by the server 800 may contain one or more databases. The data may comprise any data recorded and generated by an auricular EEG monitoring system 100 and/or an automatic detection-therapy system 101.

In this example, an auricular EEG monitoring system 100 and/or an automatic detection-therapy system 101 may comprise or be in communication with at least one client device 400 (but preferably two or more than two client devices 400) configured to be operated by one or more users 900, 950. Client devices 400 may include mobile devices, such as laptops, tablet computers, personal digital assistants, smart phones, smart watches, and the like, that are equipped with a wireless network interface 406 capable of sending data to one or more servers 800 with access to one or more data stores 308 over a network 105, such as a wireless local area network (WLAN). Additionally, client devices 400 may include fixed devices, such as desktops, workstations, and the like, that are equipped with a wireless or wired network interface capable of sending data to one or more servers 800 with access to one or more data stores 308 over a wireless or wired local area network 105. The present invention may be implemented on at least one computing device, such as a client device 400 and/or server 800, programmed to perform one or more of the steps described herein. In some embodiments, more than one client device 400 and/or server 800 may be used, with each being programmed to carry out one or more steps of a method or process described herein.

### A First Preferred Embodiment

In preferred embodiments, an automatic detection-therapy system 101 for neuropsychiatric disorders may comprise an auricular EEG monitoring system 100 having an auricular electroencephalogram (EEG) recording module 20, a neuromodulation unit 300 and a processing unit 50, 401. The auricular EEG recording module 20 may have a plurality (at least two, but preferably more than two) of miniature wired or wireless EEG sensor electrodes 12, 13, 72, 73, 82, 83 and wired or wireless EEG amplifier 21. Optionally, the EEG recording module 20 may include a wired or wireless optional reference electrode 84. (The optional reference electrode 84 would be preferred but is not always necessary. Instead, average of all of the EEG sensor electrodes 12, 13, 72, 73, 82, 83, can be used as a reference, i.e. common average reference). The EEG sensor electrodes 12, 13, 72, 73, 82, 83, and the optional reference electrode 84 may be configured to contact separate areas selected from at least one of the following: external ear canal 904 of a wearer's first ear, external ear 902 of the first ear or peri-auricular area 903 around the first ear. Preferably, the EEG sensor electrodes 12, 13, 72, 73, 82, 83, may be configured to contact separate areas of the external ear canal 904 of the wearer's first ear. Preferably, the optional reference electrode 84 may be configured to contact a tragus-concha bowl 957 of the wearer's first ear 902 or a mastoid of the peri-auricular area 903 of the wearer's first ear. The peri-auricular area 903 refers to a portion of the head around the auricle (pinna). The peri-auricular area 903 is typically hairless. The peri-auricular area includes a portion of the head in front of the auricle (pre-auricular area) and a portion of the head above and behind the auricle (post-auricular area). The pre-auricular area is small, about one inch wide and two inches long and curved along the anterior edge of the auricle. The post-auricular area is also small and is about one inch wide and about three inches long and curved along the superior and posterior edges of the auricle (pinna). The mastoid is located toward the lower end of the post-auricular area. The post-auricular area is where a behind-the-ear hearing aid is usually attached to. The preauricular area and the post-auricular area together is called “peri-auricular area” herein. (Anterior, posterior, superior, in front of and behind etc. all refer to the directions relative to the wearer's head when the wearer is in an upright position.) The optional reference electrode 84 of the EEG recording module 20 may be placed inside a tragus-conche bowl 957 or at the mastoid of the peri-auricular area 903. The EEG recording module 20 may be configured to record and generate EEG data of the wearer 900 using electrical activities of the wearer 900 that are picked up via all of the EEG sensor electrodes 12, 13, 72, 73, 82, 83, and the optional reference electrode 84 (if the reference electrode is incorporated). (FIGS. 2, 7, 11-13).

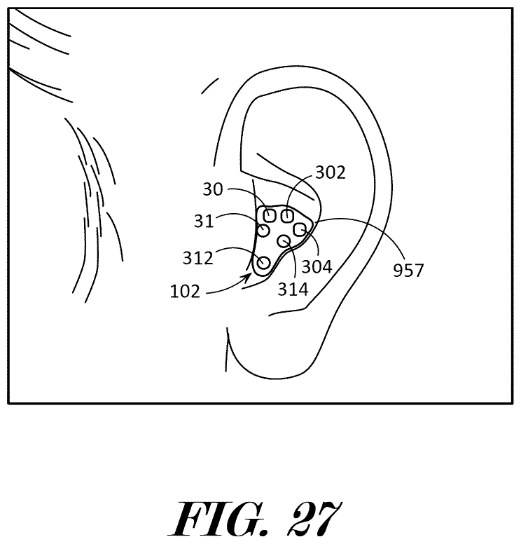

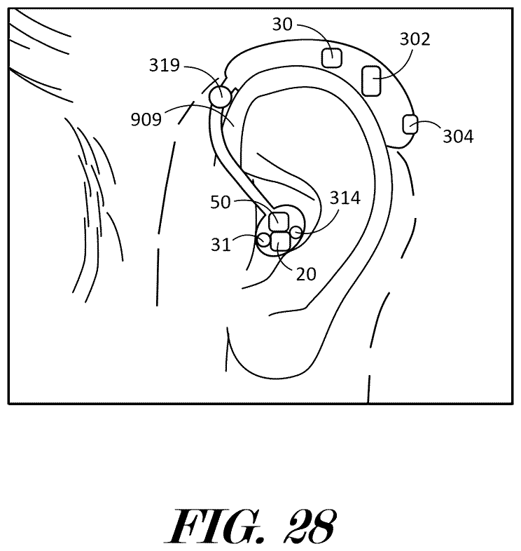

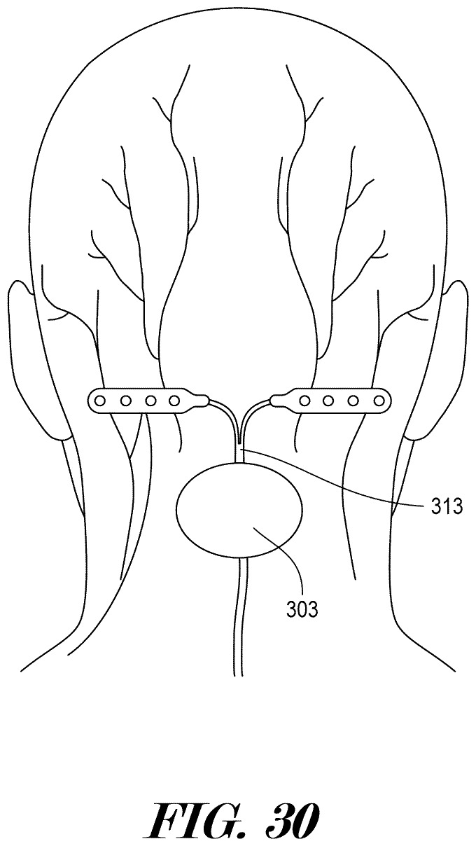

In preferred embodiments for an automatic detection-therapy system 101 for neuropsychiatric disorders, the neuromodulation unit 300 may include at least one of the following components: a transcutaneous auricular vagus nerve stimulation unit (taVNS unit) 30, a supraorbital nerve stimulation unit 301, an auriculotemporal nerve (ATN) stimulation unit 302 an occipital nerve stimulation unit 303, a greater auricular nerve (GAN) stimulation unit 304 and an infraorbital nerve stimulation unit 305. There are evidences showing the benefits of neuromodulating electric stimulation from taVNS unit 30. More recently, there are also evidences showing benefits of neuromodulating electric stimulation from non-vagus electric neuromodulation unit. As used herein, the term “non-vagus electric neuromodulation unit” refers to one of the following: a supraorbital nerve stimulation unit 301, an auriculotemporal nerve stimulation unit 302, an occipital nerve stimulation unit 303, a greater auricular nerve stimulation unit 304, an infraorbital nerve stimulation unit 305, and various combinations thereof. Non-vagus electric neuromodulation has special benefits for migraine, cluster headache, other pain and other neuropsychiatric disorders. Simultaneous stimulation from a taVNS unit 30 and a non-vagus electric neuromodulation unit has synergistic effects. The taVNS unit 30 may comprise a miniature ta VNS stimulating electrode 31. The taVNS stimulating electrode 31 may be configured to contact its target skin of vagus innervated auricular skin of the wearer's 900 first ear. The vagus innervated auricular skin (i.e. the target skin for taVNS stimulating electrode 31) that the stimulating electrode 31 is configured to contact may be selected from at least one of the following: posterior and inferior walls of external ear canal 904, inner/posterior portion of tragus 905, cymba-concha 906, and majority of cavum-concha 907. (Part of the eardrum is also innervated by the vagus nerve but eardrum is not suitable for electrode placement.) (FIGS. 2, 11-13). When prompted, the taVNS unit 30 may be configured to give neuromodulating electric stimulation through the taVNS stimulating electrode 31 to the vagus innervated auricular skin in a way similar to transcutaneous electric nerve stimulation (TENS) (taVNS and TENS as known in the art). The supraorbital nerve stimulation unit 301 may comprise a supraorbital nerve stimulation electrode 311 to be attached to supraorbital nerve innervated forehead skin. (FIG. 29). The supraorbital nerve stimulation unit 301 is configured to give neuromodulating electric stimulation through the supraorbital nerve stimulating electrode 311 to a wearer's supraorbital nerve innervated forehead skin when prompted. The auriculotemporal nerve (ATN) stimulation unit 302 may comprise an ATN stimulating electrode 312 to be attached to its target skin of the ATN innervated skin of the wearer's ear 902. (FIGS. 27, 28). The auriculotemporal nerve (ATN) innervated auricular skin (i.e. the target skin for ATN stimulating electrode 312) include anterior-superior helix 909, anterior outer part of tragus 905, anterior portion (or anterior superior part) of cavum-concha 907, anterior and superior walls of the external ear canal 904, and anterior auricle. (Part of the outer tympanic membrane is innervated by the auriculotemporal nerve but the eardrum is not suitable for electrode placement). Thus, the tragus-concha bowl 957 and the external ear canal 904 received mixed innervation from both the auricular branch of vagus nerve and the auriculotemporal nerve (ATN). The ATN stimulation unit 302 is configured to give neuromodulating electric stimulation through the ATN stimulating electrode 312 to a wearer's ATN innervated ear skin when prompted. The occipital nerve stimulation unit 303 may comprise an occipital nerve stimulating electrode 313 to be attached to occipital nerve innervated occipital region. (FIG. 30). The occipital nerve stimulation unit 303 is configured to give neuromodulating electric stimulation through the occipital nerve stimulating electrode 313 to a wearer's occipital nerve innervated occipital region when prompted. The greater auricular nerve (GAN) stimulation unit 304 may comprise a GAN stimulating electrode 314 to be attached to its target skin of GAN innervated ear skin. The GAN innervated auricular skin (i.e. the target skin for GAN stimulating electrode 314) includes lower concha (cavum concha 907), lower two thirds of anterior and posterior pinna and the mastoid process. The GAN stimulation unit 304 is configured to give neuromodulating electric stimulation through the GAN stimulating electrode 314 to a wearer's GAN innervated auricular skin (e.g. skin of cavum concha 907) when prompted. The infraorbital nerve stimulation unit 305 is configured to give neuromodulating electric stimulation through the infraorbital nerve stimulating electrode 315 to a wearer's infraorbital nerve innervated mid-facial skin when prompted.

(For comparison: The vagus-innervated auricular skin includes: inner posterior portion of tragus 905, cymba-concha 906, cavum-concha 907, posterior and inferior walls of the external ear canal 904 and small adjacent regions of the external ear. The auriculotemporal nerve innervated auricular skin includes: anterior outer part of tragus 905, anterior-superior part of cavum concha 907, anterior and superior walls of the external ear canal 904 and, anterior and superior part of pinna including anterior-superior helix 909. The greater auricular nerve innervated auricular skin includes: cavum concha, lower two thirds of anterior and posterior pinna and the mastoid process.) From the above comparison, it is obvious that the tragus-concha bowl 957 of a wearer's ear 902 received mixed and overlapping innervation from the auricular branch of vagus nerve, the auriculotemporal nerve (ATN) and the greater auricular nerve (GAN). When the taVNS stimulating electrode 31, the ATN stimulating electrode 312 and the GAN stimulating electrode 314 are located on a surface of the body-structure 67, by carefully selecting the locations for the taVNS stimulating electrode 31, the ATN stimulating electrode 312 and the GAN stimulating electrode 314 on the body-structure 67 to match the aforementioned innervation locations of the innervated skin on the tragus-concha bowl 957, these stimulating electrodes 31, 312, 314, will automatically get in close contact with their target skin on the tragus-concha bowl 957 when the body-structure 67 is placed in the tragus-concha bowl 957. Alternatively, when the ta VNS stimulating electrode 31 and the ATN stimulating electrode 312 are located on a surface of the tubular-shaped structure 66, by carefully selecting the locations for the taVNS stimulating electrode 31 and the ATN stimulating electrode 312 on the tubular-shaped structure 66 to match the aforementioned innervation locations of the innervated skin on the external ear canal 904, these stimulating electrodes 31, 312, will automatically get in close contact with their target skin on the external ear canal 904 when the tubular-shaped structure 66 is placed in the external ear canal 904.

In some embodiments for an automatic detection-therapy system 101 for neuropsychiatric disorders, the auricular EEG recording module 20 may be in electronic communication with the processing unit 50, 401, through Bluetooth, wired, wireless or other electronic connection means or methods. Each component of the neuromodulation unit 300 (including at least one of: the taVNS unit 30, the supraorbital nerve stimulation unit 301, the auriculotemporal nerve stimulation unit 302, the occipital nerve stimulation unit 303, the greater auricular nerve stimulation unit 304 and the infraorbital nerve stimulation unit 305) may also be in electronic communication with the processing unit 50, 401, through Bluetooth, wired, wireless or other connection means. The EEG recording module 20 collects the wearer's EEG data and these data are transmitted to the processing unit 50, 401. With the help of various advanced EEG analysis algorithms together with machine learning, deep learning and artificial intelligence (as known in the art), the processing unit 50, 401, is configured to analyze the EEG data to detect presence of EEG signals of wearer 900 suggestive of neuropsychiatric disorders and impending neuropsychiatric disorders. The processing unit 50, 401, may also be configured to detect cessation of EEG signals of wearer 900 suggestive of the neuropsychiatric disorder or cessation of EEG signals suggestive of the impending neuropsychiatric disorder. The neuropsychiatric disorders may include seizure, migraine, cluster headache, major depressive disorder (MDD), bipolar disorder, schizophrenia, obsessive compulsive disorder (OCD), attention deficit hyperactivity disorder (ADHD), autism spectrum disorder (ASD), post-traumatic stress disorder (PTSD), anxiety disorder and panic disorder, etc.

When presence of EEG signals suggestive of a neuropsychiatric disorder is detected by the processing unit 50, 401, the processing unit 50, 401, may be configured to automatically send signals to the neuromodulation unit 300 to actuate at least one of the following components: the taVNS unit 30, the supraorbital nerve stimulation unit 301, the auriculotemporal nerve (ATN) stimulation unit 302, the occipital nerve stimulation unit 303, the greater auricular nerve (GAN) stimulation unit 304 and the infraorbital nerve stimulation unit 305 to start sending pre-determined neuromodulating electric stimulation immediately. When presence of EEG signals suggestive of an impending neuropsychiatric disorder is detected by the processing unit 50, 401, the processing unit 50, 401, may be configured to automatically send signals to the neuromodulation unit 300 to actuate at least one of the following components: the taVNS unit 30, the supraorbital nerve stimulation unit 301, the ATN stimulation unit 302, the occipital nerve stimulation unit 303, the GAN stimulation unit 304 and the infraorbital nerve stimulation unit 305 to start sending pre-determined neuromodulating electric stimulation immediately. When cessation of EEG signals suggestive of a neuropsychiatric disorder is detected by the processing unit 50, 401, the processing unit 50, 401, may be configured to automatically send signals to the neuromodulation unit 300 to stop sending pre-determined electric stimulation from any of the following: the taVNS unit 30, the supraorbital nerve stimulation unit 301, the ATN stimulation unit 302, the occipital nerve stimulation unit 303, the GAN stimulation unit 304 and the infraorbital nerve stimulation unit 305. When cessation of EEG signals suggestive of impending neuropsychiatric disorder is detected by the processing unit 50, 401, the processing unit 50, 401, may be further configured to automatically send signals to the neuromodulation unit 300 to stop sending pre-determined electric stimuli from any of the following: the taVNS unit 30, the supraorbital nerve stimulation unit 301, the ATN stimulation unit 302, the occipital nerve stimulation unit 303, the GAN stimulation unit 304 and the infraorbital nerve stimulation unit 305.

For the taVNS unit 30, the general parameters of the electric stimulation for neuropsychiatric disorders, including the stimulating patterns, strength, duration, frequency and intervals, are predetermined, such as shown in Table 1. Each neuropsychiatric disorder may respond to partially different parameters of the electric stimulation, such as shown in Tables 3, 5, 7, 9 and 11. For the taVNS unit 30, the general parameters of the electric stimulation for impending neuropsychiatric disorders, including the stimulating patterns, strength, duration, frequency and intervals, are predetermined, such as shown in Table 2. Each impending neuropsychiatric disorder may respond to partially different parameters of the electric stimulation, such as shown in Tables 4, 6, 8, 10, 12. Some examples of stimulation parameters for other components of the neuromodulation unit 300 (such as supraorbital nerve stimulation unit 301, auriculotemporal nerve stimulation unit 302, occipital nerve stimulation unit 303, greater auricular nerve stimulation unit 304 and infraorbital nerve stimulation unit 305) for neuropsychiatric disorders were described hereinbefore and by various articles in the Background section of this invention.

For most patients with neuropsychiatric disorders, two auricular EEG recording modules 20, one on each side of the head 901 linked to each ear 902 or each periauricular area 903, may be preferred. In rare situations, only one auricular EEG recording module 20 might be enough for some patients if their neuropsychiatric disorders can be adequately detected by a partial EEG on one side. For most patients with neuropsychiatric disorders, two taVNS units 30 may be utilized, with one taVNS unit 30 on each side of the head 901 coupled to the tragus-concha bowl 957 or external ear canal 904 of each ear 902. In rare situations if the patient is unable to tolerate a taVNS unit 30 in right ear due to bradycardia or other side effects, only one taVNS unit 30 may be utilized and be placed in the left ear. Likewise, for most patients with neuropsychiatric disorders, two auriculotemporal nerve (ATN) stimulation unit 302 may be utilized, with one ATN stimulating electrode 312 attached to the tragus-concha bowl 957 or external ear canal 904 of each ear 902. Similarly, for most patients with neuropsychiatric disorders, two greater auricular nerve (GAN) stimulation unit 302 may be utilized, with one GAN stimulating electrode attached to the cavum concha 907 of each ear 902. In some embodiments, an automatic detection-therapy system 101 may comprise an auricular EEG monitoring system 100 having a first EEG recording module 20 and a second EEG recording module 20 and the automatic detection-therapy system 101 may further comprise a first neuromodulation unit 300 and a second neuromodulation unit 300. The first EEG recording module 20 may be configured to be linked to a first ear 902 of a wearer. Some components of the first neuromodulation unit 300 (including a taVNS unit 30, an ATN stimulation unit 302 and a GAN stimulation unit 304) may also be linked to the first ear 902 of the wearer. Other components of the first neuromodulation unit 300 are linked to mid-forehead (supraorbital nerve stimulation unit 301) mid-face (infraorbital nerve stimulation unit 305) or mi-occipital region (occipital nerve stimulation unit 303). The second EEG recording module 20 and the second neuromodulation unit 300 may be configured to be linked to a second ear 902 of the wearer. The first neuromodulation unit 300 may comprise at least one of the following: a first taVNS unit 30, a first auriculotemporal nerve stimulation unit 302, a first greater auricular nerve stimulation unit 304, a supraorbital nerve stimulation unit 301, an infraorbital nerve stimulation unit 305 and an occipital nerve stimulation unit 303. The second neuromodulation unit 300 comprises at least one of the following: a second transcutaneous auricular vagus nerve stimulation unit (second taVNS unit) 30, a second auriculotemporal nerve stimulation unit 302 and a second greater auricular nerve stimulation unit 304. The second taVNS unit 30 includes a second taVNS stimulating electrode 31 configured to contact vagus innervated auricular skin of the wearer's second ear 902, and the second auriculotemporal nerve stimulation unit 302 includes a second auriculotemporal nerve stimulating electrode 312 configured to contact auriculotemporal nerve innervated auricular skin of the wearer's second ear 902, while the second greater auricular nerve stimulation unit 304 includes a second greater auricular nerve stimulating electrode 314 configured to contact greater auricular nerve innervated auricular skin of the wearer's second ear 902. The processing unit 50, 401, may be configured to analyze the EEG data transmitted from the auricular EEG monitoring system 100, including EEG data recorded by the first EEG recording module 20 and EEG data recorded by the second EEG recording module 20, to detect presence or cessation of EEG signals suggestive of at least one neuropsychiatric disorder. The processing unit may be further configured to analyze the EEG data transmitted from the auricular EEG monitoring system 100, including EEG data recorded by the first EEG recording module 20 and EEG data recorded by the second EEG recording module 20, to detect presence or cessation of EEG signals suggestive of at least one impending neuropsychiatric disorder. When the processing unit 50, 401, detects at least one of the following: presence of EEG signals suggestive of at least one neuropsychiatric disorder by analyzing EEG data recorded by the first EEG recording module 20 and presence of EEG signals suggestive of at least one neuropsychiatric disorder by analyzing EEG data recorded by the second EEG recording module 20, the processing unit 50, 401, is configured to start sending predetermined neuromodulating electric stimulation from at least one of the following: the first taVNS unit 30, the first auriculotemporal nerve stimulation unit 302, the first greater auricular nerve stimulation unit 304, the second taVNS unit 30, the second auriculotemporal nerve stimulation unit 302, the second greater auricular nerve stimulation unit 304, the supraorbital nerve stimulation unit 301, the infraorbital nerve stimulation unit 305 and the occipital nerve stimulation unit 303 (including various combinations thereof). When the processing unit detects at least one of the following: presence of EEG signals suggestive of at least one impending neuropsychiatric disorder by analyzing EEG data recorded by the first EEG recording module 20 and presence of EEG signals suggestive of at least one impending neuropsychiatric disorder by analyzing EEG data recorded by the second EEG recording module 20, the processing unit 50, 401, is configured to start sending predetermined neuromodulating electric stimulation from at least one of the following: the first taVNS unit 30, the first auriculotemporal nerve stimulation unit 302, the first greater auricular nerve stimulation unit 304, the second taVNS unit 30, the second auriculotemporal nerve stimulation unit 302, the second greater auricular nerve stimulation unit 304, the supraorbital nerve stimulation unit 301, the infraorbital nerve stimulation unit 305 and the occipital nerve stimulation unit 303 (including various combinations thereof). When the processing unit 50, 401, detects all of the following: cessation of EEG signals suggestive of at least one neuropsychiatric disorder by analyzing EEG data recorded by the first EEG recording module 20, cessation of EEG signals suggestive of at least one impending neuropsychiatric disorder by analyzing EEG data recorded by the first EEG recording module 20, cessation of EEG signals suggestive of at least one neuropsychiatric disorder by analyzing EEG data recorded by the second EEG recording module 20 and cessation of EEG signals suggestive of at least one impending neuropsychiatric disorder by analyzing EEG data recorded by the second EEG recording module 20, the processing unit 50, 401, is configured to stop sending predetermined neuromodulating electric stimulation from any of the following: the first taVNS unit 30, the first auriculotemporal nerve stimulation unit 302, the first greater auricular nerve stimulation unit 304, the second taVNS unit 30, the second auriculotemporal nerve stimulation unit 302, the second greater auricular nerve stimulation unit 304, the supraorbital nerve stimulation unit 301, the infraorbital nerve stimulation unit 305 and the occipital nerve stimulation unit 303.

In some embodiments for an automatic detection-therapy system 101 for neuropsychiatric disorders, the auricular EEG recording module 20 having a plurality of EEG sensor electrodes 12, 13, 72, 73, 82, 83, the taVNS unit 30, the auriculotemporal nerve (ATN) stimulation unit 302, the greater auricular nerve (GAN) stimulation unit 304 and the processing unit 50, 401, may be housed together in a single housing structure (such as an auricular housing 11) and placed in the external ear canal 904, tragus-concha bowl 957, and/or peri-auricular area 903. Alternatively, the taVNS unit 30, the ATN stimulation unit 302, the GAN stimulation unit 304 and the EEG recording module 20 may be in separate housings and may be located adjacent to each other with each of them attached to one or more of the following: the external ear canal 904, the tragus-concha bowl 957, and the peri-auricular area 903. In some embodiments, all of the EEG sensor electrodes 12, 13, 72, 73, 82, 83, the optional reference electrode 84, the taVNS stimulating electrode 31, the auriculotemporal nerve (ATN) stimulating electrode 312 and the greater auricular neve (GAN) stimulating electrode 314 may be all housed together in a single auricular housing 11. The auricular housing 11 may be selected from one of the following: a modified earbud housing 61, a modified in-the-ear housing 62, and a behind-the-ear-hearing-aid-style housing 63, as described hereinbefore. All of these housing selections include a tubular-shaped structure 66 (configured to be inserted into an external ear canal 904 of a wearer when in use) and a body-structure 67 (configured to be placed at the opening of the external ear canal 904 and to sit or be placed inside a tragus-concha bowl 957 of the wearer's ear when in use). Preferably, all of the EEG sensor electrodes 12, 13, 72, 73, 82, 83, may be configured to be located on a surface 91 of the tubular-shaped structure 66, while the taVNS stimulating electrode 31 and the auriculotemporal nerve (ATN) stimulating electrode 312 are configured to be located on a surface of one of: the tubular-shaped structure 66 and the body-structure 67. Preferably, the optional reference electrode 84 and the greater auricular nerve (GAN) stimulating electrode 314 may be configured to be placed on a surface of the body-structure 67. All of the EEG sensor electrodes 12, 13, 72, 73, 82, 83, the optional reference electrode 84, the taVNS stimulating electrode 31, the ATN stimulating electrode 312 and the GAN stimulating electrode 314 are configured to be partially embedded in the surface with slight protrusion at the surface of one of: the tubular-shaped structure 66 and the body-structure 67. The tubular-shaped structure 66 and the body-structure 67 may be configured to comprise a flexible elastic and adaptable material and the flexible elastic and adaptable material is configured to have appropriate flexibility, elasticity and adaptability so that the tubular-shaped structure 66 will naturally adapt to the contour of the external ear canal 904 and snugly fill the interior of the wearer's external ear canal 904 when the tubular-shaped structure 66 is inserted into the external ear canal 904 of the wearer's ear 902 and, meanwhile, the body-structure 67 will naturally adapt to the contour of the tragus-concha bowl 957 of the wearer's ear 902 and snugly fill the interior of the tragus-concha bowl 957 of the wearer's ear 902 when the body-structure 67 is placed inside the tragus-concha bowl 957 of the wearer's ear 902, so that all of the EEG sensor electrodes 12, 13, 72, 73, 82, 83, are naturally snugly in contact with the skin of the external ear canal 904 of the wearer's ear 902, and, at the same time, the taVNS stimulating electrode 31 and the ATN stimulating electrode 312 are naturally and snugly in contact with the skin of the tragus-concha bowl 957 of the wearer's ear 902 or the skin of the external ear canal 904 of the wearer's ear 902, and meanwhile, the GAN stimulating electrode 314 and the optional reference electrode 84 are naturally snugly in contact with the skin of the wearer's tragus-concha bowl 957; and so that the taVNS stimulating electrode 31 is naturally and snugly in contact with its target skin of vagus-innervated auricular skin of the wearer's first ear, provided by carefully selecting a location for the taVNS stimulating electrode 31 on the tubular-shaped structure 66 or the body-structure 67 to match one of the innervation locations of the vagus innervated skin on the external ear canal 904 or the tragus-concha bowl 957, the auriculotemporal nerve (ATN) stimulating electrode 312 is naturally and snugly in contact with its target skin of the ATN innervated auricular skin of the wearer's ear 902, provided by carefully selecting a location for the ATN stimulating electrode 312 on the tubular-shaped structure 66 or the body-structure 67 to match one of the innervation locations of the ATN innervated skin on the external ear canal 904 or the tragus-concha bowl 957, and, furthermore, the greater auricular nerve (GAN) stimulating electrode 314 is naturally snugly in contact with the GAN innervated auricular skin, provided by carefully selecting a location for the GAN stimulating electrode 314 on the body-structure 67 to match one of the innervation locations of the GAN innervated skin on the tragus-concha bowl 957 when the tubular-shaped structure 66 is inserted into the wearer's external ear canal 904 and the body-structure 67 is placed in the wearer's tragus-concha bowl 957. (Please note that the above descriptions are for a neuromodulation unit 30 that includes 3 components, including a taVNS unit 30, an ATN stimulation unit 302 and a GAN stimulation unit 304. If the neuromodulation unit 300 includes only one or two of these components, the aforementioned convenience and advantages can be similarly achieved.)

Preferably, the automatic detection-therapy system 101 for neuropsychiatric disorders may further comprise a network interface 53, 406, in electronic communication with the processing unit 50, 401. The network interface 53, 406, may be configured to generate a notification (such as an audible notification via a speaker 15, 404A, or a tactile notification via a vibrator 17, 404B). When a neuropsychiatric disorder or an impending neuropsychiatric disorder is detected, the network interface 53, 406, may be configured to automatically generate a notification to a client device 400, such as to a client device 400 of the wearer 900 and/or a client device 400 of the wearer's healthcare provider 950 so that the wearer 900 or the wearer's healthcare provider 950 can take appropriate actions. Besides that, the neuromodulation unit 300 may be automatically actuated to start giving pre-determined neuromodulating stimulation to the wearer 900. The neuromodulation unit 300 includes at least one of the following components: a taVNS unit 30, a supraorbital nerve stimulation unit 301, an auriculotemporal nerve stimulation unit 302, an occipital nerve stimulation unit 303, a greater auricular nerve stimulation unit 304 and an infraorbital nerve stimulation unit 305. Thus, when a neuropsychiatric disorder or impending neuropsychiatric disorder is detected, in addition to receiving at least one neuromodulation therapy automatically, the wearer 900 can take extra dose of medications or take other actions to alleviate the condition. An example of the notification device of a client device 400 is a speaker 404A, configured to generate audible warning notification (or a vibrator 404 B, configured to generate tactile notification) when presence of a neuropsychiatric disorder or impending neuropsychiatric disorder is detected by the processing unit 50, 401. The notification function is well-known. Most smart phones, smart watches and health trackers have notification function.

In some embodiments, an automatic detection-therapy system 101 for neuropsychiatric disorders may comprise an auricular EEG recording module 20 having a plurality of EEG sensor electrodes 12, 13, 72, 73, 82, 83, a processing unit 50, 401, a taVNS unit 30, an auriculotemporal nerve stimulation unit 302, a greater auricular nerve stimulation unit 304 and a network interface 53 and all or part of them (or part of their components) may be configured to be housed in a single auricular housing 11. The auricular housing 11 may be selected from one of the following: a modified earbud housing 61, a modified in-the-ear housing 62, a behind-the-ear-hearing-aid-style housing 63 and a tubular-shaped structure 66. The modified earbud housing 61 may include a tubular-body portion 25 and a stem portion 68. (FIGS. 2, 11). The stem portion 68 is equivalent to a “stem” (or a “stalk”) of an earbud. The “stem” hangs down from the wearer's ear. The tubular-body portion 25 may include a tubular-shaped structure 66 and a body-structure 67. The tubular-shaped structure 66 is equivalent to an elongated and modified “ear-tip” and “nozzle” of an earbud (or an AirPod) and may be inserted into a wearer's external ear canal 904 when in use. The body-structure 67 is equivalent to a “body” (or a “shell”) of an earbud (or an AirPod). The body-structure 67 may be placed at the immediate opening of the wearer's external ear canal 904 and sit or be placed inside the wearer's tragus-concha bowl 957 when in use. In some embodiments, the tubular-body portion 25 may be made with or may comprise elastic flexible and adaptable material. The material for the tubular-body portion 25 is configured to have appropriate elasticity, flexibility and adaptability so that when it is placed in the wearer's external ear canal 904 and the tragus-concha bowl 957, it will adapt to the contour of the wearer's external ear canal 904 and the contour of the wearer's tragus-concha bowl 957 and naturally and snugly fill the interior of the wearer's external ear canal 904 and the interior of the tragus-concha bowl 957. All of the EEG sensor electrodes 12, 13, 72, 73, 82, 83, may be configured to be placed on a surface 91 and partially embedded in the surface 91 with slight protrusion at the surface 91 of the tubular-shaped structure 66. The taVNS stimulating electrode 31 and the auriculotemporal nerve (ATN) stimulating electrode 312 may be configured to be placed on a surface and partially embedded in the surface with slight protrusion at the surface of one of: the tubular-shaped structure 66 and the body-structure 67 of the modified earbud housing 61, while the greater auricular nerve stimulating electrode 314 and the optional reference electrode 84 may be configured to be placed on a surface and partially embedded in the surface with slight protrusion at the surface of the body-structure 67. As aforementioned, the material of the tubular-body portion 25 (including the tubular shaped structure 66 and the body-structure 67) may be configured to have appropriate elasticity, flexibility and adaptability so that when the tubular-body portion 25 of the modified earbud housing 61 is placed in a wearer's external ear canal 904 and the tragus-concha bowl 957, the tubular-body portion 25 will adapt to the contour of the wearer's external ear canal 904 and the contour of the wearer's tragus-concha bowl 957 and naturally and snugly fill the interior of the wearer's external ear canal 904 and interior of the wearer's tragus-concha bowl 957 so that all of the EEG sensor electrodes 12, 13, 72, 73, 82, 83, will be naturally in close contact with the skin of the wearer's external ear canal 904 and, meanwhile, the taVNS stimulating electrode 31 and the auriculotemporal nerve stimulating electrode 312 will be naturally in close contact with the skin of the wearer's external ear canal 904 or skin of the wearer's tragus-concha bowl 957, and, at the same time, the greater auricular nerve stimulating electrode 314 and the optional reference electrode 84 will be naturally in close contact with the skin of the wearer's tragus-concha bowl 957. At the same time, the taVNS stimulating electrode 31 will be naturally in close contact with the skin of the wearer's vagus innervated auricular skin since the skin of the external ear canal 904 and the skin of the tragus-concha bowl 957 are part of the vagus innervated auricular skin. Likewise, at the same time, the auriculotemporal nerve (ATN) stimulating electrode 312 will be naturally in close contact with the skin of the wearer's ATN innervated auricular skin since the skin of the external ear canal 904 and the skin of the tragus-concha bowl 957 are also part of the ATN innervated auricular skin. In addition, the greater auricular nerve (GAN) stimulating electrode 314 will be naturally contacting GAN innervated skin of the cavum concha 907. Per the aforementioned descriptions of the innervation patterns, it is obvious that the tragus-concha 957 of a wearer's ear 902 received mixed and overlapped innervation from the auricular branch of vagus nerve, the auriculotemporal nerve (ATN) and the greater auricular nerve (GAN), while the external ear canal 904 received mixed and overlapped innervation from the auricular branch of vagus nerve and the ATN. When the taVNS stimulating electrode 31, the auriculotemporal nerve (ATN) stimulating electrode 312 and the greater auricular nerve (GAN) stimulating electrode 314 are located in the body-shaped structure 67, by carefully selecting the locations for the taVNS stimulating electrode 31, the ATN stimulating electrode 312 and the GAN stimulating electrode 314 on the body-structure 67 to match the aforementioned innervation locations of the innervated skin on the tragus-concha bowl 957, these stimulating electrodes 31, 312, 314, will automatically get in close contact with their target skin on the tragus-concha bowl 957 when the body-structure 67 is placed in the tragus-concha bowl 957. Alternatively, when the taVNS stimulating electrode 31 and the ATN stimulating electrode 312 are located in the tubular-shaped structure 66, by carefully selecting the locations for the taVNS stimulating electrode 31 and the ATN stimulating electrode 312 on the tubular-shaped structure 66 to match the aforementioned innervation locations of the innervated skin on the external ear canal 904, these stimulating electrodes 31, 312, will automatically get in close contact with their target skin on the external ear canal 904 when the tubular-shaped structure 66 is placed in the external ear canal 904. This unique design and set-up will allow the wearer 900 to install or to remove all of the EEG sensor electrodes 12, 13, 72, 73, 82, 83, the optional reference electrode 84 (if incorporated), the taVNS stimulating electrodes 31, the ATN stimulating electrode 312 and the GAN stimulating electrode 314 as easily as installing or removing the tubular-body portion 25 of the modified earbud housing 61 in (or from) the wearer's 900 external ear canal 904 and the tragus-concha bowl 957. Thus, this automatic detection-therapy system 101 can be set up automatically and is fully wearable, self-installable, self-removable, freely ambulatory and very convenient for wearers (users). (Please note that the above descriptions are for a neuromodulation unit 30 that includes 3 components, including a taVNS unit 30, an ATN stimulation unit 302 and a GAN stimulation unit 304. If the neuromodulation unit 300 includes only one or two of these components, the aforementioned convenience and advantages can be similarly achieved.)

In other embodiments for an automatic detection-therapy system 101 for neuropsychiatric disorders, all or part of the following (or part of their components): an auricular EEG recording module 20 having a plurality of EEG sensor electrodes 12, 13, 72, 73, 82, 83, a processing unit 50, 401, a taVNS unit 30, an auriculotemporal nerve (ATN) stimulation unit 302, a greater auricular nerve (GAN) stimulation unit 304 and a network interface 53 may be configured to be housed in an auricular housing 11 (in a single shared auricular housing 11). The auricular housing 11 may be selected from a behind-the-ear-hearing-aid-style housing 63. The behind-the-ear-hearing-aid-style housing 63 includes an in-the-ear portion 27 and a behind-the-ear portion 26. The in-the-ear portion 27 is essentially the same as the tubular-body portion 25 of the modified earbud housing 61 as aforementioned. (FIG. 13). The in-the-ear portion 27 of the behind-the-ear-hearing-aid-style housing 63 may include a tubular-shaped structure 66 and a body-structure 67. The tubular-shaped structure 66 is equivalent to an elongated modified “ear-tip” and “nozzle” of an earbud and will be inserted into a wearer's external ear canal 904 when in use. The body-structure 67 is equivalent to a “body” (or a “shell”) of an earbud. The body-structure 67 will be placed at the opening of the wearer's external ear canal 904 and sit (or be placed) inside the wearer's tragus-concha bowl 957 when in use. In some embodiments, the in-the-ear portion 27 may be made with or may comprise elastic flexible and adaptable material. The material for the in-the-ear portion 27 may be configured to have appropriate elasticity, flexibility and adaptability so that when it is placed in the wearer's external ear canal 904 and the tragus-concha bowl 957, it will naturally adapt to the contour of the wearer's external ear canal 904 and contour of the wearer's tragus-concha bowl 957 and snugly fill the interior of the wearer's external ear canal 904 and the interior of the tragus-concha bowl 957. All of the EEG sensor electrodes 12, 13, 72, 73, 82, 83, may be configured to be placed on a surface and partially embedded in the surface with slight protrusion at the surface of the tubular-shaped structure 66. The taVNS stimulating electrode 31 and the auriculotemporal nerve (ATN) stimulating electrode 312 may be configured to be placed on a surface and partially embedded in the surface with slight protrusion at the surface of one of: the tubular-shaped structure 66 and the body-structure 67 of the behind-the-ear-hearing-aid-style housing 63. Meanwhile, the greater auricular nerve (GAN) stimulating electrode 314 and the optional reference electrode 84 are configured to be placed on a surface and partially embedded in the surface with slight protrusion at the surface of the body-structure 67. The material of the in-the-ear portion 27 (including the tubular shaped structure 66 and the body-structure 67) may be configured to have appropriate elasticity, flexibility and adaptability so that when the in-the-ear portion 27 of the behind-the-ear-hearing-aid-style housing is placed in a wearer's external ear canal 904 and the tragus-concha bowl 957, the tubular-shaped structure 66 will adapt to the contour and fill the interior of the wearer's external ear canal 904, and, at the same time, the body-structure 67 will adapt to the contour and fill the interior of the wearer's tragus-concha bowl 957, so that all of the EEG sensor electrodes 12, 13, 72, 73, 82, 83, will be naturally and snugly in contact with the skin of the wearer's external ear canal 904, meanwhile, the optional reference electrode 84 and the GAN stimulating electrode will be naturally snugly in contact with the skin of the wearer's tragus-concha bowl 957, and, at the same time, the taVNS stimulating electrode 31 and the ATN stimulating electrode 312 will be naturally in close contact with the skin of the wearer's external ear canal 904 or skin of the wearer's tragus-concha bowl 957. Furthermore, the taVNS stimulating electrode 31 will be naturally in close contact with the skin of the wearer's vagus innervated auricular skin since the skin of the external ear canal 904 and the skin of the tragus-concha bowl 957 are part of the vagus innervated auricular skin (by carefully matching the location of the ta VNS stimulating electrode 31 on the tubular-shaped structure 66 or body-structure 67 with one of its target skin of the vagus innervation locations on the external ear canal 904 or the tragus-concha bowl 957). Likewise, at the same time, the auriculotemporal nerve (ATN) stimulating electrode 312 will be naturally in close contact with the skin of the wearer's ATN innervated auricular skin since the skin of the external ear canal 904 and the skin of the tragus-concha bowl 957 are part of the ATN innervated auricular skin (by carefully matching the location of the ATN stimulating electrode 312 on the tubular-shaped structure 66 or the body-structure 67 with one of its target skin of the ATN innervation locations on the external ear canal 904 or the tragus-concha bowl 957). In addition, the greater auricular nerve (GAN) stimulating electrode 314 will be naturally in close contact with the skin of the GAN innervated cavum concha 907 (by carefully matching the location of the GAN stimulating electrode 314 on the body-structure 67 with one of its target skin of the GAN innervation locations on the tragus-concha bowl 957). This unique design and set-up will allow the wearer 900 to install or to remove all of the EEG sensor electrodes 12, 13, 72, 73, 82, 83, the optional reference electrode 84 (if incorporated), the taVNS stimulating electrodes 31, the ATN stimulating electrode 302 and the GAN stimulating electrode 314 as easily as placing or removing the in-the-ear portion 27 of the behind-the-ear-hearing-aid style housing 63 in (or from) the wearer's 900 external ear canal 904 and the tragus-concha bowl 957. This automatic detection-therapy system 101 can be set up automatically and is wearable, user-installable, user-removable, freely ambulatory and very convenient for wearers (users). Alternatively, the auriculotemporal nerve stimulation unit 302 may have a clip electrode 319 to be clipped manually to the wearer's anterior-superior helix 909 of the ear 902 for the auriculotemporal nerve stimulating unit 902 to deliver the electric stimuli via the clip electrode 319 (FIG. 28). (Please note that the above descriptions are for a neuromodulation unit 30 that includes 3 components, including a taVNS 30, an ATN stimulation unit 302 and a GAN stimulation unit 304. If the neuromodulation unit 300 includes only one or two of these components, the aforementioned convenience and advantages can be similarly achieved.)

In some embodiments for an automatic detection-therapy system 101 for neuropsychiatric disorders, all or part of the following (or part of their components): an auricular EEG recording module 20 having a plurality of EEG sensor electrodes 12, 13, 72, 73, 82, 83, a processing unit 50, 401, a taVNS unit 30, an auriculotemporal nerve (ATN) stimulation unit 302, a greater auricular nerve (GAN) stimulation unit 304 and a network interface 53 may be configured to be housed in an auricular housing 11 (in a single shared auricular housing 11) and the auricular housing 11 may comprise a modified in-the-ear-housing 62. As aforementioned, a modified earbud housing 61 includes a tubular-shaped structure 66 and a body-structure 67. A behind-the-ear-hearing-aid-style housing 63 also includes a tubular-shaped structure 66 and a body-structure 67. Similarly, the modified in-the-ear housing 62 also includes a tubular-shaped structure 66 and a body-structure 67. Thus, the aforementioned descriptions for the tubular-shaped structure 66 and the body-structure 67 for a modified earbud housing 61 and for a behind-the-ear-hearing-aid style housing 63 may be applied to the modified in-the-ear housing 62. In some embodiments for an automatic detection-therapy system 101, having any of these 3 types of housing (including a modified earbud housing 61, a behind-the-ear-hearing-aid-style housing 63 and a modified in-the-ear housing 62), all of the EEG sensor electrodes 12, 13, 72, 73, 82, 83, may be placed at a surface 91 and partially embedded in the surface 91 with slight protrusion at the surface 91 of the tubular-shaped structure 66, while the taVNS stimulating electrode 31 and the auriculotemporal nerve (ATN) stimulating electrode 312 may be placed in a surface and partially embedded in the surface with slight protrusion at the surface of one of: the tubular-shaped structure 66 and the body-structure 67. Meanwhile, the greater auricular nerve (GAN) stimulating electrode 314 and the optional reference electrode 84 may be placed on a surface and partially embedded in the surface with slight protrusion at the surface of the body-structure 67, similar to the aforementioned descriptions. The tubular-shaped structure 66 is configured to be made of elastic flexible and adaptable material. The material of the tubular-shaped structure 66 is configured to have appropriate elasticity, flexibility and adaptability so that when the tubular-shaped structure 66 is inserted into a wearer's external ear canal 904, the tubular-shaped structure will adapt to the contour and fill the interior of the wearer's external ear canal 904 so that all of the EEG sensor electrodes 12, 13, 72, 73, 82, 83, will be naturally in close contact with the skin of the wearer's external ear canal 904. The body-structure 67 is also configured to be made of elastic flexible and adaptable material and the material is configured to have appropriate elasticity, flexibility and adaptability so that when the body-structure 67 is placed in the wearer's tragus-concha bowl 957 it will naturally adapt to the contour and fill the interior of the wearer's tragus-concha bowl 957, so that the GAN stimulating electrode 314 and the optional reference electrode 84 will be naturally in close contact with the skin of the wearer's tragus-concha bowl 957. Meanwhile, the taVNS stimulating electrode 31 and the ATN stimulating electrode 312 will be naturally in close contact with the skin of the wearer's external ear canal 904 or the skin of the wearer's tragus-concha bowl 957. This setup will enable the taVNS stimulating electrode to be naturally in close contact with the wearer's vagus innervated auricular skin, and the ATN stimulating electrode will be naturally in close contact with ATN innervated auricular skin and the GAN stimulating electrode 314 will also be naturally in close contact with GAN innervated auricular skin, similar to the aforementioned descriptions.

In preferred embodiments for an auricular EEG monitoring system 100 and/or an automatic detection-therapy system 101, all of the EEG sensor electrodes 12, 13, 72, 73, 82, 83, may be housed at a surface 91 of the tubular-shaped structure 66 and partially embedded in the surface 91 with slight protrusion at the surface 91 of the tubular-shaped structure 66. In preferred embodiments, one or more of the EEG sensor electrodes 12, 13, 72, 73, 82, 83, is/are configured to be located at the upper surface 91 (upper surface at approximately 90 degrees above horizontal level 92, as shown by electrodes 12, 13, in FIGS. 19, 22, 25) of the tubular-shaped structure 66. In preferred embodiments, one or more of the EEG sensor electrodes 12, 13, 72, 73, 82, 83, is/are configured to be located at between 0 and 90 degrees, and more preferably at approximately 45 degrees (plus or minus fifteen degrees or 30-60 degrees) above the horizontal level 92 of the tubular-shaped structure 66 and is/are configured to face forward-upward direction 93 (e.g., as shown by electrodes 72, 82, in FIGS. 19, 21, 24). In preferred embodiments, one or more of the EEG sensor electrodes is/are configured to be located at between 90 and 180 degrees, and more preferably at approximately 135 degrees (plus or minus fifteen degrees or 120-150 degrees) above the horizontal level 92 of the tubular-shaped structure 66 and is/are configured to face backward-upward direction 94 (e.g., as shown by electrodes 73, 83, in FIGS. 19, 23, 26) (Upper surface, horizontal level, forward, backward, and upward all refer to directions relative to the head 901 of the wearer 900 with the wearer in an upright position after the tubular-shaped structure 66 has been inserted into a wearer's external ear canal 904.). These arrangements will enable the EEG sensor electrodes 12, 13, 72, 73, 82, 83, to have one of the best locations and directions from the external ear canal 904 to record the wearer's EEG activities. (Upper surface, horizontal level, forward, backward and upward all refer to directions relative to the head 901 of the wearer 900 with the wearer in upright position after the tubular-shaped structure 66 has been inserted into the wearer's external ear canal 904.)

Alternatively, a separate client device 400 may be used for housing of some of the components of the automatic detection-therapy system 101 for neuropsychiatric disorders. For example, the EEG recording module 20 and the processing unit 50, 401, may be housed in a wearable client device 400, such as a watch-type structure. All of the EEG sensor electrodes 12, 13, 72, 73, 82, 83, the optional reference electrode 84, the taVNS stimulating electrode 31, the auriculotemporal nerve stimulating electrode 312 and the greater auricular nerve stimulating electrode 314 may be housed in one of: a modified earbud housing 61, a modified in-the-ear housing 62, and a behind-the-ear-hearing-aid-style housing 63. Wireless EEG sensor electrodes may be used for all of the EEG sensor electrodes 12, 13, 72, 73, 82, 83, and the optional reference electrode 84. (Wireless dry EEG electrodes such as that described by Ryan Kaveh et al. in Nature Communications on Aug. 2, 2024. Wireless dry EEG electrodes are also available from Zeto, Inc. headquarter in Santa Clara, California.) There are well-known wired or wireless EEG amplifiers available, such as EEG Electroencephalogram Smart Amplifier (Part #: EEG100D), or preferably BioNomadix 2Ch Wireless EEG Amplifier (Part #: BN-EEG2), both being made by the same company BIOPAC Systems, Inc. (Goleta, California). By using wireless EEG sensor electrodes 12, 13, 72, 73, 82, 83, wireless optional reference electrode 84 and wireless EEG amplifier 21, the EEG recording module 20 together with the processing unit 50, 401, may be housed remotely in a watch-type, smart phone-type or tablet-type client device 400 and communicate with all of the EEG sensor electrodes 12, 13, 72, 73, 82, 83, and the optional reference electrode 84 wirelessly.

In some embodiments, an automatic detection-therapy system 101 for neuropsychiatric disorders may comprises a switch 88 and a timer 89 to enable the wearer 900 to turn on or off the system 101 manually and setting a duration, an interval or a schedule, etc. Preferably, the switch 88 may be configured as a multi-mode switch 88 and the timer 89 may be configured as a multi-mode timer 89. In some embodiments, a multi-mode switch 88 may be configured to enable the wearer to set the system 101 at an automatic mode or a manual mode. The functions and setups of the automatic mode for system 101 are as described hereinbefore. In some embodiments for an automatic detection-therapy system 101 for neuropsychiatric disorders, the multi-mode switch 88 may be configured to allow the wearer 900 to choose at least one of the following switch selections: turning on or off an auricular EEG monitoring system 100, turning on or off a taVNS unit 30, turning on or off a supraorbital nerve stimulation unit 301, turning on or off an auriculotemporal nerve stimulation unit 302, turning on or off an occipital nerve stimulation unit 303, turning on or off a greater auricular nerve stimulation unit 304, turning on or off an infraorbital nerve stimulation unit 305, including various combinations thereof, and selecting from various stimulation modes. The components of the neuromodulation unit 300 is configured to be selectable by the wearer 900 via the multi-mode switch 88, and the wearer 900 can switch back and forth among different components of the neuromodulation unit 300 via the multi-mode switch 88. The multi-mode timer 89 may be configured to function together with the multi-mode switch 88 to set each switch selection at a desirable timing course such as duration, interval, countdown, daily/weekly schedule, 10 minute/30 minutes/60 minutes (or other duration) selection, continuous operation, manual turning on and manual shutdown etc. Furthermore, the multi-mode switch 88 and multi-mode timer 89 may be configured as programmable. In some embodiments, the multi-mode switch 88 function and the multi-mode timer 89 function may be configured to be incorporated into a wearer's client device 400 and can be easily set up as programmable. In the manual mode, the wearer 900 can use at least one of: the taVNS unit 30, the supraorbital nerve stimulation unit 301, the auriculotemporal nerve stimulation unit 302, the occipital nerve stimulation unit 303, the greater auricular nerve stimulation unit 304 and the infraorbital nerve stimulation unit 305 (or various combinations thereof) for prophylactic purpose against various neuropsychiatric disorders or for health maintenance purpose. Studies have shown that the taVNS unit 30 is effective not only for therapy of neuropsychiatric disorders and impending neuropsychiatric disorders, but also for prophylaxis of various neuropsychiatric disorders. Studies have also shown that the taVNS unit, the supraorbital nerve stimulation unit 301, the auriculotemporal nerve stimulation unit 302, the occipital nerve stimulation unit 303, the greater auricular nerve stimulation unit 304 and the infraorbital nerve stimulation unit 305 (and various combinations thereof) are effective not only for therapy of neuropsychiatric disorders and impending neuropsychiatric disorders, but also for prophylaxis of various neuropsychiatric disorders. Studies have also shown that using neuromodulation with at least one of: a taVNS unit 30, a supraorbital nerve stimulation unit 301, an ATN stimulation unit 302, an occipital nerve stimulation unit 303, a GAN stimulation unit 304 and an infraorbital nerve stimulation unit 305, and various combinations thereof, has significant health benefit and can be used for health maintenance purpose. The aforementioned programmable set up for the system 101 will enable the system 101 to be used for prophylactic purpose and health maintenance purpose. The multi-mode switch 88, multi-mode timer 89 and the programmable design may enable the wearer the option to choose from one of the following: neuromodulation from one neuromodulation component (single neuromodulation), simultaneous neuromodulation from two neuromodulation components (double neuromodulation) and simultaneous neuromodulation from three neuromodulation components (triple neuromodulation). Thus, when prompted, the neuromodulation unit 300 is configured to generate neuromodulating electric stimulation to a wearer 900 according to the choice of the stimulation mode selected by the wearer 900. The choices of the stimulation mode include the following: single neuromodulation by one component of the neuromodulation unit 300, double neuromodulation by various combinations of two components of the neuromodulation unit 300, triple neuromodulation by various combinations of three components of the neuromodulation unit 300, etc. In some embodiments, the neuromodulation unit 300 is configured to be controlled by the multi-mode switch 88 and multi-mode-timer 89 to allow the wearer 900 to switch back and forth among different components of the neuromodulating unit 300 via the multi-mode switch 88 and the multi-mode-timer 89 (or multifunctional switch-timer).

There are many conventional smart watches or health trackers that contain monitoring devices 71 to monitor various body functions, including electrocardiogram (ECG), heart rate, blood oxygen, sleep, body temperature, and motion etc. (as known in the art). However, none of them can monitor the most important part of the human body, which is the brain. This automatic detection-therapy system 101 can easily combine with other monitoring devices 71 by placing their monitoring sensors in the external ear 902 or the external ear canal 904. Thus, this combined system 100 and/or system 101 can monitor not only the brain (through EEG), it can also monitor other body functions, such as ECG, heart rate, blood oxygen, motion, sleep and body temperature, etc.

### A Second Preferred Embodiment

In preferred embodiments, an auricular EEG monitoring system 100 may comprise an electroencephalogram (EEG) recording module 20 configured to be linked to a wearer's first ear 902 or a peri-auricular area 903 around the wearer's first ear. The EEG recording module 20 includes a plurality of wired or wireless EEG sensor electrodes 12, 13, 72, 73, 82, 83 and a wired or wireless EEG amplifier 21. Optionally, the EEG recording module 20 may include an optional reference electrode 84. (The optional reference electrode 84 would be desirable but is not always needed. Instead, average of all of the EEG sensor electrodes 12, 13, 72, 73, 82, 83, can be used as a reference, i.e. common average reference.) These EEG sensor electrodes 12, 13, 72, 73, 82, 83, and the optional reference electrode 84 are configured to contact separate areas selected from at least one of the following: the external ear canal 904 of the wearer's first ear, external ear 902 of the wearer's first ear, or peri-auricular area 903 around the wearer's first ear. All of the EEG sensor electrodes 12, 13, 72, 73, 82, 83 and the optional reference electrode 84 are in wired or wireless electronic communication with the EEG amplifier 21 of the EEG recording module 20. The auricular EEG monitoring system 100 may further comprise a processing unit 50, 401. The auricular EEG recording module 20 may be in electronic communication with the processing unit 50, 401, through wire, Bluetooth or other wireless connection means. The auricular EEG recording module 20 may be configured to collect EEG data of the wearer 900 via all of the EEG sensor electrodes 12, 13, 72, 73, 82, 83 and the optional reference electrode 84 (if the optional reference electrode is incorporated). These EEG data are transmitted or otherwise communicated to the processing unit 50, 401. With the help of various EEG analysis algorithms, together with machine learning, deep learning and artificial intelligence, the processing unit 50, 401, may be configured to analyze the EEG data of the wearer 900 for detection of presence of EEG signals suggestive of neuropsychiatric disorders. The processing unit 50, 401 may be also configured to analyze the EEG data for detection of presence of EEG signals suggestive of impending neuropsychiatric disorders. The processing unit 50, 401, may be further configured to detect cessation of EEG signals suggestive of neuropsychiatric disorders or cessation of EEG signals suggestive of impending neuropsychiatric disorders.

The neuropsychiatric disorders that the auricular EEG monitoring system 100 may be configured to detect may include seizure, migraine, cluster headache, neurodegenerative diseases, major depressive disorder (MDD), bipolar disorder, schizophrenia, obsessive compulsive disorder (OCD), attention deficit hyperactivity disorder (ADHD), autism spectrum disorder (ASD), post-traumatic stress disorder (PTSD), anxiety disorder and panic disorder, etc.

The auricular EEG monitoring system 100 may further comprise a network interface 53, 403, configured to generate a notification. The network interface 53, 403, may be in electronic communication with the processing unit 50, 401. When the processing unit 50, 401, detects presence of EEG signals of a wearer 900 suggestive of at least one neuropsychiatric disorder or presence of EEG signals suggestive of at least one impending neuropsychiatric disorder, the network interface 53, 403, may be configured to send warning notification to a client device for the wearer 900 of the auricular EEG monitoring system 100 and/or the wearer's healthcare provider 950 to take appropriate actions. One example of the notification device is a speaker 15, 404A, configured to generate audible warning notification when presence of EEG signals suggestive of a neuropsychiatric disorder or presence of EEG signals suggestive of an impending neuropsychiatric disorder is detected. Another example of a notification device is a vibrator 17, 404B, configured to generate tactile notification when presence of EEG signals suggestive of a neuropsychiatric disorder or impending neuropsychiatric disorder is detected. When the processing unit 50, 401, detects cessation of EEG signals of a wearer 900 suggestive of at least one neuropsychiatric disorder or cessation of EEG signals suggestive of at least one impending neuropsychiatric disorder, the network interface 53, 403, may be further configured to send notification to a client device for the wearer 900 of the auricular EEG monitoring system 100 and/or the wearer's healthcare provider 950.

In some embodiments, an auricular EEG monitoring system 100 having an auricular EEG recording module 20, a processing unit 50, 401, and a network interface 53 may be configured to be housed in one of the following housings (housing refers to protective covers or protective structures): a modified earbud housing 61 (pod/bud), a modified in-the-ear housing 62, a behind-the-ear-hearing-aid-style housing 63 and a tubular-shaped structure 66. In some embodiments, an auricular EEG monitoring system 100 may be configured to be housed in a modified earbud housing 61. The modified earbud housing 61 includes a tubular-body portion 25 and a stem portion 68. All of the EEG sensor electrodes 12, 13, 72, 73, 82, 83, and the optional reference electrode 84 may be placed on a surface 91 of a tubular-body portion 25 of the modified earbud housing 61. The tubular-body portion 25 includes a tubular-shaped structure 66 (to be inserted into a wearer's external ear canal 904 when in use) and a body-structure 67 (to be placed at the opening of the wearer's external ear canal 904 and to sit or be placed inside the tragus-concha bowl 957 when in use). In some embodiments, all of the EEG sensor electrodes 12, 13, 72, 73, 82, 83, may be configured to be placed on the surface 91 and partially embedded in the surface 91 with slight protrusion at the surface 91 of the tubular-shaped structure 66 so that all of the EEG sensor electrodes 12, 13, 72, 73, 82, 83, will be located in the wearer's external ear canal 904 when in use; while the optional reference electrode 84 may be configured to be placed on a surface and partially embedded in the surface with slight protrusion at the surface of the body-structure 67 so that the optional reference electrode 84 will be located in the tragus-concha bowl 957 when in use. (Alternatively, the optional reference electrode 84 may be configured to contact one of the following: a tragus-concha bowl 957 of a wearer's ear 902 and a mastoid of a peri-auricular area 903 around the wearer's ear 902.) Preferably the tubular-body portion 25 of the modified earbud housing 61 may be made with or may comprise an elastic flexible and adaptable material, in which the material for the tubular-body portion 25 is configured to have appropriate elasticity, flexibility and adaptability so that when the tubular-body portion 25 is placed in a wearer's external ear canal 904 and the tragus-concha bowl 957, it will naturally adapt to the contour of the wearer's external ear canal 904 and the contour of the tragus-concha bowl 957 and it may snugly fill the interior of the wearer's external ear canal 904 and the interior of the tragus-concha bowl 957. (FIG. 8). This set-up and the flexibility, elasticity and adaptability of the material will enable all of these EEG sensor electrodes 12, 13, 72, 73, 82, 83, to be naturally in close contact with the skin of the wearer's external ear canal 904 and will also enable the optional reference electrode 94 to be naturally in close contact with the skin of the wearer's tragus-concha bowl 957 when the tubular-body portion 25 of the modified earbud housing 61 is placed in the wearer's ear 902 while in use. Installing and removing all of these EEG sensor electrodes 12, 13, 72, 73, 82, 83, and the optional reference electrode 84 will be as easy as inserting and removing the tubular-body portion 25 of the modified earbud housing 61 from the wearer's external ear 902. There will be no need for a certified technologist to apply or remove all of the EEG sensor electrodes 12, 13, 72, 73, 82, 83 and the optional reference electrode 84. Applying adhesive material to secure all of these EEG sensor electrodes 12, 13, 72, 73, 82, 83, and the optional reference electrode 84 will also be unnecessary. This is due to the unique anatomical features of the human external ear canal 904 and the bowl-cavity feature of the tragus-concha bowl 957, as illustrated in FIG. 8. These features will enable this auricular EEG monitoring system 100 to be set up automatically and be fully wearable, user-installable, user-removable and freely ambulatory. This will offer huge convenience for the wearer 900. In some embodiments, all of the aforementioned setups and advantages may be similarly applied when a modified in-the-ear housing 62 is used for housing of the auricular EEG monitoring system 100. These housing designs and setups will make all of the EEG sensor electrodes 12, 13, 72, 73, 82, 83 and the optional reference electrode 84 be user-installable and user-removable, and also make the auricular EEG monitoring system 100 has automated installation feature and automated removal feature.

In some embodiments, an auricular EEG monitoring system 100 may be configured to be housed in a behind-the-ear-hearing-aid-style housing 63. The behind-the-ear-hearing-aid-style housing 63 may include a behind-the-ear portion 26 and an in-the-ear portion 27. The in-the-ear portion 27 of the behind-the-ear-hearing-aid-style housing 63 is essentially the same as the tubular-body portion 66 of a modified earbud housing 61. The in-the-ear portion 27 of the behind-the-ear-hearing-aid-style housing 63 also includes a tubular-shaped structure 66 (to be inserted into a wearer's external ear canal 904 when in use) and a body-structure 67 (to be placed at the opening of the wearer's external ear canal 904 and to sit or be placed inside the wearer's tragus-concha bowl 957 when in use). All of the EEG sensor electrodes 12, 13, 72, 73, 82, 83, may be placed on a surface 91 and partially embedded in the surface 91 with slight protrusion at the surface 91 of the tubular-shaped structure 66 while the optional reference electrode 84 may be placed on a surface and partially embedded in the surface with slight protrusion at the surface of the body-structure 67 (of the in-the-ear portion 27). The in-the-ear portion 27 of the behind-the-ear-hearing-aid-style housing 63 may comprise a flexible elastic and adaptable material, in which the material for the in-the-ear portion 27 is configured to have appropriate flexibility, elasticity and adaptability so that when the in-the-ear portion 27 is placed in a wearer's external ear canal 904 and tragus-concha bowl 957, it may naturally adapt to the contour of the wearer's external ear canal 904 and the contour of the tragus-concha bowl 957 and may snugly fill the interior of the wearer's external ear canal 904 and the interior of the tragus-concha bowl 957. This setup will enable all of the EEG sensor electrodes 12, 13, 72, 73, 82, 83, to be naturally in close contact with the skin of the wearer's external ear canal 904 and will also enable the optional reference electrode 84 to be naturally in close contact with the skin of the wearer's tragus-concha bowl 957 when the in-the-ear portion 27 is placed in the wearer's external ear 902. Alternatively, the optional reference electrode 84 may be placed in the behind-the-ear portion 26 and may be configured to contact the wearer's skin at peri-auricular area 903 (for example, the mastoid). These housing designs and setups will make all of the EEG sensor electrodes 12, 13, 72, 73, 82, 83 and the optional reference electrode 84 be user-installable and user-removable, and also make the auricular EEG monitoring system 100 has automated installation feature and automated removal feature.

In some embodiments, an auricular housing 11 of an auricular EEG monitoring system 100 may be shaped or configured as a standalone tubular-shaped structure 66. This standalone tubular-shaped structure 66 is essentially the same as the tubular-shaped structure 66 of a modified earbud housing 61 or the tubular-shaped structure 66 of an in-the-ear housing 61 or the tubular-shaped structure 66 of a behind-the-ear-hearing-aid-style housing 63. For any of the above tubular-shaped structure 66 and referring to FIG. 19, all of the EEG sensor electrodes 12, 13, 72, 73, 82, 83 may be configured to be located at the surface 91 of the tubular-shaped structure 66 and so that all of the EEG sensor electrodes 12, 13, 72, 73, 82, 83, are housed in the tubular-shaped structure 66. In preferred embodiments, one or more of the EEG sensor electrodes 12, 13, 72, 73, 82, 83, is/are configured to be located at the upper surface 91 (upper surface at approximately 90 degrees above horizontal level 92, as shown by electrodes 12, 13, in FIGS. 19, 22, 25) of the tubular-shaped structure 66. In preferred embodiments, one or more of the EEG sensor electrodes 12, 13, 72, 73, 82, 83, is/are configured to be located at between 0 and 90 degrees, and more preferably at approximately 45 degrees (plus or minus fifteen degrees) above the horizontal level 92 of the tubular-shaped structure 66 and is/are configured to face forward-upward direction 93 (e.g., as shown by electrodes 72, 82, in FIGS. 19, 21, 24). In preferred embodiments, one or more of the EEG sensor electrodes 12, 13, 72, 73, 82, 83, is/are configured to be located at between 90 and 180 degrees, and more preferably at approximately 135 degrees (plus or minus fifteen degrees) above the horizontal level 92 of the tubular-shaped structure 66 and is/are configured to face backward-upward direction 94 (e.g., as shown by electrodes 73, 83, in FIGS. 19, 23, 26). These arrangements will enable the EEG sensor electrodes 12, 13, 72, 73, 82, 83, to have one of the best locations and directions from the external ear canal 904 to record the wearer's EEG activities. (Upper surface, horizontal level, forward, backward and upward all refer to directions relative to the head 901 of the wearer 900 with the wearer in upright position after the tubular-shaped structure 66 has been inserted into a wearer's external ear canal 904.) All of the EEG sensor electrodes 12, 13, 72, 73, 82, 83, are configured to be placed at the surface 91 and partially embedded in the surface 91 with slight protrusion at the surface 91 of the tubular-shaped structure 66. The tubular-shaped structure 66 is configured to be made of elastic flexible and adaptable material, in which the material of the tubular-shaped structure 66 is configured to have appropriate elasticity, flexibility and adaptability so that when the tubular-shaped structure 66 is inserted into a wearer's external ear canal 904, the tubular-shaped structure 66 will naturally adapt to the contour of the wearer's external ear canal 904 and fill the interior of the wearer's external ear canal 904 and, thus, all of the EEG sensor electrodes 12, 13, 72, 73, 82, 83, will be naturally in close contact with the skin of the wearer's external ear canal 904. This set-up will allow the wearer 900 to install or to remove all of the EEG sensor electrodes 12, 13, 72, 73, 82, 83, as easily as inserting or removing the tubular-shaped structure 66 into (or from) the wearer's 900 external ear canal 904.

In some embodiments, an auricular EEG monitoring system 100 may further comprise a second auricular EEG recording module 20. The first EEG recording module 20 may be configured to be linked to the wearer's first ear 902 or the peri-auricular area 903 around the wearer's first ear; while the second EEG recording module 20 may be configured to be linked to the wearer's second ear 902 or the peri-auricular area 903 around the wearer's second ear. The second EEG recording module 20 may comprise a plurality (at least two, but preferably more than two) of EEG sensor electrodes 12, 13, 72, 73, 82, 83, and an optional reference electrode 84, configured to contact separate areas selected from at least one of the following: external ear canal 904 of the wearer's second ear, external ear 902 of the wearer's second ear, and peri-auricular area 903 around the wearer's second ear. The second EEG recording module 20 may be in electronic communication with the processing unit 50, 401, through Bluetooth or other connection means. The second auricular EEG recording module 20 may be configured to collect EEG data of the wearer 900 via all of its EEG sensor electrodes 12, 13, 72, 73, 82, 83 and the optional reference electrode 84. The EEG data from the first EEG recording module 20 and the second EEG recording module 20 may be transmitted to the processing unit 50, 401. With the help of various EEG analysis algorithms, together with machine learning, deep learning and artificial intelligence, the processing unit 50, 401, may be configured to analyze the EEG data transmitted from both the first and the second EEG recording modules 20 for detection of presence of EEG signals suggestive of neuropsychiatric disorders or presence of EEG signals suggestive of impending neuropsychiatric disorders. The processing unit 50, 401, may be further configured to detect cessation of EEG signals suggestive of neuropsychiatric disorders or cessation of the EEG signals suggestive of impending neuropsychiatric disorders. The neuropsychiatric disorders may include seizure, migraine, cluster headache, neurodegenerative diseases, major depressive disorder, bipolar disorder, schizophrenia, obsessive compulsive disorder, attention deficit hyperactivity disorder, autism spectrum disorder, post-traumatic stress disorder, anxiety disorder and panic disorder, etc. With bilateral EEG data collection, this auricular EEG monitoring system 100 will be able to more widely collect EEG data from both sides of the wearer's brain to help more accurately detect presence or cessation of EEG signals suggestive of neuropsychiatric disorders and presence or cessation of EEG signals suggestive of impending neuropsychiatric disorders.

There are many conventional smart watches or health trackers that contain monitoring devices 71 to monitor various body functions, including electrocardiogram (ECG), heart rate, blood oxygen, sleep, body temperature, and motion, etc. (as known in the art). However, none of them can monitor the most important part of the human body, which is the brain. This auricular EEG monitoring system 100, can easily combine with other monitoring devices 71 by placing their monitoring sensors in the external ear 902 or the external ear canal 904. Thus, this combined system 100 can monitor not only the brain (through EEG), it can also monitor other body functions, such as ECG, heart rate, blood oxygen, motion, sleep, and body temperature, etc.

### A Third Preferred Embodiment

In still another aspect consistent with the principles of this invention, a combined neuromodulation system 102 is disclosed. The combined neuromodulation system 102 comprises at least two neuromodulating components. In some embodiments, a combined neuromodulation system 102 comprises two neuromodulating components, including a transcutaneous auricular vagus nerve stimulation unit (taVNS unit) 30 and an auriculotemporal nerve (ATN) stimulation unit 302. In other embodiments, a combined neuromodulation system 102 comprises two neuromodulating components, including a transcutaneous auricular vagus nerve stimulation unit (taVNS unit) 30 and a greater auricular nerve (GAN) stimulation unit 304. In still other embodiments, a combined neuromodulation system 102 comprises two neuromodulating components, including an auriculotemporal nerve (ATN) stimulation unit 302 and a GAN stimulation unit 304. In modified embodiments, a combined neuromodulation system 102 comprises three neuromodulating components, including a taVNS unit 30, an ATN stimulation unit 302 and a GAN stimulation unit 304. (FIGS. 18, 27). The setups and functions of each neuromodulating component of the combined neuromodulation system 102 (i.e. taVNS unit 30, ATN stimulation unit 302, AGN stimulation unit 304) are as described hereinbefore.

In some embodiments, a combined neuromodulation system 102 may further comprise a multi-mode switch 88 and a multi-mode timer 89 (or an integrated multifunctional switch-timer, including a programmable multifunctional switch-timer). The multi-mode switch 88 and the multi-mode timer 89 are in electronic communication with each neuromodulating component of the combined neuromodulation system 102. The multi-mode switch 88 is configured to enable a wearer (user) 900 to select among various stimulation modes, including the following: single neuromodulation by one neuromodulating component of the neuromodulation system 102, double neuromodulation by various combinations of two neuromodulating components of the neuromodulation system 102, triple neuromodulation by all three neuromodulating components of the neuromodulation system 102. The multi-mode timer 89 is configured to enable the wearer 900 to select among various stimulation time courses. The multi-mode timer 89 may be configured to function together with the multi-mode switch 88 to allow the wearer 900 to set each switch selection of stimulation mode at a desirable time course such as duration, interval, countdown, daily/weekly schedule, 10 minute/30 minutes/60 minutes (or other duration) selection, continuous operation, manual turning on and manual shutdown etc. In some embodiments, the multi-mode switch 88 function and the multi-mode timer 89 function may be configured to be incorporated into a wearer's client device 400 and can be easily set up as programmable. The wearer (user) 900 may select among various stimulation modes and select among various time courses from the client device 400.

In some embodiments, a combined neuromodulation system 102 may be configured to be housed in an auricular housing 11 (such as a modified earbud housing 61 or a modified in-the-ear housing 62 or a behind-the-ear-hearing-aid-style housing 63) that includes a body-structure 67. The body-structure 67 is configured to be placed in the wearer's tragus-concha bowl 957 when in use. All of the stimulating electrodes, including the transcutaneous auricular vagus nerve stimulation (taVNS) stimulating electrode 31, the auriculotemporal nerve (ATN) stimulating electrode 312 and the greater auricular nerve (GAN) stimulating electrode 314, may be configured to be located on a surface of the body-structure 67 and are partially embedded in the surface with slight protrusion at the surface of the body-structure 67. The body-structure 67 is configured to be made of elastic flexible and adaptable material. The material of the body-structure 67 is configured to have appropriate elasticity, flexibility and adaptability so that the body-structure 67 will naturally adapt to the contour of the wearer's tragus-concha bowl 957 and snugly fill the interior of the wearer's tragus-concha bowl 957 when the body-structure 67 is placed in the wearer's tragus-concha bowl 67; and so that the taVNS stimulating electrode 31, the ATN stimulating electrode 312 and the GAN stimulating electrode 314 will be naturally in close contact with the skin of the wearer's tragus-concha bowl 957 when the body-structure 67 is placed in the wearer's tragus-concha bowl 957. It should be noted that the tragus-concha bowl 945 received mixed innervation from the auricular branch of vagus nerve, the auriculotemporal nerve (ATN) and the greater auricular nerve (GAN). With careful selection of the locations on the body-structure 67 for the stimulating electrodes (including the ta VNS stimulating electrode 31, the ATN stimulating electrode 312 and the GAN stimulating electrode 314) to match the innervation patterns for all of these 3 nerves (auricular branch of vagus nerve, ATN and GAN) on the tragus-concha bowl 957, the taVNS stimulating electrode 31 will be naturally in close contact with its target skin on the tragus-concha bowl 957, the ATN stimulating electrode 312 will be naturally in close contact with its target skin on the tragus-concha bowl 957 and the GAN stimulating electrode 314 will be naturally in close contact with its target skin on the tragus-concha bowl 957 when the body-structure 67 is placed in the tragus-concha bowl 957.

In modified embodiments, a combined neuromodulation system 102 may be configured to be housed in an auricular housing 11 (such as a modified earbud housing 61, a modified in-the-ear housing 62 or a behind-the-ear-hearing-aid-style housing 63) that includes a tubular-shaped structure 66 and a body-structure 67. The tubular-shaped structure 66 is configured to be inserted into a wearer's external ear canal 904 when in use. The body-structure 67 is configured to be placed in the wearer's tragus-concha bowl 957 when in use. The ta VNS stimulating electrode 31 and the ATN stimulating electrode 312 may be configured to be located on a surface (and partially embedded in the surface with slight protrusion at the surface) of the tubular-shaped structure 66, while the GAN stimulating electrode 314, may be configured to be located on a surface of the body-structure 67 and be partially embedded in the surface with slight protrusion at the surface of the body-structure 67. The tubular-shaped structure 66 and the body-structure 67 are configured to be made of elastic flexible and adaptable material and the material is configured to have appropriate elasticity, flexibility and adaptability so that the tubular-shaped structure 66 will naturally adapt to the contour of the external ear canal 904 and snugly fill the interior of the external ear canal 904 when the tubular-shaped structure 6 is inserted into the wearer's external ear canal 904, while the body-structure 67 will naturally adapt to the contour of the wearer's tragus-concha bowl 957 and snugly fill the interior of the wearer's tragus-concha bowl 957 when the body-structure 67 is placed in the wearer's tragus-concha bowl 957; and so that the taVNS stimulating electrode 31 and the ATN stimulating electrode 312 will be naturally in close contact with the skin of the wearer's external ear canal 904, while the GAN stimulating electrode 314 will be naturally in close contact with the skin of the wearer's tragus-concha bowl 957. It should be noted that the external ear canal 904 received mixed innervation from the auricular branch of vagus nerve and the ATN, while the tragus-concha bowl 945 received innervation from the greater auricular nerve (GAN). By carefully selecting the locations on the tubular-shaped structure 66 for the taVNS stimulating electrode 31 and the ATN stimulating electrode 312 to match their innervation patterns for the auricular branch of vagus nerve and ATN, the taVNS stimulating electrode 31 will be naturally in close contact with its target skin on the external ear canal 904, while the ATN stimulating electrode 312 will also be naturally in close contact with its target skin on the external ear canal 904 when the tubular-shaped structure 66 is inserted into the wearer's external ear canal 904. At the same time, by carefully selecting the location for the GAN stimulating electrode 314 on the body-structure 67 to match one of the GAN innervation locations on the tragus-concha bowl 957, the GAN stimulating electrode 314 will be naturally in close contact with its target skin on the tragus-concha bowl 957 when the body-structure 67 is placed in the tragus-concha bowl 957 of the wearer's ear 902.

In some embodiments, a combined neuromodulation system 102 is configured to be controlled by a multi-mode switch 88 and a multi-mode-timer 89 to allow the wearer 900 to switch back and forth among different components of the combined neuromodulation system 102 and among various stimulation modes via the multi-mode switch 88 and to set different time courses via the multi-mode-timer 89 (or multifunctional switch-timer). Studies have shown that using neuromodulation with one of: a taVNS unit 30, an ATN stimulation unit 302 and a GAN stimulation unit 304 has significant health benefit and benefit in prophylaxis of various neuropsychiatric disorders. There are also evidences that simultaneous neuromodulation to two different nerves has synergistic effects. This combined neuromodulation system 102 will be very useful for the wearer for health maintenance purpose and for prophylaxis purpose against many neuropsychiatric disorders. The combined neuromodulation system 102 is also very useful for prophylaxis and therapy of cardiac arrhythmia (such as atrial flutter, supraventricular tachycardia, atrial fibrillation and ventricular tachycardia, etc.)

There are evidences showing that bilateral neuromodulating electric stimulation of auricular branch of vagus nerve via taVNS is more effective than unilateral taVNS neuromodulation. Similarly, bilateral neuromodulating electric stimulation of auriculotemporal nerve (ATN) is more effective than unilateral ATN neuromodulation. Likewise, bilateral neuromodulating electric stimulation of greater auricular nerve (GAN) is more effective than unilateral GAN stimulation. In some embodiments, a combined neuromodulation system 102 may utilize two combined neuromodulation systems 102 with a first system 102 attached to a right ear 902 of a wearer, and a second system 102 attached to the left ear 902 of the wearer 900. Thus, the wearer 900 has the option of choosing among various components or various combinations of components of unilateral or bilateral neuromodulation as needed.

While some exemplary shapes and sizes have been provided for elements of the auricular EEG monitoring system 100 and automatic detection-therapy system 101 it should be understood to one of ordinary skill in the art that the elements described herein may be configured in a plurality of sizes and shapes including “T” shaped, “X” shaped, square shaped, rectangular shaped, cylinder shaped, cuboid shaped, hexagonal prism shaped, triangular prism shaped, or any other geometric or non-geometric shape, including combinations of shapes. It is not intended herein to mention all the possible alternatives, equivalent forms or ramifications of the invention. It is understood that the terms and proposed shapes used herein are merely descriptive, rather than limiting, and that various changes, such as to size and shape, may be made without departing from the spirit or scope of the invention.

Although the present invention has been illustrated and described herein with reference to preferred embodiments and specific examples thereof, it will be readily apparent to those of ordinary skill in the art that other embodiments and examples may perform similar functions and/or achieve like results. All such equivalent embodiments and examples are within the spirit and scope of the present invention, are contemplated thereby, and are intended to be covered by the following claims.

What is claimed is:

1. An automatic detection-therapy system for neuropsychiatric disorders, comprising:
an auricular electroencephalogram (EEG) monitoring system, comprising a first EEG recording module having a plurality of EEG sensor electrodes, wherein the first EEG recording module is in electronic communication with each EEG sensor electrode of the first EEG recording module, wherein each EEG sensor electrode of the first EEG recording module is configured to contact separate areas of a wearer's skin selected from at least one of the following: an external ear canal of a first ear of the wearer, an external ear of the wearer's first ear, and a peri-auricular area around the wearer's first ear, and wherein the first EEG recording module is configured to record EEG data of the wearer;
a first neuromodulation unit configured to send neuromodulating electric stimulation to the wearer when activated, wherein the first neuromodulation unit comprises at least two components selected from the following: a first transcutaneous auricular vagus nerve stimulation unit (first ta VNS unit), a supraorbital nerve stimulation unit, a first auriculotemporal nerve stimulation unit, an occipital nerve stimulation unit, a first greater auricular nerve stimulation unit and an infraorbital nerve stimulation unit, and wherein each component of the first neuromodulation unit is configured to send neuromodulating electric stimulation to the wearer when activated; and
a processing unit, wherein the processing unit is in electronic communication with the auricular EEG monitoring system and each component of the first neuromodulation unit;
wherein the processing unit is configured to analyze the EEG data recorded by the auricular EEG monitoring system to detect presence or cessation of EEG signals suggestive of at least one neuropsychiatric disorder;
wherein when presence of EEG signals suggestive of at least one neuropsychiatric disorder is detected by the processing unit, the processing unit is configured to immediately send signals to the first neuromodulation unit to automatically start sending neuromodulating electric stimulation to the wearer from the at least two components of the first neuromodulation unit;
wherein when cessation of EEG signals suggestive of at least one neuropsychiatric disorder is detected by the processing unit, the processing unit is further configured to immediately send signals to the first neuromodulation unit to automatically stop sending neuromodulating electric stimulation to the wearer from any component of the first neuromodulation unit;
wherein the processing unit is further configured to analyze the EEG data recorded by the auricular EEG monitoring system to detect presence or cessation of EEG signals suggestive of at least one impending neuropsychiatric disorder;
wherein when presence of EEG signals suggestive of at least one impending neuropsychiatric disorder is detected by the processing unit, the processing unit is configured to immediately send signals to the first neuromodulation unit to automatically start sending neuromodulating electric stimulation to the wearer from the at least two components of the first neuromodulation unit; and
wherein when cessation of EEG signals suggestive of at least one impending neuropsychiatric disorder is detected by the processing unit, the processing unit is configured to immediately send signals to the first neuromodulation unit to automatically stop sending neuromodulating electric stimulation to the wearer from any component of the first neuromodulation unit.

2. The automatic detection-therapy system for neuropsychiatric disorders of claim 1, wherein the first neuromodulation unit comprises a first transcutaneous auricular vagus nerve stimulation unit (first taVNS unit) and a first non-vagus electric neuromodulation unit, wherein the first non-vagus electric neuromodulation unit comprises at least one of the following components: the supraorbital nerve stimulation unit, the infraorbital nerve stimulation unit, the first auriculotemporal nerve stimulation unit, the occipital nerve stimulation unit and the first greater auricular nerve stimulation unit, wherein the processing unit is in electronic communication with the first taVNS unit and each component of the first non-vagus electric neuromodulation unit, wherein the processing unit is configured to analyze the EEG data to detect presence or cessation of EEG signals suggestive of at least one neuropsychiatric disorder, wherein the processing unit is further configured to analyze the EEG data to detect presence or cessation of EEG signals suggestive of at least one impending neuropsychiatric disorder,
wherein when the processing unit detects presence of EEG signals suggestive of at least one neuropsychiatric disorder the processing unit is configured to send signals to the first neuromodulation unit to prompt it to start sending predetermined neuromodulating electric stimulation from the first taVNS unit and the at least one component of the first non-vagus electric neuromodulation unit;
wherein when the processing unit detects cessation of EEG signals suggestive of at least one neuropsychiatric disorder, the processing unit is configured to send signals to the first neuromodulation unit to prompt it to stop sending neuromodulating electric stimulation from the first taVNS unit and the first non-vagus electric neuromodulation unit;
wherein when the processing unit detects presence of EEG signals suggestive of at least one impending neuropsychiatric disorder, the processing unit is configured to send signals to the first neuromodulation unit to prompt it to start sending predetermined neuromodulating electric stimulation from the first taVNS unit and the at least one component of the first non-vagus electric neuromodulation unit; and
wherein when the processing unit detects cessation of EEG signals suggestive of at least one impending neuropsychiatric disorder, the processing unit is further configured to send signals to the first neuromodulation unit to prompt it to stop sending neuromodulating electric stimulation from the first taVNS unit and the first non-vagus electric neuromodulation unit.

3. The automatic detection-therapy system for neuropsychiatric disorders of claim 1, further comprising a network interface, wherein the network interface is in electronic communication with the processing unit, wherein the network interface is configured to generate a notification to at least one of: a client device of the wearer and a client device of a healthcare provider of the wearer when the processing unit detects presence of EEG signals suggestive of at least one neuropsychiatric disorder, wherein the network interface is further configured to generate a notification to at least one of: the client device of the wearer and the client device of the healthcare provider of the wearer when the processing unit detects presence of EEG signals suggestive of at least one impending neuropsychiatric disorder, wherein the network interface is configured to generate a notification to at least one of: the client device of the wearer and the client device of the wearer's healthcare provider when the processing unit detects cessation of EEG signals suggestive of at least one neuropsychiatric disorder, and wherein the network interface is further configured to generate a notification to at least one of: the client device of the wearer and the client device of the wearer's healthcare provider when the processing unit detects cessation of EEG signals suggestive of at least one impending neuropsychiatric disorder.

4. The automatic detection-therapy system for neuropsychiatric disorders of claim 1, wherein the auricular EEG monitoring system further comprises a second EEG recording module, wherein the second EEG recording module comprises a plurality of EEG sensor electrodes, wherein the second EEG recording module is in electronic communication with each EEG sensor electrode of the second EEG recording module, wherein each EEG sensor electrode of the second EEG recording module is configured to contact separate areas of the wearer's skin selected from at least one of the following: an external ear canal of a second ear of the wearer, an external ear of the wearer's second ear, and a peri-auricular area around the wearer's second ear, and wherein the second EEG recording module is configured to record EEG data of the wearer, wherein the second EEG recording module is in electronic communication with the processing unit.

5. The automatic detection-therapy system for neuropsychiatric disorders of claim 4, further comprising a second neuromodulation unit, wherein the second neuromodulation unit comprises at least one of the following components: a second transcutaneous auricular vagus nerve stimulation unit (second taVNS unit), a second auriculotemporal nerve stimulation unit and a second greater auricular nerve stimulation unit, wherein the second taVNS unit includes a second taVNS stimulating electrode configured to contact vagus innervated auricular skin of the wearer's second ear, wherein the second auriculotemporal nerve stimulation unit includes a second auriculotemporal nerve stimulating electrode configured to contact auriculotemporal nerve innervated auricular skin of the wearer's second ear, wherein the second greater auricular nerve stimulation unit includes a second greater auricular nerve stimulating electrode configured to contact greater auricular nerve innervated auricular skin of the wearer's second ear, wherein the processing unit is configured to analyze the EEG data transmitted from the auricular EEG monitoring system, including EEG data recorded by the first EEG recording module and EEG data recorded by the second EEG recording module, to detect presence or cessation of EEG signals suggestive of at least one neuropsychiatric disorder, wherein the processing unit is further configured to analyze the EEG data transmitted from the auricular EEG monitoring system, including EEG data recorded by the first EEG recording module and EEG data recorded by the second EEG recording module, to detect presence or cessation of EEG signals suggestive of at least one impending neuropsychiatric disorder,
wherein when the processing unit detects at least one of the following: presence of EEG signals suggestive of at least one neuropsychiatric disorder by analyzing EEG data recorded by the first EEG recording module and presence of EEG signals suggestive of at least one neuropsychiatric disorder by analyzing EEG data recorded by the second EEG recording module, the processing unit is configured to start sending predetermined neuromodulating electric stimulation from at least one of the following: at least two components of the first neuromodulation unit and at least one component of the second neuromodulation unit;
wherein when the processing unit detects at least one of the following: presence of EEG signals suggestive of at least one impending neuropsychiatric disorder by analyzing EEG data recorded by the first EEG recording module and presence of EEG signals suggestive of at least one impending neuropsychiatric disorder by analyzing EEG data recorded by the second EEG recording module, the processing unit is configured to start sending predetermined neuromodulating electric stimulation from at least one of the following: at least two components of the first neuromodulation unit and at least one component of the second neuromodulation unit; and
wherein when the processing unit detects all of the following: cessation of EEG signals suggestive of at least one neuropsychiatric disorder by analyzing EEG data recorded by the first EEG recording module, cessation of EEG signals suggestive of at least one impending neuropsychiatric disorder by analyzing EEG data recorded by the first EEG recording module, cessation of EEG signals suggestive of at least one neuropsychiatric disorder by analyzing EEG data recorded by the second EEG recording module and cessation of EEG signals suggestive of at least one impending neuropsychiatric disorder by analyzing EEG data recorded by the second EEG recording module, the processing unit is configured to stop sending predetermined neuromodulating electric stimulation from any of the following: the first taVNS unit, the first auriculotemporal nerve stimulation unit, the first greater auricular nerve stimulation unit, the second taVNS unit, the second auriculotemporal nerve stimulation unit, the second greater auricular nerve stimulation unit, the supraorbital nerve stimulation unit, the infraorbital nerve stimulation unit and the occipital nerve stimulation unit.

6. The automatic detection-therapy system for neuropsychiatric disorders of claim 1, wherein the auricular EEG monitoring system comprises a plurality of EEG sensor electrodes and a first reference electrode, wherein the first neuromodulation unit comprises at least two of the following components: the first taVNS unit having a first ta VNS stimulating electrode configured to contact its target skin of vagus innervated auricular skin of the wearer's first ear, the first auriculotemporal nerve stimulation unit having a first auriculotemporal nerve stimulating electrode configured to contact its target skin of auriculotemporal nerve innervated auricular skin of the wearer's first ear, and the first greater auricular nerve stimulation unit having a first greater auricular nerve stimulating electrode configured to contact its target skin of greater auricular nerve innervated auricular skin of the wearer's first ear; wherein all of the EEG sensor electrodes, the first reference electrode, the first taVNS stimulating electrode, the first auriculotemporal nerve stimulating electrode and the first greater auricular nerve stimulating electrode are configured to be housed in a first auricular housing, wherein the first auricular housing comprises a first tubular-shaped structure and a first body-structure, wherein the first tubular-shaped structure is configured to be inserted into an external ear canal of the wearer's first ear when in use, wherein the first body-structure is configured to be placed at immediate opening of the external ear canal of the wearer's first ear and to be placed inside a tragus-concha bowl of the wearer's first ear when in use, wherein all of the EEG sensor electrodes are configured to be located on a surface and partially embedded in the surface with protrusion at the surface of the first tubular-shaped structure, wherein the first greater auricular nerve stimulating electrode and the first reference electrode are configured to be located on a surface and partially embedded in the surface with protrusion at the surface of the first body-structure, wherein the first taVNS stimulating electrode and the first auriculotemporal nerve stimulating electrode are configured to be located on the surface and partially embedded in the surface with protrusion at the surface of one of: the first tubular-shaped structure and the first body-structure, wherein the first tubular-shaped structure is configured to be made of elastic flexible adaptable material and the material is configured to have appropriate elasticity flexibility and adaptability so that the first tubular-shaped structure will naturally adapt to the contour of the external ear canal of the wearer's first ear and snugly fill the interior of the external ear canal of the wearer's first ear when the first tubular-shaped structure is inserted into the external ear canal of the wearer's first ear, and so that all of the EEG sensor electrodes will be naturally in close contact with the skin of the external ear canal of the wearer's first ear; wherein the first body-structure is configured to be made of elastic flexible adaptable material and the material is configured to have appropriate elasticity flexibility and adaptability so that the first body-structure will naturally adapt to the contour of the tragus-concha bowl of the wearer's first ear and snugly fill the interior of the tragus-concha bowl of the wearer's first ear when the first body-structure is placed inside the tragus-concha bowl of the wearer's first ear, and so that the first greater auricular nerve stimulating electrode and the first reference electrode will be naturally in close contact with the skin of the tragus-concha bowl of the wearer's first ear, and, at the same time, the first ta VNS stimulating electrode and the first auriculotemporal nerve stimulating electrode will be naturally in close contact with one of: the skin of the tragus-concha bowl of the wearer's first ear and the skin of the external ear canal of the wearer's first ear, and so that the first taVNS stimulating electrode will be naturally in close contact with the vagus innervated auricular skin of the wearer's first ear, provided by carefully selecting a location for the first taVNS stimulating electrode on the first tubular-shaped structure or the first body-structure to match one of the innervation locations of the vagus innervated auricular skin on the external ear canal of the wearer's first ear or the tragus-concha bowl of the wearer's first ear, and, at the same time, the first auriculotemporal nerves stimulating electrode will be naturally in close contact with auriculotemporal nerve innervated auricular skin of the wearer's first ear, provided by carefully selecting a location for the first auriculotemporal nerve stimulating electrode on the first tubular-shaped structure or the first body-structure to match one of the innervation locations of the auriculotemporal nerve innervated skin on the external ear canal of the wearer's first ear or the tragus-concha bowl of the wearer's first ear, and, at the same time, the first greater auricular nerve stimulating electrode will be naturally in close contact with greater auricular nerve innervated auricular skin of the wearer's first ear, provided by carefully selecting a location for the first greater auricular nerve stimulating electrode on the first body-structure to match one of the innervation locations of the greater auricular nerve innervated skin on tragus-concha bowl of the wearer's first ear.

7. The automatic detection-therapy system for neuropsychiatric disorders of claim 1, wherein each of the following is configured to be user-installable and user-removable: all of the EEG sensor electrodes, the first ta VNS stimulating electrode, the first auriculotemporal nerve stimulating electrode and the first greater auricular nerve stimulating electrode, wherein all of the following are configured to be user-installable simultaneously and user-removable simultaneously: all of the EEG sensor electrodes, the first taVNS stimulating electrode, the first auriculotemporal nerve stimulating electrode and the first greater auricular nerve stimulating electrode, and wherein any of the following is configured to have automated installation feature and automated removal feature: all of the EEG sensor electrodes, the first taVNS stimulating electrode, the first auriculotemporal nerve stimulating electrode and the first greater auricular nerve stimulating electrode.

8. The automatic detection-therapy system for neuropsychiatric disorders of claim 1, wherein the auricular electroencephalogram (EEG) monitoring system comprises a first EEG recording module having a plurality of EEG sensor electrodes, wherein the first EEG recording module is in electronic communication with each EEG sensor electrode of the first EEG recording module, wherein the first EEG recording module is configured to record EEG data of the wearer, wherein all of the EEG sensor electrodes of the first EEG recording module are configured to be housed in a first auricular housing having a first tubular-shaped structure, wherein the first tubular-shaped structure is configured to be inserted into the external ear canal of the wearer's first ear when in use, wherein all of the EEG sensor electrodes of the first EEG recording module are configured to be located on a surface and partially embedded in the surface with protrusion at the surface of the first tubular-shaped structure, wherein one or more EEG sensor electrode(s) are located at the upper surface of the first tubular-shaped structure, wherein one or more EEG sensor electrode(s) are located above the horizontal level of the first tubular-shaped structure and are facing forward-upward, wherein one or more EEG sensor electrode(s) are located above the horizontal level of the first tubular-shaped structure and are facing backward-upward, wherein the first tubular-shaped structure comprises an elastic flexible and adaptable material, and wherein the elastic flexible and adaptable material of the first tubular-shaped structure is configured to have appropriate elasticity flexibility and adaptability so that all of the EEG sensor electrodes of the first EEG recording module are naturally in close contact with the skin of the external ear canal of the wearer's first ear when the first tubular-shaped structure is inserted into the external ear canal of the wearer's first ear.

9. The automatic detection-therapy system for neuropsychiatric disorders of claim 1, further comprising at least one of the following: a multi-mode timer, a multi-mode switch, a multifunctional timer-switch and a programmable multifunctional timer-switch, to enable the wearer to do at least one of the following: to select manual or automatic control, to select various time courses, and to select at least two of the following neuromodulation components: the first ta VNS unit, the supraorbital nerve stimulation unit, the first auriculotemporal nerve stimulation unit, the occipital nerve stimulation unit, the first greater auricular nerve stimulation unit and the infraorbital nerve stimulation unit including various combinations thereof, wherein when prompted the first neuromodulation unit is configured to generate neuromodulating electric stimulation to a wearer according to the stimulation mode selected by the wearer, and wherein the selections of the stimulation mode include the following: double neuromodulation by various combinations of two components of the neuromodulation unit, and triple neuromodulation by various combinations of three components of the neuromodulation unit.

10. The automatic detection-therapy system for neuropsychiatric disorders of claim 1, wherein the neuropsychiatric disorders include at least one of the following: seizure, migraine, cluster headache, major depressive disorder, bipolar disorder, schizophrenia, obsessive-compulsive disorder, attention deficit hyperactivity disorder, autism spectrum disorder, post-traumatic stress disorder, anxiety disorder and panic disorder.

11. The automatic detection-therapy system for neuropsychiatric disorders of claim 6, wherein all of the EEG sensor electrodes of the first EEG recording module are configured as wireless EEG sensor electrodes, wherein the first reference electrode is configured as a first wireless reference electrode, wherein the first EEG recording module comprises a wireless EEG amplifier, and wherein all of the wireless EEG sensor electrodes of the first EEG recording module and the wireless first reference electrode are housed in the first auricular housing while the wireless EEG amplifier and the processing unit are housed remotely in a client device.

12. An automatic detection-therapy system for neuropsychiatric disorders, comprising:
a first neuromodulation unit having a multi-mode switch and at least one component of the following components: a first transcutaneous auricular vagus nerve stimulation unit (first taVNS unit), a first auriculotemporal nerve stimulation unit, and a first greater auricular nerve stimulation unit, wherein each component of the first neuromodulation unit is configured to give neuromodulating electric stimulation to a wearer when activated, wherein the components of the first neuromodulation unit is configured to be selectable by the wearer via the multi-mode switch, and wherein the wearer can switch back and forth among different components of the first neuromodulation unit via the multi-mode switch;
an auricular electroencephalogram (EEG) monitoring system, comprising a first EEG recording module having a plurality of EEG sensor electrodes, wherein the first EEG recording module is in electronic communication with each EEG sensor electrode of the first EEG recording module, wherein the first EEG recording module is configured to record EEG data of the wearer, wherein all of the EEG sensor electrodes of the first EEG recording module are configured to be housed in a first auricular housing having a first tubular-shaped structure, wherein the first tubular-shaped structure is configured to be inserted into an external ear canal of the wearer's first ear when in use, wherein all of the EEG sensor electrodes of the first EEG recording module are configured to be located on a surface and partially embedded in the surface with protrusion at the surface of the first tubular-shaped structure, wherein one or more EEG sensor electrode(s) are located at the upper surface of the first tubular-shaped structure, wherein one or more EEG sensor electrode(s) are located above the horizontal level of the first tubular-shaped structure and are facing forward-upward, wherein one or more EEG sensor electrode(s) are located above the horizontal level of the first tubular-shaped structure and are facing backward-upward, wherein the first tubular-shaped structure comprises an elastic flexible and adaptable material, and wherein the elastic flexible and adaptable material of the first tubular-shaped structure is configured to have appropriate elasticity flexibility and adaptability so that all of the EEG sensor electrodes of the first EEG recording module are naturally in close contact with the skin of the external ear canal of the wearer's first ear when the first tubular-shaped structure is inserted into the external ear canal of the wearer's first ear; and
a processing unit in electronic communication with the auricular EEG monitoring system and each component of the first neuromodulation unit;
wherein the processing unit is configured to analyze the EEG data recorded by the auricular EEG monitoring system to detect presence or cessation of EEG signals suggestive of at least one neuropsychiatric disorder;
wherein when presence of EEG signals suggestive of at least one neuropsychiatric disorder is detected by the processing unit, the processing unit is configured to immediately send signals to the first neuromodulation unit to prompt it to automatically start sending neuromodulating electric stimulation to the wearer from the at least one component of the first neuromodulation unit;
wherein when cessation of EEG signals suggestive of at least one neuropsychiatric disorder is detected by the processing unit, the processing unit is further configured to immediately send signals to the first neuromodulation unit to prompt it to automatically stop sending neuromodulating electric stimulation to the wearer from any component of the neuromodulation unit;
wherein the processing unit is further configured to analyze the EEG data recorded by the auricular EEG monitoring system to detect presence or cessation of EEG signals suggestive of at least one impending neuropsychiatric disorder;
wherein when presence of EEG signals suggestive of at least one impending neuropsychiatric disorder is detected by the processing unit, the processing unit is configured to immediately send signals to the first neuromodulation unit to prompt it to automatically start sending neuromodulating electric stimulation to the wearer from the at least one component of the first neuromodulation unit; and
wherein when cessation of EEG signals suggestive of at least one impending neuropsychiatric disorder is detected by the processing unit, the processing unit is configured to immediately send signals to the first neuromodulation unit to prompt it to automatically stop sending neuromodulating electric stimulation to the wearer from any component of the first neuromodulation unit.

13. The automatic detection-therapy system for neuropsychiatric disorders of claim 12, wherein the first neuromodulation unit comprises at least one component of the following components: a first transcutaneous auricular vagus nerve stimulation unit (first taVNS unit), a supraorbital nerve stimulation unit, an infraorbital nerve stimulation unit, a first auriculotemporal nerve stimulation unit, an occipital nerve stimulation unit and a first greater auricular nerve stimulation unit, wherein the components of the first neuromodulation unit is configured to be selectable by the wearer via the multi-mode switch, and wherein the wearer can switch back and forth among different components of the first neuromodulation unit via the multi-mode switch, wherein the processing unit is in electronic communication with each component of the first neuromodulation unit, wherein the processing unit is configured to analyze the EEG data to detect presence or cessation of EEG signals suggestive of at least one neuropsychiatric disorder, wherein the processing unit is further configured to analyze the EEG data to detect presence or cessation of EEG signals suggestive of at least one impending neuropsychiatric disorder, wherein when the processing unit detects presence of EEG signals suggestive of at least one neuropsychiatric disorder the processing unit is configured to send signals to the first neuromodulation unit to prompt it to start sending predetermined neuromodulating electric stimulation from at least one of the following: the first taVNS unit, the supraorbital nerve stimulation unit, the infraorbital nerve stimulation unit, the first auriculotemporal nerve stimulation unit, the occipital nerve stimulation unit and the first greater auricular nerve stimulation unit;
wherein when the processing unit detects cessation of EEG signals suggestive of at least one neuropsychiatric disorder, the processing unit is configured to send signals to the first neuromodulation unit to prompt it stop sending neuromodulating electric stimulation from any of the following: the first taVNS unit, the supraorbital nerve stimulation unit, the infraorbital nerve stimulation unit, the first auriculotemporal nerve stimulation unit, the occipital nerve stimulation unit and the first greater auricular nerve stimulation unit;
wherein when the processing unit detects presence of EEG signals suggestive of at least one impending neuropsychiatric disorder, the processing unit is configured to send signals to the first neuromodulation unit to prompt it to start sending predetermined neuromodulating electric stimulation from at least one of the following: the first taVNS unit, the supraorbital nerve stimulation unit, the infraorbital nerve stimulation unit, the first auriculotemporal nerve stimulation unit, the occipital nerve stimulation unit and the first greater auricular nerve stimulation unit; and
wherein when the processing unit detects cessation of EEG signals suggestive of at least one impending neuropsychiatric disorder, the processing unit is further configured to send signals to the first neuromodulation unit to prompt it to stop sending neuromodulating electric stimulation from any of the following: the first ta VNS unit, the supraorbital nerve stimulation unit, the infraorbital nerve stimulation unit, the first auriculotemporal nerve stimulation unit, the occipital nerve stimulation unit and the first greater auricular nerve stimulation unit.

14. The automatic detection-therapy system for neuropsychiatric disorders of claim 13, wherein the auricular EEG monitoring system further comprises a second EEG recording module, wherein the second EEG recording module comprises a plurality of EEG sensor electrodes, wherein the second EEG recording module is in electronic communication with each EEG sensor electrode of the second EEG recording module, wherein each EEG sensor electrode of the second EEG recording module is configured to contact separate areas of the wearer's skin selected from at least one of the following: an external ear canal of a second ear of the wearer, an external ear of the wearer's second ear, and a peri-auricular area around the wearer's second ear, and wherein the second EEG recording module is configured to record EEG data of the wearer, wherein the second EEG recording module is in electronic communication with the processing unit.

15. The automatic detection-therapy system for neuropsychiatric disorders of claim 14, further comprising a second neuromodulation unit, wherein the second neuromodulation unit comprises at least one of the following: a second transcutaneous auricular vagus nerve stimulation unit (second taVNS unit), a second auriculotemporal nerve stimulation unit and a second greater auricular nerve stimulation unit, wherein the second taVNS unit includes a second taVNS stimulating electrode configured to contact vagus innervated auricular skin of the wearer's second ear, wherein the second auriculotemporal nerve stimulation unit includes a second auriculotemporal nerve stimulating electrode configured to contact auriculotemporal nerve innervated auricular skin of the wearer's second ear, wherein the second greater auricular nerve stimulation unit includes a second greater auricular nerve stimulating electrode configured to contact greater auricular nerve innervated auricular skin of the wearer's second ear, wherein the processing unit is configured to analyze the EEG data transmitted from the auricular EEG monitoring system, including EEG data recorded by the first EEG recording module and EEG data recorded by the second EEG recording module, to detect presence or cessation of EEG signals suggestive of at least one neuropsychiatric disorder, wherein the processing unit is further configured to analyze the EEG data transmitted from the auricular EEG monitoring system, including EEG data recorded by the first EEG recording module and EEG data recorded by the second EEG recording module, to detect presence or cessation of EEG signals suggestive of at least one impending neuropsychiatric disorder,
wherein when the processing unit detects at least one of the following: presence of EEG signals suggestive of at least one neuropsychiatric disorder by analyzing EEG data recorded by the first EEG recording module and presence of EEG signals suggestive of at least one neuropsychiatric disorder by analyzing EEG data recorded by the second EEG recording module, the processing unit is configured to start sending predetermined neuromodulating electric stimulation from at least one of the following: the first taVNS unit, the first auriculotemporal nerve stimulation unit, the first greater auricular nerve stimulation unit, the second ta VNS unit, the second auriculotemporal nerve stimulation unit, the second greater auricular nerve stimulation unit, the supraorbital nerve stimulation unit, the infraorbital nerve stimulation unit and the occipital nerve stimulation unit;
wherein when the processing unit detects at least one of the following: presence of EEG signals suggestive of at least one impending neuropsychiatric disorder by analyzing EEG data recorded by the first EEG recording module and presence of EEG signals suggestive of at least one impending neuropsychiatric disorder by analyzing EEG data recorded by the second EEG recording module, the processing unit is configured to start sending predetermined neuromodulating electric stimulation from at least one of the following: the first taVNS unit, the first auriculotemporal nerve stimulation unit, the first greater auricular nerve stimulation unit, the second taVNS unit, the second auriculotemporal nerve stimulation unit, the second greater auricular nerve stimulation unit, the supraorbital nerve stimulation unit, the infraorbital nerve stimulation unit and the occipital nerve stimulation unit; and
wherein when the processing unit detects all of the following: cessation of EEG signals suggestive of at least one neuropsychiatric disorder by analyzing EEG data recorded by the first EEG recording module, cessation of EEG signals suggestive of at least one impending neuropsychiatric disorder by analyzing EEG data recorded by the first EEG recording module, cessation of EEG signals suggestive of at least one neuropsychiatric disorder by analyzing EEG data recorded by the second EEG recording module and cessation of EEG signals suggestive of at least one impending neuropsychiatric disorder by analyzing EEG data recorded by the second EEG recording module, the processing unit is configured to stop sending predetermined neuromodulating electric stimulation from any of the following: the first taVNS unit, the first auriculotemporal nerve stimulation unit, the first greater auricular nerve stimulation unit, the second ta VNS unit, the second auriculotemporal nerve stimulation unit, the second greater auricular nerve stimulation unit, the supraorbital nerve stimulation unit, the infraorbital nerve stimulation unit and the occipital nerve stimulation unit.

16. The automatic detection-therapy system for neuropsychiatric disorders of claim 12, wherein the auricular EEG monitoring system comprises a plurality of EEG sensor electrodes and a first reference electrode, wherein the first neuromodulation unit comprises at least one of the following: a first transcutaneous auricular vagus nerve stimulation (taVNS) unit having a first taVNS stimulating electrode for stimulation of the auricular branch of vagus nerve of the wearer's first ear, a first auriculotemporal nerve stimulation unit having a first auriculotemporal nerve stimulating electrode for stimulation of the auriculotemporal nerve of the wearer's first ear, and a first greater auricular nerve stimulation unit having a first greater auricular nerve stimulating electrode for stimulation of the greater auricular nerve of the wearer's first ear; wherein all of the EEG sensor electrodes, the first reference electrode, the first taVNS stimulating electrode, the first auriculotemporal nerve stimulating electrode and the first greater auricular nerve stimulating electrode are housed in a first auricular housing, wherein the first auricular housing comprises a first tubular-shaped structure and a first body-structure, wherein the first tubular-shaped structure is configured to be inserted into an external ear canal of the wearer's first ear when in use, wherein the first body-structure is configured to be placed at the immediate opening of the external ear canal of the wearer's first ear and to be placed inside a tragus-concha bowl of the wearer's first ear when in use, wherein all of the EEG sensor electrodes are configured to be located on a surface and partially embedded in the surface with protrusion at the surface of the first tubular-shaped structure, wherein the first greater auricular nerve stimulating electrode and the first reference electrode are configured to be located on a surface and partially embedded in the surface with protrusion at the surface of the first body-structure, wherein the first taVNS stimulating electrode and the first auriculotemporal nerve stimulating electrode are configured to be located on the surface and partially embedded in the surface with protrusion at the surface of one of: the first tubular-shaped structure and the first body-structure, wherein the first tubular-shaped structure is configured to be made of elastic flexible adaptable material and the material is configured to have appropriate elasticity flexibility and adaptability so that the first tubular-shaped structure will naturally adapt to the contour of the external ear canal of the wearer's first ear and snugly fill the interior of the external ear canal of the wearer's first ear when the tubular-shaped structure is inserted into the external ear canal of the wearer's first ear and so that all of the EEG sensor electrodes will be naturally in close contact with the skin of the external ear canal of the wearer's first ear, wherein the first body-structure is configured to be made of elastic flexible adaptable material and the material is configured to have appropriate elasticity flexibility and adaptability so that the first body-structure will naturally adapt to the contour of the tragus-concha bowl of the wearer's first ear and snugly fill the interior of the tragus-concha bowl of the wearer's first ear when the first body-structure is placed inside the tragus-concha bowl of the wearer's first ear, and so that the first greater auricular nerve stimulating electrode and the first reference electrode will be naturally in close contact with the skin of the tragus-concha bowl of the wearer's first ear, and, at the same time, the first taVNS stimulating electrode and the first auriculotemporal nerve stimulating electrode will be naturally in close contact with one of: the skin of the tragus-concha bowl of the wearer's first ear and the skin of the external ear canal of the wearer's first ear, and so that the first taVNS stimulating electrode will be naturally in close contact with the vagus innervated auricular skin of the wearer's first ear, provided by carefully selecting a location for the first taVNS stimulating electrode on the first tubular-shaped structure or the first body-structure to match one of the innervation locations of the vagus innervated auricular skin on the external ear canal of the wearer's first ear or the tragus-concha bowl of the wearer's first ear, and, at the same time, the first auriculotemporal nerves stimulating electrode will be naturally in close contact with auriculotemporal nerve innervated auricular skin of the wearer's first ear, provided by carefully selecting a location for the first auriculotemporal nerve stimulating electrode on the first tubular-shaped structure or the first body-structure to match one of the innervation locations of the auriculotemporal nerve innervated skin on the external ear canal of the wearer's first ear or the tragus-concha bowl of the wearer's first ear, and, at the same time, the first greater auricular nerve stimulating electrode will be naturally in close contact with greater auricular nerve innervated auricular skin of the wearer's first ear, provided by carefully selecting a location for the first greater auricular nerve stimulating electrode on the first body-structure to match one of the innervation locations of the greater auricular nerve innervated skin on tragus-concha bowl of the wearer's first ear.

17. The automatic detection-therapy system for neuropsychiatric disorders of claim 12, further comprises a supraorbital nerve stimulation unit, an infraorbital nerve stimulation unit and an occipital nerve stimulation unit and at least one of the following: a multi-mode timer, a multifunctional timer-switch and a programmable multifunctional timer-switch, to enable the wearer to do at least one of the following: to select manual or automatic control, to select various time courses, and to select at least one of the following neuromodulation components: the taVNS unit, the supraorbital nerve stimulation unit, the infraorbital nerve stimulation unit, the auriculotemporal nerve stimulation unit, the occipital nerve stimulation unit, the greater auricular nerve stimulation unit and various combinations thereof, wherein when prompted the first neuromodulation unit is configured to generate neuromodulating electric stimulation to a wearer according to the selection of the stimulation mode selected by the wearer, and wherein the selections of the stimulation mode include the following: single neuromodulation by one component of the neuromodulation unit, double neuromodulation by various combinations of two components of the neuromodulation unit, and triple neuromodulation by various combinations of three components of the neuromodulation unit.

18. The automatic detection-therapy system for neuropsychiatric disorders of claim 12, wherein the neuropsychiatric disorders include at least one of the following: seizure, migraine, cluster headache, major depressive disorder, bipolar disorder, schizophrenia, obsessive-compulsive disorder, attention deficit hyperactivity disorder, autism spectrum disorder, post-traumatic stress disorder, anxiety disorder and panic disorder.

19. The automatic detection-therapy system for neuropsychiatric disorders of claim 16, wherein each EEG sensor electrode of the first EEG recording module is configured as a wireless EEG sensor electrode, wherein the first reference electrode is configured as a first wireless reference electrode, wherein the first EEG recording module comprises a wireless EEG amplifier, wherein each wireless EEG sensor electrode of the first EEG recording module and the first wireless reference electrode are housed in an auricular housing configured to be placed in at least one of the following when in use: an external ear canal of the wearer and the tragus-concha bowl of the wearer, and wherein the first EEG recording module and the processing unit are housed remotely in one of the following: a wearable watch-type device, a portable smart-phone-type device and a tablet-type device.

20. The automatic detection-therapy system for neuropsychiatric disorders of claim 12, further comprising a network interface, wherein the network interface is in electronic communication with the processing unit, wherein the network interface is configured to generate a notification to at least one of: a client device of the wearer and a client device of a healthcare provider of the wearer when the processing unit detects presence of EEG signals suggestive of at least one neuropsychiatric disorder, wherein the network interface is further configured to generate a notification to at least one of: the client device of the wearer and the client device of the healthcare provider of the wearer when the processing unit detects presence of EEG signals suggestive of at least one impending neuropsychiatric disorder, wherein the network interface is configured to generate a notification to at least one of: the client device of the wearer and the client device of the wearer's healthcare provider when the processing unit detects cessation of EEG signals suggestive of at least one neuropsychiatric disorder, and wherein the network interface is further configured to generate a notification to at least one of: the client device of the wearer and the client device of the wearer's healthcare provider when the processing unit detects cessation of EEG signals suggestive of at least one impending neuropsychiatric disorder.

21. An auricular electroencephalogram (EEG) monitoring system, comprising:
a first EEG recording module having a plurality of wired or wireless EEG sensor electrodes, wherein the first EEG recording module is in electronic communication with each wired or wireless EEG sensor electrode of the first EEG recording module, wherein the first EEG recording module is configured to record EEG data of a wearer, wherein all of the wired or wireless EEG sensor electrodes of the first EEG recording module are configured to be housed in a first auricular housing having a first tubular-shaped structure, wherein the first tubular-shaped structure is configured to be inserted into an external ear canal of a wearer's first ear when in use, wherein all of the wired or wireless EEG sensor electrodes of the first EEG recording module are configured to be located on a surface and partially embedded in the surface with protrusion at the surface of the first tubular-shaped structure, wherein one or more wired or wireless EEG sensor electrode(s) are located at the upper surface of the first tubular-shaped structure, wherein one or more wired or wireless EEG sensor electrode(s) are located above the horizontal level of the first tubular-shaped structure and are facing forward-upward, wherein one or more wired or wireless EEG sensor electrode(s) are located above the horizontal level of the first tubular-shaped structure and are facing backward-upward, wherein the first tubular-shaped structure comprises an elastic flexible and adaptable material, and wherein the elastic flexible and adaptable material of the tubular-shaped structure is configured to have appropriate elasticity flexibility and adaptability so that the tubular-shaped structure will naturally adapt to the contour of the wearer's external ear canal and snugly fill the interior of the wearer's external ear canal when the tubular-shaped structure is inserted into the wearer's external ear canal, and so that all of the wired or wireless EEG sensor electrodes of the first EEG recording module are naturally in close contact with the skin of the external ear canal of the wearer's first ear when the first tubular-shaped structure is inserted into the external ear canal of the wearer's first ear;
a network interface, wherein the network interface is configured to generate a notification to at least one of: a client device of the wearer and a client device of the wearer's healthcare provider; and
a processing unit in electronic communication with the first EEG recording module and the network interface;
wherein the processing unit is configured to analyze the EEG data recorded by the first EEG recording module to detect presence or cessation of EEG signals suggestive of at least one neuropsychiatric disorder, wherein the processing unit is further configured to analyze the EEG data recorded by the first EEG recording module to detect presence or cessation of EEG signals suggestive of at least one impending neuropsychiatric disorder;
wherein when the presence of EEG signals suggestive of at least one neuropsychiatric disorder is detected by the processing unit, the processing unit is configured to immediately send signals to the network interface to generate a notification to at least one of: the client device of the wearer and the client device of the wearer's healthcare provider;
wherein when cessation of EEG signals suggestive of at least one neuropsychiatric disorder is detected by the processing unit, the processing unit is configured to immediately send signals to the network interface to generate a notification to at least one of: the client device of the wearer and the client device of the wearer's healthcare provider;
wherein when the presence of EEG signals suggestive of at least one impending neuropsychiatric disorder is detected by the processing unit, the processing unit is configured to immediately send signals to the network interface to generate a notification to at least one of: the client device of the wearer and the client device of the wearer's healthcare provider; and
wherein when cessation of EEG signals suggestive of at least one impending neuropsychiatric disorder is detected by the processing unit, the processing unit is further configured to immediately send signals to the network interface to generate a notification to at least one of: the client device of the wearer and the client device of the wearer's healthcare provider.

22. The auricular EEG monitoring system of claim 21, wherein the first EEG recording module further comprise a first wired or wireless reference electrode in electronic communication with the first EEG recording module, wherein the first auricular housing further comprises a first body-structure configured to be placed at the opening of the external ear canal of the wearer's first ear and to be placed inside a tragus-concha bowl of the wearer's first ear when in use, wherein all of the wired or wireless EEG sensor electrodes are configured to be placed at a surface of the first tubular-shaped structure and the first wired or wireless reference electrode is configured to be placed at a surface of the first body-structure, wherein all of the wired or wireless EEG sensor electrodes of the first EEG recording module are configured to be located on a surface and partially embedded in the surface with protrusion at the surface of the first tubular-shaped structure, wherein the first wired or wireless reference electrode is configured to be located at a surface and partially embedded in the surface with protrusion at the surface of the first body-structure, wherein the first tubular-shaped structure is configured to be made of an elastic flexible and adaptable material, wherein the elastic flexible and adaptable material of the first tubular-shaped structure is configured to have appropriate elasticity flexibility and adaptability so that the first tubular-shaped structure will naturally adapt to the contour of the external ear canal of the wearer's first ear and snugly fill the interior of the external ear canal of the wearer's first ear so that all of the wired or wireless EEG sensor electrodes of the first EEG recording module are naturally in close contact with the skin of the external ear canal of the wearer's first ear when the first tubular-shaped structure is inserted into the external ear canal of the wearer's first ear; wherein the first body-structure is configured to be made of an elastic flexible and adaptable material and the material is configured to have appropriate elasticity flexibility and adaptability so that the first body-structure will naturally adapt to the contour of the tragus-concha bowl of the wearer's first ear and snugly fill the interior of the tragus-concha bowl of the wearer's first ear when the body-structure is placed inside the wearer's tragus-concha bowl and so that the first wired or wireless reference electrode will be naturally in close contact with the skin of the tragus-concha bowl of the wearer's first ear when the first body-structure is placed in the tragus-concha bowl of the wearer's first ear, wherein all of the wired or wireless EEG sensor electrodes and the first wired or wireless reference electrode are user-installable and user-removable, and wherein the auricular EEG monitoring system has automated installation feature and automated removal feature.

23. The auricular EEG monitoring system of claim 21, wherein the auricular EEG monitoring system further comprises a second EEG recording module, wherein the second EEG recording module comprises a plurality of wired or wireless EEG sensor electrodes in electronic communication with the second EEG recording module, wherein each wired or wireless EEG sensor electrode of the second EEG recording module is configured to contact separate areas of the wearer's skin selected from at least one of the following: an external ear canal of a second ear of the wearer, an external ear of the wearer's second ear, and a peri-auricular area around the wearer's second ear, and wherein the second EEG recording module is configured to record EEG data of the wearer, and wherein the second EEG recording module is in electronic communication with the processing unit.

24. The auricular EEG monitoring system of claim 22, wherein each wired or wireless EEG sensor electrode of the first EEG recording module is configured as a wireless EEG sensor electrode, wherein the first wired or wireless reference electrode is configured as a first wireless reference electrode, wherein the first EEG recording module comprises a wireless EEG amplifier, wherein each wireless EEG sensor electrode of the first EEG recording module and the first wireless reference electrode are housed in an auricular housing configured to be placed in at least one of the following when in use: the external ear canal of the wearer's first ear and a tragus-concha bowl of the wearer's first ear, and wherein the first EEG recording module and the processing unit are housed remotely in one of the following: a wearable watch-type device, a portable smart-phone-type device and a tablet-type device.

25. The auricular EEG monitoring system of claim 21, wherein the neuropsychiatric disorders include at least one of the following: seizure, migraine, cluster headache, neurodegenerative diseases, major depressive disorder, bipolar disorder, schizophrenia, obsessive-compulsive disorder, attention deficit hyperactivity disorder, autism spectrum disorder, post-traumatic stress disorder, anxiety disorder and panic disorder.

26. A combined neuromodulation system, comprising:
at least two of the following neuromodulating components:
i. a first transcutaneous auricular vagus nerve stimulation unit (first taVNS unit),
ii. a first auriculotemporal nerve stimulation unit, and
iii. a first greater auricular nerve stimulation unit;
a multi-mode switch and a multi-mode timer,
wherein the first taVNS unit is configured to give neuromodulating electric stimulation to vagus-innervated auricular skin of a wearer's first ear via a first taVNS stimulating electrode, wherein the first auriculotemporal nerve stimulation unit is configured to give neuromodulating electric stimulation to auriculotemporal nerve innervated auricular skin of the wearer's first ear via a first auriculotemporal nerve stimulating electrode, wherein the first greater auricular nerve stimulation unit is configured to give neuromodulating electric stimulation to the greater auricular nerve innervated auricular skin of the wearer's first ear via a first greater auricular nerve stimulating electrode, wherein the first taVNS stimulating electrode, the first auriculotemporal nerve stimulating electrode and the first greater auricular nerve stimulating electrode are configured to be in contact with the skin of the wearer's first ear, wherein the multi-mode switch and the multi-mode timer are in electronic communication with each neuromodulating component, wherein the multi-mode switch is configured to enable the wearer to select among various stimulation modes, and wherein the multi-mode timer is configured to enable the wearer to select among various stimulation time courses, wherein the various stimulation modes selectable by the wearer include the following: single neuromodulation by one component of the neuromodulating components, double neuromodulation by various combinations of two components of the neuromodulating components, and triple neuromodulation by three components of the neuromodulating components.

27. The combined neuromodulation system of claim 26, wherein the combined neuromodulation system comprises three components, including the first ta VNS unit, the first auriculotemporal nerve stimulation unit and the first greater auricular nerve stimulation unit.

28. The combined neuromodulation system of claim 26, wherein all of the neuromodulating components of the combined neuromodulation system are configured to be housed in a first auricular housing having a first body-structure, wherein the first body-structure is configured to be placed in a tragus-concha bowl of the wearer's first ear when in use, wherein the first taVNS stimulating electrode, the first auriculotemporal nerve stimulating electrode and the first greater auricular nerve stimulating electrode are configured to be located at a surface and partially embedded in the surface with protrusion at the surface of the first body-structure of the first auricular housing, wherein the first body-structure of the first auricular housing is configured to be made of elastic flexible adaptable material and the material is configured to have appropriate elasticity flexibility and adaptability so that when the first body-structure is placed inside the tragus-concha bowl of the wearer's first ear, the first body-structure will naturally adapt to the contour of the tragus-concha bowl of the wearer's first ear and snugly fill the interior of the tragus-concha bowl of the wearer's first ear and so that the first taVNS stimulating electrode, the first auriculotemporal nerve stimulating electrode and the first greater auricular nerve stimulating electrode will be naturally in close contact with the skin of the tragus-concha bowl of the wearer's first ear when the first body-structure is placed inside the tragus-concha bowl of the wearer's first ear, and so that the first ta VNS stimulating electrode will be naturally in close contact with its target skin of the vagus innervated auricular skin on the tragus-concha bowl of the wearer's first ear, provided by carefully selecting the location for the taVNS stimulating electrode on the first body-structure to match one of the innervation locations of vagus nerve on the tragus-concha bowl of the wearer's first ear, and, at the same time, the auriculotemporal nerve stimulating electrode will be naturally in close contact with its target skin of auriculotemporal nerve innervated auricular skin on the tragus-concha bowl of the wearer's first ear, provided by carefully selecting the location of the auriculotemporal nerve stimulating electrode on the first body-structure to match one of the innervation locations of the auriculotemporal nerve on the tragus-concha bowl of the wearer's first ear, and, at the same time, the greater auricular nerve stimulating electrode will be naturally in close contact with its target skin of greater auricular nerve innervated auricular skin on the tragus-concha bowl of the wearer's first ear, provided by carefully selecting the location for the greater auricular nerve stimulating electrode on the first body-structure to match one of the innervation locations of the greater auricular nerve on the tragus-concha bowl of the wearer's first ear.
